SEQUENCE LISTING (10 sequences)

SEQ ID NO: 1 - DNA, 20 bp, Artificial Sequence
  synthetic construct
gtggctttggagttgcagtt

SEQ ID NO: 2 - DNA, 21 bp, Artificial Sequence
  synthetic construct
cagccaccatgaatattgtac

SEQ ID NO: 3 - DNA, 20 bp, Artificial Sequence
  synthetic construct
gtggctttggagttgcagtt

SEQ ID NO: 4 - DNA, 20 bp, Artificial Sequence
  synthetic construct
gaagcagatttgggtaccac

SEQ ID NO: 5 - DNA, 18 bp, Artificial Sequence
  synthetic construct
cgggtgacggggaatcag

SEQ ID NO: 6 - DNA, 17 bp, Artificial Sequence
  synthetic construct
cactacctccccgggtc

SEQ ID NO: 7 - DNA, 22 bp, Artificial Sequence
  synthetic construct
agatccctccaaaatcaagtgg

SEQ ID NO: 8 - DNA, 21 bp, Artificial Sequence
  synthetic construct
ggcagagatgatgaccctttt

SEQ ID NO: 9 - RNA, 36 nt, Artificial Sequence
  synthetic construct
guaacuagagaucccucagacccuuuuagucagaau

SEQ ID NO: 10 - DNA, 25 bp, Artificial Sequence
  synthetic construct
ttctgactaaaagggtctgagggat

(19) United States
(12) Patent Application Publication
(10) Pub. No.: US 2025/0268925 A1
(43) Pub. Date: Aug. 28, 2025

(54) METHODS OF TREATING MEDICAL CONDITIONS AND INHIBITING LINE1 REVERSE TRANSCRIPTASE USING A SUBSTITUTED ADENINYL-PROPYLOXY PHOSPHONIC ACID OR RELATED COMPOUND

(71) Applicant: ROME THERAPEUTICS, INC., Boston, MA (US)

(72) Inventors: Donna L. ROMERO, Chesterfield, MO (US); Oliver SAUNDERS, Fresno, CA (US); Gregory Stuart BISACCHI, Plymouth, MA (US); Dennis ZALLER, Lexington, MA (US); Rosana KAPELLER-LIBERMANN, Chestnut Hill, MA (US)

(21) Appl. No.: 18/572,385

(22) PCT Filed: Jun. 22, 2022
(86) PCT No.: PCT/US2022/034605

§ 371 Date: Dec. 20, 2023

Related U.S. Application Data
(60) Provisional application No. 63/213,502, filed on Jun. 22, 2021.

(51) Int. Cl. A61K 31/675

(52) U.S. Cl. CPC A61K 31/675

(57) ABSTRACT

The invention provides methods and compositions for treating medical disorders, such as cancer, and inhibiting LINE1 reverse transcriptase and/or HERV-K reverse transcriptase using a substituted adeninyl-propyloxy phosphonic acid or related compound.

### CROSS REFERENCE TO RELATED APPLICATIONS

This application is the national stage application of International (PCT) Patent Application Serial No. PCT/US2022/034605, filed Jun. 22, 2022, which claims the benefit of and priority to U.S. Provisional Patent Application Ser. No. 63/213,502, filed Jun. 22, 2021, the entire contents of these applications are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Aug. 13, 2024, is named 207081_seglist.txt and is 2,314 bytes in size.

### FIELD OF THE INVENTION

The invention provides methods and compositions for treating medical disorders, such as cancer, and inhibiting LINE1 reverse transcriptase and/or HERV-K reverse transcriptase using a substituted adeninyl-propyloxy phosphonic acid or related compound.

### BACKGROUND

Cancer continues to be a significant health problem despite the substantial research efforts and scientific advances reported in the literature for treating this disease. Solid tumors, including prostate cancer, breast cancer, and lung cancer remain highly prevalent among the world population. Leukemias and lymphomas also account for a significant proportion of new cancer diagnoses. Current treatment options for these cancers are not effective for all patients and/or can have substantial adverse side effects. New therapies are needed to address this unmet need in cancer therapy.

Accordingly, the need exists for new therapeutic methods that provide improved efficacy and/or reduced side effects for treating medical disorders, such as cancer. The present invention addresses the foregoing needs and provides other related advantages.

### SUMMARY

The invention provides methods and compositions for treating medical disorders, such as cancer, and inhibiting LINE1 reverse transcriptase and/or HERV-K reverse transcriptase using a substituted adeninyl-propyloxy phosphonic acid or related compound. In particular, one aspect of the invention provides a method of treating a disorder selected from the group consisting of cancer, an inflammatory disorder, a neurodegenerative disorder, and an immune disorder other than a viral infection. The method comprises administering to a subject in need thereof a therapeutically effective amount of a compound of Formula I to treat the disorder; wherein Formula I is represented by:

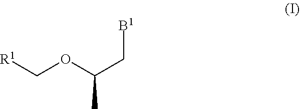

or a pharmaceutically acceptable salt thereof, where the variables are as defined in the detailed description. Further description of additional collections of substituted adeninyl-propyloxy phosphonic acid and related compounds useful in the method are described in the detailed description. The compounds may be part of a pharmaceutical composition comprising a pharmaceutically acceptable carrier. Additional features of the method are described in the detailed description.

Another aspect of the invention provides a method of inhibiting LINE1 reverse transcriptase activity in a subject suffering from a disorder selected from the group consisting of cancer, an inflammatory disorder, a neurodegenerative disorder, and an immune disorder other than a viral infection. The method comprises contacting a LINE1 reverse transcriptase with an effective amount of a compound of Formula I, in order to inhibit the activity of said LINE1 reverse transcriptase; wherein Formula I is represented by:

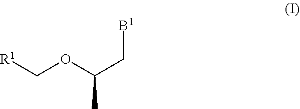

or a pharmaceutically acceptable salt thereof, where the variables are as defined in the detailed description. Further description of additional collections of substituted adeninyl-propyloxy phosphonic acid and related compounds useful in the method are described in the detailed description. Additional features of the method are described in the detailed description.

Another aspect of the invention provides a method of inhibiting HERV-K reverse transcriptase activity in a subject suffering from a disorder selected from the group consisting of cancer, an inflammatory disorder, a neurodegenerative disorder, and an immune disorder other than a viral infection. The method comprises contacting a HERV-K reverse transcriptase with an effective amount of a compound of Formula I, in order to inhibit the activity of said HERV-K reverse transcriptase; wherein Formula I is represented by:

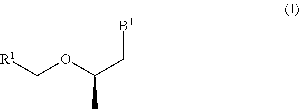

or a pharmaceutically acceptable salt thereof, where the variables are as defined in the detailed description. Further description of additional collections of substituted adeninyl-propyloxy phosphonic acid and related compounds useful in the method are described in the detailed description. Additional features of the method are described in the detailed description.

### BRIEF DESCRIPTION OF THE FIGURES

FIG. 1 is a graph depicting inhibition of IFN by compound I-1 in the cellular assay for altering IFN production in THP1 TREX1 KO cells, as described in Example 5.

FIG. 2 is a graph depicting inhibition of IFN by compound I-20 in the cellular assay for altering IFN production in THP1 TREX1 KO cells, as described in Example 5.

FIG. 3 is a graph depicting induction of IFN by compound I-21 in the cellular assay for altering IFN production in THP1 TREX1 KO cells, as described in Example 5.

FIG. 4 is a graph depicting induction of IFN by compound I-115 in the cellular assay for altering IFN production in THP1 TREX1 KO cells, as described in Example 5.

FIG. 5 is a graph depicting inhibition of IFN by compound V-1 in the cellular assay for altering IFN production in THP1 TREX1 KO cells, as described in Example 5.

FIG. 6 is a graph depicting induction of IFN by compound VI-1 in the cellular assay for altering IFN production in THP1 TREX1 KO cells, as described in Example 5.

FIG. 7 is a graph depicting interferon levels over time in THP1-Dual™ KO-TREX1 xenografts from mice treated with vehicle or decitabine (DAC) at 5 mg/kg IP, once daily, for four days, as described in Example 6.

### DETAILED DESCRIPTION

The invention provides methods and compositions for treating medical disorders, such as cancer, and inhibiting LINE1 reverse transcriptase and/or HERV-K reverse transcriptase using a substituted adeninyl-propyloxy phosphonic acid or related compound. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units of unsaturation, having one or more atoms in common between the two rings of the ring system. Thus, the term includes any permissible ring fusion, such as ortho-fused or spirocyclic. The term “ortho-fused” is art-recognized and refers to a ring fusion where the two rings have only two atoms and one bond in common. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

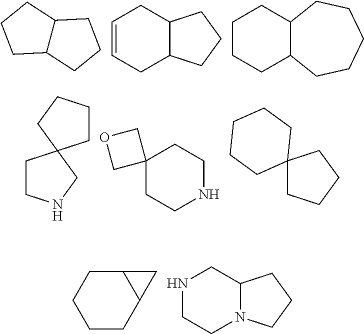

Exemplary bridged bicyclics include:

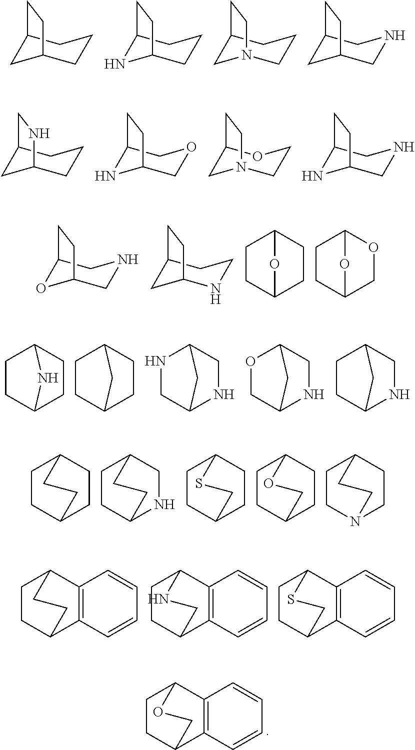

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “phenylene” refers to a multivalent phenyl group having the appropriate number of open valences to account for groups attached to it. For example, “phenylene” is a bivalent phenyl group when it has two groups attached to it (e.g.,

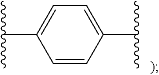 [SMILES: *c1ccc(*)cc1]

“phenylene” is a trivalent phenyl group when it has three groups attached to it (e.g.,

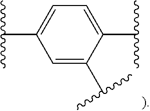 [SMILES: *c1ccc(*)c(*)c1]

The term “arylene” refers to a bivalent aryl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

The term “heteroarylene” refers to a multivalent heteroaryl group having the appropriate number of open valences to account for groups attached to it. For example, “heteroarylene” is a bivalent heteroaryl group when it has two groups attached to it; “heteroarylene” is a trivalent heteroaryl group when it has three groups attached to it. The term “pyridinylene” refers to a multivalent pyridine radical having the appropriate number of open valences to account for groups attached to it. For example, “pyridinylene” is a bivalent pyridine radical when it has two groups attached to it (e.g.,

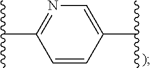 [SMILES: *c1ccc(*)nc1]

“pyridinylene” is a trivalent pyridine radical when it has three groups attached to it (e.g.,

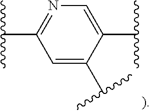 [SMILES: *c1cc(*)c(*)cn1]

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “oxo-heterocyclyl” refers to a heterocyclyl substituted by an oxo group. The term “heterocyclylene” refers to a multivalent heterocyclyl group having the appropriate number of open valences to account for groups attached to it. For example, “heterocyclylene” is a bivalent heterocyclyl group when it has two groups attached to it; “heterocyclylene” is a trivalent heterocyclyl group when it has three groups attached to it.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•}.

Each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens; or wherein an optional substituent on a saturated carbon is a divalent substituent independently selected from ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{•} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The terms “alkenyl” and “alkynyl” are art-recognized and refer to unsaturated aliphatic groups analogous in length and possible substitution to the alkyls described above, but that contain at least one double or triple bond respectively.

The term “carbocyclylene” refers to a multivalent carbocyclyl group having the appropriate number of open valences to account for groups attached to it. For example, “carbocyclylene” is a bivalent carbocyclyl group when it has two groups attached to it; “carbocyclylene” is a trivalent carbocyclyl group when it has three groups attached to it.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “═O” substituent. For example, a cyclopentane substituted with an oxo group is cyclopentanone.

The symbol “ [symbol] ” indicates a point of attachment.

When a chemical structure containing a ring is depicted with a substituent having a bond that crosses a ring bond, the substituent may be attached at any available position on the ring. For example, the chemical structure

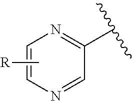

encompasses

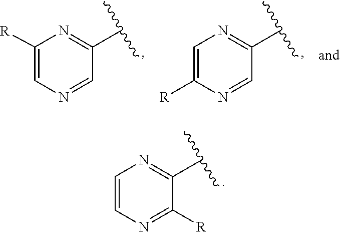

In the context of a polycyclic fused ring, when a chemical structure containing a polycyclic fused ring is depicted with one or more substituent(s) having a bond that crosses multiple rings, the one or more substituent(s) may be independently attached to any of the rings crossed by the bond. To illustrate, the chemical structure

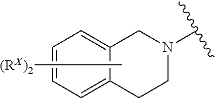

encompasses, for example,

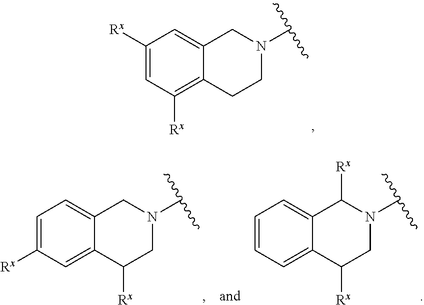

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Methods of Treating Medical Disorders

It is contemplated that the substituted adeninyl-propyloxy phosphonic acid and related compounds described herein, such as a compound of Formula I, I-A, or I-B, or other compounds in Section III, below, provide therapeutic benefits to subjects suffering from cancer and other disorders.

Accordingly, one aspect of the invention provides a method of treating a disorder selected from the group consisting of cancer, an inflammatory disorder, a neurodegenerative disorder, and an immune disorder other than a viral infection. The method comprises administering to a subject in need thereof a therapeutically effective amount of a compound of Formula I to treat the disorder; wherein Formula I is represented by:

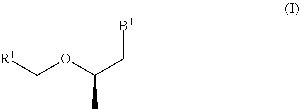

or a stereoisomer thereof; or a pharmaceutically acceptable salt of either of the foregoing; wherein:
R¹ is —P(O)(OR³)(N(R⁴)(R⁵)), —P(O)(OR³)₂, or —P(O)(N(R⁴)(R⁵))₂;
R² is hydrogen, —NH₂, or fluoro;
R³ represents independently for each occurrence:
   a. phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein said phenyl, naphthyl, 5-6 membered monocyclic heteroaryl, and 8-10 membered bicyclic heteroaryl are substituted with m instances of R⁸;
   b. hydrogen, —P(O)(OH)₂, —P(O)(OH)—O—P(O)(OH)₂, C₁₋₂₀ alkyl, C₁₋₂₀ haloalkyl, —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), —(C₁₋₁₀ alkylene)-Y—(C₂₋₂₀ alkynylene)-R¹¹, or —C(R⁶)₂—CO₂R¹⁰; wherein said C₁₋₂₀ alkyl, C₁₋₂₀ haloalkyl, and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene; or
   c. two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸;
R⁴ and R⁹ each represents independently for each occurrence hydrogen or C₁₋₄ alkyl; or R⁴ and one occurrence of R⁶ are taken together with atoms to which they are attached to form a 4-7 membered saturated heterocyclic ring containing 1 nitrogen atom; or two instances of R⁹ are taken together with the atom to which they are attached to form a 4-7 membered saturated heterocyclic ring containing 1 nitrogen atom and optionally 1 oxygen atom;
R⁵ represents independently for each occurrence:
   a. —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, —C(R¹⁰)(H)—CH₂—CO₂R¹⁰, C₁₋₂₀ alkyl, C₁₋₂₀ haloalkyl, —(C₁₋₁₀ alkylene)-X—(C₁₋₁₀ alkyl), —(C₁₋₁₀ alkylene)-phenyl, —(C₁₋₄ haloalkylene)-phenyl, —C(O)-phenyl, —C(S)-phenyl, or —C(O)-(5-6 membered monocyclic heteroaryl having 1, 2, or 3 heteroatoms independently selected from nitrogen, oxygen, and sulfur); or
   b. phenyl; naphthyl; a 5-6 membered monocyclic heteroaryl having 1, 2, or 3 heteroatoms independently selected from nitrogen, oxygen, and sulfur; or an 8-10 membered bicyclic heteroaryl having 1, 2, or 3 heteroatoms independently selected from nitrogen, oxygen, and sulfur;
   wherein said phenyl, naphthyl, 5-6 membered monocyclic heteroaryl, and 8-10 membered bicyclic heteroaryl are substituted with m instances of R⁸; and said C₁₋₂₀ alkyl, C₁₋₂₀ haloalkyl, and C₁₋₁₀ alkyl are optionally substituted with one hydroxyl;
R⁶ represents independently for each occurrence C₁₋₆ alkyl, C₁₋₆ haloalkyl, C₃₋₅ cycloalkyl, —CN, or hydrogen, wherein said C₁₋₆ alkyl is optionally substituted with —S—(C₁₋₄ alkyl), —SH, C₁₋₄ alkoxyl, C₁₋₄ haloalkoxyl, hydroxyl, —OCH₂CN, phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; or two instances of R⁶ are taken together with the carbon atom to which they are attached to form a 3-6 membered saturated ring having 0 or 1 oxygen atom;
R⁷ represents independently for each occurrence C₁₋₈ alkyl, C₁₋₆ haloalkyl, C₂₋₆ alkenyl, C₃₋₇ cycloalkyl, or a 4-7 membered saturated monocyclic heterocyclyl having one or two heteroatoms independently selected from nitrogen, oxygen, and sulfur; wherein said C₁₋₈ alkyl is optionally substituted with C₁₋₄ alkoxyl, phenyl, C₃₋₇ cycloalkyl, or a 4-7 membered saturated monocyclic heterocyclyl having one or two heteroatoms independently selected from nitrogen, oxygen, and sulfur;
R⁸ represents independently for each occurrence halo, C₁₋₄ alkyl, C₁₋₄ haloalkyl, C₁₋₄ alkoxyl, C₁₋₄ haloalkoxyl, —(C₀₋₃ alkylene)-CO₂R¹⁰, —CN, or —N(R⁹)₂;
R¹⁰ represents independently for each occurrence C₁₋₆ alkyl, C₃₋₇ cycloalkyl, or hydrogen;
R¹¹ represents independently for each occurrence C₁₋₂ haloalkyl, —SF₅, —Si(C₁₋₄ alkyl)₃, —Si(CH₃)₂(C₁₋₄ haloalkyl), —Si(CH₃)₂(C₃₋₇ cycloalkyl),

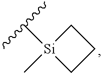 [SMILES: *[Si]1(C)CCC1]

—Si(CH₃)₂(phenyl), —S-phenyl, —O-phenyl, phenyl, thiophenyl, pyridinyl, or C₃₋₇ cycloalkyl; wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅;
X represents independently for each occurrence —O—, ψ-OC(O)—, —OC(O)O—, ψ-OC(O)—N(R⁹)—, —S—, —S—S—, or ψ-SC(O)—; wherein ψ denotes the point of attachment to C₁₋₁₀ alkylene;
Y represents independently for each occurrence —O—, —S—, or —CF₂—;
B¹ is

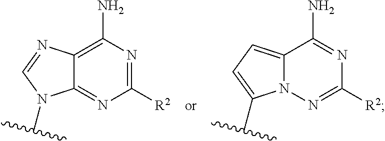

and
m and p are independently for each occurrence 0, 1, 2, or 3;
wherein one or more hydrogen atoms may be replaced with deuterium.

In certain embodiments, the particular compound of Formula I is a compound defined by one of the embodiments described in Section III, below, such as a compound of Formula I-A or I-B.

In certain embodiments, the compound of Formula I, or other compound defined by one of the embodiments described in Section III, below, such as a compound of Formula I-A or I-B, is administered in a pharmaceutical composition comprising the compound and a pharmaceutically acceptable carrier, as further described in Section V, below.

In certain embodiments, the method further comprises administering an effective amount of an additional therapeutic agent, as further described in Section IV, below.

### Cancer

In certain embodiments, the disorder is cancer. In certain embodiments, the cancer is a solid tumor or leukemia. In certain embodiments, the cancer is a solid tumor. In certain embodiments, the cancer is a carcinoma or melanoma. In certain embodiments, the cancer is a carcinoma. In certain embodiments, the cancer is a sarcoma. In certain embodiments, the cancer is a melanoma. In certain embodiments, the cancer is a lymphoma. In certain embodiments, the cancer is a leukemia.

In certain embodiments, the cancer is breast cancer, ovarian cancer, uterine cancer, cervical cancer, prostate cancer, testicular cancer, lung cancer, leukemia, head and neck cancer, oral cancer, esophageal cancer, stomach cancer, bile duct and gallbladder cancers, bladder cancer, urinary tract cancer, colon cancer, rectal cancer, thyroid cancer, pancreatic cancer, kidney cancer, liver cancer, brain cancer, skin cancer, or eye cancer.

In certain embodiments, the cancer has (i) expression of LINE1 RNA, LINE1 ORF1 polypeptide, and/or LINE1 ORF2 polypeptide; (ii) activity of LINE1 reverse transcriptase; (iii) expression of HERV-K RNA, and/or (iv) activity of HERV-K reverse transcriptase.

In certain embodiments, the cancer has (i) expression of LINE1 RNA, LINE1 ORF1 polypeptide, and/or LINE1 ORF2 polypeptide; and/or (ii) activity of LINE1 reverse transcriptase. In certain embodiments, the cancer has expression of LINE1 RNA, LINE1 ORF1 polypeptide, and/or LINE1 ORF2 polypeptide. In certain embodiments, the cancer has expression of LINE1 RNA. In certain embodiments, the cancer has expression of LINE1 ORF1 polypeptide. In certain embodiments, the cancer has expression of LINE1 ORF2 polypeptide. In certain embodiments, the cancer has activity of LINE1 reverse transcriptase.

In certain embodiments, the cancer has (i) expression of HERV-K RNA, and/or (ii) activity of HERV-K reverse transcriptase. In certain embodiments, the cancer has expression of HERV-K RNA. In certain embodiments, the cancer has activity of HERV-K reverse transcriptase.

In certain embodiments, the cancer has elevated (i) levels of LINE1 RNA, LINE1 ORF1 polypeptide, and/or LINE1 ORF2 polypeptide; (ii) activity of LINE1 reverse transcriptase; (iii) levels of HERV-K RNA, and/or (iv) activity of HERV-K reverse transcriptase.

In certain embodiments, the cancer has elevated (i) levels of LINE1 RNA, LINE1 ORF1 polypeptide, and/or LINE1 ORF2 polypeptide; and/or (ii) activity of LINE1 reverse transcriptase. In certain embodiments, the cancer has elevated levels of LINE1 RNA, LINE1 ORF1 polypeptide, and/or LINE1 ORF2 polypeptide. In certain embodiments, the cancer has elevated levels of LINE1 RNA. In certain embodiments, the cancer has elevated levels of LINE1 ORF1 polypeptide. In certain embodiments, the cancer has elevated levels of LINE1 ORF2 polypeptide. In certain embodiments, the cancer has elevated activity of LINE1 reverse transcriptase.

In certain embodiments, the cancer has elevated (i) levels of HERV-K RNA, and/or (ii) activity of HERV-K reverse transcriptase. In certain embodiments, the cancer has elevated levels of HERV-K RNA. In certain embodiments, the cancer has elevated activity of HERV-K reverse transcriptase.

In certain embodiments, the cancer is pancreatic cancer, colorectal cancer, breast cancer, prostate cancer, esophageal cancer, head and neck cancer, renal cancer, ovarian cancer, or lung cancer. In certain embodiments, the cancer is pancreatic cancer, colorectal cancer, breast cancer, prostate cancer, renal cancer, ovarian cancer, or lung cancer. In certain embodiments, the cancer is pancreatic cancer. In certain embodiments, the cancer is pancreatic adenocarcinoma. In certain embodiments, the cancer is colorectal cancer. In certain embodiments, the cancer comprises microsatellite instable (MSI) colorectal cancer or microsatellite stable (MSS) colorectal cancer. In certain embodiments, the cancer is breast cancer. In certain embodiments, the cancer is prostate cancer. In certain embodiments, the cancer is esophageal cancer. In certain embodiments, the cancer is head and neck cancer. In certain embodiments, the cancer is renal cancer. In certain embodiments, the cancer is ovarian cancer. In certain embodiments, the cancer is lung cancer. In certain embodiments, the cancer is non-small cell lung carcinoma or small cell lung carcinoma. In certain embodiments, the cancer is non-small cell lung carcinoma. In certain embodiments, the cancer is small cell lung carcinoma.

In certain embodiments, the cancer is an epithelial cancer. In certain embodiments, the epithelial cancer is pancreatic cancer, colorectal cancer, breast cancer, prostate cancer, esophageal cancer, head and neck cancer, renal cancer, ovarian cancer, or lung cancer. In certain embodiments, the epithelial cancer is pancreatic cancer, colorectal cancer, breast cancer, prostate cancer, renal cancer, ovarian cancer, or lung cancer. In certain embodiments, the colorectal cancer comprises microsatellite instable (MSI) colorectal cancer or microsatellite stable (MSS) colorectal cancer.

In certain embodiments, the cancer is a preneoplastic or early cancer lesion. In certain embodiments, the cancer is intraductal papillary mucinous neoplasm (IPMN), pancreatic intraepithelial neoplasia (PanIN), ductal carcinoma in situ (DCIS), or Barrett's Esophagus. In certain embodiments, the cancer intraductal papillary mucinous neoplasm (IPMN). In certain embodiments, the cancer is pancreatic intraepithelial neoplasia (PanIN). In certain embodiments, the cancer is ductal carcinoma in situ (DCIS). In certain embodiments, the cancer is Barrett's Esophagus.

In certain embodiments, the cancer has elevated levels of pericentrometric human satellite II (HSATII) RNA. In some embodiments, the cancer is a microsatellite instable (MSI) cancer. In some embodiments, the cancer is a microsatellite stable (MSS) cancer.

In certain embodiments, the cancer is selected from B cell lymphomas (e.g., B cell chronic lymphocytic leukemia, B cell non-Hodgkin lymphoma, cutaneous B cell lymphoma, diffuse large B cell lymphoma), basal cell carcinoma, bladder cancer, blastoma, brain metastasis, breast cancer, Burkitt lymphoma, carcinoma (e.g., adenocarcinoma (e.g., of the gastroesophageal junction)), cervical cancer, colon cancer, colorectal cancer (colon cancer and rectal cancer), endometrial carcinoma, esophageal cancer, Ewing sarcoma, follicular lymphoma, gastric cancer, gastroesophageal junction carcinoma, gastrointestinal cancer, glioblastoma (e.g., glioblastoma multiforme, e.g., newly diagnosed or recurrent), glioma, head and neck cancer (e.g., head and neck squamous cell carcinoma), hepatic metastasis, Hodgkin's and non-Hodgkin's lymphoma, kidney cancer (e.g., renal cell carcinoma and Wilms' tumors), laryngeal cancer, leukemia (e.g., chronic myelocytic leukemia, hairy cell leukemia), liver cancer (e.g., hepatic carcinoma and hepatoma), lung cancer (e.g., non-small cell lung cancer and small-cell lung cancer), lymphblastic lymphoma, lymphoma, mantle cell lymphoma, metastatic brain tumor, metastatic cancer, myeloma (e.g., multiple myeloma), neuroblastoma, ocular melanoma, oropharyngeal cancer, osteosarcoma, ovarian cancer, pancreatic cancer (e.g., pancreatis ductal adenocarcinoma), prostate cancer (e.g., hormone refractory (e.g., castration resistant), metastatic, metastatic hormone refractory (e.g., castration resistant, androgen independent)), renal cell carcinoma (e.g., metastatic), salivary gland carcinoma, sarcoma (e.g., rhabdomyosarcoma), skin cancer (e.g., melanoma (e.g., metastatic melanoma)), soft tissue sarcoma, solid tumor, squamous cell carcinoma, synovia sarcoma, testicular cancer, thyroid cancer, transitional cell cancer (urothelial cell cancer), uveal melanoma (e.g., metastatic), verrucous carcinoma, vulval cancer, and Waldenstrom macroglobulinemia.

In some embodiments, the cancer is a virus-associated cancer. As used herein, the term “virus-associated cancer” means any cancer in which a virus is known to play a role. For example, Epstein-Barr virus (EBV) has been reported to be associated with the endemic variant of Burkitt lymphoma and certain other lymphomas. Infection by human papilloma virus (HPV) is believed to be responsible for certain types of cervical and/or genital cancer. Human T-cell leukemia virus 1 (HTLV-1) has been reported to be linked adult T-cell leukemia/lymphoma (ATLL). Human T-cell leukemia virus 2 (HTLV-2) has been reported to be linked to cutaneous T-cell lymphoma. Human herpes virus 8 (HHV-8) is believed to cause Kaposi's sarcoma in patients with AIDS. In certain embodiments, the cancer is a cancer associated with EBV, HPV, HTLV-1, HTLV-2, or HHV-8. In certain embodiments, the cancer is Burkitt lymphoma, cervical cancer, genital cancer, adult T-cell leukemia/lymphoma, cutaneous T-cell lymphoma, or Kaposi's sarcoma.

In some embodiments, the cancer is a cancer other than a virus-associated cancer. In certain embodiments, the cancer is a cancer other than a cancer associated with EBV, HPV, HTLV-1, HTLV-2, or HHV-8. In certain embodiments, the cancer is a cancer other than Burkitt lymphoma, cervical cancer, genital cancer, adult T-cell leukemia/lymphoma, cutaneous T-cell lymphoma, or Kaposi's sarcoma.

In some embodiments, the cancer is mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is hepatocellular carcinoma, ovarian cancer, ovarian epithelial cancer, fallopian tube cancer, papillary serous cystadenocarcinoma, uterine papillary serous carcinoma (UPSC), prostate cancer, testicular cancer, gallbladder cancer, hepatocholangiocarcinoma, soft tissue and bone synovial sarcoma, rhabdomyosarcoma, osteosarcoma, chondrosarcoma, Ewing sarcoma, anaplastic thyroid cancer, adrenocortical adenoma, pancreatic cancer, pancreatic ductal carcinoma, pancreatic adenocarcinoma, gastrointestinal/stomach (GIST) cancer, lymphoma, squamous cell carcinoma of the head and neck (SCCHN), salivary gland cancer, glioma, or brain cancer, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; and medulloblastoma.

In some embodiments, the cancer is renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In certain embodiments, the cancer is a leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, or a solid tumor such as a sarcoma or carcinoma (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor.

### Inflammatory Disorders

In certain embodiments, the disorder is an inflammatory disorder. In certain embodiments, the inflammatory disorder is rheumatoid arthritis, osteoarthritis, ankylosing spondylitis, inflammatory bowel disease, Crohn's disease, ulcerative colitis, nonalcoholic steatohepatitis (NASH), non-alcoholic fatty liver disease (NAFLD), cholestatic liver disease, or sclerosing cholangitis, psoriasis, dermatitis, vasculitis, scleroderma, asthma, bronchitis, chronic obstructive pulmonary disease (COPD), pulmonary fibrosis, pulmonary hypertension, sarcoidosis, myocarditis, pericarditis, gout, myositis, Sjogren's syndrome, or systemic lupus erythematosus.

In certain embodiments, the inflammatory disorder is rheumatoid arthritis, osteoarthritis, or ankylosing spondylitis. In certain embodiments, the inflammatory disorder is inflammatory bowel disease, Crohn's disease, or ulcerative colitis. In certain embodiments, the inflammatory disorder is nonalcoholic steatohepatitis (NASH), non-alcoholic fatty liver disease (NAFLD), cholestatic liver disease, or sclerosing cholangitis. In certain embodiments, the inflammatory disorder is psoriasis, dermatitis, vasculitis, or scleroderma. In certain embodiments, the inflammatory disorder is asthma, bronchitis, chronic obstructive pulmonary disease (COPD), pulmonary fibrosis, pulmonary hypertension, sarcoidosis, myocarditis, or pericarditis. In certain embodiments, the inflammatory disorder is gout, myositis, Sjogren's syndrome, or systemic lupus erythematosus.

### Immune Disorders

In certain embodiments, the immune disorder is arthritis, psoriasis, systemic lupus erythematosus (SLE), graft versus host disease, scleroderma, polymyositis, inflammatory bowel disease, dermatomyositis, ulcerative colitis, Crohn's disease, vasculitis, psoriatic arthritis, Reiter's syndrome, exfoliative psoriatic dermatitis, pemphigus vulgaris, Sjogren's syndrome, autoimmune uveitis, glomerulonephritis, post myocardial infarction cardiotomy syndrome, pulmonary hemosiderosis, amyloidosis, sarcoidosis, aphthous stomatitis, thyroiditis, gastritis, adrenalitis (Addison's disease), ovaritis, primary biliary cirrhosis, myasthenia gravis, gonadal failure, hypoparathyroidism, alopecia, psoriasis, malabsorption syndrome, pernicious anemia, hepatitis, hypopituitarism, diabetes insipidus, or sicca syndrome.

In certain embodiments, the immune disorder is a type 1 interferonopathy, type 1 diabetes, Aicardi-Goutieres syndrome (AGS), arthritis, psoriasis, systemic lupus erythematosus (SLE), lupus nephritis, cutaneous lupus erythematosus (CLE), familial chilblain lupus, systemic sclerosis, STING-associated vasculopathy with onset in infancy (SAVI), graft versus host disease, scleroderma, polymyositis, inflammatory bowel disease, dermatomyositis, ulcerative colitis, Crohn's disease, vasculitis, psoriatic arthritis, Reiter's syndrome, exfoliative psoriatic dermatitis, pemphigus vulgaris, Sjogren's syndrome, autoimmune uveitis, glomerulonephritis, post myocardial infarction cardiotomy syndrome, pulmonary hemosiderosis, amyloidosis, sarcoidosis, aphthous stomatitis, thyroiditis, gastritis, adrenalitis (Addison's disease), ovaritis, primary biliary cirrhosis, myasthenia gravis, gonadal failure, hypoparathyroidism, alopecia, malabsorption syndrome, pernicious anemia, hepatitis, hypopituitarism, diabetes insipidus, or sicca syndrome.

In certain embodiments, the immune disorder is a type 1 interferonopathy, type 1 diabetes, Aicardi-Goutieres syndrome (AGS), arthritis, psoriasis, systemic lupus erythematosus (SLE), lupus nephritis, cutaneous lupus erythematosus (CLE), familial chilblain lupus, systemic sclerosis, STING-associated vasculopathy with onset in infancy (SAVI), graft versus host disease, scleroderma, polymyositis, inflammatory bowel disease, dermatomyositis, ulcerative colitis, Crohn's disease, vasculitis, psoriatic arthritis, Reiter's syndrome, exfoliative psoriatic dermatitis, pemphigus vulgaris, Sjogren's syndrome, autoimmune uveitis, glomerulonephritis, post myocardial infarction cardiotomy syndrome, pulmonary hemosiderosis, amyloidosis, sarcoidosis, aphthous stomatitis, thyroiditis, gastritis, adrenalitis (Addison's disease), ovaritis, primary biliary cirrhosis, myasthenia gravis, gonadal failure, hypoparathyroidism, alopecia, malabsorption syndrome, pernicious anemia, hypopituitarism, diabetes insipidus, or sicca syndrome.

In certain embodiments, the immune disorder is a type 1 interferonopathy, type 1 diabetes, Aicardi-Goutieres syndrome (AGS), systemic lupus erythematosus (SLE), lupus nephritis, cutaneous lupus erythematosus (CLE), familial chilblain lupus, systemic sclerosis, STING-associated vasculopathy with onset in infancy (SAVI), Sjogren's syndrome, dermatomyositis, inflammatory bowel disease, Crohn's disease, or ulcerative colitis.

In certain embodiments, the immune disorder is a type 1 interferonopathy, type 1 diabetes, Aicardi-Goutieres syndrome (AGS), systemic lupus erythematosus (SLE), lupus nephritis, cutaneous lupus erythematosus (CLE), dermatomyositis, or Sjogren's syndrome.

In certain embodiments, the immune disorder is a type 1 interferonopathy. In certain embodiments, the immune disorder is type 1 diabetes, Aicardi-Goutieres syndrome (AGS), systemic lupus erythematosus (SLE), lupus nephritis, cutaneous lupus erythematosus (CLE), familial chilblain lupus, systemic sclerosis, STING-associated vasculopathy with onset in infancy (SAVI), Sjogren's syndrome, or dermatomyositis. In certain embodiments, the immune disorder is systemic lupus erythematosus (SLE), lupus nephritis, cutaneous lupus erythematosus (CLE), or familial chilblain lupus. In certain embodiments, the immune disorder is systemic lupus erythematosus (SLE), lupus nephritis, or cutaneous lupus erythematosus (CLE). In certain embodiments, the immune disorder is type 1 diabetes, Aicardi-Goutieres syndrome (AGS), systemic sclerosis, STING-associated vasculopathy with onset in infancy (SAVI), Sjogren's syndrome, or dermatomyositis. In certain embodiments, the immune disorder is Aicardi-Goutieres syndrome (AGS), familial chilblain lupus, or STING-associated vasculopathy with onset in infancy (SAVI).

In certain embodiments, the immune disorder is type 1 diabetes. In certain embodiments, the immune disorder is Aicardi-Goutieres syndrome (AGS). In certain embodiments, the immune disorder is systemic lupus erythematosus (SLE). In certain embodiments, the immune disorder is lupus nephritis. In certain embodiments, the immune disorder is cutaneous lupus erythematosus (CLE). In certain embodiments, the immune disorder is familial chilblain lupus. In certain embodiments, the immune disorder is systemic sclerosis. In certain embodiments, the immune disorder is STING-associated vasculopathy with onset in infancy (SAVI). In certain embodiments, the immune disorder is Sjogren's syndrome. In certain embodiments, the immune disorder is dermatomyositis.

In certain embodiments, the immune disorder is inflammatory bowel disease, Crohn's disease, or ulcerative colitis. In certain embodiments, the immune disorder is inflammatory bowel disease. In certain embodiments, the immune disorder is Crohn's disease. In certain embodiments, the immune disorder is ulcerative colitis.

### Neurodegenerative Disorders

In certain embodiments, the disorder is a neurodegenerative disorder. In certain embodiments, the neurodegenerative disorder is amyotrophic lateral sclerosis (ALS), multiple sclerosis, Parkinson's disease, Huntington's disease, peripheral neuropathy, Creutzfeldt-Jacob disease, stroke, prion disease, frontotemporal dementia, Pick's disease, progressive supranuclear palsy, spinocerebellar ataxias, Lewy body disease, dementia, multiple system atrophy, epilepsy, bipolar disorder, schizophrenia, an anxiety disorder, or major depression. In certain embodiments, the neurodegenerative disorder is neurodegenerative disorder is amyotrophic lateral sclerosis (ALS), multiple sclerosis, Parkinson's disease, Huntington's disease, or dementia.

In certain embodiments, the neurodegenerative disorder is Alzheimer's disease, amyotrophic lateral sclerosis (ALS), multiple sclerosis, Parkinson's disease, Huntington's disease, peripheral neuropathy, age-related macular degeneration, Creutzfeldt-Jacob disease, stroke, prion disease, frontotemporal dementia, Pick's disease, progressive supranuclear palsy, spinocerebellar ataxias, Lewy body disease, dementia, multiple system atrophy, epilepsy, bipolar disorder, schizophrenia, an anxiety disorder, or major depression.

In certain embodiments, the neurodegenerative disorder is Alzheimer's disease, amyotrophic lateral sclerosis (ALS), multiple sclerosis, Parkinson's disease, Huntington's disease, dementia, or age-related macular degeneration. In certain embodiments, the neurodegenerative disorder is Alzheimer's disease, amyotrophic lateral sclerosis (ALS), Parkinson's disease, or age-related macular degeneration. In certain embodiments, the neurodegenerative disorder is age-related macular degeneration.

In certain embodiments, the neurodegenerative disorder is Alzheimer's disease, amyotrophic lateral sclerosis (ALS), multiple sclerosis, Parkinson's disease, Huntington's disease, or dementia. In certain embodiments, the neurodegenerative disorder is Alzheimer's disease, amyotrophic lateral sclerosis (ALS), or Parkinson's disease. In certain embodiments, the neurodegenerative disorder is Alzheimer's disease. In certain embodiments, the neurodegenerative disorder is amyotrophic lateral sclerosis (ALS). In certain embodiments, the neurodegenerative disorder is multiple sclerosis. In certain embodiments, the neurodegenerative disorder is Parkinson's disease. In certain embodiments, the neurodegenerative disorder is Huntington's disease. In certain embodiments, the neurodegenerative disorder is dementia.

### Subjects

In certain embodiments, the subject has (i) expression of LINE1 RNA, LINE1 ORF1 polypeptide, and/or LINE1 ORF2 polypeptide; (ii) activity of LINE1 reverse transcriptase; (iii) expression of HERV-K RNA, and/or (iv) activity of HERV-K reverse transcriptase.

In certain embodiments, the subject has (i) expression of LINE1 RNA, LINE1 ORF1 polypeptide, and/or LINE1 ORF2 polypeptide; and/or (ii) activity of LINE1 reverse transcriptase. In certain embodiments, the subject has expression of LINE1 RNA, LINE1 ORF1 polypeptide, and/or LINE1 ORF2 polypeptide. In certain embodiments, the subject has expression of LINE1 RNA. In certain embodiments, the subject has expression of LINE1 ORF1 polypeptide. In certain embodiments, the subject has expression of LINE1 ORF2 polypeptide. In certain embodiments, the subject has activity of LINE1 reverse transcriptase.

In certain embodiments, the subject has (i) expression of HERV-K RNA, and/or (ii) activity of HERV-K reverse transcriptase. In certain embodiments, the subject has expression of HERV-K RNA. In certain embodiments, the subject has activity of HERV-K reverse transcriptase.

In certain embodiments, the subject has elevated (i) levels of LINE1 RNA, LINE1 ORF1 polypeptide, and/or LINE1 ORF2 polypeptide; (ii) activity of LINE1 reverse transcriptase; (iii) levels of HERV-K RNA, and/or (iv) activity of HERV-K reverse transcriptase.

In certain embodiments, the subject has elevated (i) levels of LINE1 RNA, LINE1 ORF1 polypeptide, and/or LINE1 ORF2 polypeptide; and/or (ii) activity of LINE1 reverse transcriptase. In certain embodiments, the subject has elevated levels of LINE1 RNA, LINE1 ORF1 polypeptide, and/or LINE1 ORF2 polypeptide. In certain embodiments, the subject has elevated levels of LINE1 RNA. In certain embodiments, the subject has elevated levels of LINE1 ORF1 polypeptide. In certain embodiments, the subject has elevated levels of LINE1 ORF2 polypeptide. In certain embodiments, the subject has elevated activity of LINE1 reverse transcriptase.

In certain embodiments, the subject has elevated (i) levels of HERV-K RNA, and/or (ii) activity of HERV-K reverse transcriptase. In certain embodiments, the subject has elevated levels of HERV-K RNA. In certain embodiments, the subject has elevated activity of HERV-K reverse transcriptase.

In certain embodiments, the subject is a human. In certain embodiments, the subject is an adult human. In certain embodiments, the subject is a pediatric human. In certain embodiments, the subject is a companion animal. In certain embodiments, the subject is a canine, feline, or equine.

### Uses of Compounds

Another aspect of the invention provides for the use of a compound described herein (such as a compound of Formula I, or other compounds in Section III) for treating a medical disorder, such as a medical disorder described herein (for example, cancer).

Another aspect of the invention provides for the use of a compound described herein (such as a compound of Formula I, or other compounds in Section III) in the manufacture of a medicament. In certain embodiments, the medicament is for treating a disorder described herein, such as cancer.

### Biological Assays

Compounds may be tested for their ability to treat one or more of the disorders described above according to any of various assays known in the art, including those described in the Examples. For example, compounds may be tested for their ability to activate the immune system; such assays are described in the literature. Results showing activation of the immune system support use of such compounds to treat cancer. Additional specific assays of interest are described below.

Compounds may be tested for their ability to reduce cancer cell viability using a CellTiter-Glo assay with cancer cells cultured in 3D colonies. Ovarian cancer cell line SK-OV-3 cells are cultured in McCoy's 5a media containing 10% FBS. Ovarian cancer cell line OVCAR-8 cells are cultured in RPMI media containing 10% FBS. Cell colony formation is tested using a 3D methylcellulose-based CellTiter-Glo (CTG) viability assay (Cat. No: G7573, Promega). Briefly, cells are inoculated into 96-well plates (at 1,500 cells per well) into a solution of 0.65% methylcellulose in growth media and incubated overnight at 37° C. in 5% CO₂. The next day, serially diluted test compound or positive control (cisplatin, Cat. No. 6J015A89, Qilu Pharma) are added at the indicated concentrations, and the cells are incubated for 7 days. On day seven, 100 μL of CTG reagent is added, and the plates are incubated at room temperature for 20 min. Luminescence is read on an Envision Multi Label Reader according to manufacturer's instructions. IC₅₀ values are determined using the following calculation:

$$Surviving rate ( \% ) = ( \frac{LumTest article - LumMedium control}{LumNone treated - LumMedium control} ) \times 100 \% .$$

Compounds may be tested for their ability to alter interferon-stimulated gene (ISG) response in an in vivo mouse model, where decitabine is dosed to induce ISG response. Mice (9-11 week old C57BL/6), five per dosing group of test compound and five for a control group, are acclimated to the lab for at least 5 days. Test compound is prepared in an appropriate formulation for p.o. administration. Decitabine (Sigma) is dissolved in sterile PBS (pH 7.4) and dosed (i.p., 5 mg/kg) within 30 minutes of preparation of the solution. Doses of both test compound and decitabine are administered once a day, every day from Day 0 to Day 4. All mice are euthanized 1 hour after the last dose administration on Day 4. Spleens, liver, and terminal colon are collected, along with plasma from each animal. The fold changes in interferon-stimulated gene (ISG) expression are calculated by first normalizing to GAPDH gene using the Delta CT method. The CT (gene of interest)−CT (reference gene) is calculated to generate a delta CT for all samples. The fold change is then calculated by taking the Log 2(Delta CT(control)−Delta CT (experimental). The control in this example is the control animal group. The Taqman duplex assay (Thermo Fisher 4331182 and 4448489) is used according to the manufacturer's instructions to determine levels of GAPDH v. IFIT2.

Compounds may be tested for their ability to alter interferon β (IFN-β) and/or interleukin 2 (IL-2) production by human PBMC's, where decitabine is dosed to induce an interferon response. Cells are prepared for this assay as follows. EasySep buffer (32 mL, Stem Cell, cat. #20144) is used to dilute 8 mL of LRSC buffy coat (from fresh Leukopak) with gentle mixing. The diluted buffy coat (20 mL) is transferred into each of two SepMate 50 tubes, and the tubes are filled with 15 mL of Lyphoprep (Stem Cell, ct. #07851) density gradient. The SepMate tubes are then centrifuged at 1200 G for 10 minutes at room temperature with the brake on. The top layer of supernatant is collected in SepMate tubes by quickly pouring it into a new 50 mL conical tube. The PBMCs are washed with EasySep buffer ×2 by centrifuging at 300 G for 5 minutes. The cells are resuspended in 30 mL of EasySep and centrifuged at 100 G for 5 minutes with the brake off, and the platelets are removed. The cells are then resuspended in 6 mL of 1×RBC lysis buffer (InvitroGen) and incubated at 37° C. for 5 minutes. Then, 25 mL of EasySep buffer is mixed into the tube and it is centrifuged at 300 G for 5 minutes. The cells are resuspended in 10 mL of EasySep buffer and the cells are then counted with Cellometer (AO/PI). The PBMCs are resuspended in RPMI1640 (ThermoFisher)+10% FBS (HyClone)+p/s at 3×10⁶/mL. The PBMCs (100 μL, 300 k PBMCs) are then seeded in a 96-well flat bottom microplate (Corning) that is precoated with 100 μL of anti-CD3 antibody (10 μg/mL in PBS, Biolegend) or PBS at 4° C., one day before the assay is commenced.

The assay is conducted as follows. To each well, the following solutions are added: 1) 100 μL of cells (final cell number per well is 3×10⁵ cell/well); 2) 25 μL of anti-CD28 antibody at 6× (5 μg/mL final concentration, Biolegend); 3) 25 μL of decitabine at 6× (10 μM final concentration); and 4) test compound in DMSO is dispensed directly into each well with a d300e digital dispense (Tecan). The final concentration of DMSO for each well is normalized to 0.3%. The plate is incubated at 37° C. without any agitation for 5 days. Samples are collected 120 hours after incubation to determine IFN-β and IL-2 levels using a U-PLEX Human IFNb Assay Sector (5PL) (MSD, cat. #K151VIK-2). After 5 days, the plate is spun down at 100×G for 5 minutes. Supernatants (100 μL) are collected for interferon β (IFN-β) analysis using the MSD assay noted above, and any residual supernatant is stored at −80° C. Cell viability is checked to determine if cell death had an impact on the IFN-β levels detected.

Compounds may be tested for their ability to alter the immune response in an in vivo mouse model, where myelin oligodendrocyte glycoprotein (MOG) is dosed to induce an immune response. On day zero, groups of C57BL mice, six per dosing group of test compound and six for a control group, are immunized subcutaneously at 2 sites with 0.1 mL/site with MOG₃₅₋₅₅/CFA (Hooke immunization kit). Dosing of mice with test compound starts on day 0 and continues through day 11. Mice are dosed each day at approximately the same time each day. One day 11, 1 hour after receiving the last dose, plasma is collected, frozen and stored at −80° C. for analysis. At the end of the experiment, all mice are euthanized, and inguinal lymph nodes are collected and processed. Lymph node cells from each group are set up in 96-well plates with 400 k cells/well along with seven concentrations of antigen: 0, 0.07 μg/mL, 0.2 μg/mL, 0.7 μg/mL, 2.2 μg/mL, 6.6 μg/mL and 20.0 μg/mL. After 72 hours of culturing, the supernatants are collected and analyzed for IL-17A, IFNγ, and TNF using CBA kits (Becton-Dickinson). A bromodeoxyuridine (BrdU) cell proliferation assay is run on some of the lymph node cells to determine if treatment of mice with test compound alters the proliferation of CD4+ T cells in culture upon restimulation with antigen. Cultures of the cells are set up in 96-well plates, each using 400 k cells/well along with six concentrations of antigen: 0, 0.2 μg/mL, 0.7 μg/mL, 2.2 μg/mL, 6.6 μg/mL and 20.0 μg/mL, each with duplicates. The cells are cultured for approximately 40 hours, then BrdU is added to all wells at a concentration of 3 μg/mL. The cells are cultured an additional 3 hours after the addition of BrdU. Cells are then collected, stained with anti-CD4 and anti-BrdU antibodies (as per Becton Dickinson's standard protocols for BrdU labeling) and analyzed.

Compounds may be tested for their ability to alter phosphorylation of TANK-binding kinase 1 (pTBK1) in HaCaT cells, upon exposure to UVB light. HaCaT cells are plated in 6-well plates at a density of 100 k/well in HaCaT media (DMEM, optimized 1× (Addex Bio)+1% pen strep (Gibco)+5% heat inactivated fetal bovine serum (Gibco)). The cells are then cultured at 37° C. overnight. The next day, the cells are treated with the test compounds. Each test compound is diluted and added to media aliquots to provide desired concentrations. To add the test compound+media mixture, an equivalent amount of media from each well is aspirated and then replenished with the media dosed with the test compound. The cells are then cultured for an additional 96 hours with compound treatment prior to UVB exposure. The media is then aspirated from the wells, with the remaining cells at least 80% confluent in each well. One mL of PBS is then added to each well, and the plate is then placed under a UVB lamp. A UVB sensor was positioned near the plate to register the plate's exposure. The cells are exposed to the UVB light until they reach 0.1 mJ/cm². Then the plate is covered and transferred to a sterile hood for processing.

The PBS is aspirated out of the wells, and the wells are replenished with 3 mL fresh culture media. The cells are then cultured for an additional 24 hours, and samples are processed 24 hours post-UVB exposure. To process the samples, the media is aspirated, the plate placed on ice, and the cells washed with cold PBS, which is then aspirated off. Another 1 mL of cold PBS is added to each well. The cells are then scraped in the cold PBS solution and transferred to conical tubes on ice. The cells are then spun at ≥1000 RCF at 4° C. for 5 minutes. The cells are then resuspended in 1 mL of cold PBS and transferred to a microcentrifuge tube. The cells are spun at ≥1000 RCF at 4° C. for another 5 minutes, and the PBS is aspirated off. The cell pellet is prepared for lysis. A RIPA lysis buffer (#BP-115, Boston Bio-Products) is added to a Halt protease and phosphate inhibitor cocktail (#78440, ThermoFisher), and the mixture is cooled on ice. About 30 μL of the lysis buffer mix is added to the cells. The samples are briefly vortexed and then incubated on ice for at least 15 minutes. The cells are then spun ≥1000 RCF at 4° C. for 5 minutes and the supernantant is transferred to a clean tube. The protein concentration of the cell lysate is measured using Pierce™ Rapid Gold BCA Protein Assay Kit #AF3225 (ThermoFisher). ELISA analysis is run on select samples using one of the following kits:
a. FastScan™ Phospho-TBK1/NAK (Ser172) ELISA Kit #46948 (Cell Signaling Technologies)
b. FastScan™ Total TBK1/NAK ELISA Kit #15816 (Cell Signaling Technologies)
c. FastScan™ Phospho-STING (Ser366) ELISA Kit #82083 (Cell Signaling Technologies)
d. FastScan™ Phospho-TRF-3 (Ser396) ELISA Kit #50386 (Cell Signaling Technologies)
e. FastScan™ Total TRF-3 ELISA Kit #29771 (Cell Signaling Technologies)
f. Phospho-NAK/TBK1 (S172) ELISA Kit (ab279952) (Abcam)
g. Phospho-TRF3 (S386) ELISA Kit (ab279833) (Abcam)

Compounds may be tested for their ability to inhibit tumor growth in patient-derived mouse xenograft models of cancer, according to a variety of protocols known in the art. For example, balb/c mice (6-8 weeks old) are inoculated subcutaneously in the right flank with a primary human tumor xenograft model tumor fragment (2-3 mm³ in diameter) for tumor development. When mean tumor volume reaches approximately 150-200 mm³, animals are randomly allocated to treatment groups of 3 mice each to receive vehicle control or test compound. Tumors are measured twice per week using calipers to determine the ability of the test compound to inhibit growth of the xenograft tumor.

Without being bound by theory, substituted adeninyl-propyloxy phosphonic acids and related compounds, such as those described herein, are believed to undergo conversion in vivo to compound V-1 and/or the corresponding diphosphate:

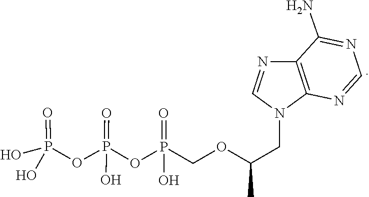 [SMILES: C[C@H](Cn1cnc2c(N)ncnc21)OCP(=O)(O)OP(=O)(O)OP(=O)(O)O]

Accordingly, assay results that support treating disease(s) with compound V-1 and/or the corresponding diphosphate, also support treating the same disease(s) with substituted adeninyl-propyloxy phosphonic acids and related compounds, such as those described herein. The rate of conversion in vivo of the substituted adeninyl-propyloxy phosphonic acids and related compounds described herein compound V-1 and/or the corresponding diphosphate, can be determined according to pharmacokinetic assay procedures described in the literature.

### Toxicity Counterscreens

Compounds may also be tested for their potential for toxicity, for example, cytotoxicity or mitochondrial toxicity, according to any of various assays known in the art. Specific assays of interest are described below, and include those described in Feng, J. Y. et al. “Role of Mitochondrial RNA Polymerase in the Toxicity of Nucleotide Inhibitors of Hepatitis C Virus,” Antimicrob. Agents Chemother. (2016) Vol. 60, No. 2, pp. 806-817; and Antes, A. et al. “Differential Regulation of Full-Length Genome and a Single-Stranded 7S DNA Along the Cell Cycle in Human Mitochondria,” Nucleic Acids Res. (2010) Vol. 38, No. 19, pp. 6466-6476.

For example, as described in Feng, J. Y. et al. compounds may be tested for cytotoxicity using CellTiter-Glo (CTG) viability assay (Cat. No: G7573, Promega). Prostate cancer PC-3 cells are cultured in F12K media containing 10% FBS. Briefly, cells are seeded into 96-well plates (at 3,000 cells per well) in 200 μL of growth media and incubated overnight at 37° C. in 5% CO₂. The next day, serially diluted test compound or positive control (chloramphenicol) is added, and the cells are incubated for 5 days. Compounds start at 100 μM, with 3-fold dilutions, and with a final DMSO volume of 0.1%. On day 5, 100 μL of medium is removed and 50 μL per well of CTG reagent is added. Plates are centrifuged at 1,000 rpm for 1 minute and then incubated at room temperature for 15 minutes. Luminescence is read on an Envision Multi Label Reader according to manufacturer's instructions. Percent survival is determined using the following calculation:

$$Surviving rate ( \% ) = ( ( \frac{( LumTest article ) - ( LumMedium control )}{( LumNone treated ) - ( LumMedium control )} ) )$$

The IC₅₀ is calculated by fitting the average of percent survival at each dose with a 4-parameter non-linear regression equation.

Additionally, as described in Feng, J. Y. et al. compounds may be tested for mitochondrial toxicity using mitochondrial protein synthesis, assessed by ELISA using MitoBiogenesis™ In-Cell ELISA Kit (Abcam ab110217). Prostate cancer PC-3 cells are cultured in F12K media containing 10% FBS. Briefly, cells are seeded into 96-well plates (at 3,000 cells per well) in 200 μL of growth media and incubated overnight at 37° C. in 5% CO₂. The next day, serially diluted test compound or positive control (chloramphenicol) is added, and the cells are incubated for 5 days. Compounds start at 100 μM, with 3-fold dilutions, and with a final DMSO volume of 0.1%. On day 5, the ELISA is conducted per manufacturer's instructions.

$$Percent of ( COX - 1 / SDH - A ) from DMSO = ( ( \frac{( Individual Test Sample ) - ( Background Avg )}{( Minimum Inhibition ( DMSO ) ) - ( Background Avg )} ) )$$

The IC₅₀ was calculated by fitting the average of percent from DMSO at each dose with a 4-parameter non-linear regression equation.

Alternatively, as described in Antes, A. et al. compounds may be tested for mitochondrial toxicity using mitochondrial DNA (mtDNA) and 7S DNA expression in prostate cancer PC-3 cells via qPCR. Prostate cancer PC-3 cells are cultured in F12K media containing 10% FBS. Mitochondrial DNA (mtDNA) and 7S DNA expression are tested by qPCR using PowerUp™ SYBR™ Green Master Mix (Applied Biosystems A25778). Briefly, cells are seeded into 6-well plates (at 50,000 cells per well) in 1 mL of growth media and incubated overnight at 37° C. in 5% CO₂. The next day, serially diluted test compound or positive control (zalcitabine, Cat. No. S1719, Selleck Chemicals) are added, and the cells are incubated for 5 days. Test compound starts at 100 μM, while positive control starts at 10 μM, both with a 10-fold dilution.

On day 5, DNA extraction is performed using DNeasy Blood and Tissue Kit (Qiagen #69504) according to the manufacturer's instruction. A total volume of 10 μL is used for the qPCR reaction. Four μL of DNA template (adjusted to 20 ng per reaction) is used from the extraction, 1 μL of primer (at a 5 μM stock concentration), and the remaining volume is the Master Mix. Settings for the QuantStudio™ 7 Flex RealTime qPCR System are as follows: 1 cycle of 50° C. for 2 minutes; 1 cycle of 95° C. for 2 minutes; 60 cycles of 95° C. for 15 seconds, and 60° C. for 60 seconds. The primer sequences are as follows:

| | Forward 7S: |
|---|---|
| (SEQ ID NO: 1) | |
| | GTGGCTTTGGAGTTGCAGTT |
| | Reverse 7S: |
| (SEQ ID NO: 2) | |
| | CAGCCACCATGAATATTGTAC |
| | Forward mtDNA: |
| (SEQ ID NO: 3) | |
| | GTGGCTTTGGAGTTGCAGTT |
| | Reverse mtDNA: |
| (SEQ ID NO: 4) | |
| | GAAGCAGATTTGGGTACCAC |
| | Forward 18S: |
| (SEQ ID NO: 5) | |
| | CGGGTGACGGGGAATCAG |
| | Reverse 18S: |
| (SEQ ID NO: 6) | |
| | CACTACCTCCCCGGGTC |
| | Forward GAPDH: |
| (SEQ ID NO: 7) | |
| | AGATCCCTCCAAAATCAAGTGG |
| | Reverse GAPDH: |
| (SEQ ID NO: 8) | |
| | GGCAGAGATGATGACCCTTTT. |

Ct values are exported into Excel. Relative mtDNA/7S DNA levels are determined with the following formulas:

ΔCt=Ct(Target gene)−Ct(Control), the control being either 18S or GAPDH.

Relative DNA level=2^{-ΔCt}

Fold change=2^{-ΔCt}(compound treated group)/2^{-ΔCt}(DMSO treated group)

Percent inhibition=1−(compound treated group)/(average of DMSO treated group)

The IC₅₀ is calculated by fitting the average of percent inhibition at each dose with a 4-parameter non-linear regression equation.

### II. Methods of Inhibiting LINE1 and/or HERV-K Reverse Transcriptase Activity in a Subject

Another aspect of the invention provides a method of inhibiting LINE1 reverse transcriptase activity in a subject suffering from a disorder selected from the group consisting of cancer, an inflammatory disorder, a neurodegenerative disorder, and an immune disorder other than a viral infection. The method comprises contacting a LINE1 reverse transcriptase with an effective amount of a compound of Formula I, in order to inhibit the activity of said LINE1 reverse transcriptase; wherein Formula I is represented by:

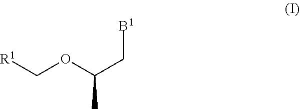

or a stereoisomer thereof; or a pharmaceutically acceptable salt of either of the foregoing; wherein:
R¹ is —P(O)(OR³)(N(R⁴)(R⁵)), —P(O)(OR³)₂, or —P(O)(N(R⁴)(R⁵))₂;
R² is hydrogen, —NH₂, or fluoro;
R³ represents independently for each occurrence:
   a. phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein said phenyl, naphthyl, 5-6 membered monocyclic heteroaryl, and 8-10 membered bicyclic heteroaryl are substituted with m instances of R⁸;
   b. hydrogen, —P(O)(OH)₂, —P(O)(OH)—O—P(O)(OH)₂, C₁₋₂₀ alkyl, C₁₋₂₀ haloalkyl, —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), —(C₁₋₁₀ alkylene)-Y—(C₂₋₂₀ alkynylene)-R¹¹, or —C(R⁶)₂—CO₂R¹⁰; wherein said C₁₋₂₀ alkyl, C₁₋₂₀ haloalkyl, and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene; or
   c. two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸;
R⁴ and R⁹ each represents independently for each occurrence hydrogen or C₁₋₄ alkyl; or R⁴ and one occurrence of R⁶ are taken together with atoms to which they are attached to form a 4-7 membered saturated heterocyclic ring containing 1 nitrogen atom; or two instances of R⁹ are taken together with the atom to which they are attached to form a 4-7 membered saturated heterocyclic ring containing 1 nitrogen atom and optionally 1 oxygen atom;
R⁵ represents independently for each occurrence:
   a. —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, —C(R¹⁰)(H)—CH₂—CO₂R¹⁰, C₁₋₂₀ alkyl, C₁₋₂₀ haloalkyl, —(C₁₋₁₀ alkylene)-X—(C₁₋₁₀ alkyl), —(C₁₋₁₀ alkylene)-phenyl, —(C₁₋₄ haloalkylene)-phenyl, —C(O)-phenyl, —C(S)-phenyl, or —C(O)-(5-6 membered monocyclic heteroaryl having 1, 2, or 3 heteroatoms independently selected from nitrogen, oxygen, and sulfur); or
   b. phenyl; naphthyl; a 5-6 membered monocyclic heteroaryl having 1, 2, or 3 heteroatoms independently selected from nitrogen, oxygen, and sulfur; or an 8-10 membered bicyclic heteroaryl having 1, 2, or 3 heteroatoms independently selected from nitrogen, oxygen, and sulfur;
   wherein said phenyl, naphthyl, 5-6 membered monocyclic heteroaryl, and 8-10 membered bicyclic heteroaryl are substituted with m instances of R⁸; and said C₁₋₂₀ alkyl, C₁₋₂₀ haloalkyl, and C₁₋₁₀ alkyl are optionally substituted with one hydroxyl;
R⁶ represents independently for each occurrence C₁₋₆ alkyl, C₁₋₆ haloalkyl, C₃₋₅ cycloalkyl, —CN, or hydrogen, wherein said C₁₋₆ alkyl is optionally substituted with —S—(C₁₋₄ alkyl), —SH, C₁₋₄ alkoxyl, C₁₋₄ haloalkoxyl, hydroxyl, —OCH₂CN, phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; or two instances of R⁶ are taken together with the carbon atom to which they are attached to form a 3-6 membered saturated ring having 0 or 1 oxygen atom;
R⁷ represents independently for each occurrence C₁₋₈ alkyl, C₁₋₆ haloalkyl, C₂₋₆ alkenyl, C₃₋₇ cycloalkyl, or a 4-7 membered saturated monocyclic heterocyclyl having one or two heteroatoms independently selected from nitrogen, oxygen, and sulfur; wherein said C₁₋₈ alkyl is optionally substituted with C₁₋₄ alkoxyl, phenyl, C₃₋₇ cycloalkyl, or a 4-7 membered saturated monocyclic heterocyclyl having one or two heteroatoms independently selected from nitrogen, oxygen, and sulfur;
R⁸ represents independently for each occurrence halo, C₁₋₄ alkyl, C₁₋₄ haloalkyl, C₁₋₄ alkoxyl, C₁₋₄ haloalkoxyl, —(C₀₋₃ alkylene)-CO₂R¹⁰, —CN, or —N(R⁹)₂;
R¹⁰ represents independently for each occurrence C₁₋₆ alkyl, C₃₋₇ cycloalkyl, or hydrogen;
R¹¹ represents independently for each occurrence C₁₋₂ haloalkyl, —SF₅, —Si(C₁₋₄ alkyl)₃, —Si(CH₃)₂(C₁₋₄ haloalkyl), —Si(CH₃)₂(C₃₋₇ cycloalkyl),

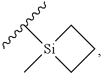 [SMILES: *[Si]1(C)CCC1]

—Si(CH₃)₂(phenyl), —S-phenyl, —O-phenyl, phenyl, thiophenyl, pyridinyl, or C₃₋₇ cycloalkyl; wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅;
X represents independently for each occurrence —O—, ψ-OC(O)—, —OC(O)O—, ψ-OC(O)—N(R⁹)—, —S—, —S—S—, or ψ-SC(O)—; wherein ψ denotes the point of attachment to C₁₋₁₀ alkylene;
Y represents independently for each occurrence —O—, —S—, or —CF₂—;
B¹ is

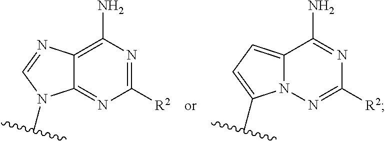

and
m and p are independently for each occurrence 0, 1, 2, or 3;
wherein one or more hydrogen atoms may be replaced with deuterium.

In certain embodiments, the particular compound of Formula I is a compound defined by one of the embodiments described in Section III, below, such as a compound of Formula I-A or I-B.

In certain embodiments, the disorder is a disorder defined by one of the embodiments described in Section I, above, such as cancer.

In certain embodiments, the method further comprises inhibiting HERV-K reverse transcriptase activity in the subject.

Another aspect of the invention provides a method of inhibiting HERV-K reverse transcriptase activity in a subject suffering from a disorder selected from the group consisting of cancer, an inflammatory disorder, a neurodegenerative disorder, and an immune disorder other than a viral infection. The method comprises contacting a HERV-K reverse erse transcriptase with an effective amount of a compound of Formula I, in order to inhibit the activity of said HERV-K reverse transcriptase; wherein Formula I is represented by:

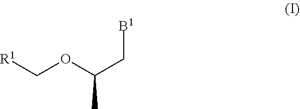

or a stereoisomer thereof; or a pharmaceutically acceptable salt of either of the foregoing; wherein:
R¹ is —P(O)(OR³)(N(R⁴)(R⁵)), —P(O)(OR³)₂, or —P(O)(N(R⁴)(R⁵))₂;
R² is hydrogen, —NH₂, or fluoro;
R³ represents independently for each occurrence:
   a. phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein said phenyl, naphthyl, 5-6 membered monocyclic heteroaryl, and 8-10 membered bicyclic heteroaryl are substituted with m instances of R⁸;
   b. hydrogen, —P(O)(OH)₂, —P(O)(OH)—O—P(O)(OH)₂, C₁₋₂₀ alkyl, C₁₋₂₀ haloalkyl, —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), —(C₁₋₁₀ alkylene)-Y—(C₂₋₂₀ alkynylene)-R¹¹, or —C(R⁶)₂—CO₂R¹⁰; wherein said C₁₋₂₀ alkyl, C₁₋₂₀ haloalkyl, and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene; or
   c. two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸;
R⁴ and R⁹ each represents independently for each occurrence hydrogen or C₁₋₄ alkyl; or R⁴ and one occurrence of R⁶ are taken together with atoms to which they are attached to form a 4-7 membered saturated heterocyclic ring containing 1 nitrogen atom; or two instances of R⁹ are taken together with the atom to which they are attached to form a 4-7 membered saturated heterocyclic ring containing 1 nitrogen atom and optionally 1 oxygen atom;
R⁵ represents independently for each occurrence:
   a. —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, —C(R¹⁰)(H)—CH₂—CO₂R¹⁰, C₁₋₂₀ alkyl, C₁₋₂₀ haloalkyl, —(C₁₋₁₀ alkylene)-X—(C₁₋₁₀ alkyl), —(C₁₋₁₀ alkylene)-phenyl, —(C₁₋₄ haloalkylene)-phenyl, —C(O)-phenyl, —C(S)-phenyl, or —C(O)-(5-6 membered monocyclic heteroaryl having 1, 2, or 3 heteroatoms independently selected from nitrogen, oxygen, and sulfur); or
   b. phenyl; naphthyl; a 5-6 membered monocyclic heteroaryl having 1, 2, or 3 heteroatoms independently selected from nitrogen, oxygen, and sulfur; or an 8-10 membered bicyclic heteroaryl having 1, 2, or 3 heteroatoms independently selected from nitrogen, oxygen, and sulfur;
   wherein said phenyl, naphthyl, 5-6 membered monocyclic heteroaryl, and 8-10 membered bicyclic heteroaryl are substituted with m instances of R⁸; and said C₁₋₂₀ alkyl, C₁₋₂₀ haloalkyl, and C₁₋₁₀ alkyl are optionally substituted with one hydroxyl;
R⁶ represents independently for each occurrence C₁₋₆ alkyl, C₁₋₆ haloalkyl, C₃₋₅ cycloalkyl, —CN, or hydrogen, wherein said C₁₋₆ alkyl is optionally substituted with —S—(C₁₋₄ alkyl), —SH, C₁₋₄ alkoxyl, C₁₋₄ haloalkoxyl, hydroxyl, —OCH₂CN, phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; or two instances of R⁶ are taken together with the carbon atom to which they are attached to form a 3-6 membered saturated ring having 0 or 1 oxygen atom;
R⁷ represents independently for each occurrence C₁₋₈ alkyl, C₁₋₆ haloalkyl, C₂₋₆ alkenyl, C₃₋₇ cycloalkyl, or a 4-7 membered saturated monocyclic heterocyclyl having one or two heteroatoms independently selected from nitrogen, oxygen, and sulfur; wherein said C₁₋₈ alkyl is optionally substituted with C₁₋₄ alkoxyl, phenyl, C₃₋₇ cycloalkyl, or a 4-7 membered saturated monocyclic heterocyclyl having one or two heteroatoms independently selected from nitrogen, oxygen, and sulfur;
R⁸ represents independently for each occurrence halo, C₁₋₄ alkyl, C₁₋₄ haloalkyl, C₁₋₄ alkoxyl, C₁₋₄ haloalkoxyl, —(C₀₋₃ alkylene)-CO₂R¹⁰, —CN, or —N(R⁹)₂;
R¹⁰ represents independently for each occurrence C₁₋₆ alkyl, C₃₋₇ cycloalkyl, or hydrogen;
R¹¹ represents independently for each occurrence C₁₋₂ haloalkyl, —SF₅, —Si(C₁₋₄ alkyl)₃, —Si(CH₃)₂(C₁₋₄ haloalkyl), —Si(CH₃)₂(C₃₋₇ cycloalkyl),

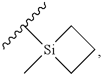 [SMILES: *[Si]1(C)CCC1]

—Si(CH₃)₂(phenyl), —S-phenyl, —O-phenyl, phenyl, thiophenyl, pyridinyl, or C₃₋₇ cycloalkyl; wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅;
X represents independently for each occurrence —O—, ψ-OC(O)—, —OC(O)O—, ψ-OC(O)—N(R⁹)—, —S—, —S—S—, or ψ-SC(O)—; wherein ψ denotes the point of attachment to C₁₋₁₀ alkylene;
Y represents independently for each occurrence —O—, —S—, or —CF₂—;
B¹ is

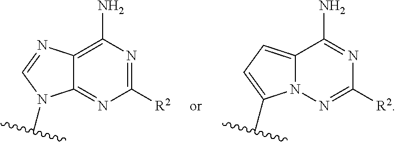

and
m and p are independently for each occurrence 0, 1, 2, or 3;
wherein one or more hydrogen atoms may be replaced with deuterium.

In certain embodiments, the particular compound of Formula I is a compound defined by one of the embodiments described in Section III, below, such as a compound of Formula I-A or I-B.

In certain embodiments, the disorder is a disorder defined by one of the embodiments described in Section I, above, such as cancer.

In certain embodiments, the method further comprises inhibiting LINE1 reverse transcriptase activity in the subject.

Compounds may be tested for ability to inhibit LINE1 reverse transcriptase activity, for example, as described in the Examples. Compounds may be tested for ability to inhibit HERV-K reverse transcriptase activity, for example, as described in the Examples.

### III. Substituted Adeninyl-Propyloxy Phosphonic Acid and Related Compounds

The methods described in Sections I and II above may be further characterized according to the substituted adeninyl-propyloxy phosphonic acid or related compound used in the methods. Exemplary compounds are described below, along with exemplary procedures for making the compounds.

In certain embodiments, the compound is a compound of Formula I represented by: B¹

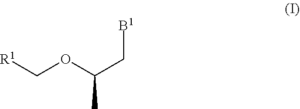

or a stereoisomer thereof; or a pharmaceutically acceptable salt of either of the foregoing; wherein:
R¹ is —P(O)(OR³)(N(R⁴)(R⁵)), —P(O)(OR³)₂, or —P(O)(N(R⁴)(R⁵))₂;
R² is hydrogen, —NH₂, or fluoro;
R³ represents independently for each occurrence:
   a. phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein said phenyl, naphthyl, 5-6 membered monocyclic heteroaryl, and 8-10 membered bicyclic heteroaryl are substituted with m instances of R⁸;
   b. hydrogen, —P(O)(OH)₂, —P(O)(OH)—O—P(O)(OH)₂, C₁₋₂₀ alkyl, C₁₋₂₀ haloalkyl, —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), —(C₁₋₁₀ alkylene)-Y—(C₂₋₂₀ alkynylene)-R¹¹, or —C(R⁶)₂—CO₂R¹⁰; wherein said C₁₋₂₀ alkyl, C₁₋₂₀ haloalkyl, and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene; or
   c. two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸;
R⁴ and R⁹ each represents independently for each occurrence hydrogen or C₁₋₄ alkyl; or R⁴ and one occurrence of R⁶ are taken together with atoms to which they are attached to form a 4-7 membered saturated heterocyclic ring containing 1 nitrogen atom; or two instances of R⁹ are taken together with the atom to which they are attached to form a 4-7 membered saturated heterocyclic ring containing 1 nitrogen atom and optionally 1 oxygen atom;
R⁵ represents independently for each occurrence:
   a. —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, —C(R¹⁰)(H)—CH₂—CO₂R¹⁰, C₁₋₂₀ alkyl, C₁₋₂₀ haloalkyl, —(C₁₋₁₀ alkylene)-X—(C₁₋₁₀ alkyl), —(C₁₋₁₀ alkylene)-phenyl, —(C₁₋₄ haloalkylene)-phenyl, —C(O)-phenyl, —C(S)-phenyl, or —C(O)-(5-6 membered monocyclic heteroaryl having 1, 2, or 3 heteroatoms independently selected from nitrogen, oxygen, and sulfur); or
   b. phenyl; naphthyl; a 5-6 membered monocyclic heteroaryl having 1, 2, or 3 heteroatoms independently selected from nitrogen, oxygen, and sulfur; or an 8-10 membered bicyclic heteroaryl having 1, 2, or 3 heteroatoms independently selected from nitrogen, oxygen, and sulfur;
   wherein said phenyl, naphthyl, 5-6 membered monocyclic heteroaryl, and 8-10 membered bicyclic heteroaryl are substituted with m instances of R⁸; and said C₁₋₂₀ alkyl, C₁₋₂₀ haloalkyl, and C₁₋₁₀ alkyl are optionally substituted with one hydroxyl;
R⁶ represents independently for each occurrence C₁₋₆ alkyl, C₁₋₆ haloalkyl, C₃₋₅ cycloalkyl, —CN, or hydrogen, wherein said C₁₋₆ alkyl is optionally substituted with —S—(C₁₋₄ alkyl), —SH, C₁₋₄ alkoxyl, C₁₋₄ haloalkoxyl, hydroxyl, —OCH₂CN, phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; or two instances of R⁶ are taken together with the carbon atom to which they are attached to form a 3-6 membered saturated ring having 0 or 1 oxygen atom;
R⁷ represents independently for each occurrence C₁₋₈ alkyl, C₁₋₆ haloalkyl, C₂₋₆ alkenyl, C₃₋₇ cycloalkyl, or a 4-7 membered saturated monocyclic heterocyclyl having one or two heteroatoms independently selected from nitrogen, oxygen, and sulfur; wherein said C₁₋₈ alkyl is optionally substituted with C₁₋₄ alkoxyl, phenyl, C₃₋₇ cycloalkyl, or a 4-7 membered saturated monocyclic heterocyclyl having one or two heteroatoms independently selected from nitrogen, oxygen, and sulfur;
R⁸ represents independently for each occurrence halo, C₁₋₄ alkyl, C₁₋₄ haloalkyl, C₁₋₄ alkoxyl, C₁₋₄ haloalkoxyl, —(C₀₋₃ alkylene)-CO₂R¹⁰, —CN, or —N(R⁹)₂;
R¹⁰ represents independently for each occurrence C₁₋₆ alkyl, C₃₋₇ cycloalkyl, or hydrogen;
R¹¹ represents independently for each occurrence C₁₋₂ haloalkyl, —SF₅, —Si(C₁₋₄ alkyl)₃, —Si(CH₃)₂(C₁₋₄ haloalkyl), —Si(CH₃)₂(C₃₋₇ cycloalkyl),

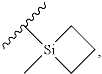 [SMILES: *[Si]1(C)CCC1]

—Si(CH₃)₂(phenyl), —S-phenyl, —O-phenyl, phenyl, thiophenyl, pyridinyl, or C₃₋₇ cycloalkyl; wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅;
X represents independently for each occurrence —O—, ψ-OC(O)—, —OC(O)O—, ψ-OC(O)—N(R⁹)—, —S—, —S—S—, or ψ-SC(O)—; wherein ψ denotes the point of attachment to C₁₋₁₀ alkylene;
Y represents independently for each occurrence —O—, —S—, or —CF₂—;
B¹ is

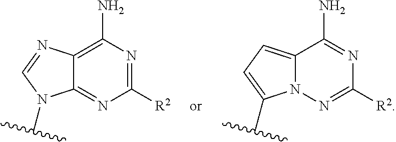

and
m and p are independently for each occurrence 0, 1, 2, or 3;
wherein one or more hydrogen atoms may be replaced with deuterium.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I, or a pharmaceutically acceptable salt thereof. In certain embodiments, the compound is a compound of Formula I. In certain embodiments, the compound is a stereoisomer of the compound of Formula I, or a pharmaceutically acceptable salt thereof. In certain embodiments, the compound is a stereoisomer of the compound of Formula I.

As defined generally above, R¹ is —P(O)(OR³)(N(R⁴)(R⁵)), —P(O)(OR³)₂, or —P(O)(N(R⁴)(R⁵))₂.

In certain embodiments, R¹ is —P(O)(OR³)(N(R⁴)(R⁵)) or —P(O)(OR³)₂. In certain embodiments, R¹ is —P(O)(OR³)(N(R⁴)(R⁵)) or —P(O)(N(R⁴)(R⁵))₂. In certain embodiments, R¹ is —P(O)(OR³)₂ or —P(O)(N(R⁴)(R⁵))₂.

In certain embodiments, R¹ is —P(O)(OR³)(N(R⁴)(R⁵)). In certain embodiments, R¹ is —P(O)(OR³)₂. In certain embodiments, R¹ is —P(O)(N(R⁴)(R⁵))₂.

In certain embodiments, R¹ is

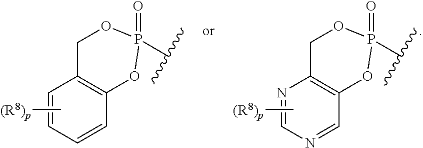

In certain embodiments, R¹ is

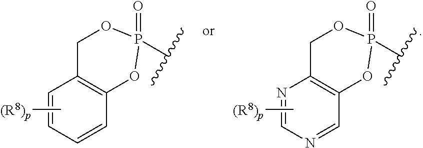

In certain embodiments, R¹ is

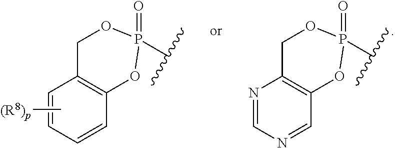

In certain embodiments R¹ is

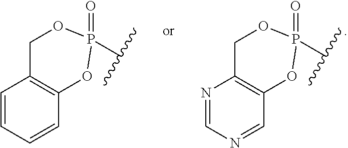 [SMILES: *P1(=O)OCc2ccccc2O1 ; *P1(=O)OCc2ncncc2O1]

In certain embodiments, R¹ is

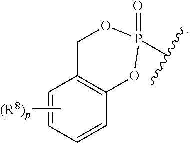

In certain embodiments, R¹ is

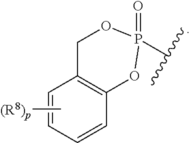

In certain embodiments, R¹ is

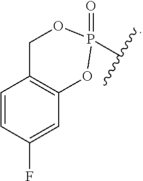 [SMILES: *P1(=O)OCc2ccc(F)cc2O1]

In certain embodiments, R¹ is

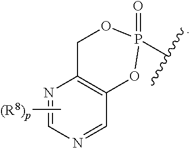

In certain embodiments, R¹ is

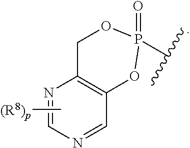

In certain embodiments, R¹ is

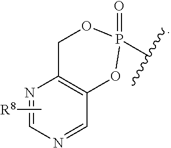

In certain embodiments, R¹ is

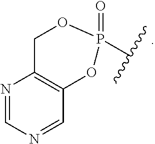 [SMILES: *P1(=O)OCc2ncncc2O1]

In certain embodiments, R¹ is selected from the groups depicted in the compounds in Tables 1, 2, 3, 4, 5, and 6, below.

As defined generally above, R² is hydrogen, —NH₂, or fluoro. In certain embodiments, R² is hydrogen or —NH₂. In certain embodiments, R² is hydrogen or fluoro. In certain embodiments, R² is —NH₂ or fluoro. In certain embodiments, R² is hydrogen. In certain embodiments, R² is —NH₂. In certain embodiments, R² is fluoro. In certain embodiments, R² is selected from the groups depicted in the compounds in Tables 1, 2, 3, 4, 5, and 6, below.

As defined generally above, R³ represents independently for each occurrence:
a. phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein said phenyl, naphthyl, 5-6 membered monocyclic heteroaryl, and 8-10 membered bicyclic heteroaryl are substituted with m instances of R⁸;
b. hydrogen, —P(O)(OH)₂, —P(O)(OH)—O—P(O)(OH)₂, C₁₋₂₀ alkyl, C₁₋₂₀ haloalkyl, —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), —(C₁₋₁₀ alkylene)-Y—(C₂₋₂₀ alkynylene)-R¹¹, or —C(R⁶)₂—CO₂R¹⁰; wherein said C₁₋₂₀ alkyl, C₁₋₂₀ haloalkyl, and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene; or
c. two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸.

In certain embodiments, R³ represents independently for each occurrence:
a. phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein said phenyl, naphthyl, 5-6 membered monocyclic heteroaryl, and 8-10 membered bicyclic heteroaryl are substituted with m instances of R⁸;
b. —P(O)(OH)₂, —P(O)(OH)—O—P(O)(OH)₂, C₁₋₂₀ alkyl, C₁₋₂₀ haloalkyl, —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), —(C₁₋₁₀ alkylene)-Y—(C₂₋₂₀ alkynylene)-R¹¹, or —C(R⁶)₂—CO₂R¹⁰; wherein said C₁₋₂₀ alkyl, C₁₋₂₀ haloalkyl, and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene; and wherein one occurrence of R³ is additionally selected from hydrogen; or
c. two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸.

In certain embodiments, R³ represents independently for each occurrence —C(R⁶)₂—CO₂R¹⁰, phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein said phenyl, naphthyl, 5-6 membered monocyclic heteroaryl, and 8-10 membered bicyclic heteroaryl are substituted with m instances of R⁸. In certain embodiments, R³ represents independently for each occurrence phenyl, naphthyl, or —C(R⁶)₂—CO₂R¹⁰; wherein said phenyl and naphthyl are substituted with m instances of R⁸. In certain embodiments, R³ represents independently for each occurrence —C(R⁶)₂—CO₂R¹⁰, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein said 5-6 membered monocyclic heteroaryl and 8-10 membered bicyclic heteroaryl are substituted with m instances of R⁸.

In certain embodiments, R³ represents independently for each occurrence phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein said phenyl, naphthyl, 5-6 membered monocyclic heteroaryl, and 8-10 membered bicyclic heteroaryl are substituted with m instances of R⁸. In certain embodiments, R³ represents independently for each occurrence phenyl or naphthyl; each of which is substituted with m instances of R⁸. In certain embodiments, R³ represents independently for each occurrence a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein said 5-6 membered monocyclic heteroaryl and 8-10 membered bicyclic heteroaryl are substituted with m instances of R⁸.

In certain embodiments, R³ represents independently for each occurrence phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur. In certain embodiments, R³ represents independently for each occurrence phenyl or naphthyl. In certain embodiments, R³ represents independently for each occurrence a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur.

In certain embodiments, R³ represents independently for each occurrence phenyl or naphthyl, each of which is substituted with m instances of R⁸. In certain embodiments, R³ represents independently for each occurrence phenyl substituted with m instances of R⁸. In certain embodiments, R³ represents independently for each occurrence naphthyl substituted with m instances of R⁸. In certain embodiments, R³ represents independently for each occurrence a 5-6 membered monocyclic heteroaryl having 1, 2, or 3 heteroatoms independently selected from nitrogen, oxygen, and sulfur; wherein said heteroaryl is substituted with m instances of R⁸. In certain embodiments, R³ represents independently for each occurrence an 8-10 membered bicyclic heteroaryl having 1, 2, or 3 heteroatoms independently selected from nitrogen, oxygen, and sulfur; wherein said heteroaryl is substituted with m instances of R⁸.

In certain embodiments, R³ represents independently for each occurrence a 5-6 membered monocyclic heteroaryl having 1, 2, or 3 heteroatoms independently selected from nitrogen, oxygen, and sulfur. In certain embodiments, R³ represents independently for each occurrence an 8-10 membered bicyclic heteroaryl having 1, 2, or 3 heteroatoms independently selected from nitrogen, oxygen, and sulfur.

In certain embodiments, R³ represents independently for each occurrence —C(R⁶)₂—CO₂R¹⁰.

In certain embodiments, R³ is phenyl, naphthyl, or —C(R⁶)₂—CO₂R¹⁰; wherein said phenyl and naphthyl are substituted with m instances of R⁸. In certain embodiments, R³ is phenyl or naphthyl, each of which is substituted with m instances of R⁸. In certain embodiments, R³ is phenyl substituted with m instances of R⁸. In certain embodiments, R³ is

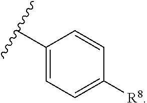

In certain embodiments, R³ is naphthyl substituted with m instances of R⁸.

In certain embodiments, R³ is phenyl or naphthyl. In certain embodiments, R³ is phenyl. In certain embodiments, R³ is naphthyl. In certain embodiments, R³ is 1-naphthyl. In certain embodiments, R³ is 2-naphthyl.

In certain embodiments, R³ is —C(R⁶)₂—CO₂R¹⁰.

In certain embodiments, R³ represents independently for each occurrence hydrogen, —P(O)(OH)₂, —P(O)(OH)—O—P(O)(OH)₂, C₁₋₂₀ alkyl, C₁₋₂₀ haloalkyl, —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), —(C₁₋₁₀ alkylene)-Y—(C₂₋₂₀ alkynylene)-R¹¹, or —C(R⁶)₂—CO₂R¹⁰; wherein said C₁₋₂₀ alkyl, C₁₋₂₀ haloalkyl, and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene; or
two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸.

In certain embodiments, R³ represents independently for each occurrence hydrogen, —P(O)(OH)₂, —P(O)(OH)—O—P(O)(OH)₂, C₁₋₂₀ alkyl, C₁₋₂₀ haloalkyl, —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), —(C₁₋₁₀ alkylene)-Y—(C₂₋₂₀ alkynylene)-R¹¹, or —C(R⁶)₂—CO₂R¹⁰; wherein said C₁₋₂₀ alkyl, C₁₋₂₀ haloalkyl, and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene.

In certain embodiments, R³ represents independently for each occurrence C₁₋₂₀ alkyl, C₁₋₂₀ haloalkyl, —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), —(C₁₋₁₀ alkylene)-Y—(C₂₋₂₀ alkynylene)-R¹¹, or —C(R⁶)₂—CO₂R¹⁰; wherein said C₁₋₂₀ alkyl, C₁₋₂₀ haloalkyl, and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene. In certain embodiments, R³ represents independently for each occurrence C₁₋₂₀ alkyl, C₁₋₂₀ haloalkyl, —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), or —(C₁₋₁₀ alkylene)-Y—(C₂₋₂₀ alkynylene)-R¹¹; wherein said C₁₋₂₀ alkyl, C₁₋₂₀ haloalkyl, and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene. In certain embodiments, R³ represents independently for each occurrence C₁₋₂₀ alkyl or C₁₋₂₀ haloalkyl; wherein said C₁₋₂₀ alkyl and C₁₋₂₀ haloalkyl are optionally substituted with one hydroxyl, —SH, C₁₋₂₀ alkoxyl, or —OC(O)—N(R⁹)₂; and wherein one methylene unit in each of said C₁₋₂₀ alkyl and C₁₋₂₀ haloalkyl is optionally replaced with a C₃₋₅ cycloalkylene or phenylene. In certain embodiments, R³ represents independently for each occurrence —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), or —(C₁₋₁₀ alkylene)-Y—(C₂₋₂₀ alkynylene)-R¹¹; wherein said C₁₋₂₀ alkyl and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene. In certain embodiments, R³ represents independently for each occurrence —(C₁₋₁₀ alkylene)-X—(C₁₋₂₀ alkyl) or —(C₁₋₁₀ alkylene)-Y—(C₁₋₂₀ alkylene)-R¹¹; wherein said C₁₋₂₀ alkyl and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene.

In certain embodiments, R³ represents independently for each occurrence C₁₋₂₀ alkyl optionally substituted with one hydroxyl, —SH, C₁₋₂₀ alkoxyl, or —OC(O)—N(R⁹)₂; wherein one methylene unit in said C₁₋₂₀ alkyl is optionally replaced with a C₃₋₅ cycloalkylene or phenylene. In certain embodiments, R³ represents independently for each occurrence C₁₋₂₀ alkyl optionally substituted with one hydroxyl, —SH, C₁₋₂₀ alkoxyl, or —OC(O)—N(R⁹)₂. In certain embodiments, R³ represents independently for each occurrence C₁₋₂₀ alkyl; wherein one methylene unit in said C₁₋₂₀ alkyl is optionally replaced with a C₃₋₅ cycloalkylene or phenylene.

In certain embodiments, R³ represents independently for each occurrence C₁₋₂₀ haloalkyl optionally substituted with one hydroxyl, —SH, C₁₋₂₀ alkoxyl, or —OC(O)—N(R⁹)₂; wherein one methylene unit in said C₁₋₂₀ haloalkyl is optionally replaced with a C₃₋₅ cycloalkylene or phenylene. In certain embodiments, R³ represents independently for each occurrence C₁₋₂₀ haloalkyl optionally substituted with one hydroxyl, —SH, C₁₋₂₀ alkoxyl, or —OC(O)—N(R⁹)₂. In certain embodiments, R³ represents independently for each occurrence C₁₋₂₀ haloalkyl; wherein one methylene unit in said C₁₋₂₀ haloalkyl is optionally replaced with a C₃₋₅ cycloalkylene or phenylene.

In certain embodiments, R³ represents independently for each occurrence —(C₁₋₁₀ alkylene)-X—(C₁₋₂₀ alkyl); wherein said C₁₋₂₀ alkyl and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl and C₁₋₁₀ alkylene is optionally replaced with a C₃₋₅ cycloalkylene or phenylene. In certain embodiments, R³ represents independently for each occurrence —(C₁₋₁₀ alkylene)-X—(C₁₋₂₀ alkyl); wherein said C₁₋₂₀ alkyl and C₁₋₁₀ alkylene are optionally substituted with one hydroxyl, —SH, C₁₋₂₀ alkoxyl, or —OC(O)—N(R⁹)₂. In certain embodiments, R³ represents independently for each occurrence —(C₁₋₁₀ alkylene)-X—(C₁₋₂₀ alkyl); wherein one methylene unit in each of said C₁₋₂₀ alkyl and C₁₋₁₀ alkylene is optionally replaced with a C₃₋₅ cycloalkylene or phenylene.

In certain embodiments, R³ represents independently for each occurrence —(C₁₋₁₀ alkylene)-Y—(C₁₋₂₀ alkylene)-R¹¹, —(C₁₋₁₀ alkylene)-Y—(C₂₋₂₀ alkynyl), or —(C₁₋₁₀ alkylene)-Y—(C₂₋₂₀ alkynylene)-R¹¹; wherein said C₁₋₁₀ alkylene is optionally substituted with one hydroxyl, —SH, C₁₋₂₀ alkoxyl, or —OC(O)—N(R⁹)₂; and wherein one methylene unit in said C₁₋₁₀ alkylene is optionally replaced with a C₃₋₅ cycloalkylene or phenylene. In certain embodiments, R³ represents independently for each occurrence —(C₁₋₁₀ alkylene)-Y—(C₁₋₂₀ alkylene)-R¹¹, —(C₁₋₁₀ alkylene)-Y—(C₂₋₂₀ alkynyl), or —(C₁₋₁₀ alkylene)-Y—(C₂₋₂₀ alkynylene)-R¹¹; wherein said C₁₋₁₀ alkylene is optionally substituted with one hydroxyl, —SH, C₁₋₂₀ alkoxyl, or —OC(O)—N(R⁹)₂. In certain embodiments, R³ represents independently for each occurrence —(C₁₋₁₀ alkylene)-Y—(C₁₋₂₀ alkylene)-R¹¹, —(C₁₋₁₀ alkylene)-Y—(C₂₋₂₀ alkynyl), or —(C₁₋₁₀ alkylene)-Y—(C₂₋₂₀ alkynylene)-R¹¹; wherein one methylene unit in said C₁₋₁₀ alkylene is optionally replaced with a C₃₋₅ cycloalkylene or phenylene.

In certain embodiments, R³ represents independently for each occurrence —(C₁₋₁₀ alkylene)-Y—(C₁₋₂₀ alkylene)-R¹¹, —(C₁₋₁₀ alkylene)-Y—(C₂₋₂₀ alkynyl), or —(C₁₋₁₀ alkylene)-Y—(C₂₋₂₀ alkynylene)-R¹¹. In certain embodiments, R³ represents independently for each occurrence —(C₁₋₄ alkylene)-Y—(C₁₋₂₀ alkylene)-R¹¹, —(C₁₋₄ alkylene)-Y—(C₂₋₂₀ alkynyl), or —(C₁₋₄ alkylene)-Y—(C₂₋₂₀ alkynylene)-R¹¹. In certain embodiments, R³ represents independently for each occurrence —(C₁₋₄ alkylene)-Y—(C₈₋₂₀ alkylene)-R¹¹, —(C₁₋₄ alkylene)-Y—(C₈₋₂₀ alkynyl), or —(C₁₋₄ alkylene)-Y—(C₈₋₂₀ alkynylene)-R¹¹.

In certain embodiments, R³ represents independently for each occurrence —(C₁₋₁₀ alkylene)-Y—(C₁₋₂₀ alkylene)-R¹¹; wherein said C₁₋₁₀ alkylene is optionally substituted with one hydroxyl, —SH, C₁₋₂₀ alkoxyl, or —OC(O)—N(R⁹)₂; and wherein one methylene unit in said C₁₋₁₀ alkylene is optionally replaced with a C₃₋₅ cycloalkylene or phenylene. In certain embodiments, R³ represents independently for each occurrence —(C₁₋₁₀ alkylene)-Y—(C₁₋₂₀ alkylene)-R¹¹; wherein said C₁₋₁₀ alkylene is optionally substituted with one hydroxyl, —SH, C₁₋₂₀ alkoxyl, or —OC(O)—N(R⁹)₂. In certain embodiments, R³ represents independently for each occurrence —(C₁₋₁₀ alkylene)-Y—(C₁₋₂₀ alkylene)-R¹¹; wherein one methylene unit in said C₁₋₁₀ alkylene is optionally replaced with a C₃₋₅ cycloalkylene or phenylene.

In certain embodiments, R³ represents independently for each occurrence —(C₁₋₁₀ alkylene)-Y—(C₁₋₂₀ alkylene)-R¹¹. In certain embodiments, R³ represents independently for each occurrence —(C₁₋₄ alkylene)-Y—(C₁₋₂₀ alkylene)-R¹¹. In certain embodiments, R³ represents independently for each occurrence —(C₁₋₄ alkylene)-Y—(C₈₋₂₀ alkylene)-R¹¹.

In certain embodiments, R³ represents independently for each occurrence C₁₋₂₀ alkyl, C₁₋₂₀ haloalkyl, —(C₁₋₁₀ alkylene)-X—(C₁₋₂₀ alkyl), hydrogen, —P(O)(OH)₂, or —P(O)(OH)—O—P(O)(OH)₂; wherein said C₁₋₂₀ alkyl, C₁₋₂₀ haloalkyl, and C₁₋₁₀ alkylene are optionally substituted with one hydroxyl or C₁₋₂₀ alkoxyl; and wherein one methylene unit in each of said C₁₋₂₀ alkyl, C₁₋₂₀ haloalkyl, and C₁₋₁₀ alkylene is optionally replaced with a C₃₋₅ cycloalkylene; or
two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸.

In certain embodiments, R³ represents independently for each occurrence C₁₋₂₀ alkyl, C₁₋₂₀ haloalkyl, —(C₁₋₁₀ alkylene)-X—(C₁₋₂₀ alkyl), —P(O)(OH)₂, or —P(O)(OH)—O—P(O)(OH)₂; wherein said C₁₋₂₀ alkyl, C₁₋₂₀ haloalkyl, and C₁₋₁₀ alkylene are optionally substituted with one hydroxyl or C₁₋₂₀ alkoxyl; and wherein one methylene unit in each of said C₁₋₂₀ alkyl, C₁₋₂₀ haloalkyl, and C₁₋₁₀ alkylene is optionally replaced with a C₃₋₅ cycloalkylene; and wherein one occurrence of R³ is additionally selected from hydrogen; or
two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸.

In certain embodiments, R³ represents independently for each occurrence C₁₋₂₀ alkyl, C₁₋₂₀ haloalkyl, —(C₁₋₁₀ alkylene)-X—(C₁₋₂₀ alkyl), hydrogen, —P(O)(OH)₂, or —P(O)(OH)—O—P(O)(OH)₂; wherein said C₁₋₂₀ alkyl, C₁₋₂₀ haloalkyl, and C₁₋₁₀ alkylene are optionally substituted with one hydroxyl or C₁₋₂₀ alkoxyl; and wherein one methylene unit in each of said C₁₋₂₀ alkyl, C₁₋₂₀ haloalkyl, and C₁₋₁₀ alkylene is optionally replaced with a C₃₋₅ cycloalkylene.

In certain embodiments, R³ represents independently for each occurrence C₁₋₂₀ alkyl, C₁₋₂₀ haloalkyl, —(C₁₋₁₀ alkylene)-X—(C₁₋₂₀ alkyl), —P(O)(OH)₂, or —P(O)(OH)—O—P(O)(OH)₂; wherein said C₁₋₂₀ alkyl, C₁₋₂₀ haloalkyl, and C₁₋₁₀ alkylene are optionally substituted with one hydroxyl or C₁₋₂₀ alkoxyl; and wherein one methylene unit in each of said C₁₋₂₀ alkyl, C₁₋₂₀ haloalkyl, and C₁₋₁₀ alkylene is optionally replaced with a C₃₋₅ cycloalkylene; and wherein one occurrence of R³ is additionally selected from hydrogen.

In certain embodiments, R³ represents independently for each occurrence C₁₋₂₀ alkyl, C₁₋₂₀ haloalkyl, or —(C₁₋₁₀ alkylene)-X—(C₁₋₂₀ alkyl); wherein said C₁₋₂₀ alkyl, C₁₋₂₀ haloalkyl, and C₁₋₁₀ alkylene are optionally substituted with one hydroxyl or C₁₋₂₀ alkoxyl; and wherein one methylene unit in each of said C₁₋₂₀ alkyl, C₁₋₂₀ haloalkyl, and C₁₋₁₀ alkylene is optionally replaced with a C₃₋₅ cycloalkylene. In certain embodiments, R³ represents independently for each occurrence C₁₋₂₀ alkyl, C₁₋₂₀ haloalkyl, or —(C₁₋₁₀ alkylene)-X—(C₁₋₂₀ alkyl); wherein said C₁₋₂₀ alkyl, C₁₋₂₀ haloalkyl, and C₁₋₁₀ alkylene are optionally substituted with one hydroxyl or C₁₋₂₀ alkoxyl. In certain embodiments, R³ represents independently for each occurrence C₁₋₂₀ alkyl, C₁₋₂₀ haloalkyl, or —(C₁₋₁₀ alkylene)-X—(C₁₋₂₀ alkyl); wherein one methylene unit in each of said C₁₋₂₀ alkyl, C₁₋₂₀ haloalkyl, and C₁₋₁₀ alkylene is optionally replaced with a C₃₋₅ cycloalkylene. In certain embodiments, R³ represents independently for each occurrence C₁₋₂₀ alkyl, C₁₋₂₀ haloalkyl, or —(C₁₋₁₀ alkylene)-X—(C₁₋₂₀ alkyl).

In certain embodiments, R³ represents independently for each occurrence —(C₁₋₁₀ alkylene)-X—(C₁₋₂₀ alkyl); wherein said C₁₋₂₀ alkyl and C₁₋₁₀ alkylene are optionally substituted with one hydroxyl or C₁₋₂₀ alkoxyl; and wherein one methylene unit in said C₁₋₂₀ alkyl and C₁₋₁₀ alkylene is optionally replaced with a C₃₋₅ cycloalkylene. In certain embodiments, R³ represents independently for each occurrence —(C₁₋₁₀ alkylene)-X—(C₁₋₂₀ alkyl); wherein said C₁₋₂₀ alkyl and C₁₋₁₀ alkylene are optionally substituted with one hydroxyl or C₁₋₂₀ alkoxyl. In certain embodiments, R³ represents independently for each occurrence —(C₁₋₁₀ alkylene)-X—(C₁₋₂₀ alkyl); wherein one methylene unit in said C₁₋₂₀ alkyl and C₁₋₁₀ alkylene is optionally replaced with a C₃₋₅ cycloalkylene. In certain embodiments, R³ represents independently for each occurrence —(C₁₋₁₀ alkylene)-X—(C₁₋₂₀ alkyl).

In certain embodiments, R³ represents independently for each occurrence —(C₁₋₁₀ alkylene)-OC(O)O—(C₁₋₁₀ alkyl) or —(C₁₋₁₀ alkylene)-OC(O)—N(R⁹)—(C₁₋₁₀ alkyl); wherein one methylene unit in each of said C₁₋₁₀ alkyl is optionally replaced with a C₃₋₅ cycloalkylene. In certain embodiments, R³ represents independently for each occurrence —(C₁₋₁₀ alkylene)-OC(O)O—(C₁₋₁₀ alkyl) or —(C₁₋₁₀ alkylene)-OC(O)—N(H)—(C₁₋₁₀ alkyl); wherein one methylene unit in each of said C₁₋₁₀ alkyl is optionally replaced with a C₃₋₅ cycloalkylene. In certain embodiments, R³ represents independently for each occurrence —CH₂—OC(O)O—(C₁₋₁₀ alkyl) or —CH₂—OC(O)—N(H)—(C₁₋₁₀ alkyl); wherein one methylene unit in each of said C₁₋₁₀ alkyl is optionally replaced with a C₃₋₅ cycloalkylene. In certain embodiments, R³ represents independently for each occurrence —CH₂—OC(O)O—(C₁₋₆ alkyl) or —CH₂—OC(O)—N(H)—(C₁₋₆ alkyl). In certain embodiments, R³ represents independently for each occurrence —CH₂—OC(O)O—(C₃₋₅ cycloalkyl) or —CH₂—OC(O)—N(H)—(C₃₋₅ cycloalkyl).

In certain embodiments, R³ represents independently for each occurrence —(C₁₋₁₀ alkylene)-OC(O)O—(C₁₋₁₀ alkyl), wherein one methylene unit in said C₁₋₁₀ alkyl is optionally replaced with a C₃₋₅ cycloalkylene. In certain embodiments, R³ represents independently for each occurrence —CH₂—OC(O)O—(C₁₋₁₀ alkyl), wherein one methylene unit in said C₁₋₁₀ alkyl is optionally replaced with a C₃₋₅ cycloalkylene. In certain embodiments, R³ represents independently for each occurrence —CH₂—OC(O)O—(C₁₋₆ alkyl). In certain embodiments, R³ represents independently for each occurrence —CH₂—OC(O)O—(C₃₋₅ cycloalkyl).

In certain embodiments, R³ represents independently for each occurrence —(C₁₋₁₀ alkylene)-OC(O)—N(R⁹)—(C₁₋₁₀ alkyl), wherein one methylene unit in said C₁₋₁₀ alkyl is optionally replaced with a C₃₋₅ cycloalkylene. In certain embodiments, R³ represents independently for each occurrence —(C₁₋₁₀ alkylene)-OC(O)—N(H)—(C₁₋₁₀ alkyl), wherein one methylene unit in said C₁₋₁₀ alkyl is optionally replaced with a C₃₋₅ cycloalkylene. In certain embodiments, R³ represents independently for each occurrence —CH₂—OC(O)—N(H)—(C₁₋₁₀ alkyl); wherein one methylene unit in said C₁₋₁₀ alkyl is optionally replaced with a C₃₋₅ cycloalkylene. In certain embodiments, R³ represents independently for each occurrence —CH₂—OC(O)—N(R⁹)—(C₁₋₆ alkyl). In certain embodiments, R³ represents independently for each occurrence —CH₂—OC(O)—N(R⁹)—(C₃₋₅ cycloalkyl).

In certain embodiments, R³ represents independently for each occurrence —(C₁₋₁₀ alkylene)-OC(O)—(C₁₋₁₀ alkyl) or —(C₁₋₁₀ alkylene)-SC(O)—(C₁₋₁₀ alkyl); wherein said C₁₋₁₀ alkyl is optionally substituted with one hydroxyl; and wherein one methylene unit in each of said C₁₋₁₀ alkyl is optionally replaced with a C₃₋₅ cycloalkylene. In certain embodiments, R³ represents independently for each occurrence —(C₁₋₁₀ alkylene)-OC(O)—(C₁₋₁₀ alkyl) or —(C₁₋₁₀ alkylene)-SC(O)—(C₁₋₁₀ alkyl); wherein said C₁₋₁₀ alkyl is substituted with one hydroxyl; and wherein one methylene unit in each of said C₁₋₁₀ alkyl is optionally replaced with a C₃₋₅ cycloalkylene. In certain embodiments, R³ represents independently for each occurrence —(C₁₋₁₀ alkylene)-OC(O)—(C₁₋₁₀ alkyl) or —(C₁₋₁₀ alkylene)-SC(O)—(C₁₋₁₀ alkyl); wherein said C₁₋₁₀ alkyl is substituted with one hydroxyl; and wherein one methylene unit in each of said C₁₋₁₀ alkyl is replaced with a C₃₋₅ cycloalkylene. In certain embodiments, R³ represents independently for each occurrence —(C₁₋₁₀ alkylene)-OC(O)—(C₁₋₁₀ alkyl) or —(C₁₋₁₀ alkylene)-SC(O)—(C₁₋₁₀ alkyl); wherein said C₁₋₁₀ alkyl is substituted with one hydroxyl.

In certain embodiments, R³ represents independently for each occurrence —(CH₂)₁₋₂—OC(O)—(C₁₋₁₀ alkyl) or —(CH₂)₁₋₂—SC(O)—(C₁₋₁₀ alkyl); wherein said C₁₋₁₀ alkyl is optionally substituted with one hydroxyl; and wherein one methylene unit in each of said C₁₋₁₀ alkyl is optionally replaced with a cyclopropylene. In certain embodiments, R³ represents independently for each occurrence —(CH₂)₁₋₂—OC(O)—(C₁₋₁₀ alkyl) or —(CH₂)₁₋₂—SC(O)—(C₁₋₁₀ alkyl); wherein said C₁₋₁₀ alkyl is substituted with one hydroxyl; and wherein one methylene unit in each of said C₁₋₁₀ alkyl is optionally replaced with a cyclopropylene. In certain embodiments, R³ represents independently for each occurrence —(CH₂)₁₋₂—OC(O)—(C₁₋₁₀ alkyl) or —(CH₂)₁₋₂—SC(O)—(C₁₋₁₀ alkyl); wherein said C₁₋₁₀ alkyl is substituted with one hydroxyl; and wherein one methylene unit in each of said C₁₋₁₀ alkyl is replaced with a cyclopropylene. In certain embodiments, R³ represents independently for each occurrence —(CH₂)₁₋₂—OC(O)—(C₁₋₁₀ alkyl) or —(CH₂)₁₋₂—SC(O)—(C₁₋₁₀ alkyl); wherein said C₁₋₁₀ alkyl is substituted with one hydroxyl.

In certain embodiments, R³ represents independently for each occurrence —(CH₂)₁₋₂—OC(O)—(C₁₋₁₀ alkyl); wherein said C₁₋₁₀ alkyl is optionally substituted with one hydroxyl; and wherein one methylene unit in said C₁₋₁₀ alkyl is optionally replaced with a cyclopropylene. In certain embodiments, R³ represents independently for each occurrence —(CH₂)₁₋₂—OC(O)—(C₁₋₁₀ alkyl); wherein said C₁₋₁₀ alkyl is substituted with one hydroxyl; and wherein one methylene unit in said C₁₋₁₀ alkyl is optionally replaced with a cyclopropylene. In certain embodiments, R³ represents independently for each occurrence —(CH₂)₁₋₂—OC(O)—(C₁₋₁₀ alkyl); wherein said C₁₋₁₀ alkyl is substituted with one hydroxyl; and wherein one methylene unit in said C₁₋₁₀ alkyl is replaced with a cyclopropylene. In certain embodiments, R³ represents independently for each occurrence —(CH₂)₁₋₂—OC(O)—(C₁₋₁₀ alkyl), wherein said C₁₋₁₀ alkyl is substituted with one hydroxyl.

In certain embodiments, R³ represents independently for each occurrence —(CH₂)₁₋₂—SC(O)—(C₁₋₁₀ alkyl); wherein said C₁₋₁₀ alkyl is optionally substituted with one hydroxyl; and wherein one methylene unit in said C₁₋₁₀ alkyl is optionally replaced with a cyclopropylene. In certain embodiments, R³ represents independently for each occurrence —(CH₂)₁₋₂—SC(O)—(C₁₋₁₀ alkyl); wherein said C₁₋₁₀ alkyl is substituted with one hydroxyl; and wherein one methylene unit in said C₁₋₁₀ alkyl is optionally replaced with a cyclopropylene. In certain embodiments, R³ represents independently for each occurrence —(CH₂)₁₋₂—SC(O)—(C₁₋₁₀ alkyl); wherein said C₁₋₁₀ alkyl is substituted with one hydroxyl; and wherein one methylene unit in said C₁₋₁₀ alkyl is replaced with a cyclopropylene. In certain embodiments, R³ represents independently for each occurrence —(CH₂)₁₋₂—SC(O)—(C₁₋₁₀ alkyl), wherein said C₁₋₁₀ alkyl is substituted with one hydroxyl.

In certain embodiments, R³ represents independently for each occurrence

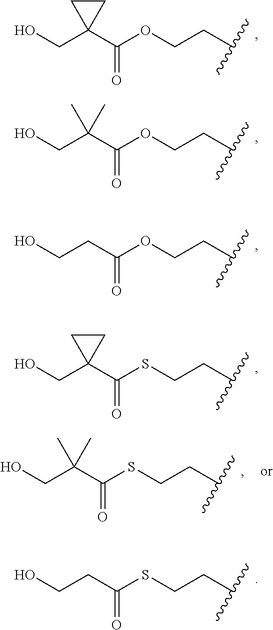

In certain embodiments, R³ represents independently for each occurrence

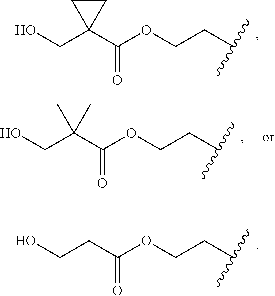

In certain embodiments, R³ represents independently for each occurrence

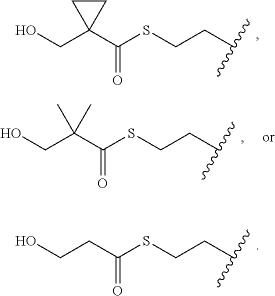

In certain embodiments, R³ is

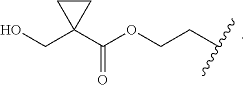 [SMILES: *CCOC(=O)C1(CO)CC1]

In certain embodiments, R³ is

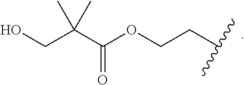 [SMILES: *CCOC(=O)C(C)(C)CO]

In certain embodiments, R³ is

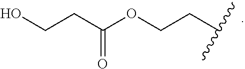 [SMILES: *CCOC(=O)CCO]

In certain embodiments, R³ is

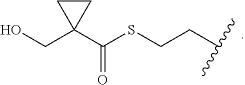 [SMILES: *CCSC(=O)C1(CO)CC1]

In certain embodiments, R³ is

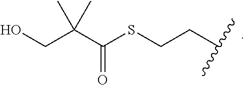 [SMILES: *CCSC(=O)C(C)(C)CO]

In certain embodiments, R³ is

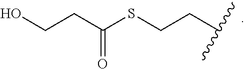 [SMILES: *CCSC(=O)CCO]

In certain embodiments, R³ represents independently for each occurrence —(C₁₋₁₀ alkylene)-O—(C₁₋₂₀ alkyl) or —(C₁₋₁₀ alkylene)-S—(C₁₋₂₀ alkyl); wherein said C₁₋₁₀ alkylene is optionally substituted with one C₁₋₂₀ alkoxyl; and wherein one occurrence of R³ is additionally selected from hydrogen. In certain embodiments, one occurrence of R³ is —(C₁₋₁₀ alkylene)-O—(C₁₋₂₀ alkyl) or —(C₁₋₁₀ alkylene)-S—(C₁₋₂₀ alkyl); wherein said C₁₋₁₀ alkylene is optionally substituted with one C₁₋₂₀ alkoxyl; and any second occurrence of R³ is hydrogen. In certain embodiments, one occurrence of R³ is —(C₁₋₁₀ alkylene)-O—(C₁₋₂₀ alkyl) or —(C₁₋₁₀ alkylene)-S—(C₁₋₂₀ alkyl); wherein said C₁₋₁₀ alkylene is substituted with one C₁₋₂₀ alkoxyl; and any second occurrence of R³ is hydrogen. In certain embodiments, one occurrence of R³ is —(C₁₋₁₀ alkylene)-O—(C₁₋₂₀ alkyl) or —(C₁₋₁₀ alkylene)-S—(C₁₋₂₀ alkyl); and any second occurrence of R³ is hydrogen.

In certain embodiments, one occurrence of R³ is —CH₂—C(H)(—O—(C₁₋₂₀ alkyl))-CH₂—O—(C₁₋₂₀ alkyl), —(CH₂)₃—O—(C₁₋₂₀ alkyl), —CH₂—C(H)(—O—(C₁₋₂₀ alkyl))-CH₂—S—(C₁₋₂₀ alkyl), or —(CH₂)₃—S—(C₁₋₂₀ alkyl); and any second occurrence of R³ is hydrogen. In certain embodiments, one occurrence of R³ is —CH₂—C(H)(—O—(C₁₋₂₀ alkyl))-CH₂—O—(C₁₋₂₀ alkyl) or —(CH₂)₃—O—(C₁₋₂₀ alkyl), and any second occurrence of R³ is hydrogen. In certain embodiments, one occurrence of R³ is —CH₂—C(H)(—O—(C₁₋₂₀ alkyl))-CH₂—O—(C₁₋₂₀ alkyl), and any second occurrence of R³ is hydrogen. In certain embodiments, one occurrence of R³ is —(CH₂)₃—O—(C₁₋₂₀ alkyl), and any second occurrence of R³ is hydrogen. In certain embodiments, one occurrence of R³ is —CH₂—C(H)(—O—(C₁₋₂₀ alkyl))-CH₂—S—(C₁₋₂₀ alkyl) or —(CH₂)₃—S—(C₁₋₂₀ alkyl), and any second occurrence of R³ is hydrogen. In certain embodiments, one occurrence of R³ is —CH₂—C(H)(—O—(C₁₋₂₀ alkyl))-CH₂—S—(C₁₋₂₀ alkyl), and any second occurrence of R³ is hydrogen. In certain embodiments, one occurrence of R³ is —(CH₂)₃—S—(C₁₋₂₀ alkyl), and any second occurrence of R³ is hydrogen.

In certain embodiments, R³ represents independently for each occurrence C₁₋₂₀ alkyl optionally substituted with one hydroxyl. In certain embodiments, R³ represents independently for each occurrence C₁₋₇ alkyl optionally substituted with one hydroxyl. In certain embodiments, R³ represents independently for each occurrence C₁₋₄ alkyl optionally substituted with one hydroxyl. In certain embodiments, R³ represents independently for each occurrence C₁₋₂₀ haloalkyl optionally substituted with one hydroxyl. In certain embodiments, R³ represents independently for each occurrence C₁₋₁₀ haloalkyl optionally substituted with one hydroxyl. In certain embodiments, R³ represents independently for each occurrence C₁₋₄ haloalkyl optionally substituted with one hydroxyl.

In certain embodiments, R³ represents independently for each occurrence —(C₁₋₁₀ alkylene)-O—(C₁₋₁₀ alkyl), wherein said C₁₋₁₀ alkyl is optionally substituted with one hydroxyl. In certain embodiments, R³ represents independently for each occurrence —(C₁₋₁₀ alkylene)-OC(O)—(C₁₋₁₀ alkyl), wherein said C₁₋₁₀ alkyl is optionally substituted with one hydroxyl. In certain embodiments, R³ represents independently for each occurrence —(C₁₋₁₀ alkylene)-S—(C₁₋₁₀ alkyl), wherein said C₁₋₁₀ alkyl is optionally substituted with one hydroxyl. In certain embodiments, R³ represents independently for each occurrence —(C₁₋₁₀ alkylene)-SC(O)—(C₁₋₁₀ alkyl), wherein said C₁₋₁₀ alkyl is optionally substituted with one hydroxyl.

In certain embodiments, R³ represents independently for each occurrence C₁₋₂₀ alkyl. In certain embodiments, R³ represents independently for each occurrence C₁₋₇ alkyl. In certain embodiments, R³ represents independently for each occurrence C₁₋₄ alkyl. In certain embodiments, R³ represents independently for each occurrence C₁₋₂₀ haloalkyl. In certain embodiments, R³ represents independently for each occurrence C₁₋₁₀ haloalkyl. In certain embodiments, R³ represents independently for each occurrence C₁₋₄ haloalkyl. In certain embodiments, R³ represents independently for each occurrence —(C₁₋₁₀ alkylene)-O—(C₁₋₁₀ alkyl). In certain embodiments, R³ represents independently for each occurrence —(C₁₋₁₀ alkylene)-OC(O)—(C₁₋₁₀ alkyl). In certain embodiments, R³ represents independently for each occurrence —(C₁₋₁₀ alkylene)-S—(C₁₋₁₀ alkyl). In certain embodiments, R³ represents independently for each occurrence —(C₁₋₁₀ alkylene)-SC(O)—(C₁₋₁₀ alkyl).

In certain embodiments, R³ represents independently for each occurrence

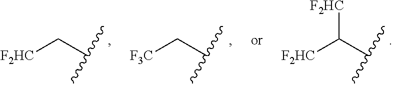 [SMILES: *CC(F)F ; *CC(F)(F)F ; *C(C(F)F)C(F)F]

In certain embodiments, R³ is

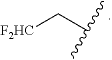 [SMILES: *CC(F)F]

In certain embodiments, R³ is

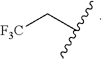 [SMILES: *CC(F)(F)F]

In certain embodiments, R³ is

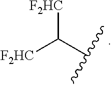 [SMILES: *C(C(F)F)C(F)F]

In certain embodiments, R³ represents independently for each occurrence hydrogen, —P(O)(OH)₂, or —P(O)(OH)—O—P(O)(OH)₂. In certain embodiments, one occurrence of R³ is hydrogen, and any second occurrence of R³ is hydrogen, —P(O)(OH)₂, or —P(O)(OH)—O—P(O)(OH)₂. In certain embodiments, R³ is hydrogen. In certain embodiments, 0 or 1 occurrence of R³ is hydrogen. In certain embodiments, one occurrence of R³ is hydrogen, and any second occurrence of R³ is —P(O)(OH)₂ or —P(O)(OH)—O—P(O)(OH)₂. In certain embodiments, one occurrence of R³ is hydrogen, and any second occurrence of R³ is —P(O)(OH)₂. In certain embodiments, one occurrence of R³ is hydrogen, and any second occurrence of R³ is —P(O)(OH)—O—P(O)(OH)₂.

In certain embodiments, two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸.

In certain embodiments, two instances of R³ are taken together to form a C₂₋₄ bivalent hydrocarbon chain substituted with p instances of R⁸. In certain embodiments, two instances of R³ are taken together to form a C₂₋₄ bivalent hydrocarbon chain substituted with one instance of —(C₁₋₃ alkylene)-CO₂R¹⁰. In certain embodiments, two instances of R³ are taken together to form a C₂₋₄ bivalent hydrocarbon chain.

In certain embodiments, two instances of R³ are taken together to form a C₂₋₄ bivalent hydrocarbon chain ortho-fused to a 6-membered aromatic ring having 0, 1, or 2 nitrogen atoms; wherein said ring is substituted with p instances of R⁸. In certain embodiments, two instances of R³ are taken together to form a C₂₋₄ bivalent hydrocarbon chain ortho-fused to a 6-membered aromatic ring having 0, 1, or 2 nitrogen atoms.

In certain embodiments, two instances of R³ are taken together to form a C₂₋₄ bivalent hydrocarbon chain ortho-fused to a 6-membered carbocyclic aromatic ring; wherein said ring is substituted with p instances of R⁸. In certain embodiments, two instances of R³ are taken together to form a C₃ bivalent hydrocarbon chain ortho-fused to a 6-membered carbocyclic aromatic ring; wherein said ring is substituted with p instances of R⁸. In certain embodiments, two instances of R³ are taken together to form a C₃ bivalent hydrocarbon chain ortho-fused to a 6-membered carbocyclic aromatic ring.

In certain embodiments, two instances of R³ are taken together to form a C₂₋₄ bivalent hydrocarbon chain ortho-fused to a 6-membered aromatic ring having 1 or 2 nitrogen atoms; wherein said ring is substituted with p instances of R⁸. In certain embodiments, two instances of R³ are taken together to form a C₃ bivalent hydrocarbon chain ortho-fused to a 6-membered aromatic ring having 1 or 2 nitrogen atoms; wherein said ring is substituted with p instances of R⁸. In certain embodiments, two instances of R³ are taken together to form a C₃ bivalent hydrocarbon chain ortho-fused to a 6-membered aromatic ring having 1 or 2 nitrogen atoms.

In certain embodiments, R³ is selected from the groups depicted in the compounds in Tables 1, 2, 3, 4, 5, and 6, below.

As defined generally above, R⁴ represents independently for each occurrence hydrogen or C₁₋₄ alkyl; or R⁴ and one occurrence of R⁶ are taken together with the atoms to which they are attached to form a 4-7 membered saturated heterocyclic ring containing 1 nitrogen atom. In certain embodiments, R⁴ represents independently for each occurrence hydrogen or C₁₋₄ alkyl. In certain embodiments, R⁴ represents independently for each occurrence C₁₋₄ alkyl.

In certain embodiments, R⁴ represents independently for each occurrence hydrogen or methyl; or R⁴ and one occurrence of R⁶ are taken together with the atoms to which they are attached to form a 4-7 membered saturated heterocyclic ring containing 1 nitrogen atom.

In certain embodiments, R⁴ represents independently for each occurrence hydrogen or methyl. In certain embodiments, R⁴ is hydrogen. In certain embodiments, R⁴ is methyl. In certain embodiments, R⁴ and one occurrence of R⁶ are taken together with the atoms to which they are attached to form a 4-7 membered saturated heterocyclic ring containing 1 nitrogen atom.

In certain embodiments, R⁴ is selected from the groups depicted in the compounds in Tables 1, 2, 3, 4, 5, and 6, below.

In certain embodiments, —N(R⁴)(R⁵) is

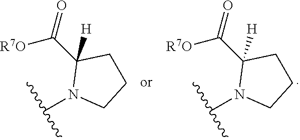

In certain embodiments, —N(R⁴)(R⁵) is

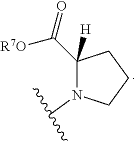

In certain embodiments, —N(R⁴)(R⁵) is

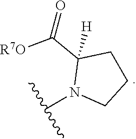

In certain embodiments, —N(R⁴)(R⁵) is selected from the groups depicted in the compounds in Tables 1, 2, 3, 4, 5, and 6, below.

As defined generally above, R⁵ represents independently for each occurrence:
a. —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, —C(R¹⁰)(H)—CH₂—CO₂R¹⁰, C₁₋₂₀ alkyl, C₁₋₂₀ haloalkyl, —(C₁₋₁₀ alkylene)-X—(C₁₋₁₀ alkyl), —(C₁₋₁₀ alkylene)-phenyl, —(C₁₋₄ haloalkylene)-phenyl, —C(O)-phenyl, —C(S)-phenyl, or —C(O)-(5-6 membered monocyclic heteroaryl having 1, 2, or 3 heteroatoms independently selected from nitrogen, oxygen, and sulfur); or
b. phenyl; naphthyl; a 5-6 membered monocyclic heteroaryl having 1, 2, or 3 heteroatoms independently selected from nitrogen, oxygen, and sulfur; or an 8-10 membered bicyclic heteroaryl having 1, 2, or 3 heteroatoms independently selected from nitrogen, oxygen, and sulfur;
   wherein said phenyl, naphthyl, 5-6 membered monocyclic heteroaryl, and 8-10 membered bicyclic heteroaryl are substituted with m instances of R⁸; and said C₁₋₂₀ alkyl, C₁₋₂₀ haloalkyl, and C₁₋₁₀ alkyl are optionally substituted with one hydroxyl;
   or R³ and R⁵ are taken together to form a C₂₋₄ bivalent hydrocarbon chain optionally ortho-fused to a 6-membered aromatic ring having 0, 1, or 2 nitrogen atoms; wherein said ring is substituted with p instances of R⁸.

In certain embodiments, R⁵ represents independently for each occurrence —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, —C(R¹⁰)(H)—CH₂—CO₂R¹⁰, C₁₋₂₀ alkyl, C₁₋₂₀ haloalkyl, —(C₁₋₁₀ alkylene)-X—(C₁₋₁₀ alkyl), —(C₁₋₁₀ alkylene)-phenyl, —(C₁₋₄ haloalkylene)-phenyl, —C(O)-phenyl, —C(S)-phenyl, or —C(O)-(5-6 membered monocyclic heteroaryl having 1, 2, or 3 heteroatoms independently selected from nitrogen, oxygen, and sulfur); wherein said phenyl and 5-6 membered monocyclic heteroaryl are substituted with m instances of R⁸; and said C₁₋₂₀ alkyl, C₁₋₂₀ haloalkyl, and C₁₋₁₀ alkyl are optionally substituted with one hydroxyl. In certain embodiments, R⁵ represents independently for each occurrence —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, —C(R¹⁰)(H)—CH₂—CO₂R¹⁰, C₁₋₂₀ alkyl, C₁₋₂₀ haloalkyl, —(C₁₋₁₀ alkylene)-X—(C₁₋₁₀ alkyl), —(C₁₋₁₀ alkylene)-phenyl, —(C₁₋₄ haloalkylene)-phenyl, —C(O)-phenyl, —C(S)-phenyl, or —C(O)-(5-6 membered monocyclic heteroaryl having 1, 2, or 3 heteroatoms independently selected from nitrogen, oxygen, and sulfur).

In certain embodiments, R⁵ represents independently for each occurrence —C(R⁶)₂—CO₂R⁷, —(C₁₋₁₀ alkylene)-phenyl, —(C₁₋₄ haloalkylene)-phenyl, —C(O)-phenyl, —C(S)-phenyl, or —C(O)-(5-6 membered monocyclic heteroaryl having 1, 2, or 3 heteroatoms independently selected from nitrogen, oxygen, and sulfur); wherein said phenyl and 5-6 membered monocyclic heteroaryl are substituted with m instances of R⁸.

In certain embodiments, R⁵ represents independently for each occurrence —C(R⁶)₂—CO₂R⁷, —(C₁₋₁₀ alkylene)-phenyl, or —(C₁₋₄ haloalkylene)-phenyl; wherein said phenyl is substituted with m instances of R⁸. In certain embodiments, R⁵ represents independently for each occurrence —C(R⁶)₂—CO₂R⁷ or —(C₁₋₄ alkylene)-phenyl; wherein said phenyl is substituted with m instances of R⁸.

In certain embodiments, R⁵ represents independently for each occurrence —C(R⁶)₂—CO₂R⁷, —C(O)-phenyl, —C(S)-phenyl, or —C(O)-(5-6 membered monocyclic heteroaryl having 1, 2, or 3 heteroatoms independently selected from nitrogen, oxygen, and sulfur); wherein said phenyl and 5-6 membered monocyclic heteroaryl are substituted with m instances of R⁸. In certain embodiments, R⁵ represents independently for each occurrence —C(R⁶)₂—CO₂R⁷ or —C(O)— phenyl; wherein said phenyl and 5-6 membered monocyclic heteroaryl are substituted with m instances of R⁸.

In certain embodiments, R⁵ represents independently for each occurrence —C(R⁶)₂—CO₂R⁷, C₁₋₂₀ alkyl, C₁₋₂₀ haloalkyl, —(C₁₋₁₀ alkylene)-X—(C₁₋₁₀ alkyl), or —(C₁₋₁₀ alkylene)-phenyl; wherein said phenyl is substituted with m instances of R⁸; and said C₁₋₂₀ alkyl, C₁₋₂₀ haloalkyl, and C₁₋₁₀ alkyl are optionally substituted with one hydroxyl. In certain embodiments, R⁵ represents independently for each occurrence —C(R⁶)₂—CO₂R⁷, C₁₋₂₀ alkyl, C₁₋₂₀ haloalkyl, or —(C₁₋₁₀ alkylene)-X—(C₁₋₁₀ alkyl); wherein said C₁₋₂₀ alkyl, C₁₋₂₀ haloalkyl, and C₁₋₁₀ alkyl are optionally substituted with one hydroxyl.

In certain embodiments, R⁵ represents independently for each occurrence —C(R⁶)₂—CO₂R⁷, C₁₋₂₀ alkyl, C₁₋₂₀ haloalkyl, —(C₁₋₁₀ alkylene)-X—(C₁₋₁₀ alkyl), or —(C₁₋₁₀ alkylene)-phenyl. In certain embodiments, R⁵ represents independently for each occurrence —C(R⁶)₂—CO₂R⁷, C₁₋₂₀ alkyl, C₁₋₂₀ haloalkyl, or —(C₁₋₁₀ alkylene)-X—(C₁₋₁₀ alkyl).

In certain embodiments, R⁵ represents independently for each occurrence —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, or —C(R¹⁰)(H)—CH₂—CO₂R¹⁰. In certain embodiments, R⁵ represents independently for each occurrence —C(R⁶)₂—CO₂R⁷, —C(R⁶)₂—C(O)N(R⁹)₂, or —C(R⁶)₂—C(O)SR¹⁰. In certain embodiments, R⁵ represents independently for each occurrence —C(R⁶)₂—C(O)N(R⁹)₂, —C(R⁶)₂—C(O)SR¹⁰, —CH₂—C(R¹⁰)(H)—CO₂R¹⁰, or —C(R¹⁰)(H)—CH₂—CO₂R¹⁰. In certain embodiments, R⁵ represents independently for each occurrence —C(R⁶)₂—C(O)N(R⁹)₂ or —C(R⁶)₂—C(O)SR¹⁰. In certain embodiments, R⁵ represents independently for each occurrence —CH₂—C(R¹⁰)(H)—CO₂R¹⁰ or —C(R¹⁰)(H)—CH₂—CO₂R¹⁰.

In certain embodiments, R⁵ represents independently for each occurrence —C(R⁶)₂—C(O)N(R⁹)₂. In certain embodiments, R⁵ represents independently for each occurrence —C(R⁶)₂—C(O)SR¹⁰. In certain embodiments, R⁵ represents independently for each occurrence —CH₂—C(R¹⁰)(H)—CO₂R¹⁰. In certain embodiments, R⁵ represents independently for each occurrence —C(R¹⁰)(H)—CH₂—CO₂R¹⁰.

In certain embodiments, R⁵ represents independently for each occurrence —C(R⁶)₂—CO₂R⁷. In certain embodiments, R⁵ represents independently for each occurrence —C(H)(R⁶)—CO₂R⁷. In certain embodiments, R⁵ represents independently for each occurrence

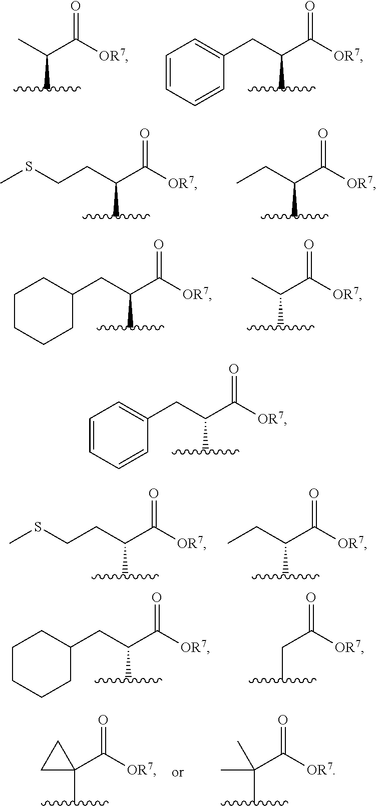

In certain embodiments, R⁵ represents independently for each occurrence

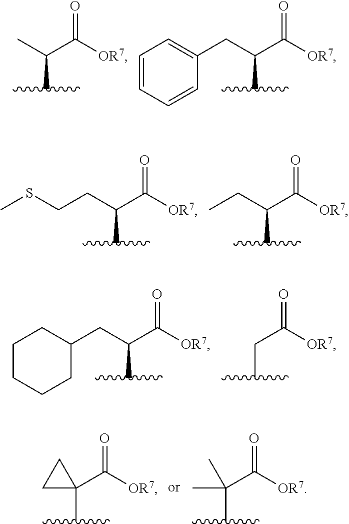

In certain embodiments, R⁵ represents independently for each occurrence

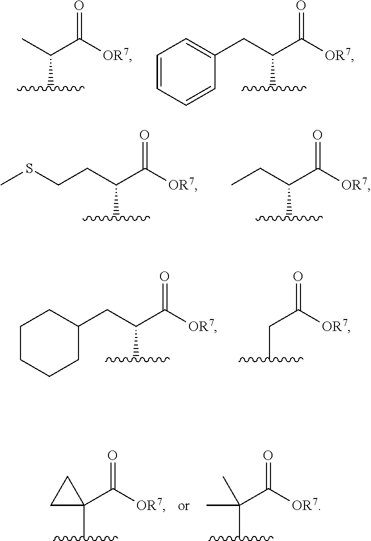

In certain embodiments, R⁵ is

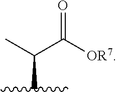

In certain embodiments, R⁵ is

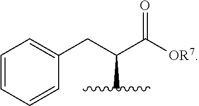

In certain embodiments, R⁵ is

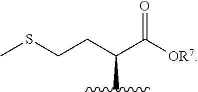

In certain embodiments, R⁵ is

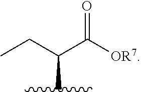

In certain embodiments, R⁵ is

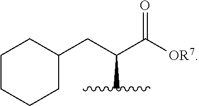

In certain embodiments, R⁵ is

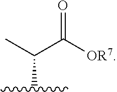

In certain embodiments, R⁵ is

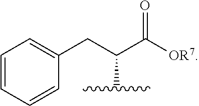

In certain embodiments, R⁵ is

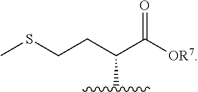

In certain embodiments, R⁵ is

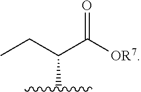

In certain embodiments, R⁵ is

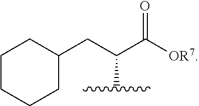

In certain embodiments, R⁵ is

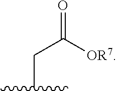

In certain embodiments, R⁵ is

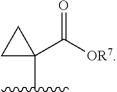

In certain embodiments, R⁵ is

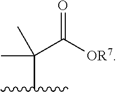

In certain embodiments, R⁵ represents independently for each occurrence C₁₋₂₀ alkyl, C₁₋₂₀ haloalkyl, or —(C₁₋₁₀ alkylene)-X—(C₁₋₁₀ alkyl); wherein said C₁₋₂₀ alkyl, C₁₋₂₀ haloalkyl, and C₁₋₁₀ alkyl are optionally substituted with one hydroxyl. In certain embodiments, R⁵ represents independently for each occurrence —(C₁₋₁₀ alkylene)-X—(C₁₋₁₀ alkyl); wherein said C₁₋₁₀ alkyl is optionally substituted with one hydroxyl.

In certain embodiments, R⁵ represents independently for each occurrence C₁₋₂₀ alkyl, C₁₋₂₀ haloalkyl, or —(C₁₋₁₀ alkylene)-X—(C₁₋₁₀ alkyl). In certain embodiments, R⁵ represents independently for each occurrence —(C₁₋₁₀ alkylene)-X—(C₁₋₁₀ alkyl).

In certain embodiments, R⁵ is C₁₋₂₀ alkyl optionally substituted with one hydroxyl. In certain embodiments, R⁵ is C₁₋₇ alkyl optionally substituted with one hydroxyl. In certain embodiments, R⁵ is C₁₋₄ alkyl optionally substituted with one hydroxyl. In certain embodiments, R⁵ is C₁₋₂₀ haloalkyl optionally substituted with one hydroxyl. In certain embodiments, R⁵ is —(C₁₋₁₀ alkylene)-O—(C₁₋₁₀ alkyl), wherein said C₁₋₁₀ alkyl is optionally substituted with one hydroxyl. In certain embodiments, R⁵ is —(C₁₋₁₀ alkylene)-OC(O)—(C₁₋₁₀ alkyl), wherein said C₁₋₁₀ alkyl is optionally substituted with one hydroxyl. In certain embodiments, R⁵ is —(C₁₋₁₀ alkylene)-S—(C₁₋₁₀ alkyl), wherein said C₁₋₁₀ alkyl is optionally substituted with one hydroxyl. In certain embodiments, R⁵ is —(C₁₋₁₀ alkylene)-SC(O)—(C₁₋₁₀ alkyl), wherein said C₁₋₁₀ alkyl is optionally substituted with one hydroxyl.

In certain embodiments, R⁵ is C₁₋₂₀ alkyl. In certain embodiments, R⁵ is C₁₋₇ alkyl. In certain embodiments, R⁵ is C₁₋₄ alkyl. In certain embodiments, R⁵ is C₁₋₂₀ haloalkyl. In certain embodiments, R⁵ is —(C₁₋₁₀ alkylene)-O—(C₁₋₁₀ alkyl). In certain embodiments, R⁵ is —(C₁₋₁₀ alkylene)-OC(O)—(C₁₋₁₀ alkyl). In certain embodiments, R⁵ is —(C₁₋₁₀ alkylene)-S—(C₁₋₁₀ alkyl). In certain embodiments, R⁵ is —(C₁₋₁₀ alkylene)-SC(O)—(C₁₋₁₀ alkyl).

In certain embodiments, R⁵ is —(C₁₋₁₀ alkylene)-phenyl, phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein each of said phenyl, naphthyl, 5-6 membered monocyclic heteroaryl, and 8-10 membered bicyclic heteroaryl is substituted with m instances of R⁸. In certain embodiments, R⁵ is —(C₁₋₁₀ alkylene)-phenyl, phenyl, or naphthyl; wherein each of said phenyl and naphthyl is substituted with m instances of R⁸. In certain embodiments, R⁵ is a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; each of which is substituted with m instances of R⁸.

In certain embodiments, R⁵ is —(C₁₋₁₀ alkylene)-phenyl, phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur. In certain embodiments, R⁵ is —(C₁₋₁₀ alkylene)-phenyl, phenyl, or naphthyl. In certain embodiments, R⁵ is a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur.

In certain embodiments, R⁵ is —(C₁₋₁₀ alkylene)-phenyl, wherein said phenyl is substituted with m instances of R⁸. In certain embodiments, R⁵ is phenyl substituted with m instances of R⁸. In certain embodiments, R⁵ is naphthyl substituted with m instances of R⁸. In certain embodiments, R⁵ is a 5-6 membered monocyclic heteroaryl having 1, 2, or 3 heteroatoms independently selected from nitrogen, oxygen, and sulfur; wherein said heteroaryl is substituted with m instances of R⁸. In certain embodiments, R⁵ is an 8-10 membered bicyclic heteroaryl having 1, 2, or 3 heteroatoms independently selected from nitrogen, oxygen, and sulfur; wherein said heteroaryl is substituted with m instances of R⁸.

In certain embodiments, R⁵ is —(C₁₋₁₀ alkylene)-phenyl. In certain embodiments, R⁵ is phenyl. In certain embodiments, R⁵ is naphthyl. In certain embodiments, R⁵ is a 5-6 membered monocyclic heteroaryl having 1, 2, or 3 heteroatoms independently selected from nitrogen, oxygen, and sulfur. In certain embodiments, R⁵ is an 8-10 membered bicyclic heteroaryl having 1, 2, or 3 heteroatoms independently selected from nitrogen, oxygen, and sulfur.

In certain embodiments, R⁵ is selected from the groups depicted in the compounds in Tables 1, 2, 3, 4, 5, and 6, below.

As defined generally above, R⁶ represents independently for each occurrence C₁₋₆ alkyl, C₁₋₆ haloalkyl, C₃₋₅ cycloalkyl, —CN, or hydrogen, wherein said C₁₋₆ alkyl is optionally substituted with —S—(C₁₋₄ alkyl), —SH, C₁₋₄ alkoxyl, C₁₋₄ haloalkoxyl, hydroxyl, —OCH₂CN, phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; or two instances of R⁶ are taken together with the carbon atom to which they are attached to form a 3-6 membered saturated ring having 0 or 1 oxygen atom; or R⁴ and one occurrence of R⁶ are taken together with the atom or atoms to which they are attached to form a 4-7 membered saturated heterocyclic ring containing 1 nitrogen atom.

In certain embodiments, R⁶ represents independently for each occurrence C₁₋₆ alkyl, C₁₋₆ haloalkyl, C₃₋₅ cycloalkyl, or hydrogen, wherein said C₁₋₆ alkyl is optionally substituted with —S—(C₁₋₄ alkyl), —SH, C₁₋₄ alkoxyl, hydroxyl, phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; or two instances of R⁶ are taken together with the carbon atom to which they are attached to form a 3-5 membered saturated carbocyclic ring; or R⁴ and one occurrence of R⁶ are taken together with the atom or atoms to which they are attached to form a 4-7 membered saturated heterocyclic ring containing 1 nitrogen atom.

In certain embodiments, one occurrence of R⁶ is C₁₋₆ alkyl, C₁₋₆ haloalkyl, or hydrogen, wherein said C₁₋₆ alkyl is optionally substituted with —S—(C₁₋₄ alkyl), —SH, C₁₋₄ alkoxyl, C₁₋₄ haloalkoxyl, hydroxyl, —OCH₂CN, phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; or R⁴ and one occurrence of R⁶ are taken together with the atom or atoms to which they are attached to form a 4-7 membered saturated heterocyclic ring containing 1 nitrogen atom; and any geminal occurrence of R⁶ is hydrogen.

In certain embodiments, R⁶ represents independently for each occurrence hydrogen or C₁₋₆ alkyl optionally substituted with —S—(C₁₋₄ alkyl), —SH, C₁₋₄ alkoxyl, C₁₋₄ haloalkoxyl, hydroxyl, —OCH₂CN, phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur. In certain embodiments, R⁶ represents independently for each occurrence hydrogen or C₁₋₆ alkyl optionally substituted with —S—(C₁₋₄ alkyl) or —SH. In certain embodiments, R⁶ represents independently for each occurrence hydrogen or C₁₋₆ alkyl optionally substituted with C₁₋₄ alkoxyl, C₁₋₄ haloalkoxyl, hydroxyl, or —OCH₂CN.

In certain embodiments, R⁶ represents independently for each occurrence hydrogen or C₁₋₆ alkyl optionally substituted with phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur. In certain embodiments, R⁶ represents independently for each occurrence hydrogen or C₁₋₆ alkyl optionally substituted with phenyl or C₃₋₇ cycloalkyl. In certain embodiments, R⁶ represents independently for each occurrence hydrogen or C₁₋₆ alkyl optionally substituted with phenyl or a 5-6 membered monocyclic heteroaryl having 1, 2, or 3 heteroatoms independently selected from nitrogen, oxygen, and sulfur. In certain embodiments, R⁶ represents independently for each occurrence hydrogen or C₁₋₆ alkyl optionally substituted with an 8-10 membered bicyclic heteroaryl having 1, 2, or 3 heteroatoms independently selected from nitrogen, oxygen, and sulfur.

In certain embodiments, one occurrence of R⁶ is C₁₋₆ alkyl substituted with —S—(C₁₋₄ alkyl), —SH, C₁₋₄ alkoxyl, C₁₋₄ haloalkoxyl, hydroxyl, —OCH₂CN, phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; and any geminal occurrence of R⁶ is hydrogen. In certain embodiments, one occurrence of R⁶ is C₁₋₆ alkyl substituted with —S—(C₁₋₄ alkyl) or —SH; and any geminal occurrence of R⁶ is hydrogen. In certain embodiments, one occurrence of R⁶ is C₁₋₆ alkyl substituted with C₁₋₄ alkoxyl, C₁₋₄ haloalkoxyl, hydroxyl, or —OCH₂CN; and any geminal occurrence of R⁶ is hydrogen.

In certain embodiments, one occurrence of R⁶ is C₁₋₆ alkyl substituted with phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; and any geminal occurrence of R⁶ is hydrogen. In certain embodiments, one occurrence of R⁶ is C₁₋₆ alkyl substituted with phenyl, C₃₋₇ cycloalkyl, or a 5-6 membered monocyclic heteroaryl having 1, 2, or 3 heteroatoms independently selected from nitrogen, oxygen, and sulfur; and any geminal occurrence of R⁶ is hydrogen. In certain embodiments, one occurrence of R⁶ is C₁₋₆ alkyl substituted with phenyl or C₃₋₇ cycloalkyl; and any geminal occurrence of R⁶ is hydrogen. In certain embodiments, one occurrence of R⁶ is C₁₋₆ alkyl substituted with phenyl or a 5-6 membered monocyclic heteroaryl having 1, 2, or 3 heteroatoms independently selected from nitrogen, oxygen, and sulfur; and any geminal occurrence of R⁶ is hydrogen. In certain embodiments, one occurrence of R⁶ is C₁₋₆ alkyl substituted with an 8-10 membered bicyclic heteroaryl having 1, 2, or 3 heteroatoms independently selected from nitrogen, oxygen, and sulfur; and any geminal occurrence of R⁶ is hydrogen.

In certain embodiments, R⁶ represents independently for each occurrence hydrogen or C₁₋₆ alkyl optionally substituted with —S—(C₁₋₄ alkyl), phenyl, or C₃₋₇ cycloalkyl; or R⁴ and one occurrence of R⁶ are taken together with the atoms to which they are attached to form a 4-7 membered saturated heterocyclic ring containing 1 nitrogen atom. In certain embodiments, R⁶ is hydrogen or C₁₋₆ alkyl optionally substituted with —S—(C₁₋₄ alkyl), phenyl, or C₃₋₇ cycloalkyl; or R⁴ and one occurrence of R⁶ are taken together with the atoms to which they are attached to form a 4-7 membered saturated heterocyclic ring containing 1 nitrogen atom. In certain embodiments, R⁴ and one occurrence of R⁶ are taken together with the atoms to which they are attached to form a 4-7 membered saturated heterocyclic ring containing 1 nitrogen atom; and any geminal occurrence of R⁶ is hydrogen.

In certain embodiments, two instances of R⁶ are taken together with the carbon atom to which they are attached to form a 3-6 membered saturated ring having 0 or 1 oxygen atom. In certain embodiments, two instances of R⁶ are taken together with the carbon atom to which they are attached to form a 3-6 membered saturated ring having 1 oxygen atom. In certain embodiments, two instances of R⁶ are taken together with the carbon atom to which they are attached to form a 3-6 membered saturated carbocyclic ring.

In certain embodiments, R⁶ represents independently for each occurrence C₁₋₆ alkyl, C₁₋₆ haloalkyl, C₃₋₅ cycloalkyl, or hydrogen, wherein said C₁₋₆ alkyl is optionally substituted with —S—(C₁₋₄ alkyl), phenyl, or C₃₋₇ cycloalkyl; or two instances of R⁶ are taken together with the carbon atom to which they are attached to form a 3-5 membered saturated carbocyclic ring. In certain embodiments, R⁶ represents independently for each occurrence hydrogen or C₁₋₆ alkyl optionally substituted with —S—(C₁₋₄ alkyl), phenyl, or C₃₋₇ cycloalkyl; or two instances of R⁶ are taken together with the carbon atom to which they are attached to form a 3-5 membered saturated carbocyclic ring. In certain embodiments, R⁶ represents independently for each occurrence C₁₋₆ alkyl optionally substituted with —S—(C₁₋₄ alkyl), phenyl, or C₃₋₇ cycloalkyl; or two instances of R⁶ are taken together with the carbon atom to which they are attached to form a 3-5 membered saturated carbocyclic ring.

In certain embodiments, R⁶ represents independently for each occurrence C₁₋₆ alkyl, C₁₋₆ haloalkyl, C₃₋₅ cycloalkyl, or hydrogen, wherein said C₁₋₆ alkyl is optionally substituted with —S—(C₁₋₄ alkyl), phenyl, or C₃₋₇ cycloalkyl. In certain embodiments, R⁶ represents independently for each occurrence hydrogen or C₁₋₆ alkyl optionally substituted with —S—(C₁₋₄ alkyl), phenyl, or C₃₋₇ cycloalkyl. In certain embodiments, R⁶ represents independently for each occurrence C₁₋₆ alkyl optionally substituted with —S—(C₁₋₄ alkyl), phenyl, or C₃₋₇ cycloalkyl.

In certain embodiments, one occurrence of R⁶ is hydrogen or C₁₋₆ alkyl optionally substituted with —S—(C₁₋₄ alkyl), phenyl, or C₃₋₇ cycloalkyl; and any geminal occurrence of R⁶ is hydrogen. In certain embodiments, one occurrence of R⁶ is C₁₋₆ alkyl optionally substituted with —S—(C₁₋₄ alkyl), phenyl, or C₃₋₇ cycloalkyl; and any geminal occurrence of R⁶ is hydrogen. In certain embodiments, one occurrence of R⁶ is C₁₋₆ alkyl substituted with —S—(C₁₋₄ alkyl), phenyl, or C₃₋₇ cycloalkyl; and any geminal occurrence of R⁶ is hydrogen. In certain embodiments, one occurrence of R⁶ is C₁₋₆ alkyl substituted with —S—(C₁₋₄ alkyl); and any geminal occurrence of R⁶ is hydrogen. In certain embodiments, one occurrence of R⁶ is C₁₋₆ alkyl substituted with phenyl; and any geminal occurrence of R⁶ is hydrogen. In certain embodiments, one occurrence of R⁶ is C₁₋₆ alkyl substituted with C₃₋₇ cycloalkyl; and any geminal occurrence of R⁶ is hydrogen.

In certain embodiments, R⁶ represents independently for each occurrence C₁₋₆ alkyl, C₁₋₆ haloalkyl, C₃₋₅ cycloalkyl, or hydrogen. In certain embodiments, R⁶ represents independently for each occurrence C₁₋₆ alkyl, C₁₋₆ haloalkyl, or C₃₋₅ cycloalkyl. In certain embodiments, R⁶ represents independently for each occurrence C₁₋₆ alkyl, C₁₋₆ haloalkyl, or hydrogen. In certain embodiments, R⁶ represents independently for each occurrence C₁₋₆ alkyl, C₁₋₆ haloalkyl, or hydrogen. In certain embodiments, R⁶ represents independently for each occurrence C₁₋₆ haloalkyl. In certain embodiments, R⁶ represents independently for each occurrence C₃₋₅ cycloalkyl.

In certain embodiments, R⁶ represents independently for each occurrence C₁₋₆ alkyl or hydrogen. In certain embodiments, R⁶ represents independently for each occurrence C₁₋₆ alkyl. In certain embodiments, R⁶ represents independently for each occurrence C₁₋₄ alkyl.

In certain embodiments, one occurrence of R⁶ is C₁₋₆ alkyl or hydrogen, and any geminal occurrence of R⁶ is hydrogen. In certain embodiments, one occurrence of R⁶ is C₁₋₆ alkyl, and any geminal occurrence of R⁶ is hydrogen. In certain embodiments, one occurrence of R⁶ is C₁₋₄ alkyl, and any geminal occurrence of R⁶ is hydrogen. In certain embodiments, one occurrence of R⁶ is methyl, and any geminal occurrence of R⁶ is hydrogen. In certain embodiments, R⁶ is methyl. In certain embodiments, R⁶ is hydrogen.

In certain embodiments, two instances of R⁶ are taken together with the carbon atom to which they are attached to form a 3-5 membered saturated carbocyclic ring. In certain embodiments, two instances of R⁶ are taken together with the carbon atom to which they are attached to form a 3-membered saturated carbocyclic ring.

In certain embodiments, R⁶ is selected from the groups depicted in the compounds in Tables 1, 2, 3, 4, 5, and 6, below.

As defined generally above, R⁷ represents independently for each occurrence C₁₋₈ alkyl, C₁₋₆ haloalkyl, C₂₋₆ alkenyl, C₃₋₇ cycloalkyl, or a 4-7 membered saturated monocyclic heterocyclyl having one or two heteroatoms independently selected from nitrogen, oxygen, and sulfur; wherein said C₁₋₈ alkyl is optionally substituted with C₁₋₄ alkoxyl, phenyl, C₃₋₇ cycloalkyl, or a 4-7 membered saturated monocyclic heterocyclyl having one or two heteroatoms independently selected from nitrogen, oxygen, and sulfur.

In certain embodiments, R⁷ represents independently for each occurrence C₁₋₆ alkyl, C₁₋₆ haloalkyl, C₂₋₆ alkenyl, C₃₋₇ cycloalkyl, or a 4-7 membered saturated monocyclic heterocyclyl having one or two heteroatoms independently selected from nitrogen, oxygen, and sulfur; wherein said C₁₋₆ alkyl is optionally substituted with C₁₋₄ alkoxyl, phenyl, C₃₋₇ cycloalkyl, or a 4-7 membered saturated monocyclic heterocyclyl having one or two heteroatoms independently selected from nitrogen, oxygen, and sulfur.

In certain embodiments, R⁷ represents independently for each occurrence C₁₋₆ alkyl, C₁₋₆ haloalkyl, C₂₋₆ alkenyl, C₃₋₇ cycloalkyl, or a 4-7 membered saturated monocyclic heterocyclyl having one or two heteroatoms independently selected from nitrogen, oxygen, and sulfur. In certain embodiments, R⁷ represents independently for each occurrence C₁₋₆ haloalkyl. In certain embodiments, R⁷ represents independently for each occurrence a 4-7 membered saturated monocyclic heterocyclyl having one or two heteroatoms independently selected from nitrogen, oxygen, and sulfur.

In certain embodiments, R⁷ represents independently for each occurrence C₁₋₈ alkyl optionally substituted with C₁₋₄ alkoxyl, phenyl, C₃₋₇ cycloalkyl, or a 4-7 membered saturated monocyclic heterocyclyl having one or two heteroatoms independently selected from nitrogen, oxygen, and sulfur. In certain embodiments, R⁷ represents independently for each occurrence C₁₋₆ alkyl optionally substituted with C₁₋₄ alkoxyl, phenyl, C₃₋₇ cycloalkyl, or a 4-7 membered saturated monocyclic heterocyclyl having one or two heteroatoms independently selected from nitrogen, oxygen, and sulfur. In certain embodiments, R⁷ represents independently for each occurrence C₁₋₆ alkyl optionally substituted with a 4-7 membered saturated monocyclic heterocyclyl having one or two heteroatoms independently selected from nitrogen, oxygen, and sulfur.

In certain embodiments, R⁷ represents independently for each occurrence C₁₋₈ alkyl substituted with C₁₋₄ alkoxyl, phenyl, C₃₋₇ cycloalkyl, or a 4-7 membered saturated monocyclic heterocyclyl having one or two heteroatoms independently selected from nitrogen, oxygen, and sulfur. In certain embodiments, R⁷ represents independently for each occurrence C₁₋₆ alkyl substituted with C₁₋₄ alkoxyl, phenyl, C₃₋₇ cycloalkyl, or a 4-7 membered saturated monocyclic heterocyclyl having one or two heteroatoms independently selected from nitrogen, oxygen, and sulfur. In certain embodiments, R⁷ represents independently for each occurrence C₁₋₆ alkyl substituted with a 4-7 membered saturated monocyclic heterocyclyl having one or two heteroatoms independently selected from nitrogen, oxygen, and sulfur. In certain embodiments, R⁷ represents independently for each occurrence C₁₋₈ alkyl.

In certain embodiments, R⁷ represents independently for each occurrence C₁₋₆ alkyl, C₂₋₆ alkenyl, C₃₋₇ cycloalkyl, or a 4-7 membered saturated monocyclic heterocyclyl having one nitrogen or oxygen atom; wherein said C₁₋₆ alkyl is optionally substituted with C₁₋₄ alkoxyl, phenyl, C₃₋₇ cycloalkyl, or a 4-7 membered saturated monocyclic heterocyclyl having one nitrogen or oxygen atom.

In certain embodiments, R⁷ represents independently for each occurrence C₁₋₆ alkyl, allyl, C₃₋₅ cycloalkyl,

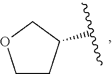 [SMILES: *[C@@H]1CCOC1]

—CH₂-phenyl, or —CH₂—(C₃₋₅ cycloalkyl). In certain embodiments, R⁷ represents independently for each occurrence C₃₋₅ cycloalkyl,

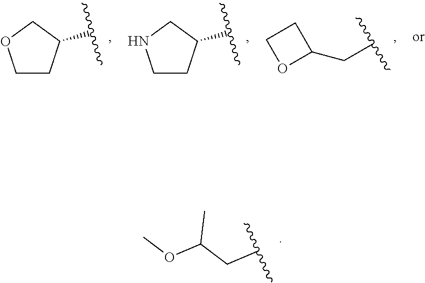

In certain embodiments, R⁷ represents independently for each occurrence C₁₋₆ alkyl or C₃₋₅ cycloalkyl. In certain embodiments, R⁷ represents independently for each occurrence C₁₋₄ alkyl or C₃₋₅ cycloalkyl.

In certain embodiments, R⁷ represents independently for each occurrence C₁₋₆ alkyl optionally substituted with C₁₋₄ alkoxyl, phenyl, C₃₋₇ cycloalkyl, or a 4-7 membered saturated monocyclic heterocyclyl having one nitrogen or oxygen atom. In certain embodiments, R⁷ represents independently for each occurrence C₁₋₆ alkyl optionally substituted with C₁₋₄ alkoxyl. In certain embodiments, R⁷ represents independently for each occurrence C₁₋₆ alkyl optionally substituted with phenyl, C₃₋₇ cycloalkyl, or a 4-7 membered saturated monocyclic heterocyclyl having one nitrogen or oxygen atom. In certain embodiments, R⁷ represents independently for each occurrence C₁₋₆ alkyl optionally substituted with phenyl or C₃₋₇ cycloalkyl. In certain embodiments, R⁷ represents independently for each occurrence C₁₋₆ alkyl optionally substituted with phenyl. In certain embodiments, R⁷ represents independently for each occurrence C₁₋₆ alkyl optionally substituted with C₃₋₇ cycloalkyl. In certain embodiments, R⁷ represents independently for each occurrence C₁₋₆ alkyl optionally substituted with a 4-7 membered saturated monocyclic heterocyclyl having one nitrogen or oxygen atom.

In certain embodiments, R⁷ represents independently for each occurrence C₁₋₆ alkyl substituted with C₁₋₄ alkoxyl, phenyl, C₃₋₇ cycloalkyl, or a 4-7 membered saturated monocyclic heterocyclyl having one nitrogen or oxygen atom. In certain embodiments, R⁷ represents independently for each occurrence C₁₋₆ alkyl substituted with C₁₋₄ alkoxyl. In certain embodiments, R⁷ represents independently for each occurrence C₁₋₆ alkyl substituted with phenyl, C₃₋₇ cycloalkyl, or a 4-7 membered saturated monocyclic heterocyclyl having one nitrogen or oxygen atom. In certain embodiments, R⁷ represents independently for each occurrence C₁₋₆ alkyl substituted with phenyl or C₃₋₇ cycloalkyl. In certain embodiments, R⁷ represents independently for each occurrence C₁₋₆ alkyl substituted with phenyl. In certain embodiments, R⁷ represents independently for each occurrence C₁₋₆ alkyl substituted with C₃₋₇ cycloalkyl. In certain embodiments, R⁷ represents independently for each occurrence C₁₋₆ alkyl substituted with a 4-7 membered saturated monocyclic heterocyclyl having one nitrogen or oxygen atom.

In certain embodiments, R⁷ represents independently for each occurrence C₁₋₆ alkyl. In certain embodiments, R⁷ represents independently for each occurrence C₁₋₄ alkyl. In certain embodiments, R⁷ represents independently for each occurrence methyl, ethyl, or isopropyl. In certain embodiments, R⁷ represents independently for each occurrence methyl or ethyl. In certain embodiments, R⁷ represents independently for each occurrence ethyl or isopropyl.

In certain embodiments, R⁷ represents independently for each occurrence C₂₋₆ alkenyl. In certain embodiments, R⁷ represents independently for each occurrence C₃₋₇ cycloalkyl. In certain embodiments, R⁷ represents independently for each occurrence C₃₋₅ cycloalkyl. In certain embodiments, R⁷ represents independently for each occurrence a 4-7 membered saturated monocyclic heterocyclyl having one nitrogen or oxygen atom.

In certain embodiments, R⁷ is selected from the groups depicted in the compounds in Tables 1, 2, 3, 4, 5, and 6, below.

As defined generally above, R⁸ represents independently for each occurrence halo, C₁. 4 alkyl, C₁₋₄ haloalkyl, C₁₋₄ alkoxyl, C₁₋₄ haloalkoxyl, —(C_{0.3} alkylene)-CO₂R¹⁰, —CN, or —N(R⁹)₂.

In certain embodiments, R⁸ represents independently for each occurrence halo, C₁₋₄ alkyl, C₁₋₄ haloalkyl, C₁₋₄ alkoxyl, —(C_{0.3} alkylene)-CO₂R¹⁰, or —N(R⁹)₂. In certain embodiments, R⁸ represents independently for each occurrence halo, C₁₋₄ alkyl, C₁₋₄ haloalkyl, C₁₋₄ alkoxyl, or —N(R⁹)₂. In certain embodiments, R⁸ represents independently for each occurrence halo, C₁₋₄ alkyl, C₁₋₄ haloalkyl, or C₁₋₄ alkoxyl. In certain embodiments, R⁸ represents independently for each occurrence halo, C₁₋₄ alkyl, or C₁₋₄ haloalkyl.

In certain embodiments, R⁸ represents independently for each occurrence halo. In certain embodiments, R⁸ represents independently for each occurrence C₁₋₄ alkyl. In certain embodiments, R⁸ represents independently for each occurrence C₁₋₄ haloalkyl. In certain embodiments, R⁸ represents independently for each occurrence C₁₋₄ alkoxyl. In certain embodiments, R⁸ represents independently for each occurrence C₁₋₄ haloalkoxyl. In certain embodiments, R⁸ represents independently for each occurrence —(C₁₋₃ alkylene)-CO₂R¹⁰. In certain embodiments, R⁸ represents independently for each occurrence —CO₂R¹⁰. In certain embodiments, R⁸ represents independently for each occurrence —(C₁₋₃ alkylene)-CO₂R¹⁰. In certain embodiments, R⁸ is —CN. In certain embodiments, R⁸ represents independently for each occurrence —N(R⁹)₂. In certain embodiments, R⁸ is selected from the groups depicted in the compounds in Tables 1, 2, 3, 4, 5, and 6, below.

As defined generally above, R⁹ represents independently for each occurrence hydrogen or C₁₋₄ alkyl, or two instances of R⁹ are taken together with the atom to which they are attached to form a 4-7 membered saturated heterocyclic ring containing 1 nitrogen atom and optionally 1 oxygen atom. In certain embodiments, R⁹ represents independently for each occurrence hydrogen or C₁₋₄ alkyl, or two instances of R⁹ are taken together with the atom to which they are attached to form a 4-7 membered saturated heterocyclic ring containing 1 nitrogen atom.

In certain embodiments, R⁹ represents independently for each occurrence hydrogen or C₁₋₄ alkyl. In certain embodiments, R⁹ represents independently for each occurrence hydrogen or methyl. In certain embodiments, R⁹ represents independently for each occurrence C₁₋₄ alkyl. In certain embodiments, R⁹ is methyl. In certain embodiments, R⁹ is hydrogen.

In certain embodiments, two instances of R⁹ are taken together with the atom to which they are attached to form a 4-7 membered saturated heterocyclic ring containing 1 nitrogen atom and optionally 1 oxygen atom. In certain embodiments, two instances of R⁹ are taken together with the atom to which they are attached to form a 4-7 membered saturated heterocyclic ring containing 1 nitrogen atom and 1 oxygen atom. In certain embodiments, two instances of R⁹ are taken together with the atom to which they are attached to form a 4-7 membered saturated heterocyclic ring containing 1 nitrogen atom. In certain embodiments, R⁹ is selected from the groups depicted in the compounds in Tables 1, 2, 3, 4, 5, and 6, below.

As defined generally above, R¹⁰ represents independently for each occurrence C₁₋₆ alkyl, C₃₋₇ cycloalkyl, or hydrogen. In certain embodiments, R¹⁰ represents independently for each occurrence C₁₋₆ alkyl or hydrogen. In certain embodiments, R¹⁰ represents independently for each occurrence C₁₋₄ alkyl or hydrogen. In certain embodiments, R¹⁰ represents independently for each occurrence C₁₋₆ alkyl or C₃₋₇ cycloalkyl. In certain embodiments, R¹⁰ represents independently for each occurrence C₃₋₇ cycloalkyl or hydrogen.

In certain embodiments, R¹⁰ represents independently for each occurrence C₁₋₆ alkyl. In certain embodiments, R¹⁰ represents independently for each occurrence C₁₋₄ alkyl. In certain embodiments, R¹⁰ is methyl. In certain embodiments, R¹⁰ represents independently for each occurrence C₃₋₇ cycloalkyl. In certain embodiments, R¹⁰ represents independently for each occurrence C₃₋₅ cycloalkyl. In certain embodiments, R¹⁰ is hydrogen. In certain embodiments,
R¹⁰ is selected from the groups depicted in the compounds in Tables 1, 2, 3, 4, 5, and 6, below.

As defined generally above, R¹¹ represents independently for each occurrence C₁₋₂ haloalkyl, —SF₅, —Si(C₁₋₄ alkyl)₃, —Si(CH₃)₂(C₁₋₄ haloalkyl), —Si(CH₃)₂(C₃₋₇ cycloalkyl),

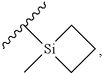 [SMILES: *[Si]1(C)CCC1]

—Si(CH₃)₂(phenyl), —S-phenyl, —O-phenyl, phenyl, thiophenyl, pyridinyl, or C₃₋₇ cycloalkyl; wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅.

In certain embodiments, R¹¹ represents independently for each occurrence —Si(C₁₋₄ alkyl)₃, —Si(CH₃)₂(C₁₋₄ haloalkyl), —Si(CH₃)₂(C₃₋₇ cycloalkyl),

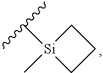 [SMILES: *[Si]1(C)CCC1]

or —Si(CH₃)₂(phenyl), wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅. In certain embodiments, R¹¹ represents independently for each occurrence —Si(C₁₋₄ alkyl)₃ or —Si(CH₃)₂(C₁₋₄ haloalkyl). In certain embodiments, R¹¹ represents independently for each occurrence —Si(CH₃)₂(C₃₋₇ cycloalkyl),

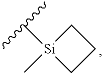 [SMILES: *[Si]1(C)CCC1]

or —Si(CH₃)₂(phenyl), wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅.

In certain embodiments, R¹¹ represents independently for each occurrence —Si(CH₃)₂(phenyl), —S-phenyl, —O-phenyl, phenyl, thiophenyl, pyridinyl, or C₃₋₇ cycloalkyl; wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅. In certain embodiments, R¹¹ represents independently for each occurrence —Si(CH₃)₂(phenyl), —S-phenyl, —O-phenyl; wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅. In certain embodiments, R¹¹ represents independently for each occurrence phenyl, thiophenyl, pyridinyl, or C₃₋₇ cycloalkyl; wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅. In certain embodiments, R¹¹ represents independently for each occurrence phenyl, thiophenyl, or pyridinyl; wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅. In certain embodiments, R¹¹ represents independently for each occurrence phenyl or C₃₋₇ cycloalkyl; wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅.

In certain embodiments, R¹¹ represents independently for each occurrence C₁₋₂ haloalkyl. In certain embodiments, R¹¹ represents independently for each occurrence —SF₅. In certain embodiments, R¹¹ represents independently for each occurrence —Si(C₁₋₄ alkyl)₃. In certain embodiments, R¹¹ represents independently for each occurrence —Si(CH₃)₂(C₁₋₄ haloalkyl). In certain embodiments, R¹¹ represents independently for each occurrence —Si(CH₃)₂(C₃₋₇ cycloalkyl). In certain embodiments, R¹¹ represents independently for each occurrence

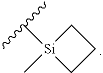 [SMILES: *[Si]1(C)CCC1]

In certain embodiments, R¹¹ represents independently for each occurrence —Si(CH₃)₂(phenyl), wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅. In certain embodiments, R¹¹ represents independently for each occurrence —S-phenyl, wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅. In certain embodiments, R¹¹ represents independently for each occurrence —O-phenyl, wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅. In certain embodiments, R¹¹ represents independently for each occurrence phenyl, wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅.

In certain embodiments, R¹¹ is —Si(CH₃)₂(phenyl). In certain embodiments, R¹¹ is —S— phenyl. In certain embodiments, R¹¹ is —O-phenyl. In certain embodiments, R¹¹ is phenyl. In certain embodiments, R¹¹ is thiophenyl. In certain embodiments, R¹¹ is pyridinyl. In certain embodiments, R¹¹ is C₃₋₇ cycloalkyl.

In certain embodiments, R¹¹ is selected from the groups depicted in the compounds in Tables 1, 2, 3, 4, 5, and 6, below.

As defined generally above, X represents independently for each occurrence —O—, ψ-OC(O)—, —OC(O)O—, ψ-OC(O)—N(R⁹)—, —S—, —S—S—, or ψ-SC(O)—; wherein ψ denotes the point of attachment to C₁₋₁₀ alkylene. In certain embodiments, X represents independently for each occurrence —O—, ψ-OC(O)—, —OC(O)O—, ψ-OC(O)—N(R⁹)—, —S—, or ψ-SC(O)—; wherein ψ denotes the point of attachment to C₁₋₁₀ alkylene.

In certain embodiments, X represents independently for each occurrence —O— or —S—. In certain embodiments, X represents independently for each occurrence ψ-OC(O)— or ψ-SC(O)—; wherein ψ denotes the point of attachment to C₁₋₁₀ alkylene. In certain embodiments, X represents independently for each occurrence —OC(O)O— or ψ-OC(O)—N(R⁹)—; wherein ψ denotes the point of attachment to C₁₋₁₀ alkylene.

In certain embodiments, X represents independently for each occurrence —O—, ψ—OC(O)—, —OC(O)O—, or ψ-OC(O)—N(R⁹)—; wherein ψ denotes the point of attachment to C₁₋₁₀ alkylene. In certain embodiments, X represents independently for each occurrence —S—, —S—S—, or ψ-SC(O)—; wherein ψ denotes the point of attachment to C₁₋₁₀ alkylene. In certain embodiments, X represents independently for each occurrence —S— or ψ-SC(O)—; wherein ψ denotes the point of attachment to C₁₋₁₀ alkylene.

In certain embodiments, X is —O—. In certain embodiments, X is ψ-OC(O)—; wherein ψ denotes the point of attachment to C₁₋₁₀ alkylene. In certain embodiments, X is —OC(O)O—. In certain embodiments, X is ψ-OC(O)—N(R⁹)—; wherein ψ denotes the point of attachment to C₁₋₁₀ alkylene. In certain embodiments, X is —S—. In certain embodiments, X is —S—S—. In certain embodiments, X is ψ-SC(O)—; wherein ψ denotes the point of attachment to C₁₋₁₀ alkylene.

In certain embodiments, X is selected from the groups depicted in the compounds in Tables 1, 2, 3, 4, 5, and 6, below.

As defined generally above, Y represents independently for each occurrence —O—, —S—, or —CF₂—. In certain embodiments, Y represents independently for each occurrence —O— or —S—. In certain embodiments, Y represents independently for each occurrence —O— or —CF₂—. In certain embodiments, Y represents independently for each occurrence —S— or —CF₂—. In certain embodiments, Y is —O—. In certain embodiments, Y is —S—. In certain embodiments, Y is —CF₂—. In certain embodiments, X is selected from the groups depicted in the compounds in Tables 1, 2, 3, 4, 5, and 6, below.

As defined generally above, B¹ is

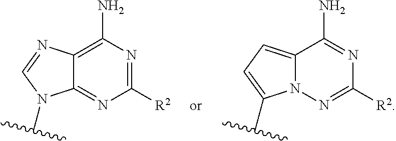

In certain embodiments, B¹ is

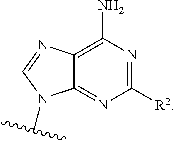

In certain embodiments B¹ is

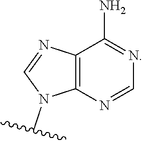 [SMILES: *n1cnc2c(N)ncnc21]

In certain embodiments, B¹ is

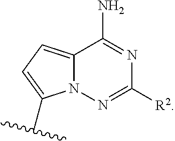

In certain embodiments, B¹ is selected from the groups depicted in the compounds in Tables 1, 2, 3, 4, 5, and 6, below.

As defined generally above, m is independently for each occurrence 0, 1, 2, or 3. In certain embodiments, m is 0, 1, 2, or 3. In certain embodiments, m is 0. In certain embodiments, m is 1. In certain embodiments, m is 2. In certain embodiments, m is 3. In certain embodiments, m is independently for each occurrence 0 or 1. In certain embodiments, m is independently for each occurrence 1 or 2. In certain embodiments, m is independently for each occurrence 2 or 3. In certain embodiments, m is independently for each occurrence 0, 1, or 2. In certain embodiments m is independently for each occurrence 1, 2, or 3. In certain embodiments, m is selected from the values represented in the compounds in Tables 1, 2, 3, 4, 5, and 6, below.

As defined generally above, p is 0, 1, 2, or 3. In certain embodiments, p is 0. In certain embodiments, p is 1. In certain embodiments, p is 2. In certain embodiments, p is 3. In certain embodiments, p is 0 or 1. In certain embodiments, p is 1 or 2. In certain embodiments, p is 2 or 3. In certain embodiments, p is 0, 1, or 2. In certain embodiments p is 1, 2, or 3. In certain embodiments, p is selected from the values represented in the compounds in Tables 1, 2, 3, 4, 5, and 6, below.

As defined generally above, one or more hydrogen atoms may be replaced with deuterium. In certain embodiments, no hydrogen atoms are replaced with deuterium. In certain embodiments, one or two hydrogen atoms are replaced with deuterium. In certain embodiments, a hydrogen atom is replaced with deuterium at a position depicted in one of the compounds in Tables 1, 2, 3, 4, 5, and 6, below.

The description above describes multiple embodiments relating to compounds of Formula I. The patent application specifically contemplates all combinations of the embodiments.

In certain embodiments, the compound is a compound of Formula I-A:

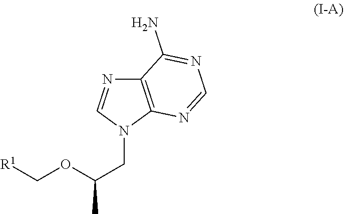

or a pharmaceutically acceptable salt thereof; wherein:
R¹ is —P(O)(OR³)(N(R⁴)(R⁵)) or —P(O)(N(R⁴)(R⁵))₂;
R³ is phenyl, naphthyl, or —C(R⁶)₂—CO₂R¹⁰; wherein said phenyl and naphthyl are substituted with m instances of R⁸;
R⁴ represents independently for each occurrence hydrogen or C₁₋₄ alkyl; or R⁴ and one occurrence of R⁶ are taken together with the atoms to which they are attached to form a 4-7 membered saturated heterocyclic ring containing 1 nitrogen atom;
R⁵ represents independently for each occurrence —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, or —C(R¹⁰)(H)—CH₂—CO₂R¹⁰;
R⁶ represents independently for each occurrence C₁₋₆ alkyl, C₁₋₆ haloalkyl, C₃₋₅ cycloalkyl, or hydrogen, wherein said C₁₋₆ alkyl is optionally substituted with —S—(C₁₋₄ alkyl), phenyl, or C₃₋₇ cycloalkyl; or two instances of R⁶ are taken together with the carbon atom to which they are attached to form a 3-5 membered saturated carbocyclic ring;
R⁷ represents independently for each occurrence C₁₋₆ alkyl, C₂₋₆ alkenyl, C₃₋₇ cycloalkyl, or a 4-7 membered saturated monocyclic heterocyclyl having one nitrogen or oxygen atom; wherein said C₁₋₆ alkyl is optionally substituted with C₁₋₄ alkoxyl, phenyl, C₃₋₇ cycloalkyl, or a 4-7 membered saturated monocyclic heterocyclyl having one nitrogen or oxygen atom;
R⁸ represents independently for each occurrence halo, C₁₋₄ alkyl, C₁₋₄ haloalkyl, or C₁₋₄ alkoxyl;
R⁹ and R¹⁰ represents independently for each occurrence C₁₋₄ alkyl or hydrogen; and
m is 0, 1, 2, or 3.

The definitions of variables in Formula I-A above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-A.

As defined generally above, R¹ is —P(O)(OR³)(N(R⁴)(R⁵)) or —P(O)(N(R⁴)(R⁵))₂. In certain embodiments, R¹ is —P(O)(OR³)(N(R⁴)(R⁵)). In certain embodiments, R¹ is —P(O)(N(R⁴)(R⁵))₂.

As defined generally above, R³ is phenyl, naphthyl, or —C(R⁶)₂—CO₂R¹⁰; wherein said phenyl and naphthyl are substituted with m instances of R⁸. In certain embodiments, R³ is phenyl or naphthyl, each of which is substituted with m instances of R⁸. In certain embodiments, R³ is phenyl substituted with m instances of R⁸. In certain embodiments, R³ is

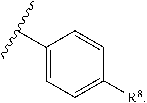

In certain embodiments, R³ is naphthyl substituted with m instances of R⁸.

In certain embodiments, R³ is phenyl or naphthyl. In certain embodiments, R³ is phenyl. In certain embodiments, R³ is naphthyl. In certain embodiments, R³ is 1-naphthyl. In certain embodiments, R³ is 2-naphthyl.

In certain embodiments, R³ is —C(R⁶)₂—CO₂R¹⁰.

As defined generally above, R⁴ represents independently for each occurrence hydrogen or C₁₋₄ alkyl; or R⁴ and one occurrence of R⁶ are taken together with the atoms to which they are attached to form a 4-7 membered saturated heterocyclic ring containing 1 nitrogen atom. In certain embodiments, R⁴ represents independently for each occurrence hydrogen or C₁₋₄ alkyl. In certain embodiments, R⁴ represents independently for each occurrence C₁₋₄ alkyl.

In certain embodiments, R⁴ represents independently for each occurrence hydrogen or methyl; or R⁴ and one occurrence of R⁶ are taken together with the atoms to which they are attached to form a 4-7 membered saturated heterocyclic ring containing 1 nitrogen atom.

In certain embodiments, R⁴ represents independently for each occurrence hydrogen or methyl. In certain embodiments, R⁴ is hydrogen. In certain embodiments, R⁴ is methyl. In certain embodiments, R⁴ and one occurrence of R⁶ are taken together with the atoms to which they are attached to form a 4-7 membered saturated heterocyclic ring containing 1 nitrogen atom.

In certain embodiments, —N(R⁴)(R⁵) is

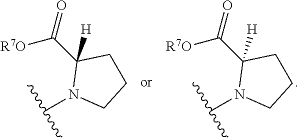

In certain embodiments, —N(R⁴)(R⁵) is

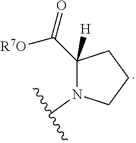

In certain embodiments, —N(R⁴)(R⁵) is

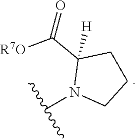

As defined generally above, R⁵ represents independently for each occurrence —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, or —C(R¹⁰)(H)—CH₂—CO₂R¹⁰. In certain embodiments, R⁵ represents independently for each occurrence —C(R⁶)₂—CO₂R⁷, —C(R⁶)₂—C(O)N(R⁹)₂, or —C(R⁶)₂—C(O)SR¹⁰. In certain embodiments, R⁵ represents independently for each occurrence —C(R⁶)₂—C(O)N(R⁹)₂, —C(R⁶)₂—C(O)SR¹⁰, —CH₂—C(R¹⁰)(H)—CO₂R¹⁰, or —C(R¹⁰)(H)—CH₂—CO₂R¹⁰. In certain embodiments, R⁵ represents independently for each occurrence —C(R⁶)₂—C(O)N(R⁹)₂ or —C(R⁶)₂—C(O)SR¹⁰. In certain embodiments, R⁵ represents independently for each occurrence —CH₂—C(R¹⁰)(H)—CO₂R¹⁰ or —C(R¹⁰)(H)—CH₂—CO₂R¹⁰.

In certain embodiments, R⁵ represents independently for each occurrence —C(R⁶)₂—CO₂R⁷. In certain embodiments, R⁵ represents independently for each occurrence —C(H)(R⁶)—CO₂R⁷. In certain embodiments, R⁵ represents independently for each occurrence

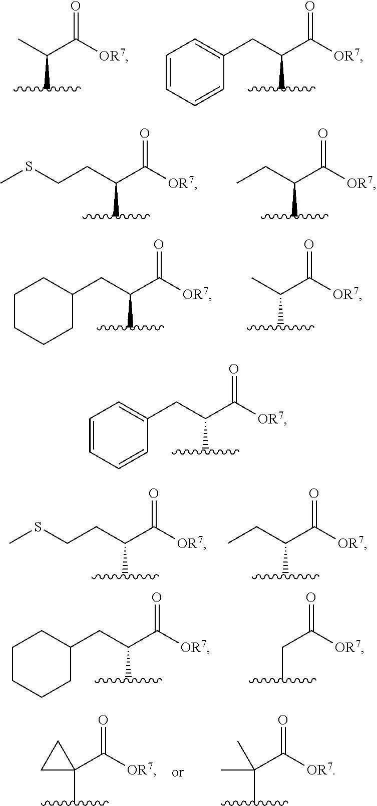

In certain embodiments, R⁵ represents independently for each occurrence

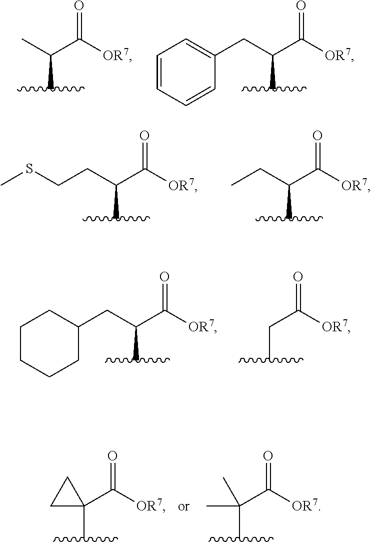

In certain embodiments, R⁵ represents independently for each occurrence

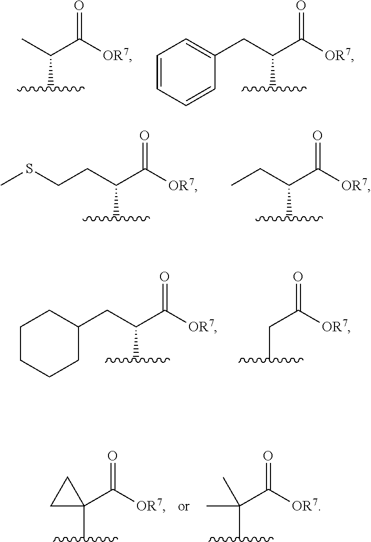

In certain embodiments, R⁵ is

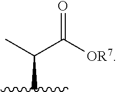

In certain embodiments, R⁵ is

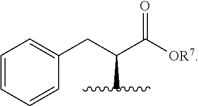

In certain embodiments, R⁵ is

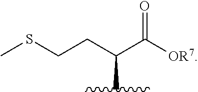

In certain embodiments, R⁵ is

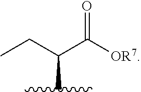

In certain embodiments, R⁵ is

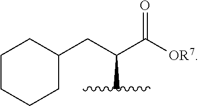

In certain embodiments, R⁵ is

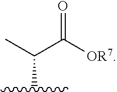

In certain embodiments, R⁵ is

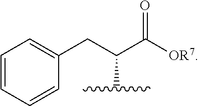

In certain embodiments, R⁵ is

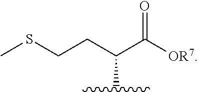

In certain embodiments, R⁵ is

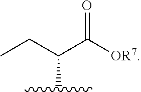

In certain embodiments, R⁵ is

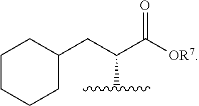

In certain embodiments, R⁵ is

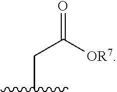

In certain embodiments, R⁵ is

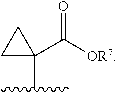

In certain embodiments, R⁵ is

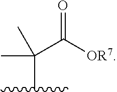

In certain embodiments, R⁵ represents independently for each occurrence —C(R⁶)₂—C(O)N(R⁹)₂. In certain embodiments, R⁵ represents independently for each occurrence —C(R⁶)₂—C(O)SR¹⁰. In certain embodiments, R⁵ represents independently for each occurrence —CH₂—C(R¹⁰)(H)—CO₂R¹⁰. In certain embodiments, R⁵ represents independently for each occurrence —C(R¹⁰)(H)—CH₂—CO₂R¹⁰.

As defined generally above, R⁶ represents independently for each occurrence C₁₋₆ alkyl, C₁₋₆ haloalkyl, C₃₋₅ cycloalkyl, or hydrogen, wherein said C₁₋₆ alkyl is optionally substituted with —S—(C₁₋₄ alkyl), phenyl, or C₃₋₇ cycloalkyl; or two instances of R⁶ are taken together with the carbon atom to which they are attached to form a 3-5 membered saturated carbocyclic ring. In certain embodiments, R⁶ represents independently for each occurrence hydrogen or C₁₋₆ alkyl optionally substituted with —S—(C₁₋₄ alkyl), phenyl, or C₃₋₇ cycloalkyl; or two instances of R⁶ are taken together with the carbon atom to which they are attached to form a 3-5 membered saturated carbocyclic ring. In certain embodiments, R⁶ represents independently for each occurrence C₁₋₆ alkyl optionally substituted with —S—(C₁₋₄ alkyl), phenyl, or C₃₋₇ cycloalkyl; or two instances of R⁶ are taken together with the carbon atom to which they are attached to form a 3-5 membered saturated carbocyclic ring.

In certain embodiments, R⁶ represents independently for each occurrence C₁₋₆ alkyl, C₁₋₆ haloalkyl, C₃₋₅ cycloalkyl, or hydrogen, wherein said C₁₋₆ alkyl is optionally substituted with —S—(C₁₋₄ alkyl), phenyl, or C₃₋₇ cycloalkyl. In certain embodiments, R⁶ represents independently for each occurrence hydrogen or C₁₋₆ alkyl optionally substituted with —S—(C₁₋₄ alkyl), phenyl, or C₃₋₇ cycloalkyl. In certain embodiments, R⁶ represents independently for each occurrence C₁₋₆ alkyl optionally substituted with —S—(C₁₋₄ alkyl), phenyl, or C₃₋₇ cycloalkyl.

In certain embodiments, one occurrence of R⁶ is hydrogen or C₁₋₆ alkyl optionally substituted with —S—(C₁₋₄ alkyl), phenyl, or C₃₋₇ cycloalkyl; and any geminal occurrence of R⁶ is hydrogen. In certain embodiments, one occurrence of R⁶ is C₁₋₆ alkyl optionally substituted with —S—(C₁₋₄ alkyl), phenyl, or C₃₋₇ cycloalkyl; and any geminal occurrence of R⁶ is hydrogen. In certain embodiments, one occurrence of R⁶ is C₁₋₆ alkyl substituted with —S—(C₁₋₄ alkyl), phenyl, or C₃₋₇ cycloalkyl; and any geminal occurrence of R⁶ is hydrogen. In certain embodiments, one occurrence of R⁶ is C₁₋₆ alkyl substituted with —S—(C₁₋₄ alkyl); and any geminal occurrence of R⁶ is hydrogen. In certain embodiments, one occurrence of R⁶ is C₁₋₆ alkyl substituted with phenyl; and any geminal occurrence of R⁶ is hydrogen. In certain embodiments, one occurrence of R⁶ is C₁₋₆ alkyl substituted with C₃₋₇ cycloalkyl; and any geminal occurrence of R⁶ is hydrogen.

In certain embodiments, R⁶ represents independently for each occurrence C₁₋₆ alkyl, C₁₋₆ haloalkyl, C₃₋₅ cycloalkyl, or hydrogen. In certain embodiments, R⁶ represents independently for each occurrence C₁₋₆ alkyl, C₁₋₆ haloalkyl, or C₃₋₅ cycloalkyl. In certain embodiments, R⁶ represents independently for each occurrence C₁₋₆ alkyl, C₁₋₆ haloalkyl, or hydrogen. In certain embodiments, R⁶ represents independently for each occurrence C₁₋₆ alkyl, C₁₋₆ haloalkyl, or hydrogen. In certain embodiments, R⁶ represents independently for each occurrence C₁₋₆ haloalkyl. In certain embodiments, R⁶ represents independently for each occurrence C₃₋₅ cycloalkyl.

In certain embodiments, R⁶ represents independently for each occurrence C₁₋₆ alkyl or hydrogen. In certain embodiments, R⁶ represents independently for each occurrence C₁₋₆ alkyl. In certain embodiments, R⁶ represents independently for each occurrence C₁₋₄ alkyl.

In certain embodiments, one occurrence of R⁶ is C₁₋₆ alkyl or hydrogen, and any geminal occurrence of R⁶ is hydrogen. In certain embodiments, one occurrence of R⁶ is C₁₋₆ alkyl, and any geminal occurrence of R⁶ is hydrogen. In certain embodiments, one occurrence of R⁶ is C₁₋₄ alkyl, and any geminal occurrence of R⁶ is hydrogen. In certain embodiments, one occurrence of R⁶ is methyl, and any geminal occurrence of R⁶ is hydrogen. In certain embodiments, R⁶ is methyl. In certain embodiments, R⁶ is hydrogen.

In certain embodiments, two instances of R⁶ are taken together with the carbon atom to which they are attached to form a 3-5 membered saturated carbocyclic ring. In certain embodiments, two instances of R⁶ are taken together with the carbon atom to which they are attached to form a 3-membered saturated carbocyclic ring.

As defined generally above, R⁷ represents independently for each occurrence C₁₋₆ alkyl, C₂₋₆ alkenyl, C₃₋₇ cycloalkyl, or a 4-7 membered saturated monocyclic heterocyclyl having one nitrogen or oxygen atom; wherein said C₁₋₆ alkyl is optionally substituted with C₁₋₄ alkoxyl, phenyl, C₃₋₇ cycloalkyl, or a 4-7 membered saturated monocyclic heterocyclyl having one nitrogen or oxygen atom.

In certain embodiments, R⁷ represents independently for each occurrence C₁₋₆ alkyl, allyl, C₃₋₅ cycloalkyl,

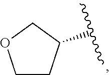 [SMILES: *[C@@H]1CCOC1]

—CH₂-phenyl, or —CH₂—(C₃₋₅ cycloalkyl). In certain embodiments, R⁷ represents independently for each occurrence C₃₋₅ cycloalkyl,

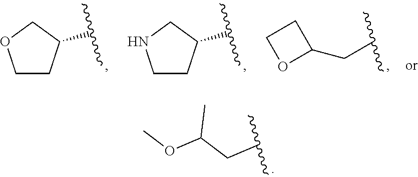

In certain embodiments, R⁷ represents independently for each occurrence C₁₋₆ alkyl or C₃₋₅ cycloalkyl. In certain embodiments, R⁷ represents independently for each occurrence C₁₋₄ alkyl or C₃₋₅ cycloalkyl.

In certain embodiments, R⁷ represents independently for each occurrence C₁₋₆ alkyl optionally substituted with C₁₋₄ alkoxyl, phenyl, C₃₋₇ cycloalkyl, or a 4-7 membered saturated monocyclic heterocyclyl having one nitrogen or oxygen atom. In certain embodiments, R⁷ represents independently for each occurrence C₁₋₆ alkyl optionally substituted with C₁₋₄ alkoxyl. In certain embodiments, R⁷ represents independently for each occurrence C₁₋₆ alkyl optionally substituted with phenyl, C₃₋₇ cycloalkyl, or a 4-7 membered saturated monocyclic heterocyclyl having one nitrogen or oxygen atom. In certain embodiments, R⁷ represents independently for each occurrence C₁₋₆ alkyl optionally substituted with phenyl or C₃₋₇ cycloalkyl. In certain embodiments, R⁷ represents independently for each occurrence C₁₋₆ alkyl optionally substituted with phenyl. In certain embodiments, R⁷ represents independently for each occurrence C₁₋₆ alkyl optionally substituted with C₃₋₇ cycloalkyl. In certain embodiments, R⁷ represents independently for each occurrence C₁₋₆ alkyl optionally substituted with a 4-7 membered saturated monocyclic heterocyclyl having one nitrogen or oxygen atom.

In certain embodiments, R⁷ represents independently for each occurrence C₁₋₆ alkyl substituted with C₁₋₄ alkoxyl, phenyl, C₃₋₇ cycloalkyl, or a 4-7 membered saturated monocyclic heterocyclyl having one nitrogen or oxygen atom. In certain embodiments, R⁷ represents independently for each occurrence C₁₋₆ alkyl substituted with C₁₋₄ alkoxyl. In certain embodiments, R⁷ represents independently for each occurrence C₁₋₆ alkyl substituted with phenyl, C₃₋₇ cycloalkyl, or a 4-7 membered saturated monocyclic heterocyclyl having one nitrogen or oxygen atom. In certain embodiments, R⁷ represents independently for each occurrence C₁₋₆ alkyl substituted with phenyl or C₃₋₇ cycloalkyl. In certain embodiments, R⁷ represents independently for each occurrence C₁₋₆ alkyl substituted with phenyl. In certain embodiments, R⁷ represents independently for each occurrence C₁₋₆ alkyl substituted with C₃₋₇ cycloalkyl. In certain embodiments, R⁷ represents independently for each occurrence C₁₋₆ alkyl substituted with a 4-7 membered saturated monocyclic heterocyclyl having one nitrogen or oxygen atom.

In certain embodiments, R⁷ represents independently for each occurrence C₁₋₆ alkyl. In certain embodiments, R⁷ represents independently for each occurrence C₁₋₄ alkyl. In certain embodiments, R⁷ represents independently for each occurrence methyl, ethyl, or isopropyl. In certain embodiments, R⁷ represents independently for each occurrence methyl or ethyl. In certain embodiments, R⁷ represents independently for each occurrence ethyl or isopropyl.

In certain embodiments, R⁷ represents independently for each occurrence C₂₋₆ alkenyl. In certain embodiments, R⁷ represents independently for each occurrence C₃₋₇ cycloalkyl. In certain embodiments, R⁷ represents independently for each occurrence C₃₋₅ cycloalkyl. In certain embodiments, R⁷ represents independently for each occurrence a 4-7 membered saturated monocyclic heterocyclyl having one nitrogen or oxygen atom.

As defined generally above, R⁸ represents independently for each occurrence halo, C₁. 4 alkyl, C₁₋₄ haloalkyl, or C₁₋₄ alkoxyl. In certain embodiments, R⁸ represents independently for each occurrence halo, C₁₋₄ alkyl, or C₁₋₄ haloalkyl.

In certain embodiments, R⁸ represents independently for each occurrence halo. In certain embodiments, R⁸ represents independently for each occurrence C₁₋₄ alkyl. In certain embodiments, R⁸ represents independently for each occurrence C₁₋₄ haloalkyl. In certain embodiments, R⁸ represents independently for each occurrence C₁₋₄ alkoxyl.

As defined generally above, R⁹ represents independently for each occurrence hydrogen or C₁₋₄ alkyl. In certain embodiments, R⁹ represents independently for each occurrence hydrogen or methyl. In certain embodiments, R⁹ represents independently for each occurrence C₁₋₄ alkyl. In certain embodiments, R⁹ is methyl. In certain embodiments, R⁹ is hydrogen.

As defined generally above, R¹⁰ represents independently for each occurrence C₁₋₄ alkyl or hydrogen. In certain embodiments, R¹⁰ represents independently for each occurrence C₁. 4 alkyl. In certain embodiments, R¹⁰ is methyl. In certain embodiments, R¹⁰ is hydrogen.

As defined generally above, m is 0, 1, 2, or 3. In certain embodiments, m is 0. In certain embodiments, m is 1. In certain embodiments, m is 2. In certain embodiments, m is 3. In certain embodiments, m is 0 or 1. In certain embodiments, m is 1 or 2. In certain embodiments, m is 2 or 3. In certain embodiments, m is 0, 1, or 2. In certain embodiments m is 1, 2, or 3.

The description above describes multiple embodiments relating to compounds of Formula I-A. The patent application specifically contemplates all combinations of the embodiments.

In certain embodiments, the compound is a compound of Formula I-B:

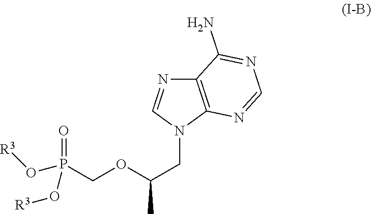

or a pharmaceutically acceptable salt thereof; wherein:
R³ represents independently for each occurrence C₁₋₂₀ alkyl, C₁₋₂₀ haloalkyl, —(C₁₋₁₀ alkylene)-X—(C₁₋₂₀ alkyl), hydrogen, —P(O)(OH)₂, or —P(O)(OH)—O—P(O)(OH)₂; wherein said C₁₋₂₀ alkyl, C₁₋₂₀ haloalkyl, and C₁₋₁₀ alkylene are optionally substituted with one hydroxyl or C₁₋₂₀ alkoxyl; and wherein one methylene unit in each of said C₁₋₂₀ alkyl, C₁₋₂₀ haloalkyl, and C₁₋₁₀ alkylene is optionally replaced with a C₃₋₅ cycloalkylene; or
two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸;
R⁸ represents independently for each occurrence halo, C₁₋₄ alkyl, C₁₋₄ haloalkyl, C₁₋₄ alkoxyl, —(C₀₋₃ alkylene)-CO₂R¹⁰, or —N(R⁹)₂;
R⁹ represents independently for each occurrence hydrogen or C₁₋₄ alkyl;
R¹⁰ represents independently for each occurrence C₁₋₆ alkyl, C₃₋₇ cycloalkyl, or hydrogen;
X represents independently for each occurrence —O—, ψ-OC(O)—, —OC(O)O—, ψ-OC(O)—N(R⁹)—, —S—, —S—S—, or ψ-SC(O)—; wherein ψ denotes the point of attachment to C₁₋₁₀ alkylene; and
p is 0, 1, 2, or 3.

The definitions of variables in Formula I-B above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I-B.

As defined generally above, R³ represents independently for each occurrence C₁₋₂₀ alkyl, C₁₋₂₀ haloalkyl, —(C₁₋₁₀ alkylene)-X—(C₁₋₂₀ alkyl), hydrogen, —P(O)(OH)₂, or —P(O)(OH)—O—P(O)(OH)₂; wherein said C₁₋₂₀ alkyl, C₁₋₂₀ haloalkyl, and C₁₋₁₀ alkylene are optionally substituted with one hydroxyl or C₁₋₂₀ alkoxyl; and wherein one methylene unit in each of said C₁₋₂₀ alkyl, C₁₋₂₀ haloalkyl, and C₁₋₁₀ alkylene is optionally replaced with a C₃₋₅ cycloalkylene; or
two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸.

In certain embodiments, R³ represents independently for each occurrence C₁₋₂₀ alkyl, C₁₋₂₀ haloalkyl, —(C₁₋₁₀ alkylene)-X—(C₁₋₂₀ alkyl), —P(O)(OH)₂, or —P(O)(OH)—O—P(O)(OH)₂; wherein said C₁₋₂₀ alkyl, C₁₋₂₀ haloalkyl, and C₁₋₁₀ alkylene are optionally substituted with one hydroxyl or C₁₋₂₀ alkoxyl; and wherein one methylene unit in each of said C₁₋₂₀ alkyl, C₁₋₂₀ haloalkyl, and C₁₋₁₀ alkylene is optionally replaced with a C₃₋₅ cycloalkylene; and wherein one occurrence of R³ is additionally selected from hydrogen; or two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸.

In certain embodiments, R³ represents independently for each occurrence C₁₋₂₀ alkyl, C₁₋₂₀ haloalkyl, —(C₁₋₁₀ alkylene)-X—(C₁₋₂₀ alkyl), hydrogen, —P(O)(OH)₂, or —P(O)(OH)—O—P(O)(OH)₂; wherein said C₁₋₂₀ alkyl, C₁₋₂₀ haloalkyl, and C₁₋₁₀ alkylene are optionally substituted with one hydroxyl or C₁₋₂₀ alkoxyl; and wherein one methylene unit in each of said C₁₋₂₀ alkyl, C₁₋₂₀ haloalkyl, and C₁₋₁₀ alkylene is optionally replaced with a C₃₋₅ cycloalkylene.

In certain embodiments, R³ represents independently for each occurrence C₁₋₂₀ alkyl, C₁₋₂₀ haloalkyl, —(C₁₋₁₀ alkylene)-X—(C₁₋₂₀ alkyl), —P(O)(OH)₂, or —P(O)(OH)—O—P(O)(OH)₂; wherein said C₁₋₂₀ alkyl, C₁₋₂₀ haloalkyl, and C₁₋₁₀ alkylene are optionally substituted with one hydroxyl or C₁₋₂₀ alkoxyl; and wherein one methylene unit in each of said C₁₋₂₀ alkyl, C₁₋₂₀ haloalkyl, and C₁₋₁₀ alkylene is optionally replaced with a C₃₋₅ cycloalkylene; and wherein one occurrence of R³ is additionally selected from hydrogen.

In certain embodiments, R³ represents independently for each occurrence C₁₋₂₀ alkyl, C₁₋₂₀ haloalkyl, or —(C₁₋₁₀ alkylene)-X—(C₁₋₂₀ alkyl); wherein said C₁₋₂₀ alkyl, C₁₋₂₀ haloalkyl, and C₁₋₁₀ alkylene are optionally substituted with one hydroxyl or C₁₋₂₀ alkoxyl; and wherein one methylene unit in each of said C₁₋₂₀ alkyl, C₁₋₂₀ haloalkyl, and C₁₋₁₀ alkylene is optionally replaced with a C₃₋₅ cycloalkylene. In certain embodiments, R³ represents independently for each occurrence C₁₋₂₀ alkyl, C₁₋₂₀ haloalkyl, or —(C₁₋₁₀ alkylene)-X—(C₁₋₂₀ alkyl); wherein said C₁₋₂₀ alkyl, C₁₋₂₀ haloalkyl, and C₁₋₁₀ alkylene are optionally substituted with one hydroxyl or C₁₋₂₀ alkoxyl. In certain embodiments, R³ represents independently for each occurrence C₁₋₂₀ alkyl, C₁₋₂₀ haloalkyl, or —(C₁₋₁₀ alkylene)-X—(C₁₋₂₀ alkyl); wherein one methylene unit in each of said C₁₋₂₀ alkyl, C₁₋₂₀ haloalkyl, and C₁₋₁₀ alkylene is optionally replaced with a C₃₋₅ cycloalkylene. In certain embodiments, R³ represents independently for each occurrence C₁₋₂₀ alkyl, C₁₋₂₀ haloalkyl, or —(C₁₋₁₀ alkylene)-X—(C₁₋₂₀ alkyl).

In certain embodiments, R³ represents independently for each occurrence —(C₁₋₁₀ alkylene)-X—(C₁₋₂₀ alkyl); wherein said C₁₋₂₀ alkyl and C₁₋₁₀ alkylene are optionally substituted with one hydroxyl or C₁₋₂₀ alkoxyl; and wherein one methylene unit in said C₁₋₂₀ alkyl and C₁₋₁₀ alkylene is optionally replaced with a C₃₋₅ cycloalkylene. In certain embodiments, R³ represents independently for each occurrence —(C₁₋₁₀ alkylene)-X—(C₁₋₂₀ alkyl); wherein said C₁₋₂₀ alkyl and C₁₋₁₀ alkylene are optionally substituted with one hydroxyl or C₁₋₂₀ alkoxyl. In certain embodiments, R³ represents independently for each occurrence —(C₁₋₁₀ alkylene)-X—(C₁₋₂₀ alkyl); wherein one methylene unit in said C₁₋₂₀ alkyl and C₁₋₁₀ alkylene is optionally replaced with a C₃₋₅ cycloalkylene. In certain embodiments, R³ represents independently for each occurrence —(C₁₋₁₀ alkylene)-X—(C₁₋₂₀ alkyl).

In certain embodiments, R³ represents independently for each occurrence —(C₁₋₁₀ alkylene)-OC(O)O—(C₁₋₁₀ alkyl) or —(C₁₋₁₀ alkylene)-OC(O)—N(R⁹)—(C₁₋₁₀ alkyl); wherein one methylene unit in each of said C₁₋₁₀ alkyl is optionally replaced with a C₃₋₅ cycloalkylene. In certain embodiments, R³ represents independently for each occurrence —(C₁₋₁₀ alkylene)-OC(O)O—(C₁₋₁₀ alkyl) or —(C₁₋₁₀ alkylene)-OC(O)—N(H)—(C₁₋₁₀ alkyl); wherein one methylene unit in each of said C₁₋₁₀ alkyl is optionally replaced with a C₃₋₅ cycloalkylene. In certain embodiments, R³ represents independently for each occurrence —CH₂—OC(O)O—(C₁₋₁₀ alkyl) or —CH₂—OC(O)—N(H)—(C₁₋₁₀ alkyl); wherein one methylene unit in each of said C₁₋₁₀ alkyl is optionally replaced with a C₃₋₅ cycloalkylene. In certain embodiments, R³ represents independently for each occurrence —CH₂—OC(O)O—(C₁₋₆ alkyl) or —CH₂—OC(O)—N(H)—(C₁₋₆ alkyl). In certain embodiments, R³ represents independently for each occurrence —CH₂—OC(O)O—(C₃₋₅ cycloalkyl) or —CH₂—OC(O)—N(H)—(C₃₋₅ cycloalkyl).

In certain embodiments, R³ represents independently for each occurrence —(C₁₋₁₀ alkylene)-OC(O)O—(C₁₋₁₀ alkyl), wherein one methylene unit in said C₁₋₁₀ alkyl is optionally replaced with a C₃₋₅ cycloalkylene. In certain embodiments, R³ represents independently for each occurrence —CH₂—OC(O)O—(C₁₋₁₀ alkyl), wherein one methylene unit in said C₁₋₁₀ alkyl is optionally replaced with a C₃₋₅ cycloalkylene. In certain embodiments, R³ represents independently for each occurrence —CH₂—OC(O)O—(C₁₋₆ alkyl). In certain embodiments, R³ represents independently for each occurrence —CH₂—OC(O)O—(C₃₋₅ cycloalkyl).

In certain embodiments, R³ represents independently for each occurrence —(C₁₋₁₀ alkylene)-OC(O)—N(R⁹)—(C₁₋₁₀ alkyl), wherein one methylene unit in said C₁₋₁₀ alkyl is optionally replaced with a C₃₋₅ cycloalkylene. In certain embodiments, R³ represents independently for each occurrence —(C₁₋₁₀ alkylene)-OC(O)—N(H)—(C₁₋₁₀ alkyl), wherein one methylene unit in said C₁₋₁₀ alkyl is optionally replaced with a C₃₋₅ cycloalkylene. In certain embodiments, R³ represents independently for each occurrence —CH₂—OC(O)—N(H)—(C₁₋₁₀ alkyl); wherein one methylene unit in said C₁₋₁₀ alkyl is optionally replaced with a C₃₋₅ cycloalkylene. In certain embodiments, R³ represents independently for each occurrence —CH₂—OC(O)—N(R⁹)—(C₁₋₆ alkyl). In certain embodiments, R³ represents independently for each occurrence —CH₂—OC(O)—N(R⁹)—(C₃₋₅ cycloalkyl).

In certain embodiments, R³ represents independently for each occurrence —(C₁₋₁₀ alkylene)-OC(O)—(C₁₋₁₀ alkyl) or —(C₁₋₁₀ alkylene)-SC(O)—(C₁₋₁₀ alkyl); wherein said C₁₋₁₀ alkyl is optionally substituted with one hydroxyl; and wherein one methylene unit in each of said C₁₋₁₀ alkyl is optionally replaced with a C₃₋₅ cycloalkylene. In certain embodiments, R³ represents independently for each occurrence —(C₁₋₁₀ alkylene)-OC(O)—(C₁₋₁₀ alkyl) or —(C₁₋₁₀ alkylene)-SC(O)—(C₁₋₁₀ alkyl); wherein said C₁₋₁₀ alkyl is substituted with one hydroxyl; and wherein one methylene unit in each of said C₁₋₁₀ alkyl is optionally replaced with a C₃₋₅ cycloalkylene. In certain embodiments, R³ represents independently for each occurrence —(C₁₋₁₀ alkylene)-OC(O)—(C₁₋₁₀ alkyl) or —(C₁₋₁₀ alkylene)-SC(O)—(C₁₋₁₀ alkyl); wherein said C₁₋₁₀ alkyl is substituted with one hydroxyl; and wherein one methylene unit in each of said C₁₋₁₀ alkyl is replaced with a C₃₋₅ cycloalkylene. In certain embodiments, R³ represents independently for each occurrence —(C₁₋₁₀ alkylene)-OC(O)—(C₁₋₁₀ alkyl) or —(C₁₋₁₀ alkylene)-SC(O)—(C₁₋₁₀ alkyl); wherein said C₁₋₁₀ alkyl is substituted with one hydroxyl.

In certain embodiments, R³ represents independently for each occurrence —(CH₂)₁₋₂—OC(O)—(C₁₋₁₀ alkyl) or —(CH₂)₁₋₂—SC(O)—(C₁₋₁₀ alkyl); wherein said C₁₋₁₀ alkyl is optionally substituted with one hydroxyl; and wherein one methylene unit in each of said C₁₋₁₀ alkyl is optionally replaced with a cyclopropylene. In certain embodiments, R³ represents independently for each occurrence —(CH₂)₁₋₂—OC(O)—(C₁₋₁₀ alkyl) or —(CH₂)₁₋₂—SC(O)—(C₁₋₁₀ alkyl); wherein said C₁₋₁₀ alkyl is substituted with one hydroxyl; and wherein one methylene unit in each of said C₁₋₁₀ alkyl is optionally replaced with a cyclopropylene. In certain embodiments, R³ represents independently for each occurrence —(CH₂)₁₋₂—OC(O)—(C₁₋₁₀ alkyl) or —(CH₂)₁₋₂—SC(O)—(C₁₋₁₀ alkyl); wherein said C₁₋₁₀ alkyl is substituted with one hydroxyl; and wherein one methylene unit in each of said C₁₋₁₀ alkyl is replaced with a cyclopropylene. In certain embodiments, R³ represents independently for each occurrence —(CH₂)₁₋₂—OC(O)—(C₁₋₁₀ alkyl) or —(CH₂)₁₋₂—SC(O)—(C₁₋₁₀ alkyl); wherein said C₁₋₁₀ alkyl is substituted with one hydroxyl.

In certain embodiments, R³ represents independently for each occurrence —(CH₂)₁₋₂—OC(O)—(C₁₋₁₀ alkyl); wherein said C₁₋₁₀ alkyl is optionally substituted with one hydroxyl; and wherein one methylene unit in said C₁₋₁₀ alkyl is optionally replaced with a cyclopropylene. In certain embodiments, R³ represents independently for each occurrence —(CH₂)₁₋₂—OC(O)—(C₁₋₁₀ alkyl); wherein said C₁₋₁₀ alkyl is substituted with one hydroxyl; and wherein one methylene unit in said C₁₋₁₀ alkyl is optionally replaced with a cyclopropylene. In certain embodiments, R³ represents independently for each occurrence —(CH₂)₁₋₂—OC(O)—(C₁₋₁₀ alkyl); wherein said C₁₋₁₀ alkyl is substituted with one hydroxyl; and wherein one methylene unit in said C₁₋₁₀ alkyl is replaced with a cyclopropylene. In certain embodiments, R³ represents independently for each occurrence —(CH₂)₁₋₂—OC(O)—(C₁₋₁₀ alkyl), wherein said C₁₋₁₀ alkyl is substituted with one hydroxyl.

In certain embodiments, R³ represents independently for each occurrence —(CH₂)₁₋₂—SC(O)—(C₁₋₁₀ alkyl); wherein said C₁₋₁₀ alkyl is optionally substituted with one hydroxyl; and wherein one methylene unit in said C₁₋₁₀ alkyl is optionally replaced with a cyclopropylene. In certain embodiments, R³ represents independently for each occurrence —(CH₂)₁₋₂—SC(O)—(C₁₋₁₀ alkyl); wherein said C₁₋₁₀ alkyl is substituted with one hydroxyl; and wherein one methylene unit in said C₁₋₁₀ alkyl is optionally replaced with a cyclopropylene. In certain embodiments, R³ represents independently for each occurrence —(CH₂)₁₋₂—SC(O)—(C₁₋₁₀ alkyl); wherein said C₁₋₁₀ alkyl is substituted with one hydroxyl; and wherein one methylene unit in said C₁₋₁₀ alkyl is replaced with a cyclopropylene. In certain embodiments, R³ represents independently for each occurrence —(CH₂)₁₋₂—SC(O)—(C₁₋₁₀ alkyl), wherein said C₁₋₁₀ alkyl is substituted with one hydroxyl.

In certain embodiments, R³ represents independently for each occurrence

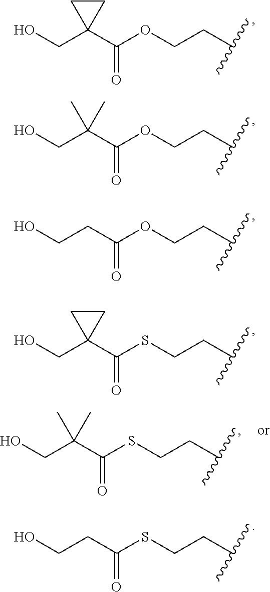

In certain embodiments, R³ represents independently for each occurrence

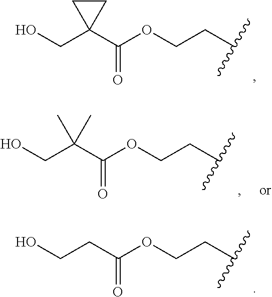

In certain embodiments, R³ represents independently for each occurrence

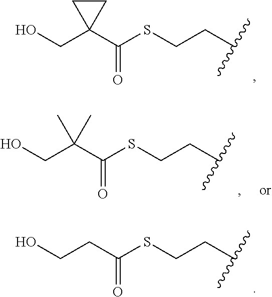

In certain embodiments, R³ is

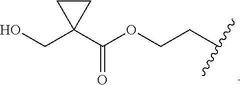 [SMILES: *CCOC(=O)C1(CO)CC1]

In certain embodiments, R³ is

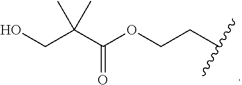 [SMILES: *CCOC(=O)C(C)(C)CO]

In certain embodiments, R³ is

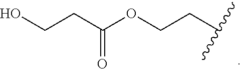 [SMILES: *CCOC(=O)CCO]

In certain embodiments, R³ is

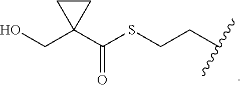 [SMILES: *CCSC(=O)C1(CO)CC1]

In certain embodiments, R³ is

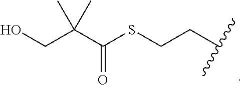 [SMILES: *CCSC(=O)C(C)(C)CO]

In certain embodiments, R³ is

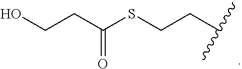 [SMILES: *CCSC(=O)CCO]

In certain embodiments, R³ represents independently for each occurrence —(C₁₋₁₀ alkylene)-O—(C₁₋₂₀ alkyl) or —(C₁₋₁₀ alkylene)-S—(C₁₋₂₀ alkyl); wherein said C₁₋₁₀ alkylene is optionally substituted with one C₁₋₂₀ alkoxyl; and wherein one occurrence of R³ is additionally selected from hydrogen. In certain embodiments, one occurrence of R³ is —(C₁₋₁₀ alkylene)-O—(C₁₋₂₀ alkyl) or —(C₁₋₁₀ alkylene)-S—(C₁₋₂₀ alkyl); wherein said C₁₋₁₀ alkylene is optionally substituted with one C₁₋₂₀ alkoxyl; and any second occurrence of R³ is hydrogen. In certain embodiments, one occurrence of R³ is —(C₁₋₁₀ alkylene)-O—(C₁₋₂₀ alkyl) or —(C₁₋₁₀ alkylene)-S—(C₁₋₂₀ alkyl); wherein said C₁₋₁₀ alkylene is substituted with one C₁₋₂₀ alkoxyl; and any second occurrence of R³ is hydrogen. In certain embodiments, one occurrence of R³ is —(C₁₋₁₀ alkylene)-O—(C₁₋₂₀ alkyl) or —(C₁₋₁₀ alkylene)-S—(C₁₋₂₀ alkyl); and any second occurrence of R³ is hydrogen.

In certain embodiments, one occurrence of R³ is —CH₂—C(H)(—O—(C₁₋₂₀ alkyl))-CH₂—O—(C₁₋₂₀ alkyl), —(CH₂)₃—O—(C₁₋₂₀ alkyl), —CH₂—C(H)(—O—(C₁₋₂₀ alkyl))-CH₂—S—(C₁₋₂₀ alkyl), or —(CH₂)₃—S—(C₁₋₂₀ alkyl); and any second occurrence of R³ is hydrogen. In certain embodiments, one occurrence of R³ is —CH₂—C(H)(—O—(C₁₋₂₀ alkyl))-CH₂—O—(C₁₋₂₀ alkyl) or —(CH₂)₃—O—(C₁₋₂₀ alkyl), and any second occurrence of R³ is hydrogen. In certain embodiments, one occurrence of R³ is —CH₂—C(H)(—O—(C₁₋₂₀ alkyl))-CH₂—O—(C₁₋₂₀ alkyl), and any second occurrence of R³ is hydrogen. In certain embodiments, one occurrence of R³ is —(CH₂)₃—O—(C₁₋₂₀ alkyl), and any second occurrence of R³ is hydrogen. In certain embodiments, one occurrence of R³ is —CH₂—C(H)(—O—(C₁₋₂₀ alkyl))-CH₂—S—(C₁₋₂₀ alkyl) or —(CH₂)₃—S—(C₁₋₂₀ alkyl), and any second occurrence of R³ is hydrogen. In certain embodiments, one occurrence of R³ is —CH₂—C(H)(—O—(C₁₋₂₀ alkyl))-CH₂—S—(C₁₋₂₀ alkyl), and any second occurrence of R³ is hydrogen. In certain embodiments, one occurrence of R³ is —(CH₂)₃—S—(C₁₋₂₀ alkyl), and any second occurrence of R³ is hydrogen.

In certain embodiments, R³ represents independently for each occurrence C₁₋₂₀ alkyl optionally substituted with one hydroxyl. In certain embodiments, R³ represents independently for each occurrence C₁₋₇ alkyl optionally substituted with one hydroxyl. In certain embodiments, R³ represents independently for each occurrence C₁₋₄ alkyl optionally substituted with one hydroxyl. In certain embodiments, R³ represents independently for each occurrence C₁₋₂₀ haloalkyl optionally substituted with one hydroxyl. In certain embodiments, R³ represents independently for each occurrence C₁₋₁₀ haloalkyl optionally substituted with one hydroxyl. In certain embodiments, R³ represents independently for each occurrence C₁₋₄ haloalkyl optionally substituted with one hydroxyl.

In certain embodiments, R³ represents independently for each occurrence —(C₁₋₁₀ alkylene)-O—(C₁₋₁₀ alkyl), wherein said C₁₋₁₀ alkyl is optionally substituted with one hydroxyl. In certain embodiments, R³ represents independently for each occurrence —(C₁₋₁₀ alkylene)-OC(O)—(C₁₋₁₀ alkyl), wherein said C₁₋₁₀ alkyl is optionally substituted with one hydroxyl. In certain embodiments, R³ represents independently for each occurrence —(C₁₋₁₀ alkylene)-S—(C₁₋₁₀ alkyl), wherein said C₁₋₁₀ alkyl is optionally substituted with one hydroxyl. In certain embodiments, R³ represents independently for each occurrence —(C₁₋₁₀ alkylene)-SC(O)—(C₁₋₁₀ alkyl), wherein said C₁₋₁₀ alkyl is optionally substituted with one hydroxyl.

In certain embodiments, R³ represents independently for each occurrence C₁₋₂₀ alkyl. In certain embodiments, R³ represents independently for each occurrence C₁₋₇ alkyl. In certain embodiments, R³ represents independently for each occurrence C₁₋₄ alkyl. In certain embodiments, R³ represents independently for each occurrence C₁₋₂₀ haloalkyl. In certain embodiments, R³ represents independently for each occurrence C₁₋₁₀ haloalkyl. In certain embodiments, R³ represents independently for each occurrence C₁₋₄ haloalkyl. In certain embodiments, R³ represents independently for each occurrence —(C₁₋₁₀ alkylene)-O—(C₁₋₁₀ alkyl). In certain embodiments, R³ represents independently for each occurrence —(C₁₋₁₀ alkylene)-OC(O)—(C₁₋₁₀ alkyl). In certain embodiments, R³ represents independently for each occurrence —(C₁₋₁₀ alkylene)-S—(C₁₋₁₀ alkyl). In certain embodiments, R³ represents independently for each occurrence —(C₁₋₁₀ alkylene)-SC(O)—(C₁₋₁₀ alkyl).

In certain embodiments, R³ represents independently for each occurrence

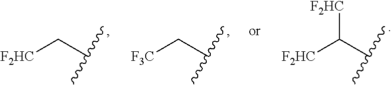 [SMILES: *CC(F)F ; *CC(F)(F)F ; *C(C(F)F)C(F)F]

In certain embodiments, R³ is

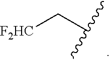 [SMILES: *CC(F)F]

In certain embodiments, R³ is

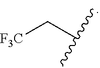 [SMILES: *CC(F)(F)F]

In certain embodiments, R³ is

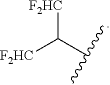 [SMILES: *C(C(F)F)C(F)F]

In certain embodiments, R³ represents independently for each occurrence hydrogen, —P(O)(OH)₂, or —P(O)(OH)—O—P(O)(OH)₂. In certain embodiments, one occurrence of R³ is hydrogen, and any second occurrence of R³ is hydrogen, —P(O)(OH)₂, or —P(O)(OH)—O—P(O)(OH)₂. In certain embodiments, R³ is hydrogen. In certain embodiments, 0 or 1 occurrence of R³ is hydrogen. In certain embodiments, one occurrence of R³ is hydrogen, and any second occurrence of R³ is —P(O)(OH)₂ or —P(O)(OH)—O—P(O)(OH)₂. In certain embodiments, one occurrence of R³ is hydrogen, and any second occurrence of R³ is —P(O)(OH)₂. In certain embodiments, one occurrence of R³ is hydrogen, and any second occurrence of R³ is —P(O)(OH)—O—P(O)(OH)₂.

In certain embodiments, two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸.

In certain embodiments, two instances of R³ are taken together to form a C₂₋₄ bivalent hydrocarbon chain substituted with p instances of R⁸. In certain embodiments, two instances of R³ are taken together to form a C₂₋₄ bivalent hydrocarbon chain substituted with one instance of —(C₁₋₃ alkylene)-CO₂R¹⁰. In certain embodiments, two instances of R³ are taken together to form a C₂₋₄ bivalent hydrocarbon chain.

In certain embodiments, two instances of R³ are taken together to form a C₂₋₄ bivalent hydrocarbon chain ortho-fused to a 6-membered aromatic ring having 0, 1, or 2 nitrogen atoms; wherein said ring is substituted with p instances of R⁸. In certain embodiments, two instances of R³ are taken together to form a C₂₋₄ bivalent hydrocarbon chain ortho-fused to a 6-membered aromatic ring having 0, 1, or 2 nitrogen atoms.

In certain embodiments, two instances of R³ are taken together to form a C₂₋₄ bivalent hydrocarbon chain ortho-fused to a 6-membered carbocyclic aromatic ring; wherein said ring is substituted with p instances of R⁸. In certain embodiments, two instances of R³ are taken together to form a C₃ bivalent hydrocarbon chain ortho-fused to a 6-membered carbocyclic aromatic ring; wherein said ring is substituted with p instances of R⁸. In certain embodiments, two instances of R³ are taken together to form a C₃ bivalent hydrocarbon chain ortho-fused to a 6-membered carbocyclic aromatic ring.

In certain embodiments, two instances of R³ are taken together to form a C₂₋₄ bivalent hydrocarbon chain ortho-fused to a 6-membered aromatic ring having 1 or 2 nitrogen atoms; wherein said ring is substituted with p instances of R⁸. In certain embodiments, two instances of R³ are taken together to form a C₃ bivalent hydrocarbon chain ortho-fused to a 6-membered aromatic ring having 1 or 2 nitrogen atoms; wherein said ring is substituted with p instances of R⁸. In certain embodiments, two instances of R³ are taken together to form a C₃ bivalent hydrocarbon chain ortho-fused to a 6-membered aromatic ring having 1 or 2 nitrogen atoms.

As defined generally above, R⁸ represents independently for each occurrence halo, C₁. 4 alkyl, C₁₋₄ haloalkyl, C₁₋₄ alkoxyl, —(C_{0.3} alkylene)-CO₂R¹⁰, or —N(R⁹)₂. In certain embodiments, R⁸ represents independently for each occurrence halo, C₁₋₄ alkyl, C₁₋₄ haloalkyl, C₁₋₄ alkoxyl, or —N(R⁹)₂. In certain embodiments, R⁸ represents independently for each occurrence halo, C₁₋₄ alkyl, C₁₋₄ haloalkyl, or C₁₋₄ alkoxyl. In certain embodiments, R⁸ represents independently for each occurrence halo, C₁₋₄ alkyl, or C₁₋₄ haloalkyl.

In certain embodiments, R⁸ represents independently for each occurrence halo. In certain embodiments, R⁸ represents independently for each occurrence C₁₋₄ alkyl. In certain embodiments, R⁸ represents independently for each occurrence C₁₋₄ haloalkyl. In certain embodiments, R⁸ represents independently for each occurrence C₁₋₄ alkoxyl. In certain embodiments, R⁸ represents independently for each occurrence —(C_{0.3} alkylene)-CO₂R¹⁰. In certain embodiments, R⁸ represents independently for each occurrence —CO₂R¹⁰. In certain embodiments, R⁸ represents independently for each occurrence —(C₁₋₃ alkylene)-CO₂R¹⁰. In certain embodiments, R⁸ represents independently for each occurrence —N(R⁹)₂.

As defined generally above, R⁹ represents independently for each occurrence hydrogen or C₁₋₄ alkyl. In certain embodiments, R⁹ represents independently for each occurrence hydrogen or methyl. In certain embodiments, R⁹ represents independently for each occurrence C₁₋₄ alkyl. In certain embodiments, R⁹ is methyl. In certain embodiments, R⁹ is hydrogen.

As defined generally above, R¹⁰ represents independently for each occurrence C₁₋₆ alkyl, C₃₋₇ cycloalkyl, or hydrogen. In certain embodiments, R¹⁰ represents independently for each occurrence C₁₋₆ alkyl or hydrogen. In certain embodiments, R¹⁰ represents independently for each occurrence C₁₋₄ alkyl or hydrogen. In certain embodiments, R¹⁰ represents independently for each occurrence C₁₋₆ alkyl or C₃₋₇ cycloalkyl. In certain embodiments, R¹⁰ represents independently for each occurrence C₃₋₇ cycloalkyl or hydrogen.

In certain embodiments, R¹⁰ represents independently for each occurrence C₁₋₆ alkyl. In certain embodiments, R¹⁰ represents independently for each occurrence C₁₋₄ alkyl. In certain embodiments, R¹⁰ is methyl. In certain embodiments, R¹⁰ represents independently for each occurrence C₃₋₇ cycloalkyl. In certain embodiments, R¹⁰ represents independently for each occurrence C₃₋₅ cycloalkyl. In certain embodiments, R¹⁰ is hydrogen.

As defined generally above, X represents independently for each occurrence —O—, ψ-OC(O)—, —OC(O)O—, ψ-OC(O)—N(R⁹)—, —S—, —S—S—, or ψ-SC(O)—; wherein ψ denotes the point of attachment to C₁₋₁₀ alkylene. In certain embodiments, X represents independently for each occurrence —O—, ψ-OC(O)—, —OC(O)O—, ψ-OC(O)—N(R⁹)—, —S—, or ψ-SC(O)—; wherein ψ denotes the point of attachment to C₁₋₁₀ alkylene.

In certain embodiments, X represents independently for each occurrence —O— or —S—. In certain embodiments, X represents independently for each occurrence ψ-OC(O)— or ψ-SC(O)—; wherein ψ denotes the point of attachment to C₁₋₁₀ alkylene. In certain embodiments, X represents independently for each occurrence —OC(O)O— or ψ-OC(O)—N(R⁹)—; wherein ψ denotes the point of attachment to C₁₋₁₀ alkylene.

In certain embodiments, X represents independently for each occurrence —O—, ψ—OC(O)—, —OC(O)O—, or ψ-OC(O)—N(R⁹)—; wherein ψ denotes the point of attachment to C₁₋₁₀ alkylene. In certain embodiments, X represents independently for each occurrence —S—, —S—S—, or ψ-SC(O)—; wherein ψ denotes the point of attachment to C₁₋₁₀ alkylene. In certain embodiments, X represents independently for each occurrence —S— or ψ-SC(O)—; wherein ψ denotes the point of attachment to C₁₋₁₀ alkylene.

In certain embodiments, X is —O—. In certain embodiments, X is ψ-OC(O)—; wherein ψ denotes the point of attachment to C₁₋₁₀ alkylene. In certain embodiments, X is —OC(O)O—. In certain embodiments, X is ψ-OC(O)—N(R⁹)—; wherein ψ denotes the point of attachment to C₁₋₁₀ alkylene. In certain embodiments, X is —S—. In certain embodiments, X is —S—S—. In certain embodiments, X is ψ-SC(O)—; wherein ψ denotes the point of attachment to C₁₋₁₀ alkylene.

As defined generally above, p is 0, 1, 2, or 3. In certain embodiments, p is 0. In certain embodiments, p is 1. In certain embodiments, p is 2. In certain embodiments, p is 3. In certain embodiments, p is 0 or 1. In certain embodiments, p is 1 or 2. In certain embodiments, p is 2 or 3. In certain embodiments, p is 0, 1, or 2. In certain embodiments p is 1, 2, or 3.

The description above describes multiple embodiments relating to compounds of Formula I-B. The patent application specifically contemplates all combinations of the embodiments.

In certain other embodiments, the compound is a compound in Table 1, 1-A, 2, 3, 4, 5, or 6, below, or a pharmaceutically acceptable salt thereof. In certain embodiments, the compound is a compound in Table 1, 1-A, 2, 3, 4, 5, or 6, below. In certain other embodiments, the compound is a compound in Table 1, 2, 3, 4, 5, or 6, below, or a pharmaceutically acceptable salt thereof. In certain embodiments, the compound is a compound in Table 1, 2, 3, 4, 5, or 6, below. In certain other embodiments, the compound is a compound in Table 1, 1-A, or 2 below, or a pharmaceutically acceptable salt thereof. In certain embodiments, the compound is a compound in Table 1, 1-A, or 2 below. In certain other embodiments, the compound is a compound in Table 1 or 2 below, or a pharmaceutically acceptable salt thereof. In certain embodiments, the compound is a compound in Table 1 or 2 below. In certain other embodiments, the compound is a compound in Table 3 or 4 below, or a pharmaceutically acceptable salt thereof. In certain embodiments, the compound is a compound in Table 3 or 4 below. In certain other embodiments, the compound is a compound in Table 5 or 6 below, or a pharmaceutically acceptable salt thereof. In certain embodiments, the compound is a compound in Table 5 or 6 below.

In certain other embodiments, the compound is compound I-1, I-20, I-21, I-115, V-1, V-7, V-105, V-106, V-118, or VI-1, below, or a pharmaceutically acceptable salt thereof. In certain embodiments, the compound is compound I-1, I-20, I-21, I-115, V-1, V-7, V-105, V-106, V-118, or VI-1, below. In certain other embodiments, the compound is compound I-1, I-20, I-21, V-1, V-7, V-105, V-106, V-118, or VI-1, below, or a pharmaceutically acceptable salt thereof. In certain embodiments, the compound is compound I-1, I-20, I-21, V-1, V-7, V-105, V-106, V-118, or VI-1, below. In certain other embodiments, the compound is compound I-1, I-20, I-21, V-1, V-7, V-118, or VI-1, below, or a pharmaceutically acceptable salt thereof. In certain embodiments, the compound is compound I-1, I-20, I-21, V-1, V-7, V-118, or VI-1, below.

In certain embodiments, the compound is a compound in Table 1 below, or a pharmaceutically acceptable salt thereof. In certain embodiments, the compound is a compound in Table 1 below. Compounds in Table 1 below can be prepared based on procedures described in, for example, WO 02/08241, WO 2014/032481, WO 2015/197006, CN 108101943 A, WO 2017/219915, WO 2019/120071, and US 2019/0015432; and related patents and applications, such as U.S. Pat. Nos. 7,390,791; 9,908,908; EP 3719027 A1; and US 2021/0093650 A1.

TABLE 1

| Compound No. | Chemical Structure |
|---|---|
| I-1 | 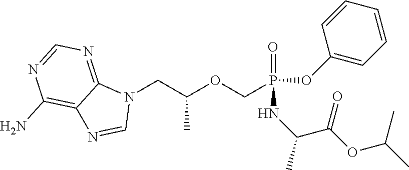 [SMILES: CC(C)OC(=O)[C@H](C)N[P@](=O)(CO[C@H](C)Cn1cnc2c(N)ncnc21)Oc1ccccc1] |
| I-2 | 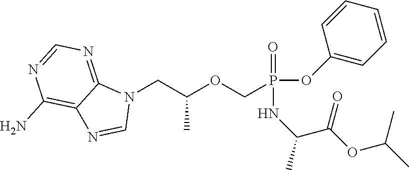 [SMILES: CC(C)OC(=O)[C@H](C)NP(=O)(CO[C@H](C)Cn1cnc2c(N)ncnc21)Oc1ccccc1] |
| I-3 | 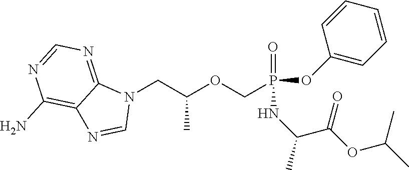 [SMILES: CC(C)OC(=O)[C@H](C)N[P@@](=O)(CO[C@H](C)Cn1cnc2c(N)ncnc21)Oc1ccccc1] |
| I-4 | 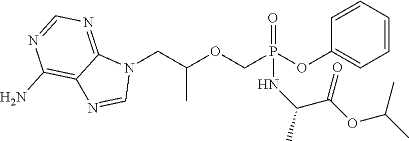 [SMILES: CC(C)OC(=O)[C@H](C)NP(=O)(COC(C)Cn1cnc2c(N)ncnc21)Oc1ccccc1] |
| I-5 | 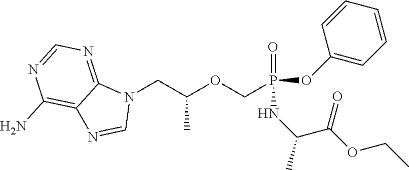 [SMILES: CCOC(=O)[C@H](C)N[P@@](=O)(CO[C@H](C)Cn1cnc2c(N)ncnc21)Oc1ccccc1] |
| I-6 | 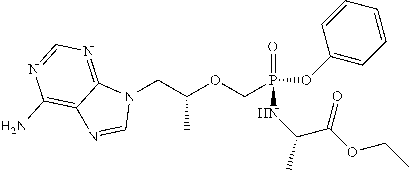 [SMILES: CCOC(=O)[C@H](C)N[P@](=O)(CO[C@H](C)Cn1cnc2c(N)ncnc21)Oc1ccccc1] |
| I-7 | 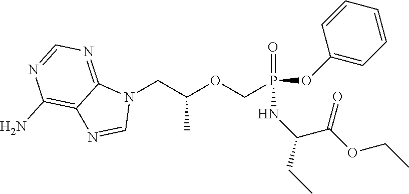 [SMILES: CCOC(=O)[C@H](CC)N[P@@](=O)(CO[C@H](C)Cn1cnc2c(N)ncnc21)Oc1ccccc1] |
| I-8 | 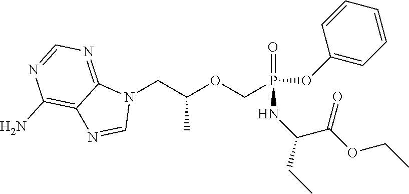 [SMILES: CCOC(=O)[C@H](CC)N[P@](=O)(CO[C@H](C)Cn1cnc2c(N)ncnc21)Oc1ccccc1] |
| I-9 | 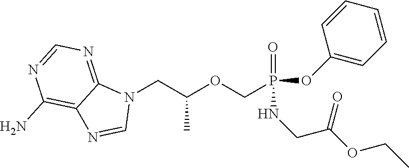 [SMILES: CCOC(=O)CN[P@@](=O)(CO[C@H](C)Cn1cnc2c(N)ncnc21)Oc1ccccc1] |
| I-10 | 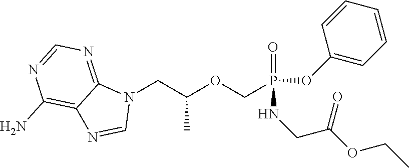 [SMILES: CCOC(=O)CN[P@](=O)(CO[C@H](C)Cn1cnc2c(N)ncnc21)Oc1ccccc1] |
| I-11 | 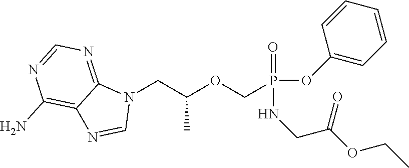 [SMILES: CCOC(=O)CNP(=O)(CO[C@H](C)Cn1cnc2c(N)ncnc21)Oc1ccccc1] |
| I-12 | 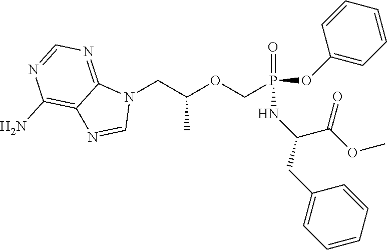 [SMILES: COC(=O)[C@H](Cc1ccccc1)N[P@@](=O)(CO[C@H](C)Cn1cnc2c(N)ncnc21)Oc1ccccc1] |
| I-13 | 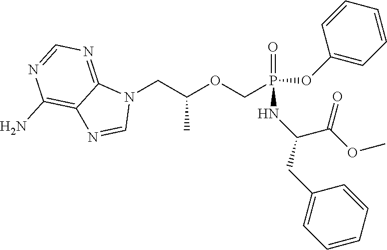 [SMILES: COC(=O)[C@H](Cc1ccccc1)N[P@](=O)(CO[C@H](C)Cn1cnc2c(N)ncnc21)Oc1ccccc1] |
| I-14 | 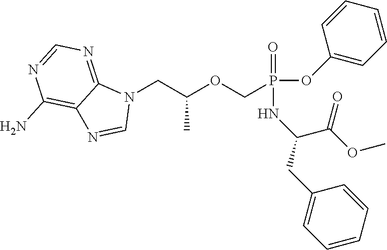 [SMILES: COC(=O)[C@H](Cc1ccccc1)NP(=O)(CO[C@H](C)Cn1cnc2c(N)ncnc21)Oc1ccccc1] |
| I-15 | 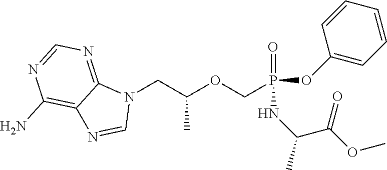 [SMILES: COC(=O)[C@H](C)N[P@@](=O)(CO[C@H](C)Cn1cnc2c(N)ncnc21)Oc1ccccc1] |
| I-16 | 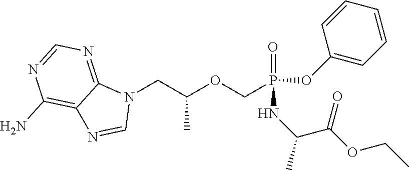 [SMILES: CCOC(=O)[C@H](C)N[P@](=O)(CO[C@H](C)Cn1cnc2c(N)ncnc21)Oc1ccccc1] |
| I-17 | 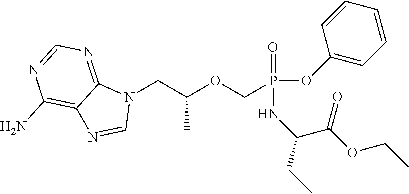 [SMILES: CCOC(=O)[C@H](CC)NP(=O)(CO[C@H](C)Cn1cnc2c(N)ncnc21)Oc1ccccc1] |
| I-18 | 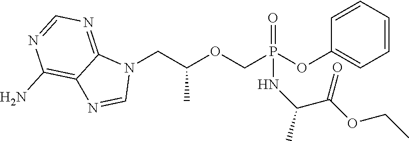 [SMILES: CCOC(=O)[C@H](C)NP(=O)(CO[C@H](C)Cn1cnc2c(N)ncnc21)Oc1ccccc1] |
| I-19 | 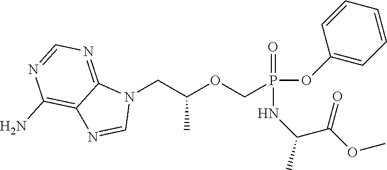 [SMILES: COC(=O)[C@H](C)NP(=O)(CO[C@H](C)Cn1cnc2c(N)ncnc21)Oc1ccccc1] |
| I-20 | 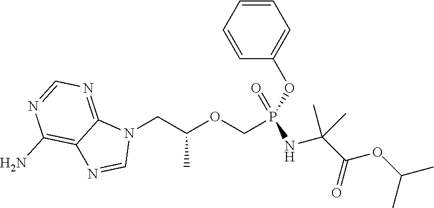 [SMILES: CC(C)OC(=O)C(C)(C)N[P@](=O)(CO[C@H](C)Cn1cnc2c(N)ncnc21)Oc1ccccc1] |
| I-21 | 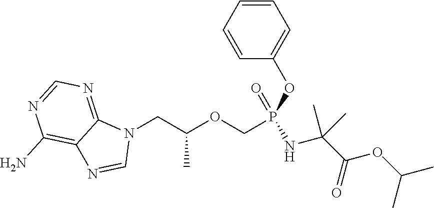 [SMILES: CC(C)OC(=O)C(C)(C)N[P@@](=O)(CO[C@H](C)Cn1cnc2c(N)ncnc21)Oc1ccccc1] |
| I-22 | 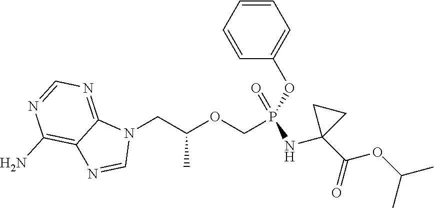 [SMILES: CC(C)OC(=O)C1(N[P@](=O)(CO[C@H](C)Cn2cnc3c(N)ncnc32)Oc2ccccc2)CC1] |
| I-23 | 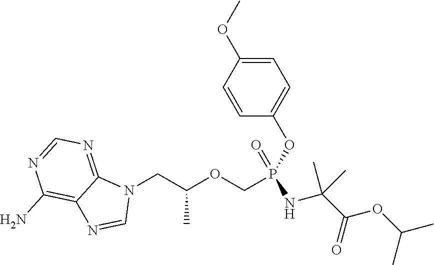 [SMILES: COc1ccc(O[P@@](=O)(CO[C@H](C)Cn2cnc3c(N)ncnc32)NC(C)(C)C(=O)OC(C)C)cc1] |
| I-24 | 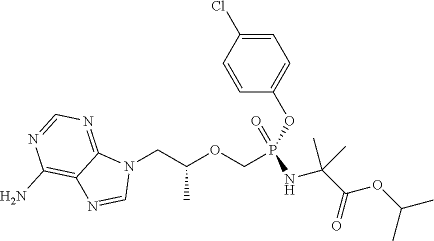 [SMILES: CC(C)OC(=O)C(C)(C)N[P@](=O)(CO[C@H](C)Cn1cnc2c(N)ncnc21)Oc1ccc(Cl)cc1] |
| I-25 | 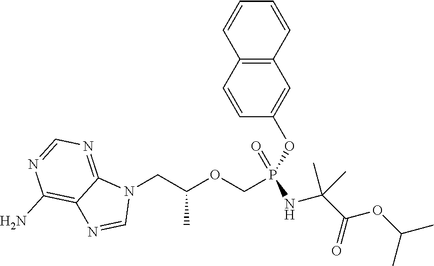 [SMILES: CC(C)OC(=O)C(C)(C)N[P@](=O)(CO[C@H](C)Cn1cnc2c(N)ncnc21)Oc1ccc2ccccc2c1] |
| I-26 | 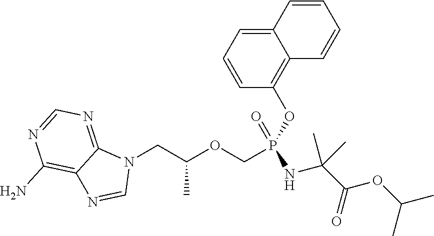 [SMILES: CC(C)OC(=O)C(C)(C)N[P@](=O)(CO[C@H](C)Cn1cnc2c(N)ncnc21)Oc1cccc2ccccc12] |
| I-27 | 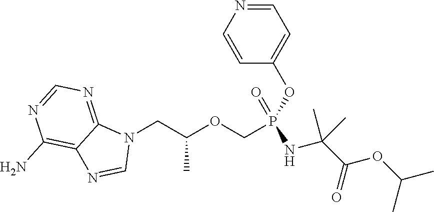 [SMILES: CC(C)OC(=O)C(C)(C)N[P@](=O)(CO[C@H](C)Cn1cnc2c(N)ncnc21)Oc1ccncc1] |
| I-28 | 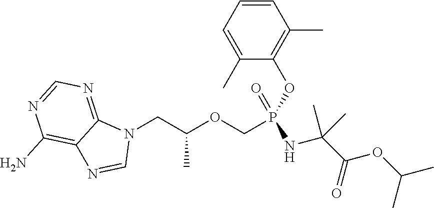 [SMILES: Cc1cccc(C)c1O[P@@](=O)(CO[C@H](C)Cn1cnc2c(N)ncnc21)NC(C)(C)C(=O)OC(C)C] |
| I-29 | 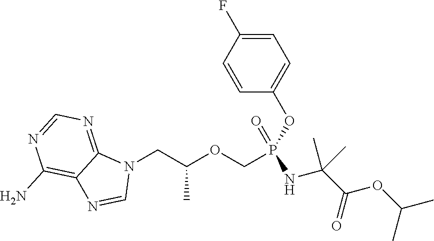 [SMILES: CC(C)OC(=O)C(C)(C)N[P@](=O)(CO[C@H](C)Cn1cnc2c(N)ncnc21)Oc1ccc(F)cc1] |
| I-30 | 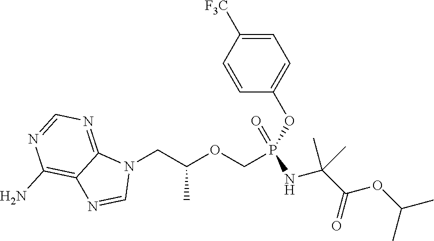 [SMILES: CC(C)OC(=O)C(C)(C)N[P@](=O)(CO[C@H](C)Cn1cnc2c(N)ncnc21)Oc1ccc(C(F)(F)F)cc1] |
| I-31 | 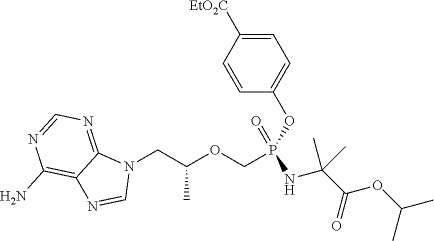 [SMILES: CCOC(=O)c1ccc(O[P@@](=O)(CO[C@H](C)Cn2cnc3c(N)ncnc32)NC(C)(C)C(=O)OC(C)C)cc1] |
| I-32 | 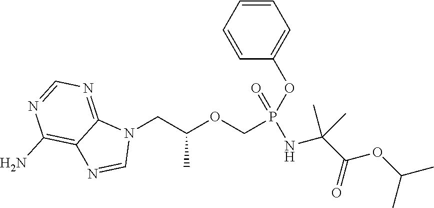 [SMILES: CC(C)OC(=O)C(C)(C)NP(=O)(CO[C@H](C)Cn1cnc2c(N)ncnc21)Oc1ccccc1] |
| I-33 | 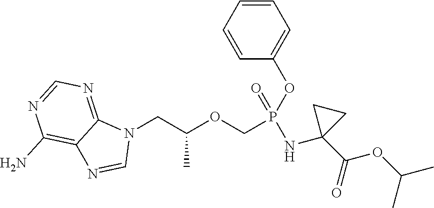 [SMILES: CC(C)OC(=O)C1(NP(=O)(CO[C@H](C)Cn2cnc3c(N)ncnc32)Oc2ccccc2)CC1] |
| I-34 | 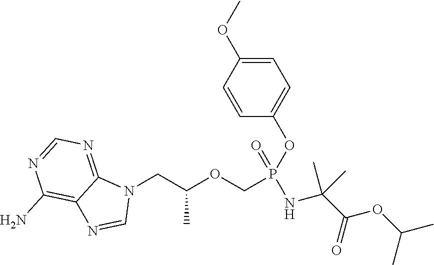 [SMILES: COc1ccc(OP(=O)(CO[C@H](C)Cn2cnc3c(N)ncnc32)NC(C)(C)C(=O)OC(C)C)cc1] |
| I-35 | 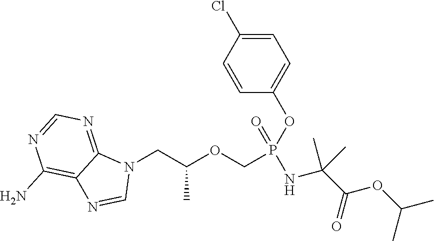 [SMILES: CC(C)OC(=O)C(C)(C)NP(=O)(CO[C@H](C)Cn1cnc2c(N)ncnc21)Oc1ccc(Cl)cc1] |
| I-36 | 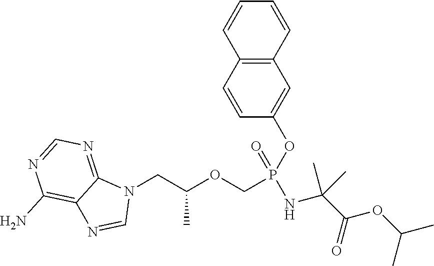 [SMILES: CC(C)OC(=O)C(C)(C)NP(=O)(CO[C@H](C)Cn1cnc2c(N)ncnc21)Oc1ccc2ccccc2c1] |
| I-37 | 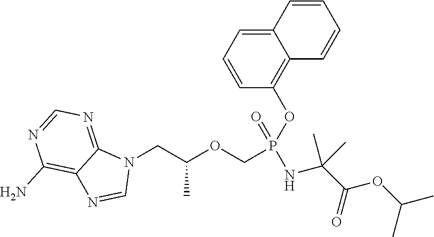 [SMILES: CC(C)OC(=O)C(C)(C)NP(=O)(CO[C@H](C)Cn1cnc2c(N)ncnc21)Oc1cccc2ccccc12] |
| I-38 | 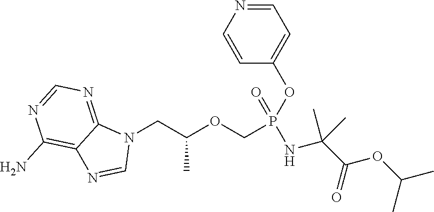 [SMILES: CC(C)OC(=O)C(C)(C)NP(=O)(CO[C@H](C)Cn1cnc2c(N)ncnc21)Oc1ccncc1] |
| I-39 | 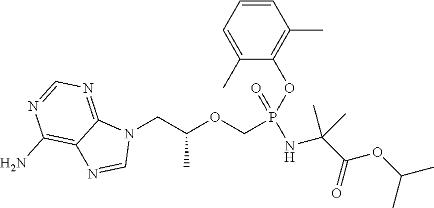 [SMILES: Cc1cccc(C)c1OP(=O)(CO[C@H](C)Cn1cnc2c(N)ncnc21)NC(C)(C)C(=O)OC(C)C] |
| I-40 | 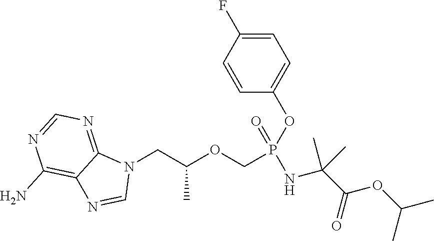 [SMILES: CC(C)OC(=O)C(C)(C)NP(=O)(CO[C@H](C)Cn1cnc2c(N)ncnc21)Oc1ccc(F)cc1] |
| I-41 | 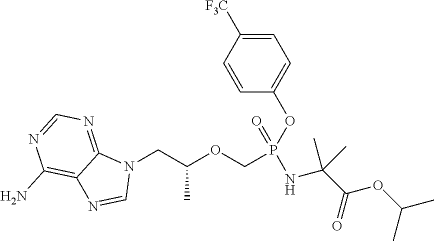 [SMILES: CC(C)OC(=O)C(C)(C)NP(=O)(CO[C@H](C)Cn1cnc2c(N)ncnc21)Oc1ccc(C(F)(F)F)cc1] |
| I-42 | 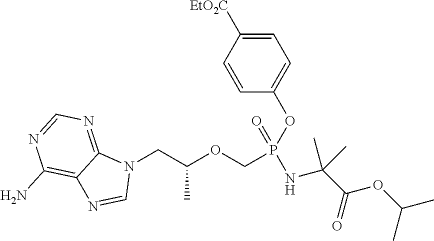 [SMILES: CCOC(=O)c1ccc(OP(=O)(CO[C@H](C)Cn2cnc3c(N)ncnc32)NC(C)(C)C(=O)OC(C)C)cc1] |
| I-43 | 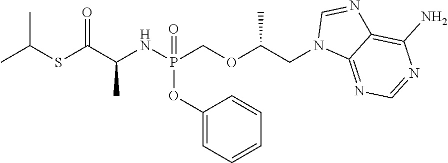 [SMILES: CC(C)SC(=O)[C@H](C)NP(=O)(CO[C@H](C)Cn1cnc2c(N)ncnc21)Oc1ccccc1] |
| I-44 | 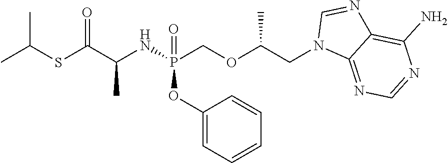 [SMILES: CC(C)SC(=O)[C@H](C)N[P@](=O)(CO[C@H](C)Cn1cnc2c(N)ncnc21)Oc1ccccc1] |
| I-45 | 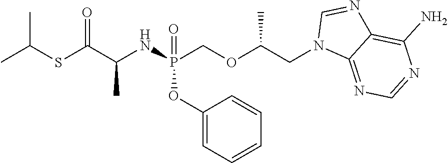 [SMILES: CC(C)SC(=O)[C@H](C)N[P@@](=O)(CO[C@H](C)Cn1cnc2c(N)ncnc21)Oc1ccccc1] |
| I-46 | 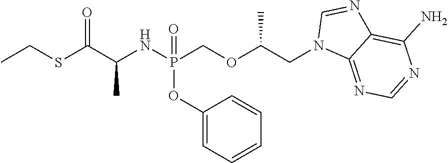 [SMILES: CCSC(=O)[C@H](C)NP(=O)(CO[C@H](C)Cn1cnc2c(N)ncnc21)Oc1ccccc1] |
| I-47 | 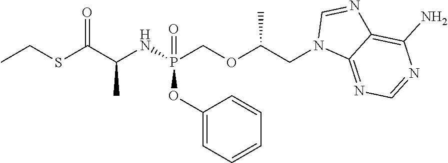 [SMILES: CCSC(=O)[C@H](C)N[P@](=O)(CO[C@H](C)Cn1cnc2c(N)ncnc21)Oc1ccccc1] |
| I-48 | 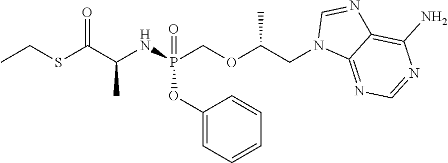 [SMILES: CCSC(=O)[C@H](C)N[P@@](=O)(CO[C@H](C)Cn1cnc2c(N)ncnc21)Oc1ccccc1] |
| I-49 | 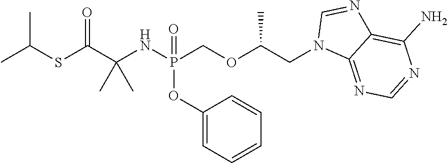 [SMILES: CC(C)SC(=O)C(C)(C)NP(=O)(CO[C@H](C)Cn1cnc2c(N)ncnc21)Oc1ccccc1] |
| I-50 | 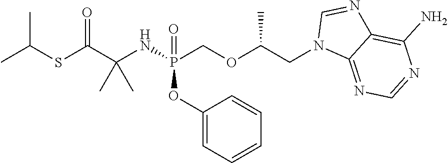 [SMILES: CC(C)SC(=O)C(C)(C)N[P@](=O)(CO[C@H](C)Cn1cnc2c(N)ncnc21)Oc1ccccc1] |
| I-51 | 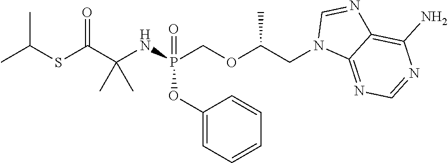 [SMILES: CC(C)SC(=O)C(C)(C)N[P@@](=O)(CO[C@H](C)Cn1cnc2c(N)ncnc21)Oc1ccccc1] |
| I-52 | 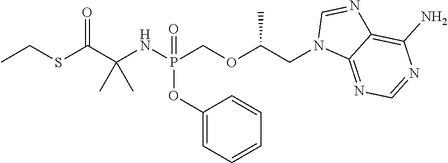 [SMILES: CCSC(=O)C(C)(C)NP(=O)(CO[C@H](C)Cn1cnc2c(N)ncnc21)Oc1ccccc1] |
| I-53 | 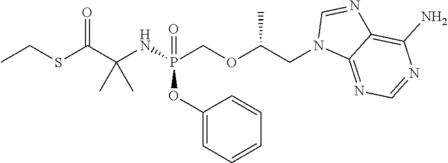 [SMILES: CCSC(=O)C(C)(C)N[P@](=O)(CO[C@H](C)Cn1cnc2c(N)ncnc21)Oc1ccccc1] |
| I-54 | 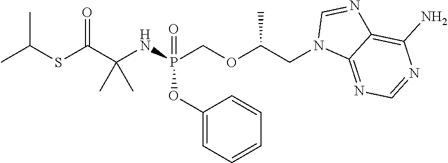 [SMILES: CC(C)SC(=O)C(C)(C)N[P@@](=O)(CO[C@H](C)Cn1cnc2c(N)ncnc21)Oc1ccccc1] |
| I-55 | 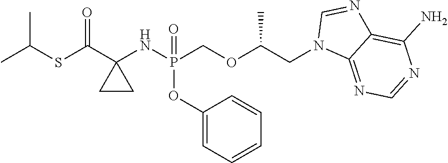 [SMILES: CC(C)SC(=O)C1(NP(=O)(CO[C@H](C)Cn2cnc3c(N)ncnc32)Oc2ccccc2)CC1] |
| I-56 | 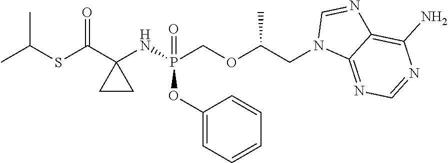 [SMILES: CC(C)SC(=O)C1(N[P@](=O)(CO[C@H](C)Cn2cnc3c(N)ncnc32)Oc2ccccc2)CC1] |
| I-57 | 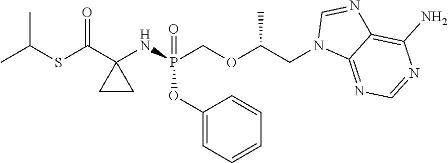 [SMILES: CC(C)SC(=O)C1(N[P@@](=O)(CO[C@H](C)Cn2cnc3c(N)ncnc32)Oc2ccccc2)CC1] |
| I-58 | 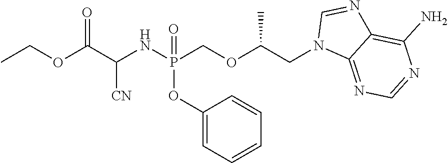 [SMILES: CCOC(=O)C(C#N)NP(=O)(CO[C@H](C)Cn1cnc2c(N)ncnc21)Oc1ccccc1] |
| I-59 | 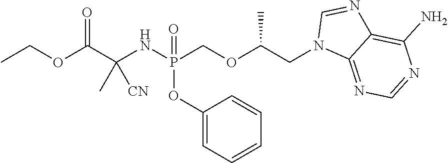 [SMILES: CCOC(=O)C(C)(C#N)NP(=O)(CO[C@H](C)Cn1cnc2c(N)ncnc21)Oc1ccccc1] |
| I-60 | 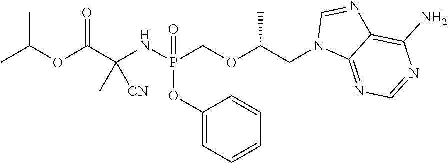 [SMILES: CC(C)OC(=O)C(C)(C#N)NP(=O)(CO[C@H](C)Cn1cnc2c(N)ncnc21)Oc1ccccc1] |
| I-61 | 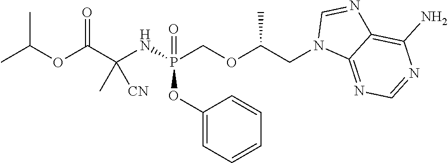 [SMILES: CC(C)OC(=O)C(C)(C#N)N[P@](=O)(CO[C@H](C)Cn1cnc2c(N)ncnc21)Oc1ccccc1] |
| I-62 | 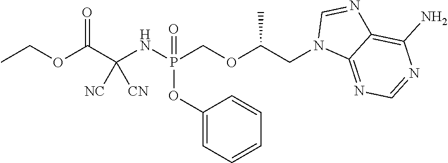 [SMILES: CCOC(=O)C(C#N)(C#N)NP(=O)(CO[C@H](C)Cn1cnc2c(N)ncnc21)Oc1ccccc1] |
| I-63 | 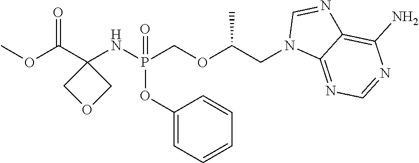 [SMILES: COC(=O)C1(NP(=O)(CO[C@H](C)Cn2cnc3c(N)ncnc32)Oc2ccccc2)COC1] |
| I-64 | 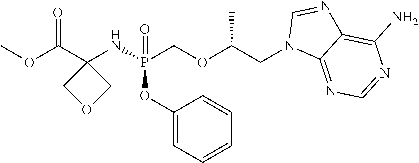 [SMILES: COC(=O)C1(N[P@](=O)(CO[C@H](C)Cn2cnc3c(N)ncnc32)Oc2ccccc2)COC1] |
| I-65 | 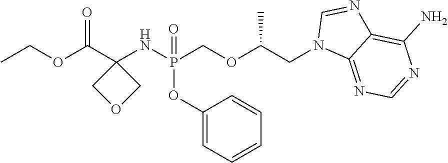 [SMILES: CCOC(=O)C1(NP(=O)(CO[C@H](C)Cn2cnc3c(N)ncnc32)Oc2ccccc2)COC1] |
| I-66 | 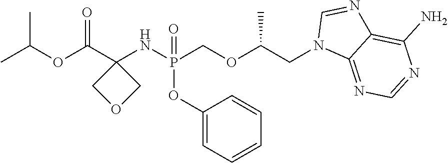 [SMILES: CC(C)OC(=O)C1(NP(=O)(CO[C@H](C)Cn2cnc3c(N)ncnc32)Oc2ccccc2)COC1] |
| I-67 | 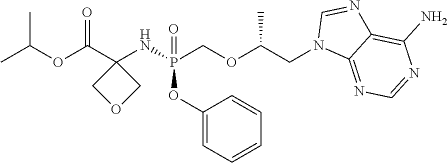 [SMILES: CC(C)OC(=O)C1(N[P@](=O)(CO[C@H](C)Cn2cnc3c(N)ncnc32)Oc2ccccc2)COC1] |
| I-68 | 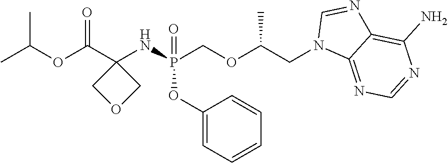 [SMILES: CC(C)OC(=O)C1(N[P@@](=O)(CO[C@H](C)Cn2cnc3c(N)ncnc32)Oc2ccccc2)COC1] |
| I-69 | 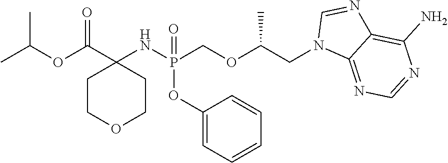 [SMILES: CC(C)OC(=O)C1(NP(=O)(CO[C@H](C)Cn2cnc3c(N)ncnc32)Oc2ccccc2)CCOCC1] |
| I-70 | 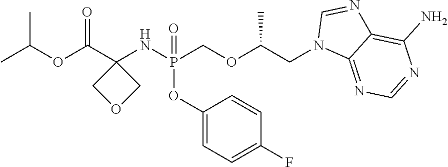 [SMILES: CC(C)OC(=O)C1(NP(=O)(CO[C@H](C)Cn2cnc3c(N)ncnc32)Oc2ccc(F)cc2)COC1] |
| I-71 | 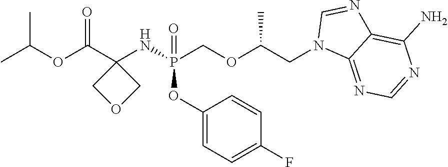 [SMILES: CC(C)OC(=O)C1(N[P@](=O)(CO[C@H](C)Cn2cnc3c(N)ncnc32)Oc2ccc(F)cc2)COC1] |
| I-72 | 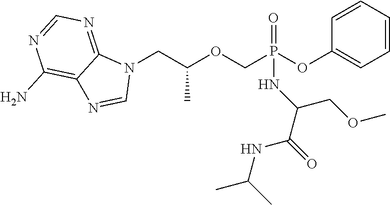 [SMILES: COCC(NP(=O)(CO[C@H](C)Cn1cnc2c(N)ncnc21)Oc1ccccc1)C(=O)NC(C)C] |
| I-73 | 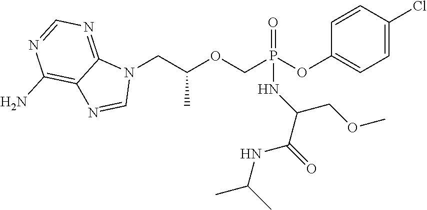 [SMILES: COCC(NP(=O)(CO[C@H](C)Cn1cnc2c(N)ncnc21)Oc1ccc(Cl)cc1)C(=O)NC(C)C] |
| I-74 | 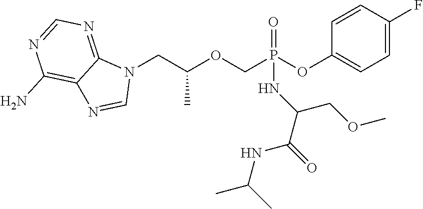 [SMILES: COCC(NP(=O)(CO[C@H](C)Cn1cnc2c(N)ncnc21)Oc1ccc(F)cc1)C(=O)NC(C)C] |
| I-75 | 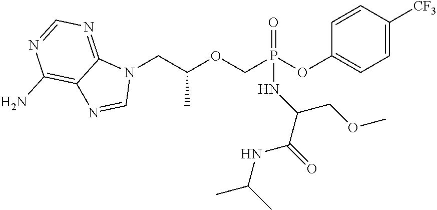 [SMILES: COCC(NP(=O)(CO[C@H](C)Cn1cnc2c(N)ncnc21)Oc1ccc(C(F)(F)F)cc1)C(=O)NC(C)C] |
| I-76 | 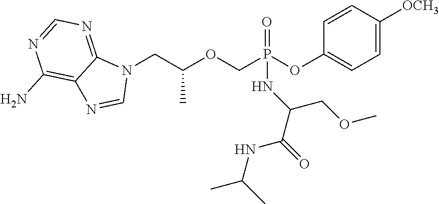 [SMILES: COCC(NP(=O)(CO[C@H](C)Cn1cnc2c(N)ncnc21)Oc1ccc(OC)cc1)C(=O)NC(C)C] |
| I-77 | 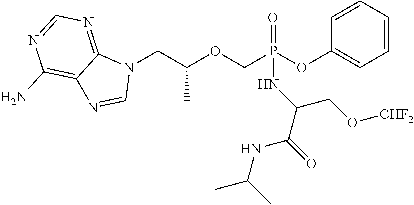 [SMILES: CC(C)NC(=O)C(COC(F)F)NP(=O)(CO[C@H](C)Cn1cnc2c(N)ncnc21)Oc1ccccc1] |
| I-78 | 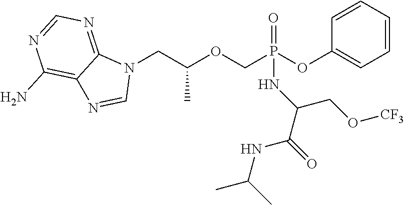 [SMILES: CC(C)NC(=O)C(COC(F)(F)F)NP(=O)(CO[C@H](C)Cn1cnc2c(N)ncnc21)Oc1ccccc1] |
| I-79 | 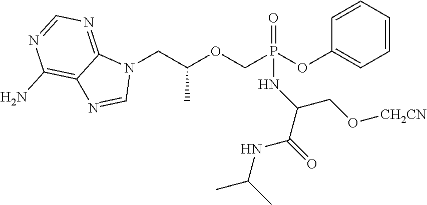 |
| I-80 | 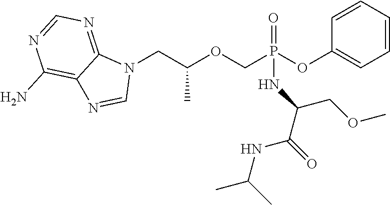 [SMILES: COC[C@H](NP(=O)(CO[C@H](C)Cn1cnc2c(N)ncnc21)Oc1ccccc1)C(=O)NC(C)C] |
| I-81 | 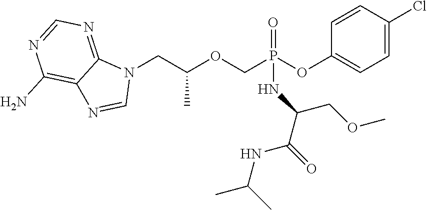 [SMILES: COC[C@H](NP(=O)(CO[C@H](C)Cn1cnc2c(N)ncnc21)Oc1ccc(Cl)cc1)C(=O)NC(C)C] |
| I-82 | 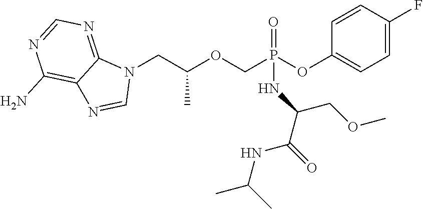 [SMILES: COC[C@H](NP(=O)(CO[C@H](C)Cn1cnc2c(N)ncnc21)Oc1ccc(F)cc1)C(=O)NC(C)C] |
| I-83 | 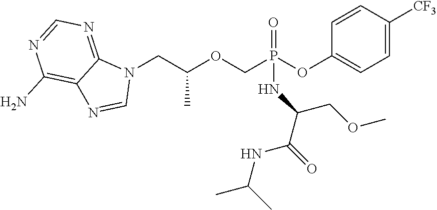 [SMILES: COC[C@H](NP(=O)(CO[C@H](C)Cn1cnc2c(N)ncnc21)Oc1ccc(C(F)(F)F)cc1)C(=O)NC(C)C] |
| I-84 | 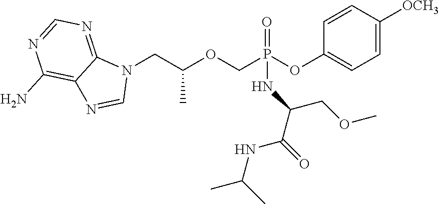 [SMILES: COC[C@H](NP(=O)(CO[C@H](C)Cn1cnc2c(N)ncnc21)Oc1ccc(OC)cc1)C(=O)NC(C)C] |
| I-85 | 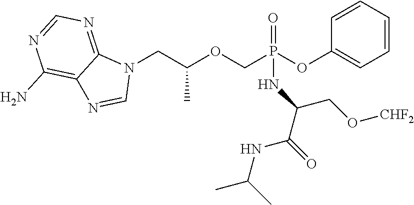 [SMILES: CC(C)NC(=O)[C@H](COC(F)F)NP(=O)(CO[C@H](C)Cn1cnc2c(N)ncnc21)Oc1ccccc1] |
| I-86 | 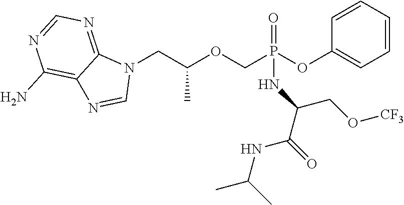 [SMILES: CC(C)NC(=O)[C@H](COC(F)(F)F)NP(=O)(CO[C@H](C)Cn1cnc2c(N)ncnc21)Oc1ccccc1] |
| I-87 | 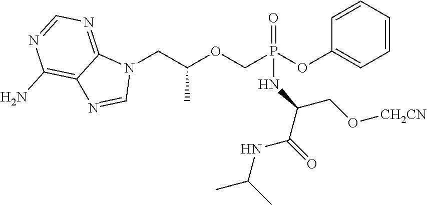 |
| I-88 | 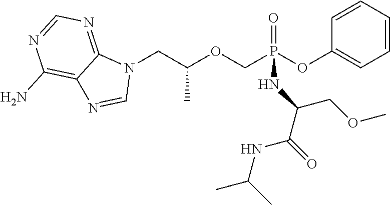 [SMILES: COC[C@H](N[P@](=O)(CO[C@H](C)Cn1cnc2c(N)ncnc21)Oc1ccccc1)C(=O)NC(C)C] |
| I-89 | 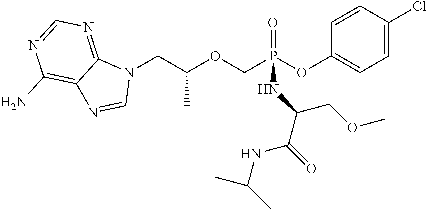 [SMILES: COC[C@H](N[P@](=O)(CO[C@H](C)Cn1cnc2c(N)ncnc21)Oc1ccc(Cl)cc1)C(=O)NC(C)C] |
| I-90 | 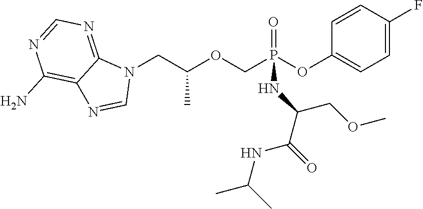 [SMILES: COC[C@H](N[P@](=O)(CO[C@H](C)Cn1cnc2c(N)ncnc21)Oc1ccc(F)cc1)C(=O)NC(C)C] |
| I-91 | 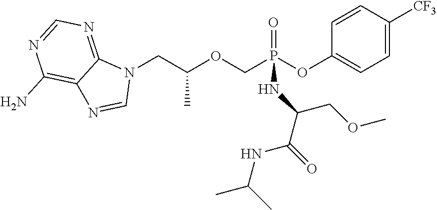 [SMILES: COC[C@H](N[P@](=O)(CO[C@H](C)Cn1cnc2c(N)ncnc21)Oc1ccc(C(F)(F)F)cc1)C(=O)NC(C)C] |
| I-92 | 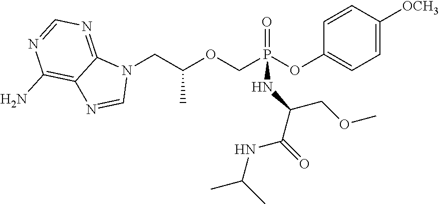 [SMILES: COC[C@H](N[P@](=O)(CO[C@H](C)Cn1cnc2c(N)ncnc21)Oc1ccc(OC)cc1)C(=O)NC(C)C] |
| I-93 | 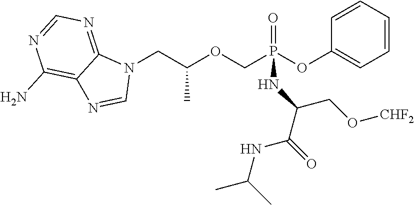 [SMILES: CC(C)NC(=O)[C@H](COC(F)F)N[P@](=O)(CO[C@H](C)Cn1cnc2c(N)ncnc21)Oc1ccccc1] |
| I-94 | 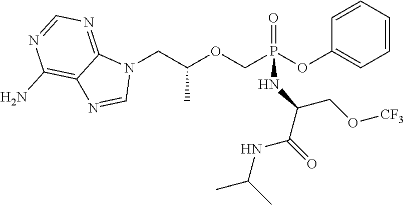 [SMILES: CC(C)NC(=O)[C@H](COC(F)(F)F)N[P@](=O)(CO[C@H](C)Cn1cnc2c(N)ncnc21)Oc1ccccc1] |
| I-95 | 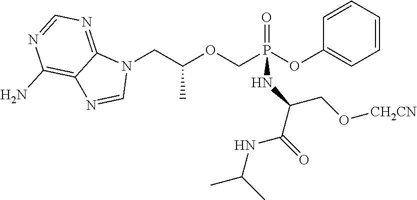 |
| I-96 | 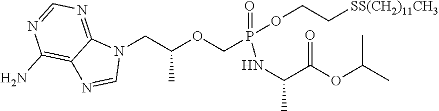 |
| I-97 | 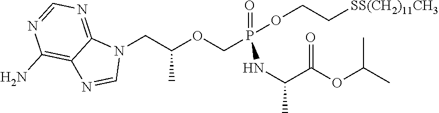 |
| I-98 | 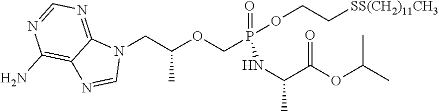 |
| I-99 | 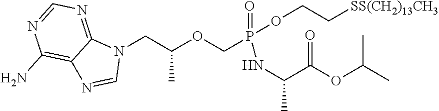 |
| I-100 | 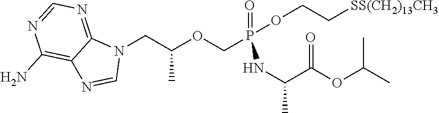 |
| I-101 | 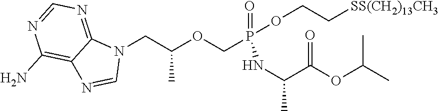 |
| I-102 | 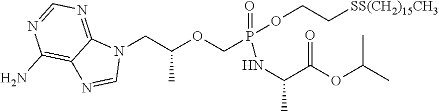 |
| I-103 | 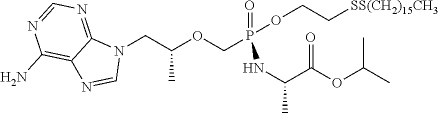 |
| I-104 | 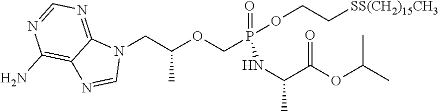 |
| I-105 | 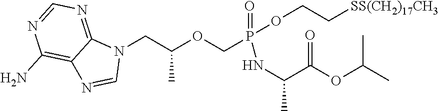 |
| I-106 | 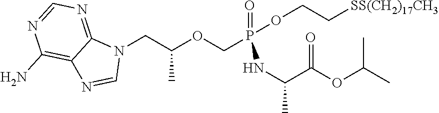 |
| I-107 | 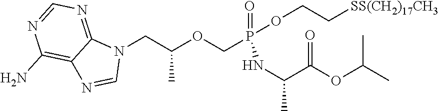 |
| I-108 | 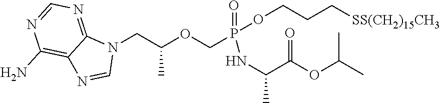 |
| I-109 | 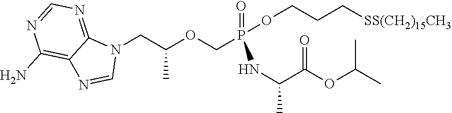 |
| I-110 | 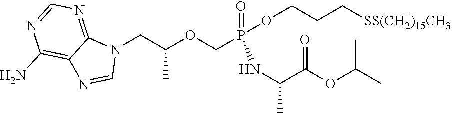 |
| I-111 | 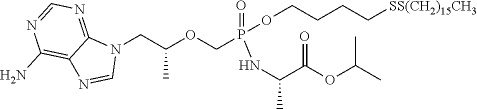 |
| I-112 | 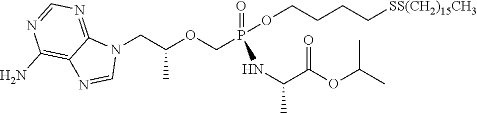 |
| I-113 | 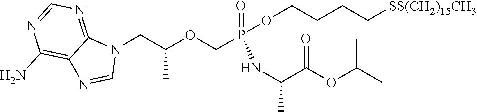 |
| I-114 | 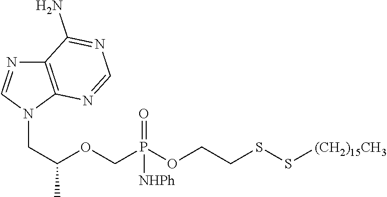 |

In certain embodiments, the compound is a compound in Table 1-A below, or a pharmaceutically acceptable salt thereof. In certain embodiments, the compound is a compound in Table 1-A below. The compound in Table 1-A below can be prepared based on procedures described in, for example, WO 2017/156262 and related patents and applications, such as U.S. Pat. No. 10,745,427.

TABLE 1-A

| Compound No. | Chemical Structure |
|---|---|
| I-115 | 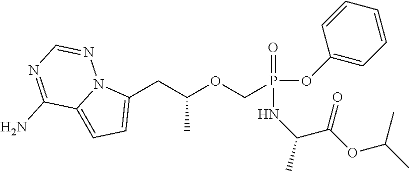 [SMILES: CC(C)OC(=O)[C@H](C)NP(=O)(CO[C@H](C)Cc1ccc2c(N)ncnn12)Oc1ccccc1] |

In certain embodiments, the compound is a compound in Table 2 below, or a pharmaceutically acceptable salt thereof. In certain embodiments, the compound is a compound in Table 2 below. Compounds in Table 2 below can be prepared based on procedures described in, for example, WO 2018/039157; and related patents and applications, such as U.S. Pat. No. 10,449,208; each of which is hereby incorporated by reference.

TABLE 2

| Compound No. | Chemical Structure |
|---|---|
| II-1 | 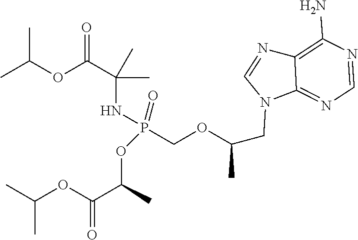 [SMILES: CC(C)OC(=O)[C@H](C)OP(=O)(CO[C@H](C)Cn1cnc2c(N)ncnc21)NC(C)(C)C(=O)OC(C)C] |
| II-2 | 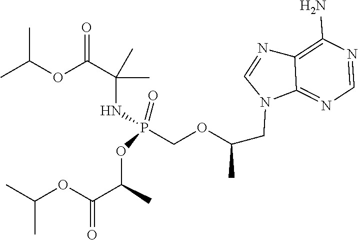 [SMILES: CC(C)OC(=O)[C@H](C)O[P@@](=O)(CO[C@H](C)Cn1cnc2c(N)ncnc21)NC(C)(C)C(=O)OC(C)C] |
| II-3 | 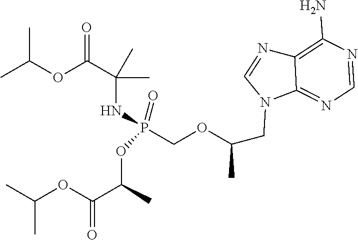 [SMILES: CC(C)OC(=O)[C@H](C)O[P@](=O)(CO[C@H](C)Cn1cnc2c(N)ncnc21)NC(C)(C)C(=O)OC(C)C] |
| II-4 | 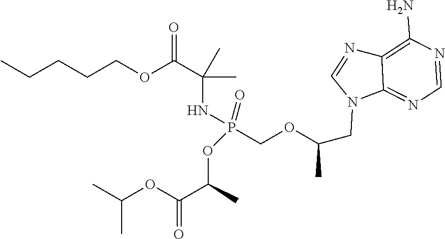 [SMILES: CCCCCOC(=O)C(C)(C)NP(=O)(CO[C@H](C)Cn1cnc2c(N)ncnc21)O[C@@H](C)C(=O)OC(C)C] |
| II-5 | 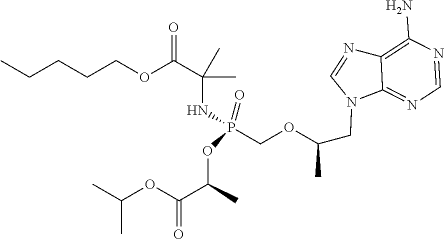 [SMILES: CCCCCOC(=O)C(C)(C)N[P@](=O)(CO[C@H](C)Cn1cnc2c(N)ncnc21)O[C@@H](C)C(=O)OC(C)C] |
| II-6 | 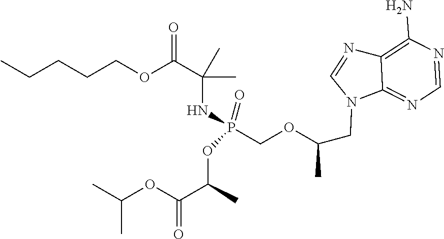 [SMILES: CCCCCOC(=O)C(C)(C)N[P@@](=O)(CO[C@H](C)Cn1cnc2c(N)ncnc21)O[C@@H](C)C(=O)OC(C)C] |
| II-7 | 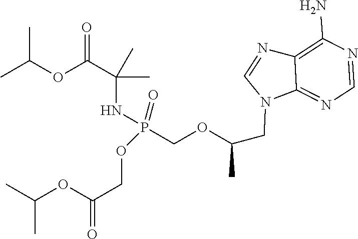 [SMILES: CC(C)OC(=O)COP(=O)(CO[C@H](C)Cn1cnc2c(N)ncnc21)NC(C)(C)C(=O)OC(C)C] |
| II-8 | 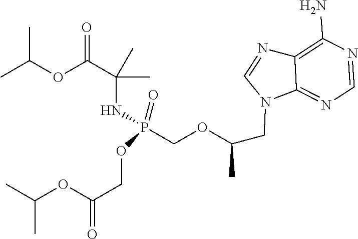 [SMILES: CC(C)OC(=O)CO[P@@](=O)(CO[C@H](C)Cn1cnc2c(N)ncnc21)NC(C)(C)C(=O)OC(C)C] |
| II-9 | 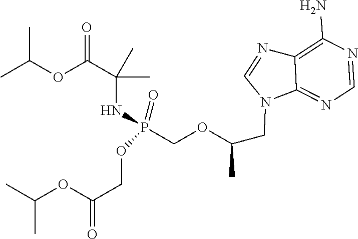 [SMILES: CC(C)OC(=O)CO[P@](=O)(CO[C@H](C)Cn1cnc2c(N)ncnc21)NC(C)(C)C(=O)OC(C)C] |
| II-10 | 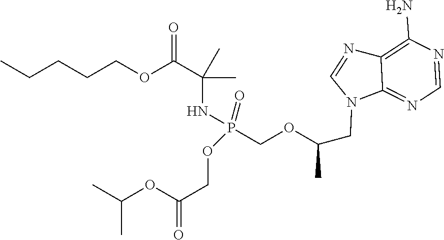 [SMILES: CCCCCOC(=O)C(C)(C)NP(=O)(CO[C@H](C)Cn1cnc2c(N)ncnc21)OCC(=O)OC(C)C] |
| II-11 | 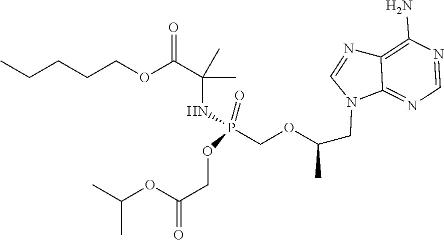 [SMILES: CCCCCOC(=O)C(C)(C)N[P@](=O)(CO[C@H](C)Cn1cnc2c(N)ncnc21)OCC(=O)OC(C)C] |
| II-12 | 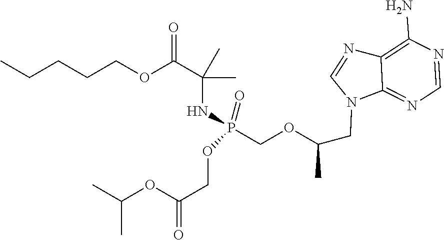 [SMILES: CCCCCOC(=O)C(C)(C)N[P@@](=O)(CO[C@H](C)Cn1cnc2c(N)ncnc21)OCC(=O)OC(C)C] |
| II-13 | 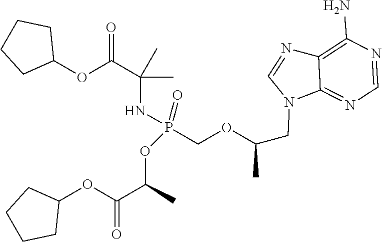 [SMILES: C[C@H](Cn1cnc2c(N)ncnc21)OCP(=O)(NC(C)(C)C(=O)OC1CCCC1)O[C@@H](C)C(=O)OC1CCCC1] |
| II-14 | 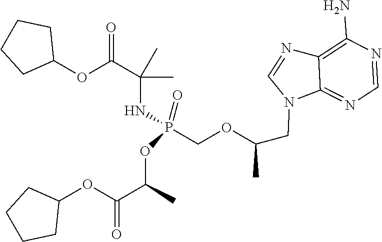 [SMILES: C[C@H](Cn1cnc2c(N)ncnc21)OC[P@@](=O)(NC(C)(C)C(=O)OC1CCCC1)O[C@@H](C)C(=O)OC1CCCC1] |
| II-15 | 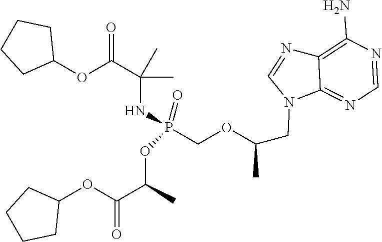 [SMILES: C[C@H](Cn1cnc2c(N)ncnc21)OC[P@](=O)(NC(C)(C)C(=O)OC1CCCC1)O[C@@H](C)C(=O)OC1CCCC1] |
| II-16 | 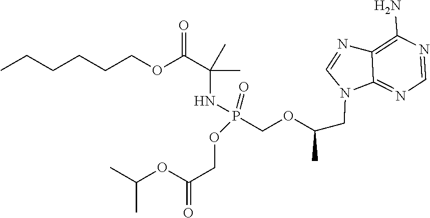 [SMILES: CCCCCCOC(=O)C(C)(C)NP(=O)(CO[C@H](C)Cn1cnc2c(N)ncnc21)OCC(=O)OC(C)C] |
| II-17 | 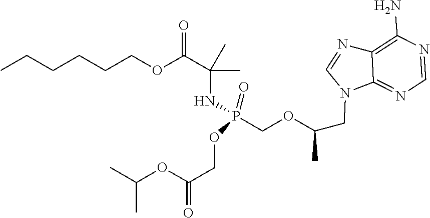 [SMILES: CCCCCCOC(=O)C(C)(C)N[P@](=O)(CO[C@H](C)Cn1cnc2c(N)ncnc21)OCC(=O)OC(C)C] |
| II-18 | 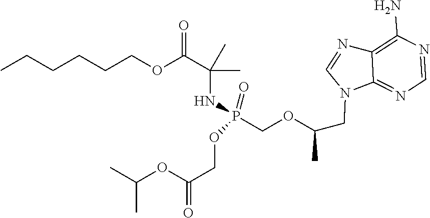 [SMILES: CCCCCCOC(=O)C(C)(C)N[P@@](=O)(CO[C@H](C)Cn1cnc2c(N)ncnc21)OCC(=O)OC(C)C] |
| II-19 | 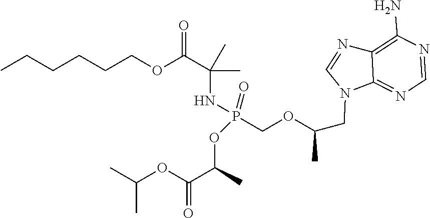 [SMILES: CCCCCCOC(=O)C(C)(C)NP(=O)(CO[C@H](C)Cn1cnc2c(N)ncnc21)O[C@@H](C)C(=O)OC(C)C] |
| II-20 | 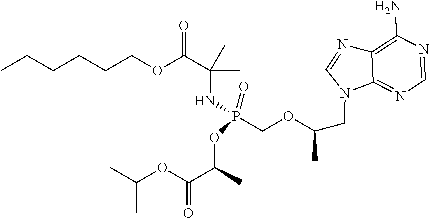 [SMILES: CCCCCCOC(=O)C(C)(C)N[P@](=O)(CO[C@H](C)Cn1cnc2c(N)ncnc21)O[C@@H](C)C(=O)OC(C)C] |
| II-21 | 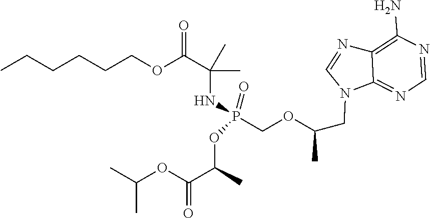 [SMILES: CCCCCCOC(=O)C(C)(C)N[P@@](=O)(CO[C@H](C)Cn1cnc2c(N)ncnc21)O[C@@H](C)C(=O)OC(C)C] |
| II-22 | 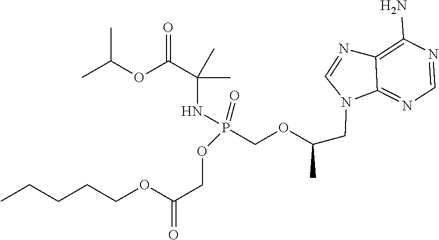 [SMILES: CCCCCOC(=O)COP(=O)(CO[C@H](C)Cn1cnc2c(N)ncnc21)NC(C)(C)C(=O)OC(C)C] |
| II-23 | 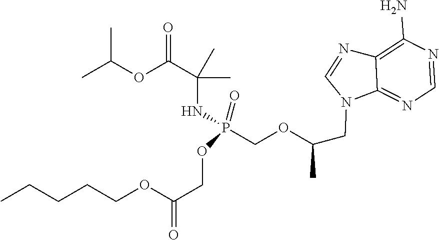 [SMILES: CCCCCOC(=O)CO[P@@](=O)(CO[C@H](C)Cn1cnc2c(N)ncnc21)NC(C)(C)C(=O)OC(C)C] |
| II-24 | 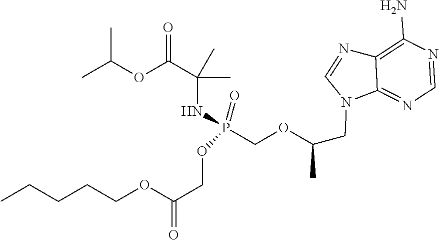 [SMILES: CCCCCOC(=O)CO[P@](=O)(CO[C@H](C)Cn1cnc2c(N)ncnc21)NC(C)(C)C(=O)OC(C)C] |
| II-25 | 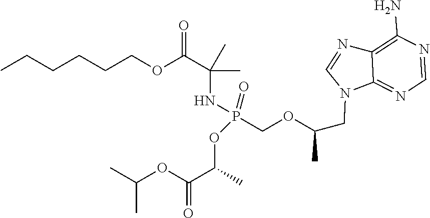 [SMILES: CCCCCCOC(=O)C(C)(C)NP(=O)(CO[C@H](C)Cn1cnc2c(N)ncnc21)O[C@H](C)C(=O)OC(C)C] |
| II-26 | 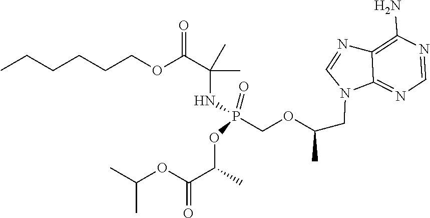 [SMILES: CCCCCCOC(=O)C(C)(C)N[P@](=O)(CO[C@H](C)Cn1cnc2c(N)ncnc21)O[C@H](C)C(=O)OC(C)C] |
| II-27 | 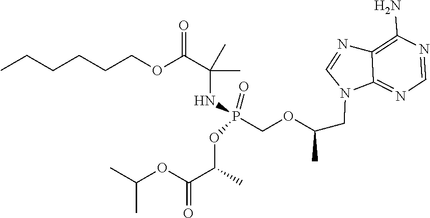 [SMILES: CCCCCCOC(=O)C(C)(C)N[P@@](=O)(CO[C@H](C)Cn1cnc2c(N)ncnc21)O[C@H](C)C(=O)OC(C)C] |
| II-28 | 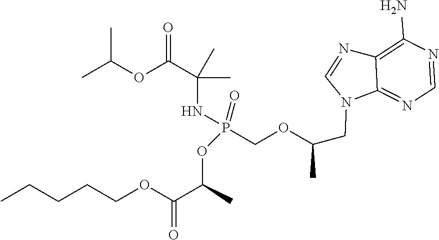 [SMILES: CCCCCOC(=O)[C@H](C)OP(=O)(CO[C@H](C)Cn1cnc2c(N)ncnc21)NC(C)(C)C(=O)OC(C)C] |
| II-29 | 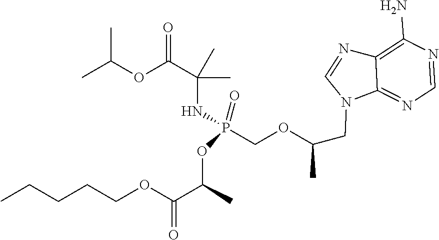 [SMILES: CCCCCOC(=O)[C@H](C)O[P@@](=O)(CO[C@H](C)Cn1cnc2c(N)ncnc21)NC(C)(C)C(=O)OC(C)C] |
| II-30 | 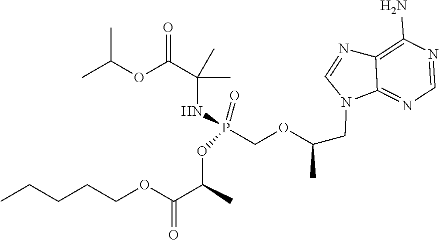 [SMILES: CCCCCOC(=O)[C@H](C)O[P@](=O)(CO[C@H](C)Cn1cnc2c(N)ncnc21)NC(C)(C)C(=O)OC(C)C] |
| II-31 | 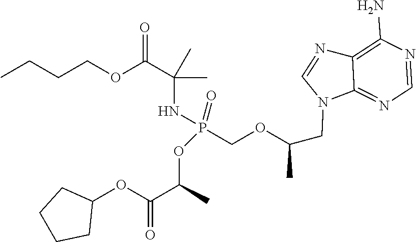 [SMILES: CCCCOC(=O)C(C)(C)NP(=O)(CO[C@H](C)Cn1cnc2c(N)ncnc21)O[C@@H](C)C(=O)OC1CCCC1] |
| II-32 | 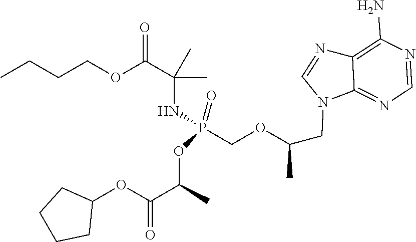 [SMILES: CCCCOC(=O)C(C)(C)N[P@](=O)(CO[C@H](C)Cn1cnc2c(N)ncnc21)O[C@@H](C)C(=O)OC1CCCC1] |
| II-33 | 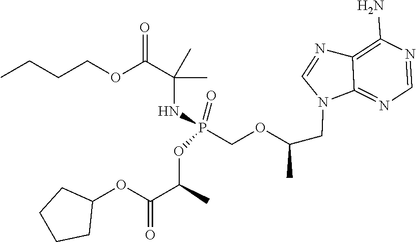 [SMILES: CCCCOC(=O)C(C)(C)N[P@@](=O)(CO[C@H](C)Cn1cnc2c(N)ncnc21)O[C@@H](C)C(=O)OC1CCCC1] |
| II-34 | 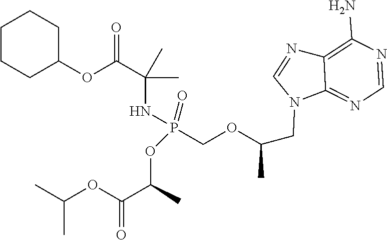 [SMILES: CC(C)OC(=O)[C@H](C)OP(=O)(CO[C@H](C)Cn1cnc2c(N)ncnc21)NC(C)(C)C(=O)OC1CCCCC1] |
| II-35 | 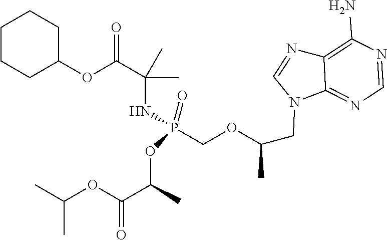 [SMILES: CC(C)OC(=O)[C@H](C)O[P@@](=O)(CO[C@H](C)Cn1cnc2c(N)ncnc21)NC(C)(C)C(=O)OC1CCCCC1] |
| II-36 | 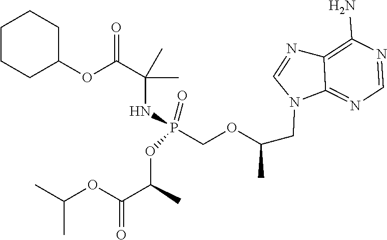 [SMILES: CC(C)OC(=O)[C@H](C)O[P@](=O)(CO[C@H](C)Cn1cnc2c(N)ncnc21)NC(C)(C)C(=O)OC1CCCCC1] |
| II-37 | 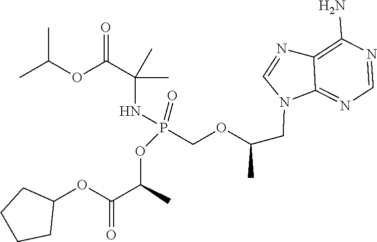 [SMILES: CC(C)OC(=O)C(C)(C)NP(=O)(CO[C@H](C)Cn1cnc2c(N)ncnc21)O[C@@H](C)C(=O)OC1CCCC1] |
| II-38 | 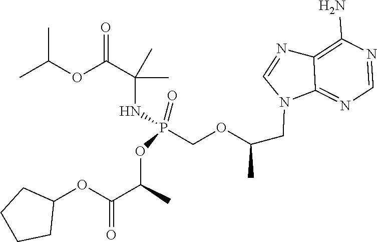 [SMILES: CC(C)OC(=O)C(C)(C)N[P@](=O)(CO[C@H](C)Cn1cnc2c(N)ncnc21)O[C@@H](C)C(=O)OC1CCCC1] |
| II-39 | 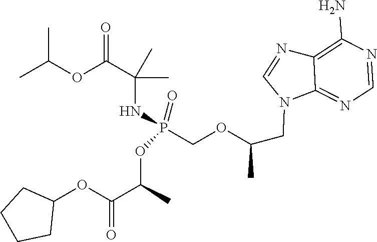 [SMILES: CC(C)OC(=O)C(C)(C)N[P@@](=O)(CO[C@H](C)Cn1cnc2c(N)ncnc21)O[C@@H](C)C(=O)OC1CCCC1] |
| II-40 | 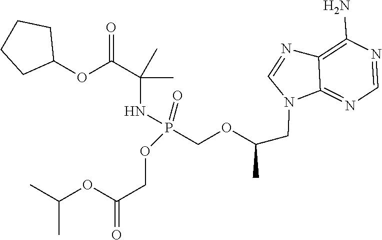 [SMILES: CC(C)OC(=O)COP(=O)(CO[C@H](C)Cn1cnc2c(N)ncnc21)NC(C)(C)C(=O)OC1CCCC1] |
| II-41 | 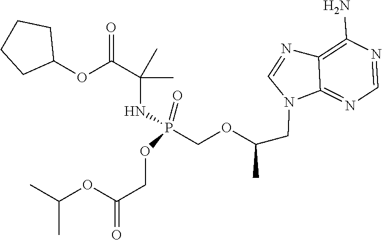 [SMILES: CC(C)OC(=O)CO[P@@](=O)(CO[C@H](C)Cn1cnc2c(N)ncnc21)NC(C)(C)C(=O)OC1CCCC1] |
| II-42 | 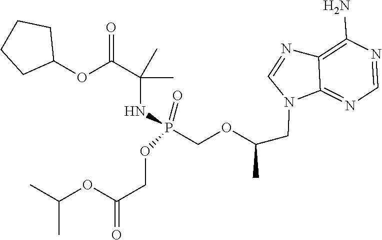 [SMILES: CC(C)OC(=O)CO[P@](=O)(CO[C@H](C)Cn1cnc2c(N)ncnc21)NC(C)(C)C(=O)OC1CCCC1] |
| II-43 | 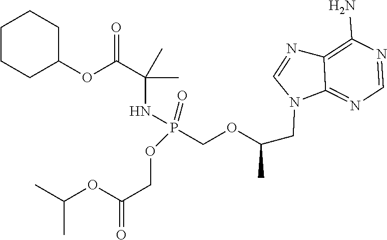 [SMILES: CC(C)OC(=O)COP(=O)(CO[C@H](C)Cn1cnc2c(N)ncnc21)NC(C)(C)C(=O)OC1CCCCC1] |
| II-44 | 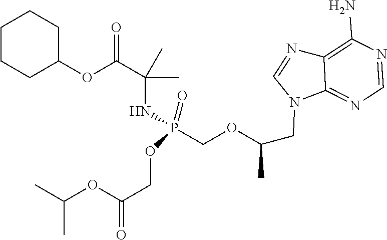 [SMILES: CC(C)OC(=O)CO[P@@](=O)(CO[C@H](C)Cn1cnc2c(N)ncnc21)NC(C)(C)C(=O)OC1CCCCC1] |
| II-45 | 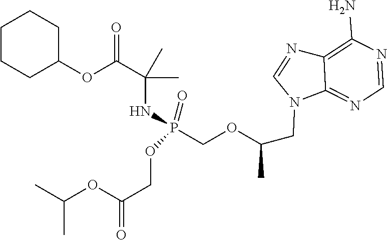 [SMILES: CC(C)OC(=O)CO[P@](=O)(CO[C@H](C)Cn1cnc2c(N)ncnc21)NC(C)(C)C(=O)OC1CCCCC1] |
| II-46 | 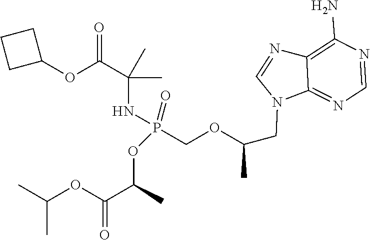 [SMILES: CC(C)OC(=O)[C@H](C)OP(=O)(CO[C@H](C)Cn1cnc2c(N)ncnc21)NC(C)(C)C(=O)OC1CCC1] |
| II-47 | 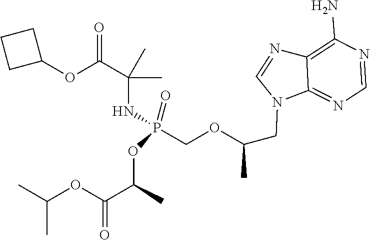 [SMILES: CC(C)OC(=O)[C@H](C)O[P@@](=O)(CO[C@H](C)Cn1cnc2c(N)ncnc21)NC(C)(C)C(=O)OC1CCC1] |
| II-48 | 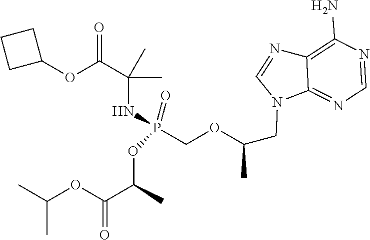 [SMILES: CC(C)OC(=O)[C@H](C)O[P@](=O)(CO[C@H](C)Cn1cnc2c(N)ncnc21)NC(C)(C)C(=O)OC1CCC1] |
| II-49 | 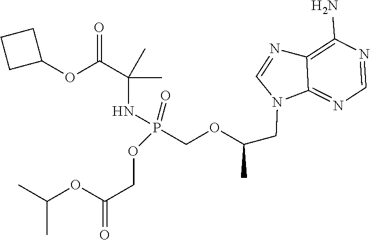 [SMILES: CC(C)OC(=O)COP(=O)(CO[C@H](C)Cn1cnc2c(N)ncnc21)NC(C)(C)C(=O)OC1CCC1] |
| II-50 | 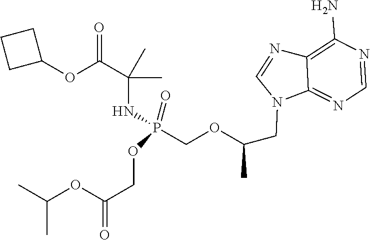 [SMILES: CC(C)OC(=O)CO[P@@](=O)(CO[C@H](C)Cn1cnc2c(N)ncnc21)NC(C)(C)C(=O)OC1CCC1] |
| II-51 | 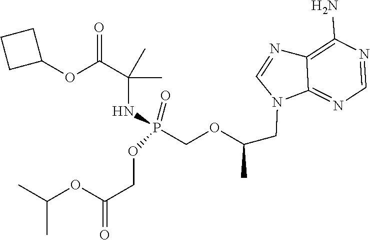 [SMILES: CC(C)OC(=O)CO[P@](=O)(CO[C@H](C)Cn1cnc2c(N)ncnc21)NC(C)(C)C(=O)OC1CCC1] |
| II-52 | 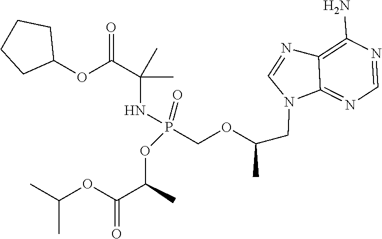 [SMILES: CC(C)OC(=O)[C@H](C)OP(=O)(CO[C@H](C)Cn1cnc2c(N)ncnc21)NC(C)(C)C(=O)OC1CCCC1] |
| II-53 | 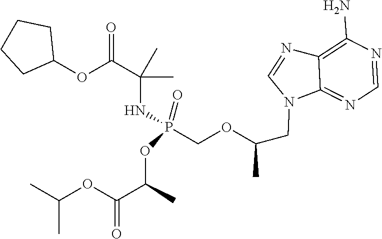 [SMILES: CC(C)OC(=O)[C@H](C)O[P@@](=O)(CO[C@H](C)Cn1cnc2c(N)ncnc21)NC(C)(C)C(=O)OC1CCCC1] |
| II-54 | 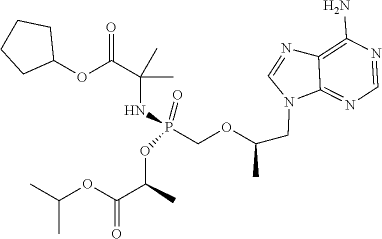 [SMILES: CC(C)OC(=O)[C@H](C)O[P@](=O)(CO[C@H](C)Cn1cnc2c(N)ncnc21)NC(C)(C)C(=O)OC1CCCC1] |
| II-55 | 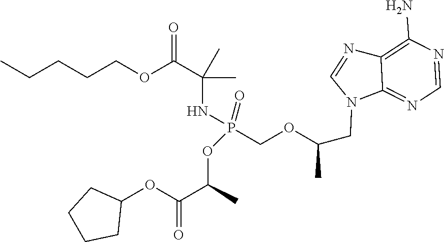 [SMILES: CCCCCOC(=O)C(C)(C)NP(=O)(CO[C@H](C)Cn1cnc2c(N)ncnc21)O[C@@H](C)C(=O)OC1CCCC1] |
| II-56 | 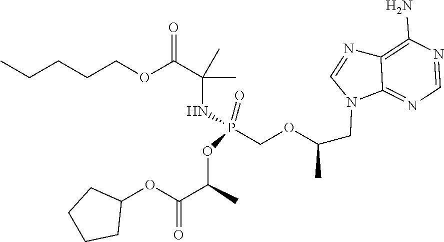 [SMILES: CCCCCOC(=O)C(C)(C)N[P@](=O)(CO[C@H](C)Cn1cnc2c(N)ncnc21)O[C@@H](C)C(=O)OC1CCCC1] |
| II-57 | 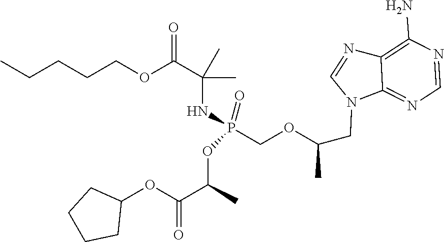 [SMILES: CCCCCOC(=O)C(C)(C)N[P@@](=O)(CO[C@H](C)Cn1cnc2c(N)ncnc21)O[C@@H](C)C(=O)OC1CCCC1] |
| II-58 | 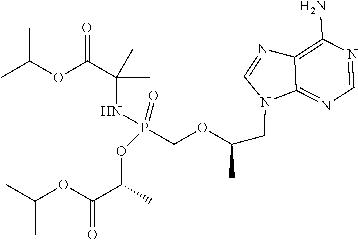 [SMILES: CC(C)OC(=O)[C@@H](C)OP(=O)(CO[C@H](C)Cn1cnc2c(N)ncnc21)NC(C)(C)C(=O)OC(C)C] |
| II-59 | 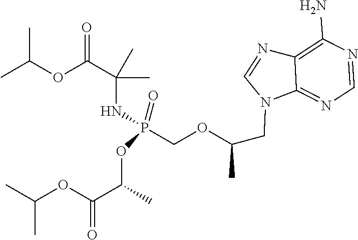 [SMILES: CC(C)OC(=O)[C@@H](C)O[P@@](=O)(CO[C@H](C)Cn1cnc2c(N)ncnc21)NC(C)(C)C(=O)OC(C)C] |
| II-60 | 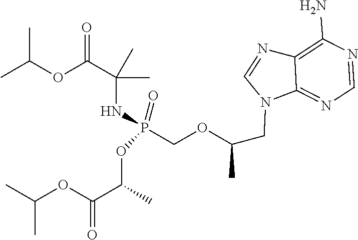 [SMILES: CC(C)OC(=O)[C@@H](C)O[P@](=O)(CO[C@H](C)Cn1cnc2c(N)ncnc21)NC(C)(C)C(=O)OC(C)C] |
| II-61 | 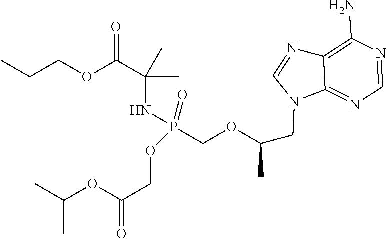 [SMILES: CCCOC(=O)C(C)(C)NP(=O)(CO[C@H](C)Cn1cnc2c(N)ncnc21)OCC(=O)OC(C)C] |
| II-62 | 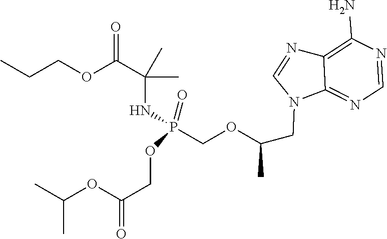 [SMILES: CCCOC(=O)C(C)(C)N[P@](=O)(CO[C@H](C)Cn1cnc2c(N)ncnc21)OCC(=O)OC(C)C] |
| II-63 | 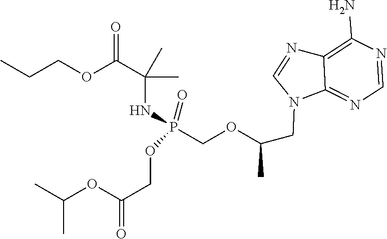 [SMILES: CCCOC(=O)C(C)(C)N[P@@](=O)(CO[C@H](C)Cn1cnc2c(N)ncnc21)OCC(=O)OC(C)C] |
| II-64 | 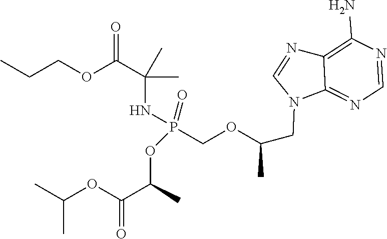 [SMILES: CCCOC(=O)C(C)(C)NP(=O)(CO[C@H](C)Cn1cnc2c(N)ncnc21)O[C@@H](C)C(=O)OC(C)C] |
| II-65 | 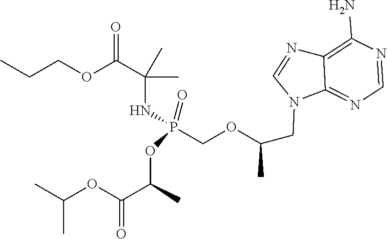 [SMILES: CCCOC(=O)C(C)(C)N[P@](=O)(CO[C@H](C)Cn1cnc2c(N)ncnc21)O[C@@H](C)C(=O)OC(C)C] |
| II-66 | 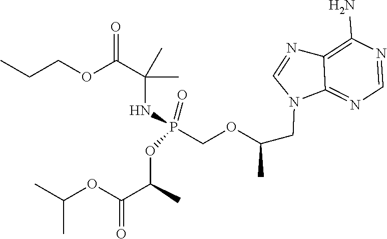 [SMILES: CCCOC(=O)C(C)(C)N[P@@](=O)(CO[C@H](C)Cn1cnc2c(N)ncnc21)O[C@@H](C)C(=O)OC(C)C] |
| II-67 | 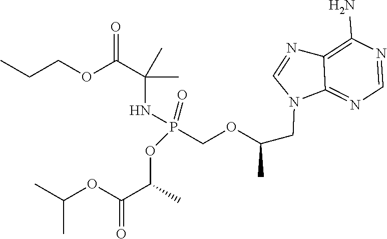 [SMILES: CCCOC(=O)C(C)(C)NP(=O)(CO[C@H](C)Cn1cnc2c(N)ncnc21)O[C@H](C)C(=O)OC(C)C] |
| II-68 | 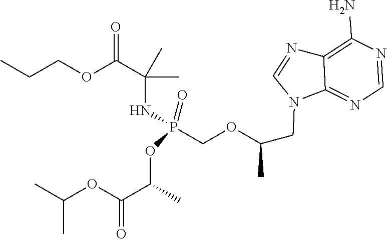 [SMILES: CCCOC(=O)C(C)(C)N[P@](=O)(CO[C@H](C)Cn1cnc2c(N)ncnc21)O[C@H](C)C(=O)OC(C)C] |
| II-69 | 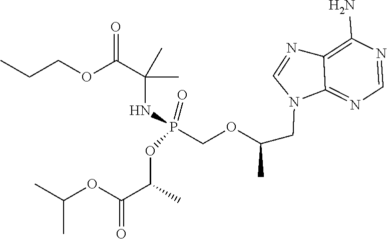 [SMILES: CCCOC(=O)C(C)(C)N[P@@](=O)(CO[C@H](C)Cn1cnc2c(N)ncnc21)O[C@H](C)C(=O)OC(C)C] |
| II-70 | 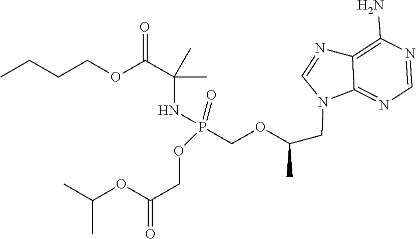 [SMILES: CCCCOC(=O)C(C)(C)NP(=O)(CO[C@H](C)Cn1cnc2c(N)ncnc21)OCC(=O)OC(C)C] |
| II-71 | 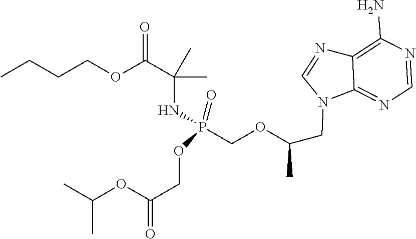 [SMILES: CCCCOC(=O)C(C)(C)N[P@](=O)(CO[C@H](C)Cn1cnc2c(N)ncnc21)OCC(=O)OC(C)C] |
| II-72 | 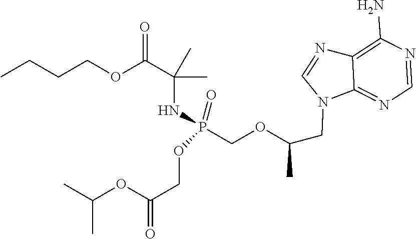 [SMILES: CCCCOC(=O)C(C)(C)N[P@@](=O)(CO[C@H](C)Cn1cnc2c(N)ncnc21)OCC(=O)OC(C)C] |
| II-73 | 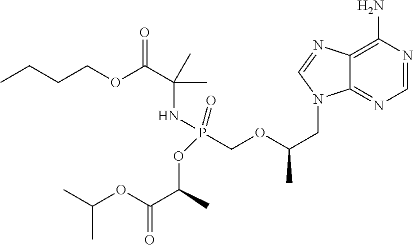 [SMILES: CCCCOC(=O)C(C)(C)NP(=O)(CO[C@H](C)Cn1cnc2c(N)ncnc21)O[C@@H](C)C(=O)OC(C)C] |
| II-74 | 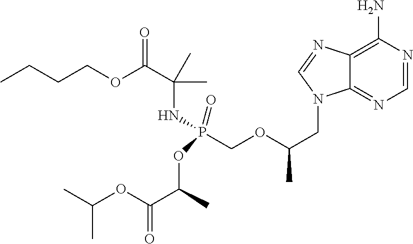 [SMILES: CCCCOC(=O)C(C)(C)N[P@](=O)(CO[C@H](C)Cn1cnc2c(N)ncnc21)O[C@@H](C)C(=O)OC(C)C] |
| II-75 | 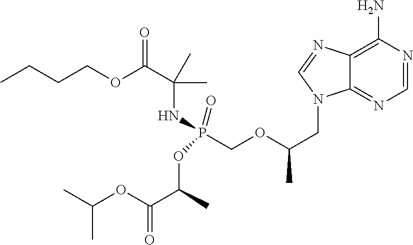 [SMILES: CCCCOC(=O)C(C)(C)N[P@@](=O)(CO[C@H](C)Cn1cnc2c(N)ncnc21)O[C@@H](C)C(=O)OC(C)C] |
| II-76 | 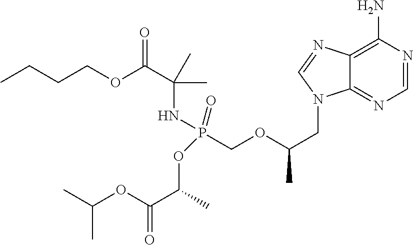 [SMILES: CCCCOC(=O)C(C)(C)NP(=O)(CO[C@H](C)Cn1cnc2c(N)ncnc21)O[C@H](C)C(=O)OC(C)C] |
| II-77 | 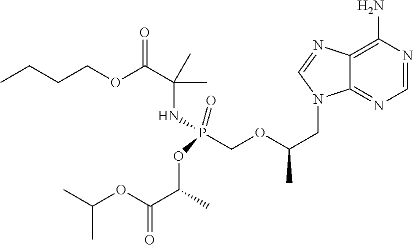 [SMILES: CCCCOC(=O)C(C)(C)N[P@](=O)(CO[C@H](C)Cn1cnc2c(N)ncnc21)O[C@H](C)C(=O)OC(C)C] |
| II-78 | 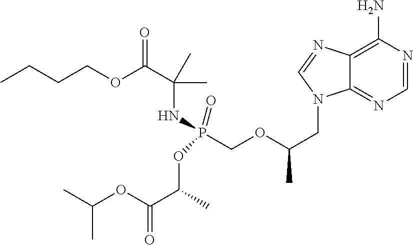 [SMILES: CCCCOC(=O)C(C)(C)N[P@@](=O)(CO[C@H](C)Cn1cnc2c(N)ncnc21)O[C@H](C)C(=O)OC(C)C] |
| II-79 | 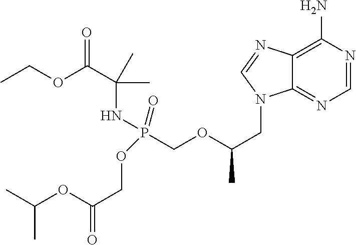 [SMILES: CCOC(=O)C(C)(C)NP(=O)(CO[C@H](C)Cn1cnc2c(N)ncnc21)OCC(=O)OC(C)C] |
| II-80 | 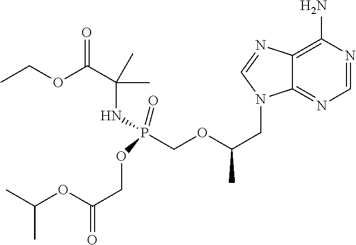 [SMILES: CCOC(=O)C(C)(C)N[P@](=O)(CO[C@H](C)Cn1cnc2c(N)ncnc21)OCC(=O)OC(C)C] |
| II-81 | 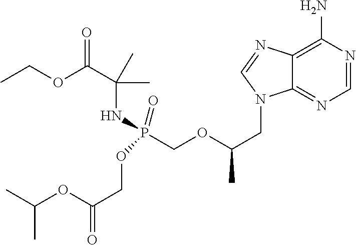 [SMILES: CCOC(=O)C(C)(C)N[P@@](=O)(CO[C@H](C)Cn1cnc2c(N)ncnc21)OCC(=O)OC(C)C] |
| II-82 | 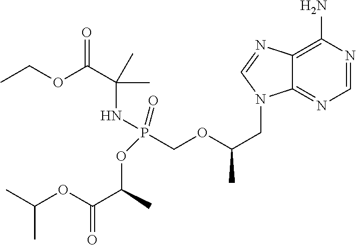 [SMILES: CCOC(=O)C(C)(C)NP(=O)(CO[C@H](C)Cn1cnc2c(N)ncnc21)O[C@@H](C)C(=O)OC(C)C] |
| II-83 | 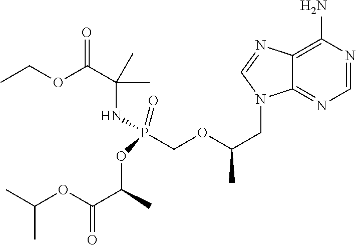 [SMILES: CCOC(=O)C(C)(C)N[P@](=O)(CO[C@H](C)Cn1cnc2c(N)ncnc21)O[C@@H](C)C(=O)OC(C)C] |
| II-84 | 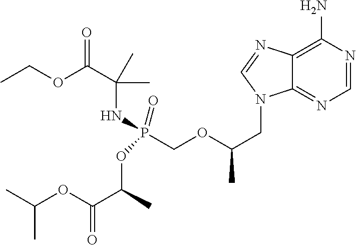 [SMILES: CCOC(=O)C(C)(C)N[P@@](=O)(CO[C@H](C)Cn1cnc2c(N)ncnc21)O[C@@H](C)C(=O)OC(C)C] |
| II-85 | 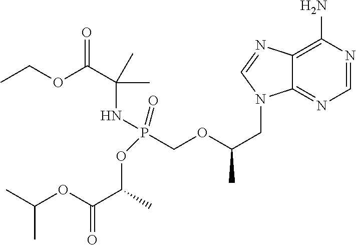 [SMILES: CCOC(=O)C(C)(C)NP(=O)(CO[C@H](C)Cn1cnc2c(N)ncnc21)O[C@H](C)C(=O)OC(C)C] |
| II-86 | 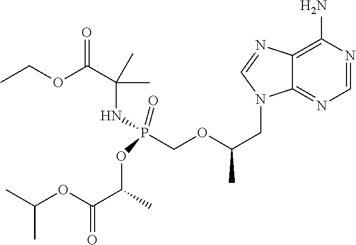 [SMILES: CCOC(=O)C(C)(C)N[P@](=O)(CO[C@H](C)Cn1cnc2c(N)ncnc21)O[C@H](C)C(=O)OC(C)C] |
| II-87 | 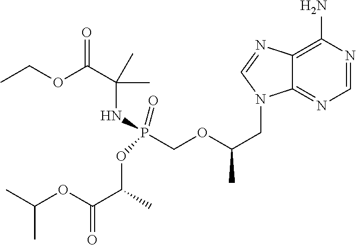 [SMILES: CCOC(=O)C(C)(C)N[P@@](=O)(CO[C@H](C)Cn1cnc2c(N)ncnc21)O[C@H](C)C(=O)OC(C)C] |
| II-88 | 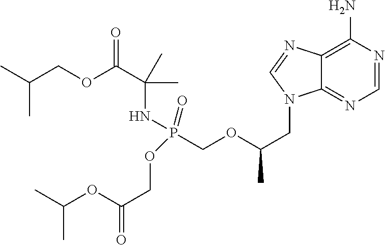 [SMILES: CC(C)COC(=O)C(C)(C)NP(=O)(CO[C@H](C)Cn1cnc2c(N)ncnc21)OCC(=O)OC(C)C] |
| II-89 | 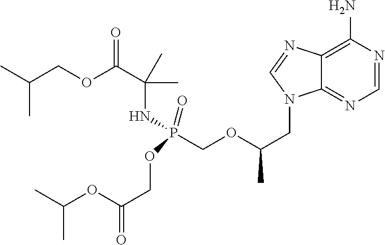 [SMILES: CC(C)COC(=O)C(C)(C)N[P@](=O)(CO[C@H](C)Cn1cnc2c(N)ncnc21)OCC(=O)OC(C)C] |
| II-90 | 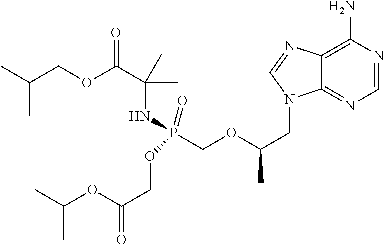 [SMILES: CC(C)COC(=O)C(C)(C)N[P@@](=O)(CO[C@H](C)Cn1cnc2c(N)ncnc21)OCC(=O)OC(C)C] |
| II-91 | 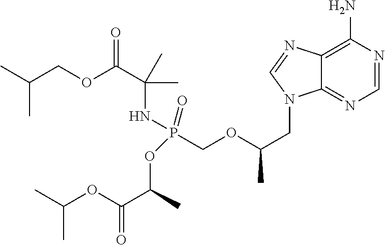 [SMILES: CC(C)COC(=O)C(C)(C)NP(=O)(CO[C@H](C)Cn1cnc2c(N)ncnc21)O[C@@H](C)C(=O)OC(C)C] |
| II-92 | 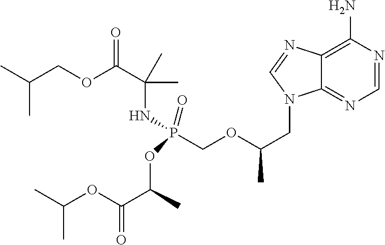 [SMILES: CC(C)COC(=O)C(C)(C)N[P@](=O)(CO[C@H](C)Cn1cnc2c(N)ncnc21)O[C@@H](C)C(=O)OC(C)C] |
| II-93 | 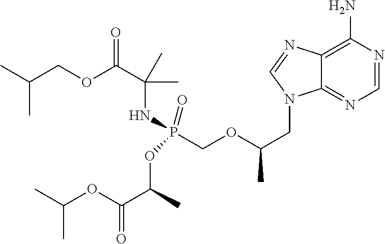 [SMILES: CC(C)COC(=O)C(C)(C)N[P@@](=O)(CO[C@H](C)Cn1cnc2c(N)ncnc21)O[C@@H](C)C(=O)OC(C)C] |
| II-94 | 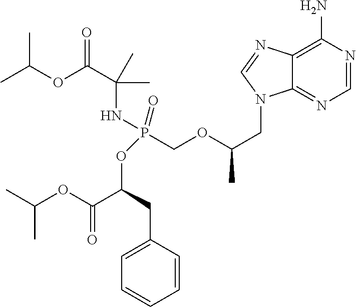 [SMILES: CC(C)OC(=O)[C@H](Cc1ccccc1)OP(=O)(CO[C@H](C)Cn1cnc2c(N)ncnc21)NC(C)(C)C(=O)OC(C)C] |
| II-95 | 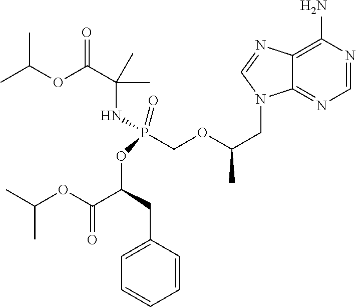 [SMILES: CC(C)OC(=O)[C@H](Cc1ccccc1)O[P@@](=O)(CO[C@H](C)Cn1cnc2c(N)ncnc21)NC(C)(C)C(=O)OC(C)C] |
| II-96 | 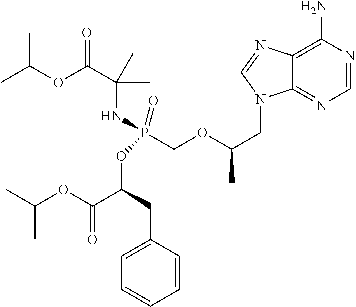 [SMILES: CC(C)OC(=O)[C@H](Cc1ccccc1)O[P@](=O)(CO[C@H](C)Cn1cnc2c(N)ncnc21)NC(C)(C)C(=O)OC(C)C] |
| II-97 | 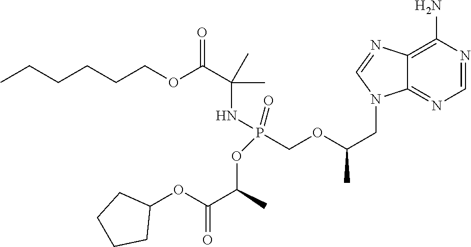 [SMILES: CCCCCCOC(=O)C(C)(C)NP(=O)(CO[C@H](C)Cn1cnc2c(N)ncnc21)O[C@@H](C)C(=O)OC1CCCC1] |
| II-98 | 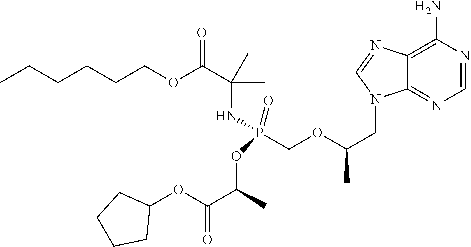 [SMILES: CCCCCCOC(=O)C(C)(C)N[P@](=O)(CO[C@H](C)Cn1cnc2c(N)ncnc21)O[C@@H](C)C(=O)OC1CCCC1] |
| II-99 | 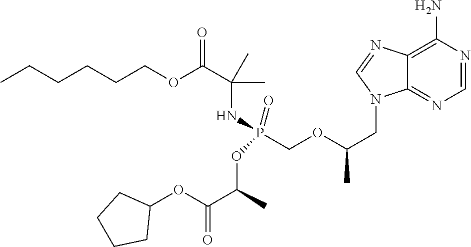 [SMILES: CCCCCCOC(=O)C(C)(C)N[P@@](=O)(CO[C@H](C)Cn1cnc2c(N)ncnc21)O[C@@H](C)C(=O)OC1CCCC1] |
| II-100 | 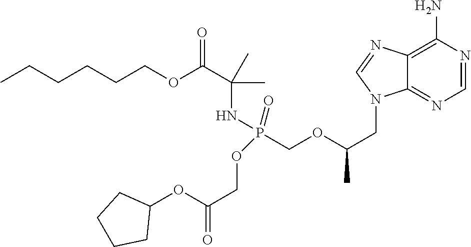 [SMILES: CCCCCCOC(=O)C(C)(C)NP(=O)(CO[C@H](C)Cn1cnc2c(N)ncnc21)OCC(=O)OC1CCCC1] |
| II-101 | 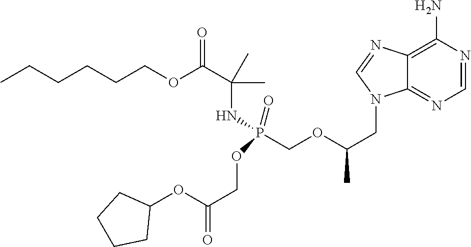 [SMILES: CCCCCCOC(=O)C(C)(C)N[P@](=O)(CO[C@H](C)Cn1cnc2c(N)ncnc21)OCC(=O)OC1CCCC1] |
| II-102 | 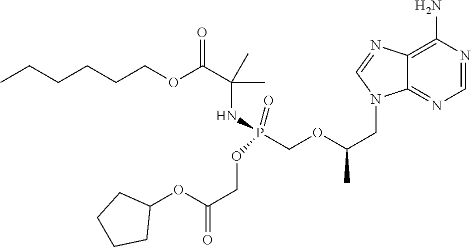 [SMILES: CCCCCCOC(=O)C(C)(C)N[P@@](=O)(CO[C@H](C)Cn1cnc2c(N)ncnc21)OCC(=O)OC1CCCC1] |
| II-103 | 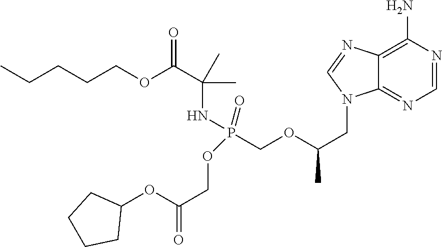 [SMILES: CCCCCOC(=O)C(C)(C)NP(=O)(CO[C@H](C)Cn1cnc2c(N)ncnc21)OCC(=O)OC1CCCC1] |
| II-104 | 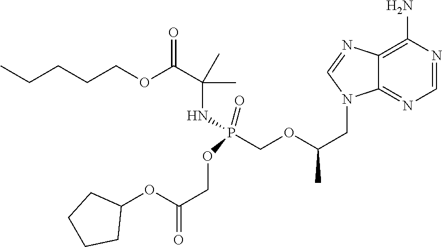 [SMILES: CCCCCOC(=O)C(C)(C)N[P@](=O)(CO[C@H](C)Cn1cnc2c(N)ncnc21)OCC(=O)OC1CCCC1] |
| II-105 | 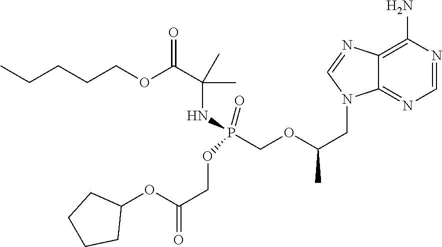 [SMILES: CCCCCOC(=O)C(C)(C)N[P@@](=O)(CO[C@H](C)Cn1cnc2c(N)ncnc21)OCC(=O)OC1CCCC1] |
| II-106 | 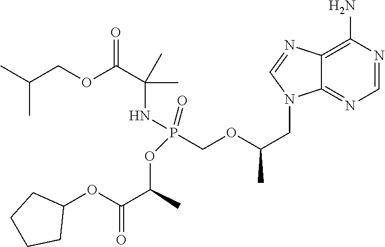 [SMILES: CC(C)COC(=O)C(C)(C)NP(=O)(CO[C@H](C)Cn1cnc2c(N)ncnc21)O[C@@H](C)C(=O)OC1CCCC1] |
| II-107 | 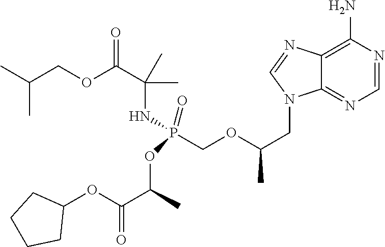 [SMILES: CC(C)COC(=O)C(C)(C)N[P@](=O)(CO[C@H](C)Cn1cnc2c(N)ncnc21)O[C@@H](C)C(=O)OC1CCCC1] |
| II-108 | 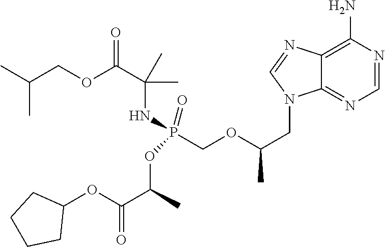 [SMILES: CC(C)COC(=O)C(C)(C)N[P@@](=O)(CO[C@H](C)Cn1cnc2c(N)ncnc21)O[C@@H](C)C(=O)OC1CCCC1] |
| II-109 | 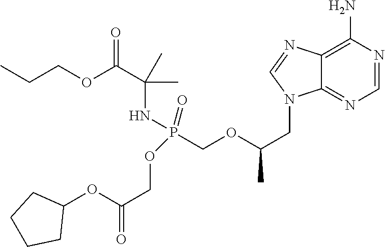 [SMILES: CCCOC(=O)C(C)(C)NP(=O)(CO[C@H](C)Cn1cnc2c(N)ncnc21)OCC(=O)OC1CCCC1] |
| II-110 | 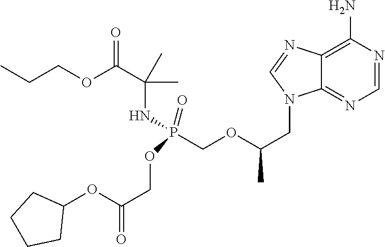 [SMILES: CCCOC(=O)C(C)(C)N[P@](=O)(CO[C@H](C)Cn1cnc2c(N)ncnc21)OCC(=O)OC1CCCC1] |
| II-111 | 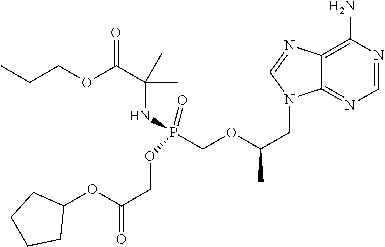 [SMILES: CCCOC(=O)C(C)(C)N[P@@](=O)(CO[C@H](C)Cn1cnc2c(N)ncnc21)OCC(=O)OC1CCCC1] |
| II-112 | 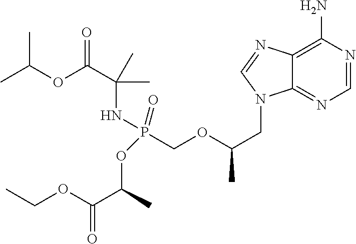 [SMILES: CCOC(=O)[C@H](C)OP(=O)(CO[C@H](C)Cn1cnc2c(N)ncnc21)NC(C)(C)C(=O)OC(C)C] |
| II-113 | 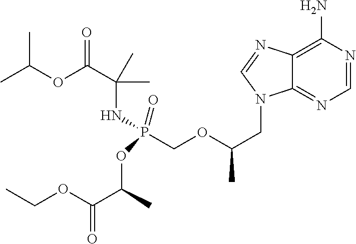 [SMILES: CCOC(=O)[C@H](C)O[P@@](=O)(CO[C@H](C)Cn1cnc2c(N)ncnc21)NC(C)(C)C(=O)OC(C)C] |
| II-114 | 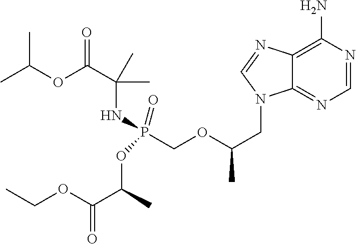 [SMILES: CCOC(=O)[C@H](C)O[P@](=O)(CO[C@H](C)Cn1cnc2c(N)ncnc21)NC(C)(C)C(=O)OC(C)C] |
| II-115 | 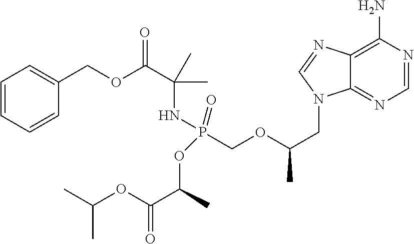 [SMILES: CC(C)OC(=O)[C@H](C)OP(=O)(CO[C@H](C)Cn1cnc2c(N)ncnc21)NC(C)(C)C(=O)OCc1ccccc1] |
| II-116 | 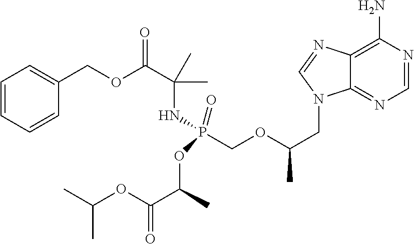 [SMILES: CC(C)OC(=O)[C@H](C)O[P@@](=O)(CO[C@H](C)Cn1cnc2c(N)ncnc21)NC(C)(C)C(=O)OCc1ccccc1] |
| II-117 | 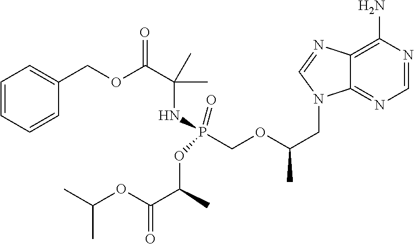 [SMILES: CC(C)OC(=O)[C@H](C)O[P@](=O)(CO[C@H](C)Cn1cnc2c(N)ncnc21)NC(C)(C)C(=O)OCc1ccccc1] |
| II-118 | 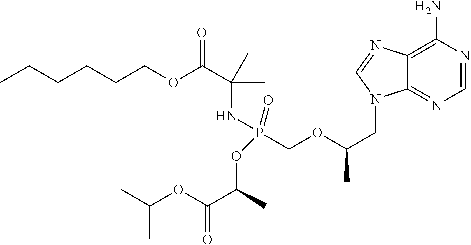 [SMILES: CCCCCCOC(=O)C(C)(C)NP(=O)(CO[C@H](C)Cn1cnc2c(N)ncnc21)O[C@@H](C)C(=O)OC(C)C] |
| II-119 | 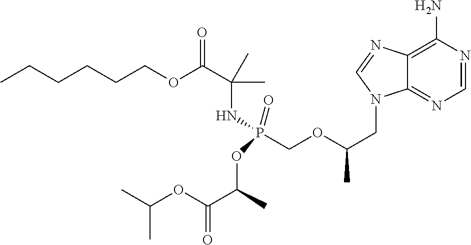 [SMILES: CCCCCCOC(=O)C(C)(C)N[P@](=O)(CO[C@H](C)Cn1cnc2c(N)ncnc21)O[C@@H](C)C(=O)OC(C)C] |
| II-120 | 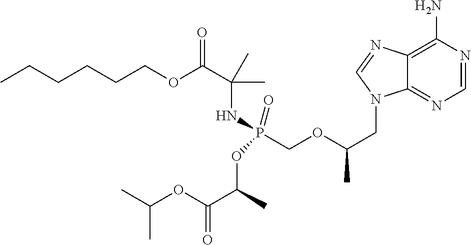 [SMILES: CCCCCCOC(=O)C(C)(C)N[P@@](=O)(CO[C@H](C)Cn1cnc2c(N)ncnc21)O[C@@H](C)C(=O)OC(C)C] |
| II-121 | 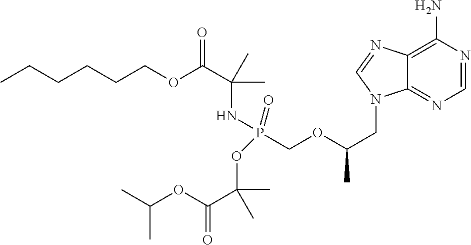 [SMILES: CCCCCCOC(=O)C(C)(C)NP(=O)(CO[C@H](C)Cn1cnc2c(N)ncnc21)OC(C)(C)C(=O)OC(C)C] |
| II-122 | 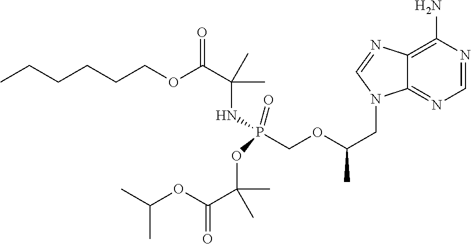 [SMILES: CCCCCCOC(=O)C(C)(C)N[P@](=O)(CO[C@H](C)Cn1cnc2c(N)ncnc21)OC(C)(C)C(=O)OC(C)C] |
| II-123 | 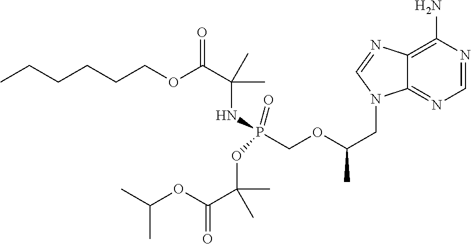 [SMILES: CCCCCCOC(=O)C(C)(C)N[P@@](=O)(CO[C@H](C)Cn1cnc2c(N)ncnc21)OC(C)(C)C(=O)OC(C)C] |
| II-124 | 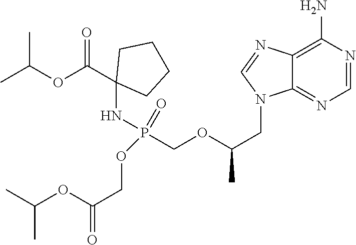 [SMILES: CC(C)OC(=O)COP(=O)(CO[C@H](C)Cn1cnc2c(N)ncnc21)NC1(C(=O)OC(C)C)CCCC1] |
| II-125 | 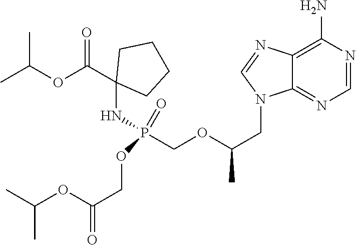 [SMILES: CC(C)OC(=O)CO[P@@](=O)(CO[C@H](C)Cn1cnc2c(N)ncnc21)NC1(C(=O)OC(C)C)CCCC1] |
| II-126 | 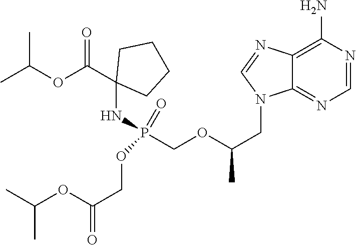 [SMILES: CC(C)OC(=O)CO[P@](=O)(CO[C@H](C)Cn1cnc2c(N)ncnc21)NC1(C(=O)OC(C)C)CCCC1] |
| II-127 | 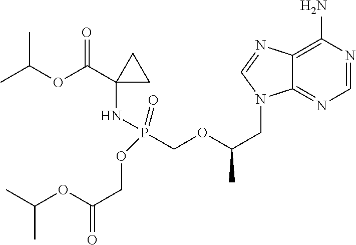 [SMILES: CC(C)OC(=O)COP(=O)(CO[C@H](C)Cn1cnc2c(N)ncnc21)NC1(C(=O)OC(C)C)CC1] |
| II-128 | 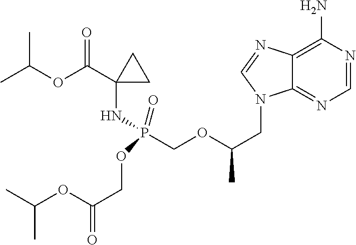 [SMILES: CC(C)OC(=O)CO[P@@](=O)(CO[C@H](C)Cn1cnc2c(N)ncnc21)NC1(C(=O)OC(C)C)CC1] |
| II-129 | 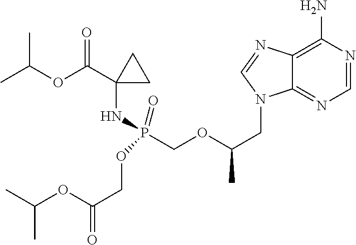 [SMILES: CC(C)OC(=O)CO[P@](=O)(CO[C@H](C)Cn1cnc2c(N)ncnc21)NC1(C(=O)OC(C)C)CC1] |
| II-130 | 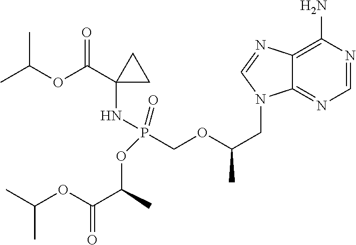 [SMILES: CC(C)OC(=O)[C@H](C)OP(=O)(CO[C@H](C)Cn1cnc2c(N)ncnc21)NC1(C(=O)OC(C)C)CC1] |
| II-131 | 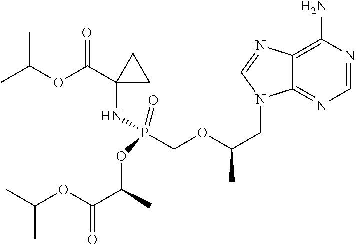 [SMILES: CC(C)OC(=O)[C@H](C)O[P@@](=O)(CO[C@H](C)Cn1cnc2c(N)ncnc21)NC1(C(=O)OC(C)C)CC1] |
| II-132 | 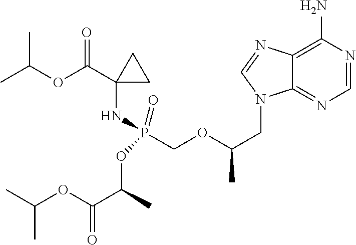 [SMILES: CC(C)OC(=O)[C@H](C)O[P@](=O)(CO[C@H](C)Cn1cnc2c(N)ncnc21)NC1(C(=O)OC(C)C)CC1] |
| II-133 | 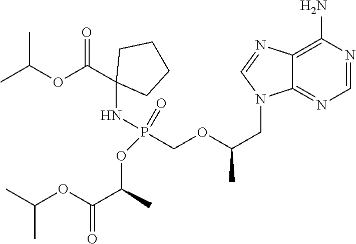 [SMILES: CC(C)OC(=O)[C@H](C)OP(=O)(CO[C@H](C)Cn1cnc2c(N)ncnc21)NC1(C(=O)OC(C)C)CCCC1] |
| II-134 | 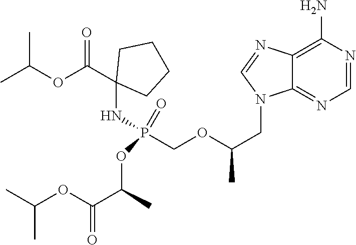 [SMILES: CC(C)OC(=O)[C@H](C)O[P@@](=O)(CO[C@H](C)Cn1cnc2c(N)ncnc21)NC1(C(=O)OC(C)C)CCCC1] |
| II-135 | 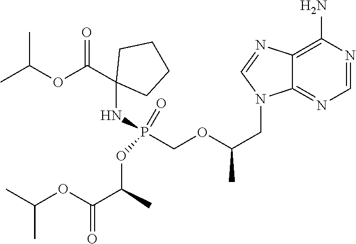 [SMILES: CC(C)OC(=O)[C@H](C)O[P@](=O)(CO[C@H](C)Cn1cnc2c(N)ncnc21)NC1(C(=O)OC(C)C)CCCC1] |
| II-136 | 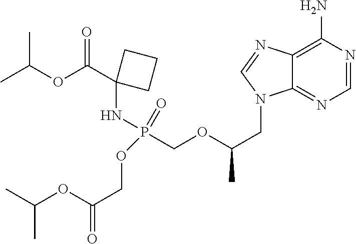 [SMILES: CC(C)OC(=O)COP(=O)(CO[C@H](C)Cn1cnc2c(N)ncnc21)NC1(C(=O)OC(C)C)CCC1] |
| II-137 | 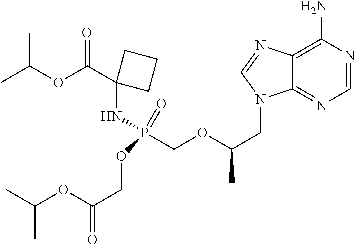 [SMILES: CC(C)OC(=O)CO[P@@](=O)(CO[C@H](C)Cn1cnc2c(N)ncnc21)NC1(C(=O)OC(C)C)CCC1] |
| II-138 | 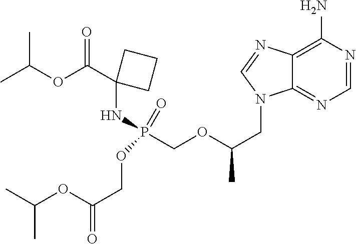 [SMILES: CC(C)OC(=O)CO[P@](=O)(CO[C@H](C)Cn1cnc2c(N)ncnc21)NC1(C(=O)OC(C)C)CCC1] |
| II-139 | 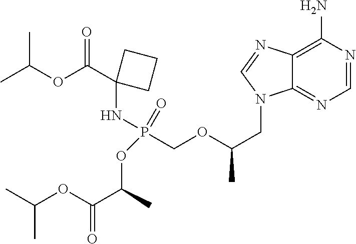 [SMILES: CC(C)OC(=O)[C@H](C)OP(=O)(CO[C@H](C)Cn1cnc2c(N)ncnc21)NC1(C(=O)OC(C)C)CCC1] |
| II-140 | 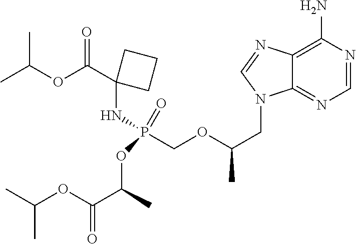 [SMILES: CC(C)OC(=O)[C@H](C)O[P@@](=O)(CO[C@H](C)Cn1cnc2c(N)ncnc21)NC1(C(=O)OC(C)C)CCC1] |
| II-141 | 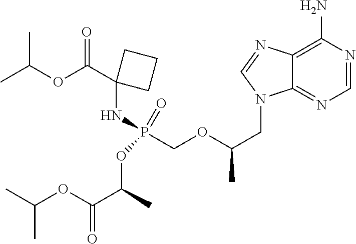 [SMILES: CC(C)OC(=O)[C@H](C)O[P@](=O)(CO[C@H](C)Cn1cnc2c(N)ncnc21)NC1(C(=O)OC(C)C)CCC1] |
| II-142 | 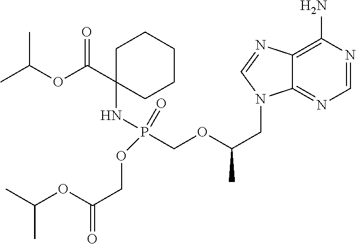 [SMILES: CC(C)OC(=O)COP(=O)(CO[C@H](C)Cn1cnc2c(N)ncnc21)NC1(C(=O)OC(C)C)CCCCC1] |
| II-143 | 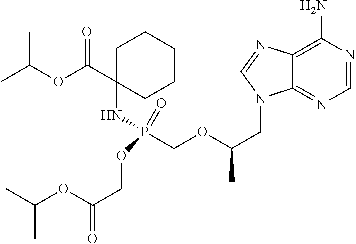 [SMILES: CC(C)OC(=O)CO[P@@](=O)(CO[C@H](C)Cn1cnc2c(N)ncnc21)NC1(C(=O)OC(C)C)CCCCC1] |
| II-144 | 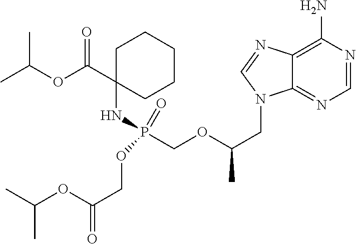 [SMILES: CC(C)OC(=O)CO[P@](=O)(CO[C@H](C)Cn1cnc2c(N)ncnc21)NC1(C(=O)OC(C)C)CCCCC1] |
| II-145 | 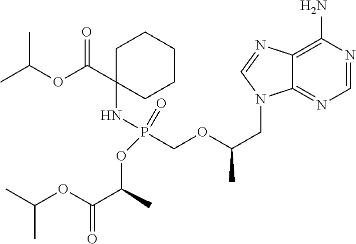 [SMILES: CC(C)OC(=O)[C@H](C)OP(=O)(CO[C@H](C)Cn1cnc2c(N)ncnc21)NC1(C(=O)OC(C)C)CCCCC1] |
| II-146 | 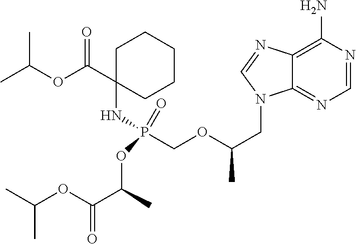 [SMILES: CC(C)OC(=O)[C@H](C)O[P@@](=O)(CO[C@H](C)Cn1cnc2c(N)ncnc21)NC1(C(=O)OC(C)C)CCCCC1] |
| II-147 | 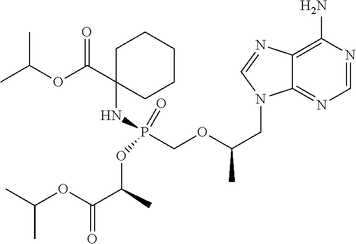 [SMILES: CC(C)OC(=O)[C@H](C)O[P@](=O)(CO[C@H](C)Cn1cnc2c(N)ncnc21)NC1(C(=O)OC(C)C)CCCCC1] |

In certain embodiments, the compound is a compound in Table 3 below, or a pharmaceutically acceptable salt thereof. In certain embodiments, the compound is a compound in Table 3 below. Compounds in Table 3 below can be prepared based on procedures described in, for example, WO 2017/007701, WO 2017/027434, and WO 2017/100108; and related patents and applications, such as U.S. Pat. Nos. 9,822,138 and 10,745,428; each of which is hereby incorporated by reference.

TABLE 3

| Compound No. | Chemical Structure |
|---|---|
| III-1 | 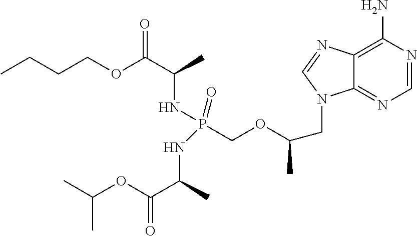 [SMILES: CCCCOC(=O)[C@@H](C)NP(=O)(CO[C@H](C)Cn1cnc2c(N)ncnc21)N[C@@H](C)C(=O)OC(C)C] |
| III-2 | 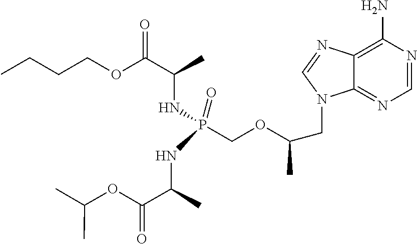 [SMILES: CCCCOC(=O)[C@@H](C)N[P@](=O)(CO[C@H](C)Cn1cnc2c(N)ncnc21)N[C@@H](C)C(=O)OC(C)C] |
| III-3 | 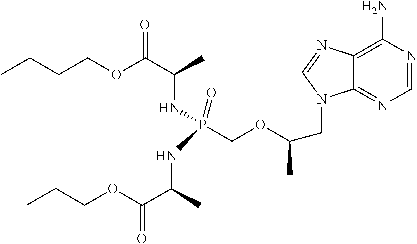 [SMILES: CCCCOC(=O)[C@@H](C)N[P@](=O)(CO[C@H](C)Cn1cnc2c(N)ncnc21)N[C@@H](C)C(=O)OCCC] |
| III-4 | 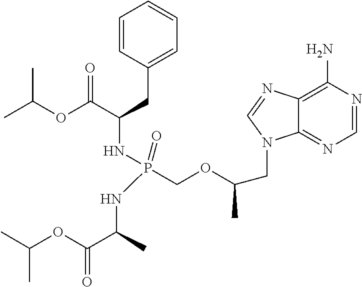 [SMILES: CC(C)OC(=O)[C@H](C)NP(=O)(CO[C@H](C)Cn1cnc2c(N)ncnc21)N[C@H](Cc1ccccc1)C(=O)OC(C)C] |
| III-5 | 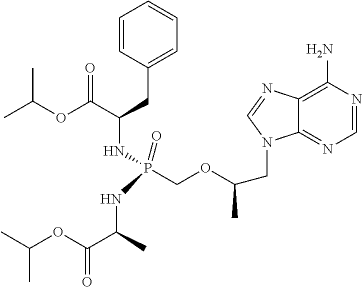 [SMILES: CC(C)OC(=O)[C@H](C)N[P@@](=O)(CO[C@H](C)Cn1cnc2c(N)ncnc21)N[C@H](Cc1ccccc1)C(=O)OC(C)C] |
| III-6 | 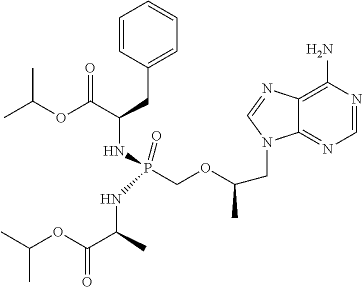 [SMILES: CC(C)OC(=O)[C@H](C)N[P@](=O)(CO[C@H](C)Cn1cnc2c(N)ncnc21)N[C@H](Cc1ccccc1)C(=O)OC(C)C] |
| III-7 | 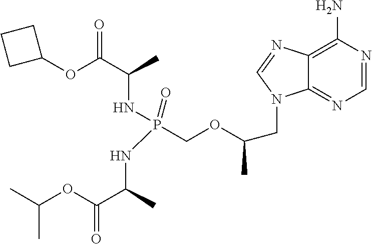 [SMILES: CC(C)OC(=O)[C@H](C)NP(=O)(CO[C@H](C)Cn1cnc2c(N)ncnc21)N[C@H](C)C(=O)OC1CCC1] |
| III-8 | 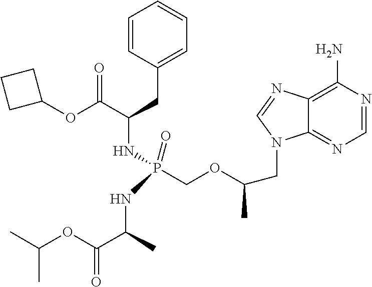 [SMILES: CC(C)OC(=O)[C@H](C)N[P@@](=O)(CO[C@H](C)Cn1cnc2c(N)ncnc21)N[C@H](Cc1ccccc1)C(=O)OC1CCC1] |
| III-9 | 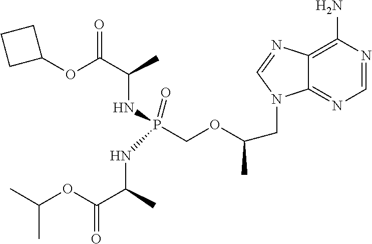 [SMILES: CC(C)OC(=O)[C@H](C)N[P@](=O)(CO[C@H](C)Cn1cnc2c(N)ncnc21)N[C@H](C)C(=O)OC1CCC1] |
| III-10 | 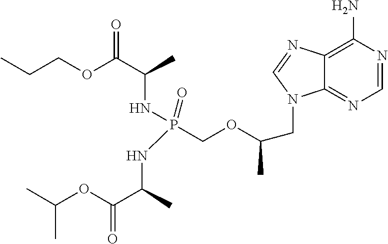 [SMILES: CCCOC(=O)[C@@H](C)NP(=O)(CO[C@H](C)Cn1cnc2c(N)ncnc21)N[C@@H](C)C(=O)OC(C)C] |
| III-11 | 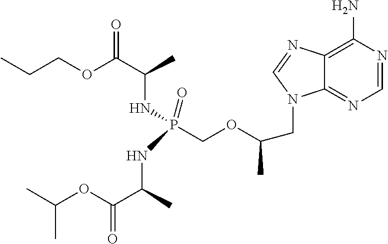 [SMILES: CCCOC(=O)[C@@H](C)N[P@](=O)(CO[C@H](C)Cn1cnc2c(N)ncnc21)N[C@@H](C)C(=O)OC(C)C] |
| III-12 | 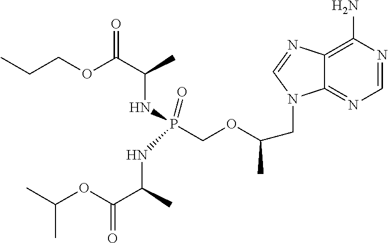 [SMILES: CCCOC(=O)[C@@H](C)N[P@@](=O)(CO[C@H](C)Cn1cnc2c(N)ncnc21)N[C@@H](C)C(=O)OC(C)C] |
| III-13 | 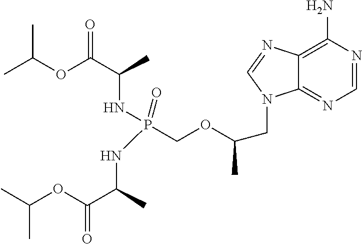 [SMILES: CC(C)OC(=O)[C@H](C)NP(=O)(CO[C@H](C)Cn1cnc2c(N)ncnc21)N[C@H](C)C(=O)OC(C)C] |
| III-14 | 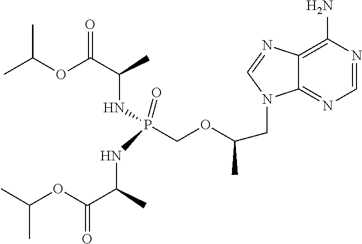 [SMILES: CC(C)OC(=O)[C@H](C)N[P@@](=O)(CO[C@H](C)Cn1cnc2c(N)ncnc21)N[C@H](C)C(=O)OC(C)C] |
| III-15 | 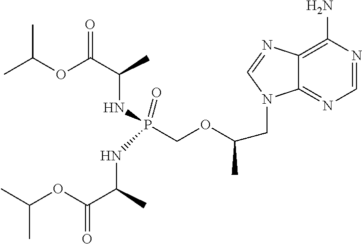 [SMILES: CC(C)OC(=O)[C@H](C)N[P@](=O)(CO[C@H](C)Cn1cnc2c(N)ncnc21)N[C@H](C)C(=O)OC(C)C] |
| III-16 | 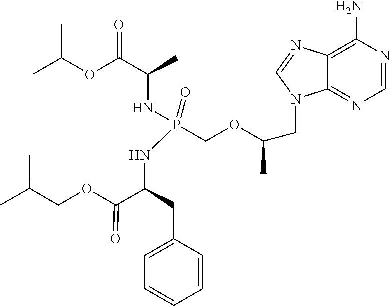 [SMILES: CC(C)COC(=O)[C@H](Cc1ccccc1)NP(=O)(CO[C@H](C)Cn1cnc2c(N)ncnc21)N[C@H](C)C(=O)OC(C)C] |
| III-17 | 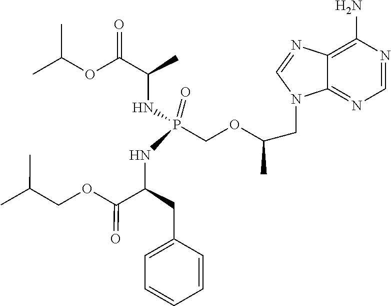 [SMILES: CC(C)COC(=O)[C@H](Cc1ccccc1)N[P@@](=O)(CO[C@H](C)Cn1cnc2c(N)ncnc21)N[C@H](C)C(=O)OC(C)C] |
| III-18 | 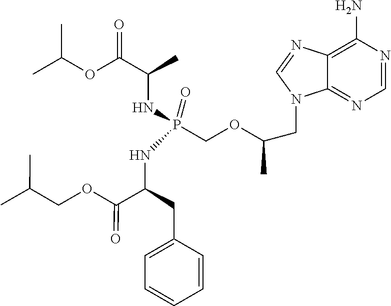 [SMILES: CC(C)COC(=O)[C@H](Cc1ccccc1)N[P@](=O)(CO[C@H](C)Cn1cnc2c(N)ncnc21)N[C@H](C)C(=O)OC(C)C] |
| III-19 | 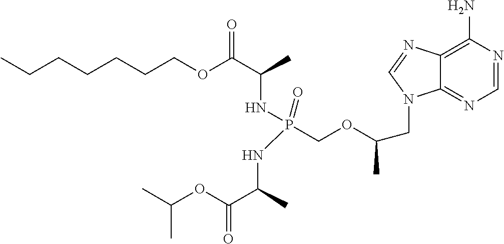 [SMILES: CCCCCCCOC(=O)[C@@H](C)NP(=O)(CO[C@H](C)Cn1cnc2c(N)ncnc21)N[C@@H](C)C(=O)OC(C)C] |
| III-20 | 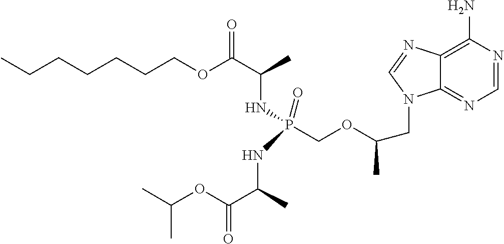 [SMILES: CCCCCCCOC(=O)[C@@H](C)N[P@](=O)(CO[C@H](C)Cn1cnc2c(N)ncnc21)N[C@@H](C)C(=O)OC(C)C] |
| III-21 | 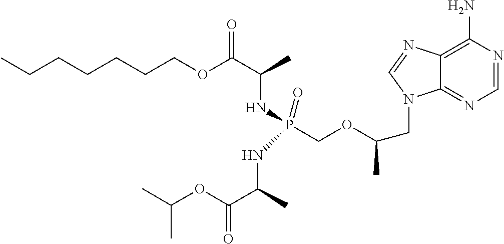 [SMILES: CCCCCCCOC(=O)[C@@H](C)N[P@@](=O)(CO[C@H](C)Cn1cnc2c(N)ncnc21)N[C@@H](C)C(=O)OC(C)C] |
| III-22 | 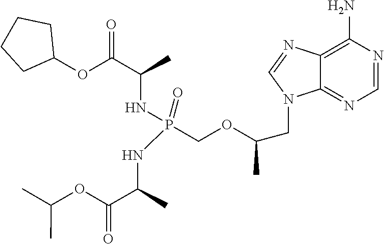 [SMILES: CC(C)OC(=O)[C@H](C)NP(=O)(CO[C@H](C)Cn1cnc2c(N)ncnc21)N[C@H](C)C(=O)OC1CCCC1] |
| III-23 | 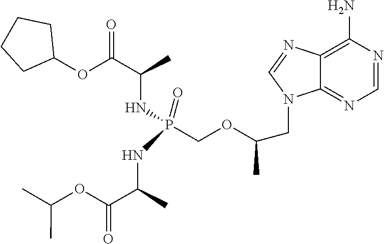 [SMILES: CC(C)OC(=O)[C@H](C)N[P@@](=O)(CO[C@H](C)Cn1cnc2c(N)ncnc21)N[C@H](C)C(=O)OC1CCCC1] |
| III-24 | 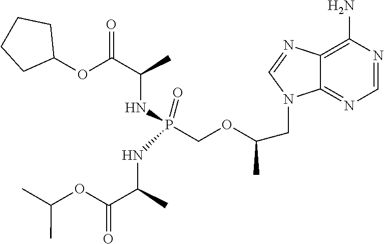 [SMILES: CC(C)OC(=O)[C@H](C)N[P@](=O)(CO[C@H](C)Cn1cnc2c(N)ncnc21)N[C@H](C)C(=O)OC1CCCC1] |
| III-25 | 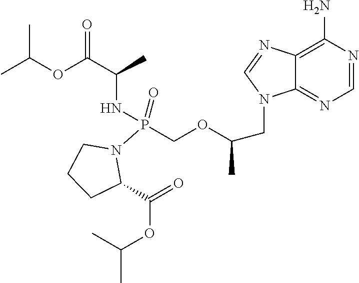 [SMILES: CC(C)OC(=O)[C@@H](C)NP(=O)(CO[C@H](C)Cn1cnc2c(N)ncnc21)N1CCC[C@H]1C(=O)OC(C)C] |
| III-26 | 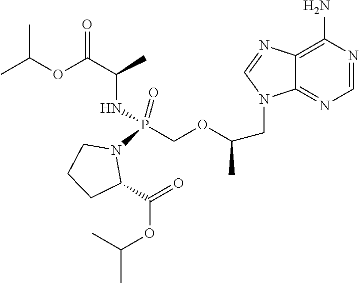 [SMILES: CC(C)OC(=O)[C@@H](C)N[P@](=O)(CO[C@H](C)Cn1cnc2c(N)ncnc21)N1CCC[C@H]1C(=O)OC(C)C] |
| III-27 | 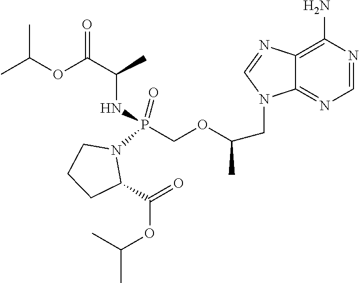 [SMILES: CC(C)OC(=O)[C@@H](C)N[P@@](=O)(CO[C@H](C)Cn1cnc2c(N)ncnc21)N1CCC[C@H]1C(=O)OC(C)C] |
| III-28 | 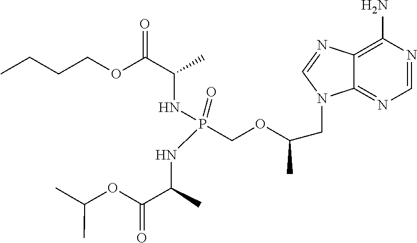 [SMILES: CCCCOC(=O)[C@H](C)NP(=O)(CO[C@H](C)Cn1cnc2c(N)ncnc21)N[C@@H](C)C(=O)OC(C)C] |
| III-29 | 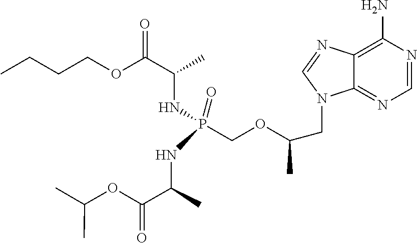 [SMILES: CCCCOC(=O)[C@H](C)N[P@](=O)(CO[C@H](C)Cn1cnc2c(N)ncnc21)N[C@@H](C)C(=O)OC(C)C] |
| III-30 | 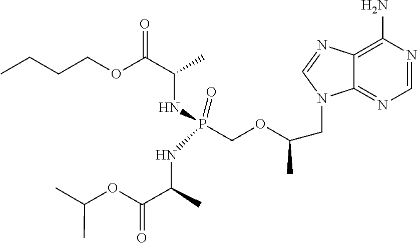 [SMILES: CCCCOC(=O)[C@H](C)N[P@@](=O)(CO[C@H](C)Cn1cnc2c(N)ncnc21)N[C@@H](C)C(=O)OC(C)C] |
| III-31 | 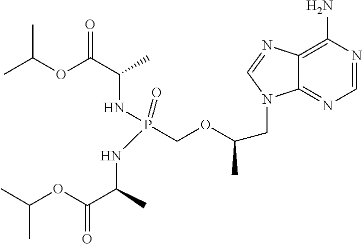 [SMILES: CC(C)OC(=O)[C@H](C)NP(=O)(CO[C@H](C)Cn1cnc2c(N)ncnc21)N[C@@H](C)C(=O)OC(C)C] |
| III-32 | 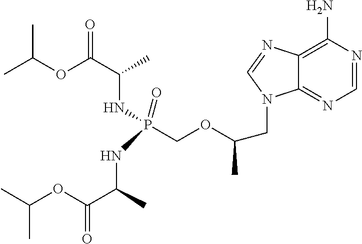 |
| III-33 | 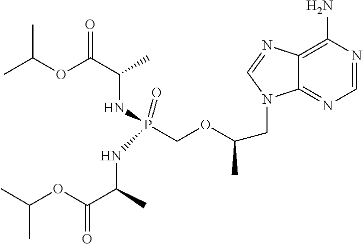 |
| III-34 | 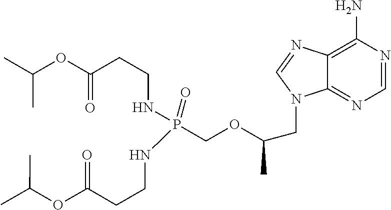 [SMILES: CC(C)OC(=O)CCNP(=O)(CO[C@H](C)Cn1cnc2c(N)ncnc21)NCCC(=O)OC(C)C] |
| III-35 | 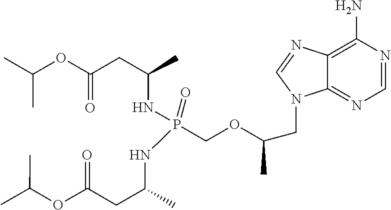 [SMILES: CC(C)OC(=O)C[C@@H](C)NP(=O)(CO[C@H](C)Cn1cnc2c(N)ncnc21)N[C@H](C)CC(=O)OC(C)C] |
| III-36 | 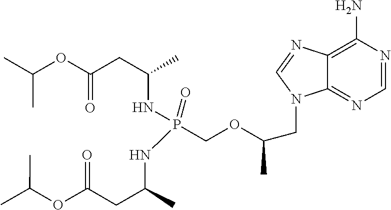 [SMILES: CC(C)OC(=O)C[C@H](C)NP(=O)(CO[C@H](C)Cn1cnc2c(N)ncnc21)N[C@@H](C)CC(=O)OC(C)C] |
| III-37 | 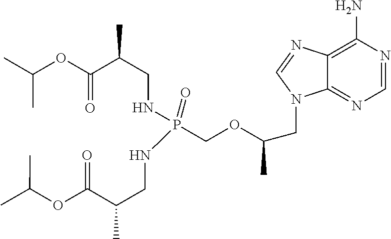 [SMILES: CC(C)OC(=O)[C@@H](C)CNP(=O)(CO[C@H](C)Cn1cnc2c(N)ncnc21)NC[C@H](C)C(=O)OC(C)C] |
| III-38 | 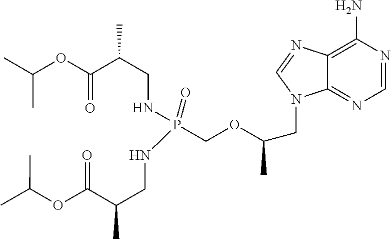 [SMILES: CC(C)OC(=O)[C@H](C)CNP(=O)(CO[C@H](C)Cn1cnc2c(N)ncnc21)NC[C@@H](C)C(=O)OC(C)C] |
| III-39 | 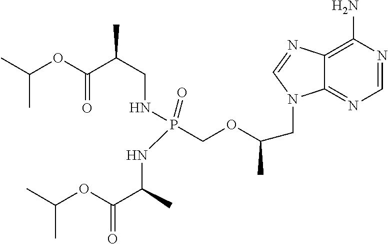 [SMILES: CC(C)OC(=O)[C@H](C)NP(=O)(CO[C@H](C)Cn1cnc2c(N)ncnc21)NC[C@H](C)C(=O)OC(C)C] |
| III-40 | 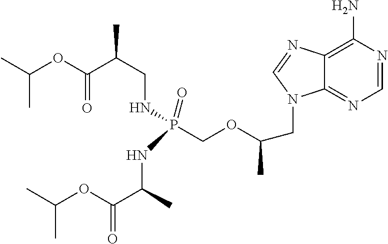 [SMILES: CC(C)OC(=O)[C@H](C)N[P@@](=O)(CO[C@H](C)Cn1cnc2c(N)ncnc21)NC[C@H](C)C(=O)OC(C)C] |
| III-41 | 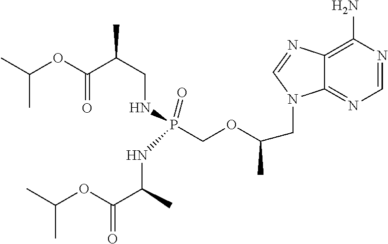 [SMILES: CC(C)OC(=O)[C@H](C)N[P@](=O)(CO[C@H](C)Cn1cnc2c(N)ncnc21)NC[C@H](C)C(=O)OC(C)C] |
| III-42 | 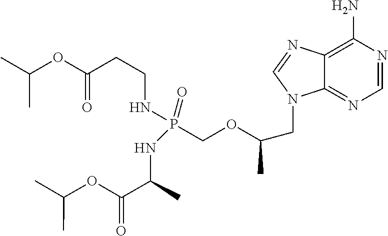 [SMILES: CC(C)OC(=O)CCNP(=O)(CO[C@H](C)Cn1cnc2c(N)ncnc21)N[C@@H](C)C(=O)OC(C)C] |
| III-43 | 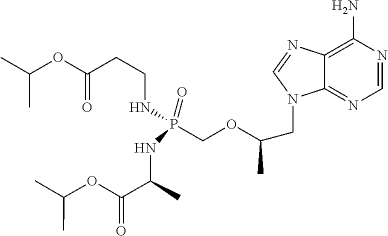 [SMILES: CC(C)OC(=O)CCN[P@](=O)(CO[C@H](C)Cn1cnc2c(N)ncnc21)N[C@@H](C)C(=O)OC(C)C] |
| III-44 | 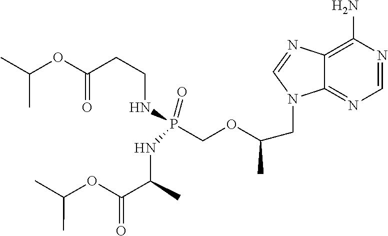 [SMILES: CC(C)OC(=O)CCN[P@@](=O)(CO[C@H](C)Cn1cnc2c(N)ncnc21)N[C@@H](C)C(=O)OC(C)C] |
| III-45 | 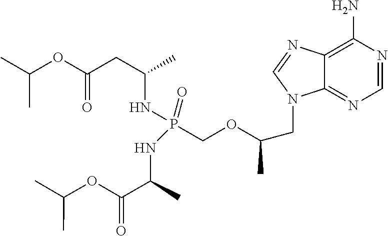 [SMILES: CC(C)OC(=O)C[C@H](C)NP(=O)(CO[C@H](C)Cn1cnc2c(N)ncnc21)N[C@@H](C)C(=O)OC(C)C] |
| III-46 | 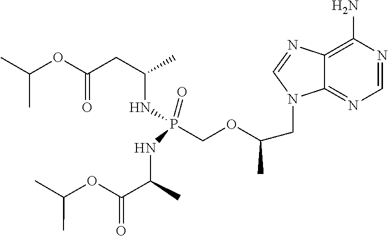 [SMILES: CC(C)OC(=O)C[C@H](C)N[P@](=O)(CO[C@H](C)Cn1cnc2c(N)ncnc21)N[C@@H](C)C(=O)OC(C)C] |
| III-47 | 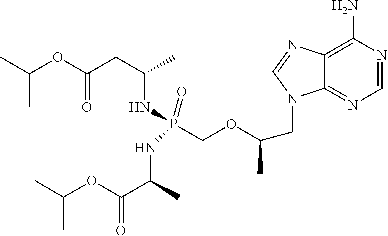 [SMILES: CC(C)OC(=O)C[C@H](C)N[P@@](=O)(CO[C@H](C)Cn1cnc2c(N)ncnc21)N[C@@H](C)C(=O)OC(C)C] |
| III-48 | 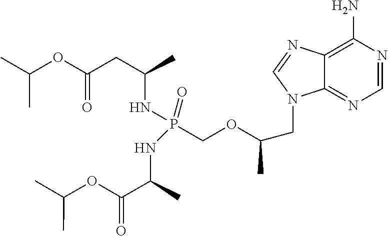 [SMILES: CC(C)OC(=O)C[C@@H](C)NP(=O)(CO[C@H](C)Cn1cnc2c(N)ncnc21)N[C@@H](C)C(=O)OC(C)C] |
| III-49 | 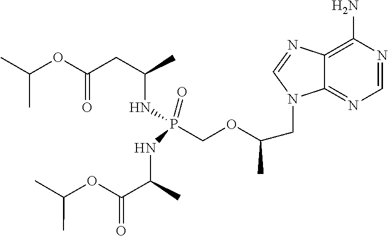 [SMILES: CC(C)OC(=O)C[C@@H](C)N[P@](=O)(CO[C@H](C)Cn1cnc2c(N)ncnc21)N[C@@H](C)C(=O)OC(C)C] |
| III-50 | 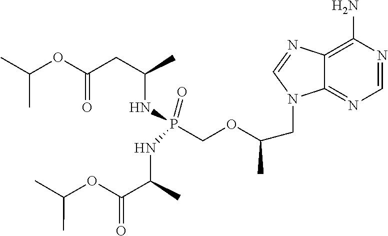 [SMILES: CC(C)OC(=O)C[C@@H](C)N[P@@](=O)(CO[C@H](C)Cn1cnc2c(N)ncnc21)N[C@@H](C)C(=O)OC(C)C] |
| III-51 | 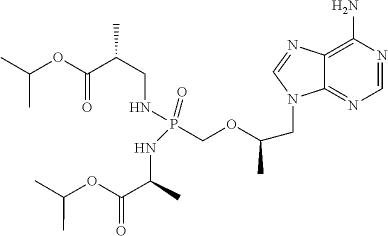 [SMILES: CC(C)OC(=O)[C@H](C)CNP(=O)(CO[C@H](C)Cn1cnc2c(N)ncnc21)N[C@@H](C)C(=O)OC(C)C] |
| III-52 | 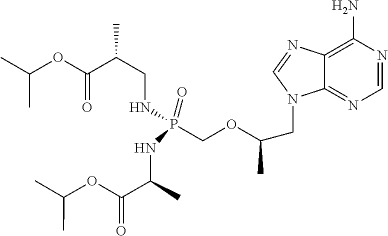 [SMILES: CC(C)OC(=O)[C@H](C)CN[P@](=O)(CO[C@H](C)Cn1cnc2c(N)ncnc21)N[C@@H](C)C(=O)OC(C)C] |
| III-53 | 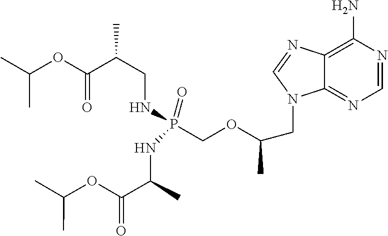 [SMILES: CC(C)OC(=O)[C@H](C)CN[P@@](=O)(CO[C@H](C)Cn1cnc2c(N)ncnc21)N[C@@H](C)C(=O)OC(C)C] |
| III-54 | 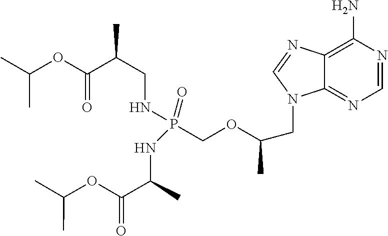 [SMILES: CC(C)OC(=O)[C@H](C)NP(=O)(CO[C@H](C)Cn1cnc2c(N)ncnc21)NC[C@H](C)C(=O)OC(C)C] |
| III-55 | 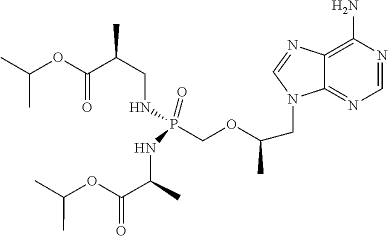 [SMILES: CC(C)OC(=O)[C@H](C)N[P@@](=O)(CO[C@H](C)Cn1cnc2c(N)ncnc21)NC[C@H](C)C(=O)OC(C)C] |
| III-56 | 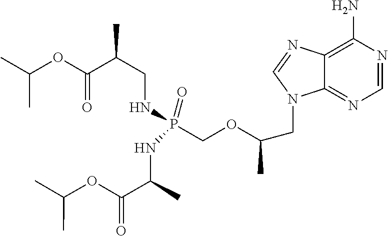 [SMILES: CC(C)OC(=O)[C@H](C)N[P@](=O)(CO[C@H](C)Cn1cnc2c(N)ncnc21)NC[C@H](C)C(=O)OC(C)C] |
| III-57 | 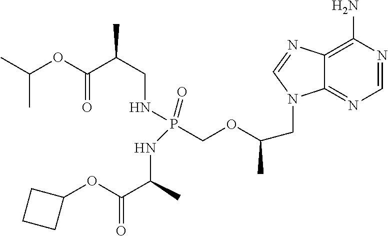 [SMILES: CC(C)OC(=O)[C@@H](C)CNP(=O)(CO[C@H](C)Cn1cnc2c(N)ncnc21)N[C@@H](C)C(=O)OC1CCC1] |
| III-58 | 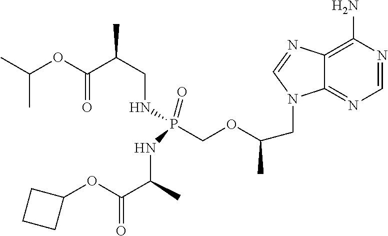 [SMILES: CC(C)OC(=O)[C@@H](C)CN[P@](=O)(CO[C@H](C)Cn1cnc2c(N)ncnc21)N[C@@H](C)C(=O)OC1CCC1] |
| III-59 | 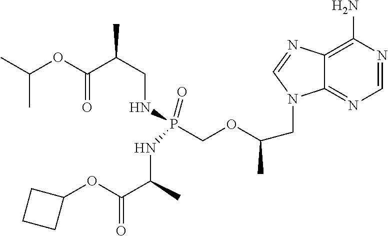 [SMILES: CC(C)OC(=O)[C@@H](C)CN[P@@](=O)(CO[C@H](C)Cn1cnc2c(N)ncnc21)N[C@@H](C)C(=O)OC1CCC1] |
| III-60 | 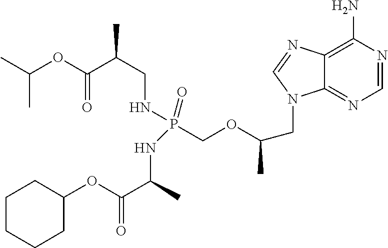 [SMILES: CC(C)OC(=O)[C@@H](C)CNP(=O)(CO[C@H](C)Cn1cnc2c(N)ncnc21)N[C@@H](C)C(=O)OC1CCCCC1] |
| III-61 | 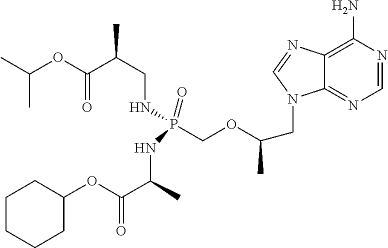 [SMILES: CC(C)OC(=O)[C@@H](C)CN[P@](=O)(CO[C@H](C)Cn1cnc2c(N)ncnc21)N[C@@H](C)C(=O)OC1CCCCC1] |
| III-62 | 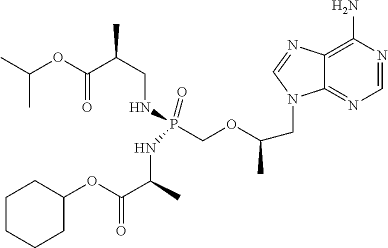 [SMILES: CC(C)OC(=O)[C@@H](C)CN[P@@](=O)(CO[C@H](C)Cn1cnc2c(N)ncnc21)N[C@@H](C)C(=O)OC1CCCCC1] |
| III-63 | 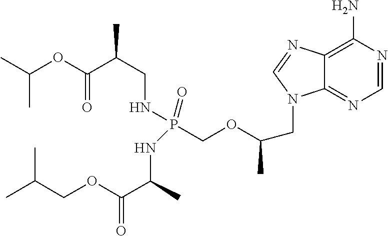 [SMILES: CC(C)COC(=O)[C@H](C)NP(=O)(CO[C@H](C)Cn1cnc2c(N)ncnc21)NC[C@H](C)C(=O)OC(C)C] |
| III-64 | 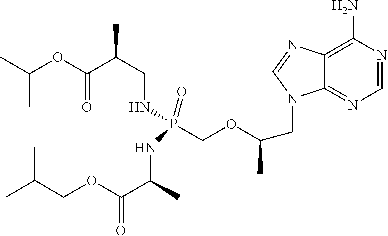 [SMILES: CC(C)COC(=O)[C@H](C)N[P@@](=O)(CO[C@H](C)Cn1cnc2c(N)ncnc21)NC[C@H](C)C(=O)OC(C)C] |
| III-65 | 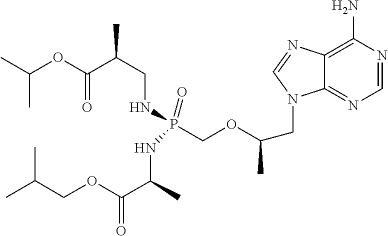 [SMILES: CC(C)COC(=O)[C@H](C)N[P@](=O)(CO[C@H](C)Cn1cnc2c(N)ncnc21)NC[C@H](C)C(=O)OC(C)C] |
| III-66 | 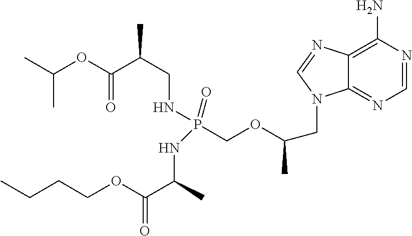 [SMILES: CCCCOC(=O)[C@H](C)NP(=O)(CO[C@H](C)Cn1cnc2c(N)ncnc21)NC[C@H](C)C(=O)OC(C)C] |
| III-67 | 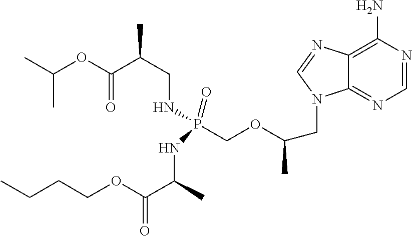 [SMILES: CCCCOC(=O)[C@H](C)N[P@@](=O)(CO[C@H](C)Cn1cnc2c(N)ncnc21)NC[C@H](C)C(=O)OC(C)C] |
| III-68 | 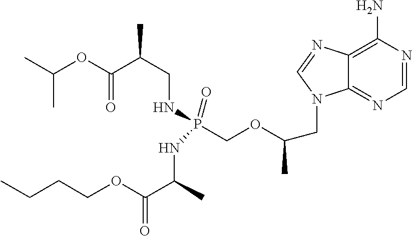 [SMILES: CCCCOC(=O)[C@H](C)N[P@](=O)(CO[C@H](C)Cn1cnc2c(N)ncnc21)NC[C@H](C)C(=O)OC(C)C] |
| III-69 | 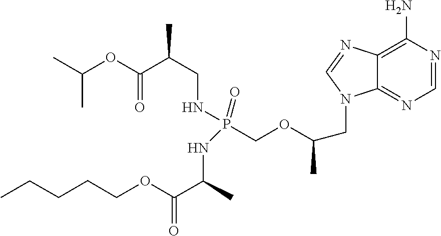 [SMILES: CCCCCOC(=O)[C@H](C)NP(=O)(CO[C@H](C)Cn1cnc2c(N)ncnc21)NC[C@H](C)C(=O)OC(C)C] |
| III-70 | 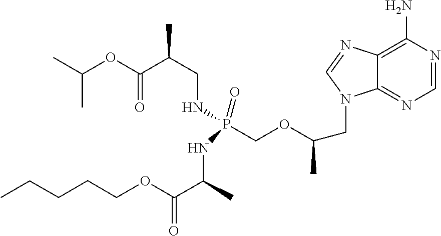 [SMILES: CCCCCOC(=O)[C@H](C)N[P@@](=O)(CO[C@H](C)Cn1cnc2c(N)ncnc21)NC[C@H](C)C(=O)OC(C)C] |
| III-71 | 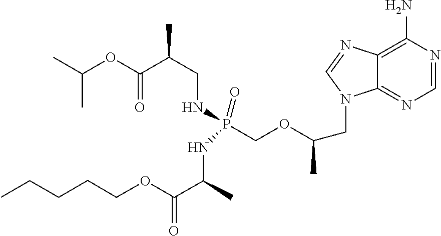 [SMILES: CCCCCOC(=O)[C@H](C)N[P@](=O)(CO[C@H](C)Cn1cnc2c(N)ncnc21)NC[C@H](C)C(=O)OC(C)C] |
| III-72 | 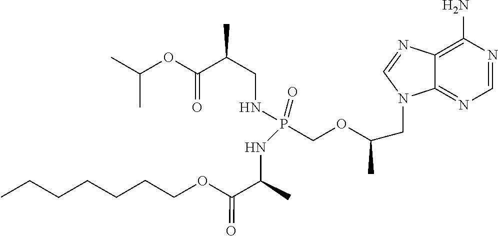 [SMILES: CCCCCCCOC(=O)[C@H](C)NP(=O)(CO[C@H](C)Cn1cnc2c(N)ncnc21)NC[C@H](C)C(=O)OC(C)C] |
| III-73 | 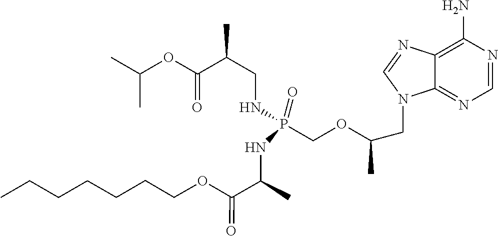 [SMILES: CCCCCCCOC(=O)[C@H](C)N[P@@](=O)(CO[C@H](C)Cn1cnc2c(N)ncnc21)NC[C@H](C)C(=O)OC(C)C] |
| III-74 | 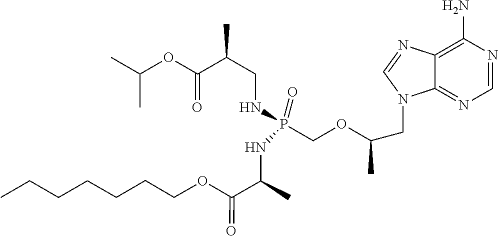 [SMILES: CCCCCCCOC(=O)[C@H](C)N[P@](=O)(CO[C@H](C)Cn1cnc2c(N)ncnc21)NC[C@H](C)C(=O)OC(C)C] |
| III-75 | 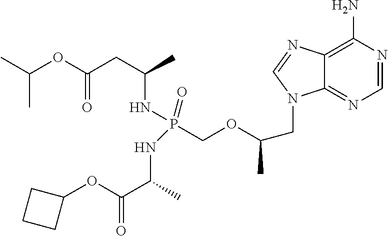 [SMILES: CC(C)OC(=O)C[C@@H](C)NP(=O)(CO[C@H](C)Cn1cnc2c(N)ncnc21)N[C@H](C)C(=O)OC1CCC1] |
| III-76 | 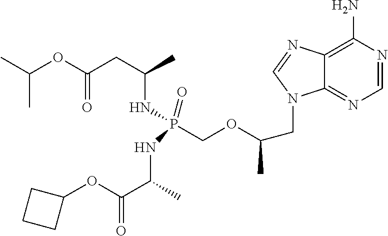 [SMILES: CC(C)OC(=O)C[C@@H](C)N[P@](=O)(CO[C@H](C)Cn1cnc2c(N)ncnc21)N[C@H](C)C(=O)OC1CCC1] |
| III-77 | 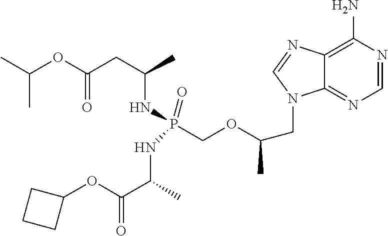 [SMILES: CC(C)OC(=O)C[C@@H](C)N[P@@](=O)(CO[C@H](C)Cn1cnc2c(N)ncnc21)N[C@H](C)C(=O)OC1CCC1] |
| III-78 | 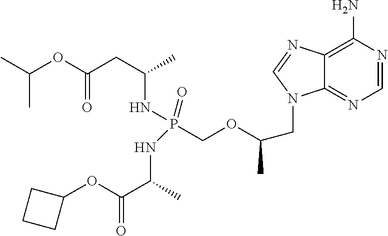 [SMILES: CC(C)OC(=O)C[C@H](C)NP(=O)(CO[C@H](C)Cn1cnc2c(N)ncnc21)N[C@H](C)C(=O)OC1CCC1] |
| III-79 | 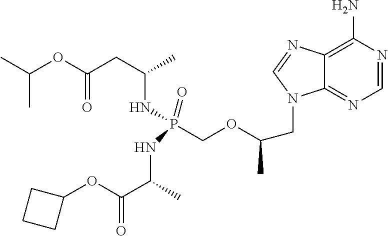 [SMILES: CC(C)OC(=O)C[C@H](C)N[P@](=O)(CO[C@H](C)Cn1cnc2c(N)ncnc21)N[C@H](C)C(=O)OC1CCC1] |
| III-80 | 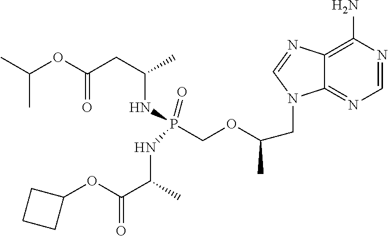 [SMILES: CC(C)OC(=O)C[C@H](C)N[P@@](=O)(CO[C@H](C)Cn1cnc2c(N)ncnc21)N[C@H](C)C(=O)OC1CCC1] |
| III-81 | 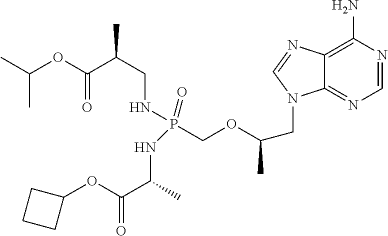 [SMILES: CC(C)OC(=O)[C@@H](C)CNP(=O)(CO[C@H](C)Cn1cnc2c(N)ncnc21)N[C@H](C)C(=O)OC1CCC1] |
| III-82 | 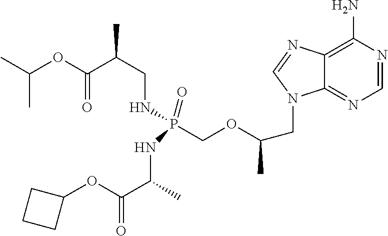 [SMILES: CC(C)OC(=O)[C@@H](C)CN[P@](=O)(CO[C@H](C)Cn1cnc2c(N)ncnc21)N[C@H](C)C(=O)OC1CCC1] |
| III-83 | 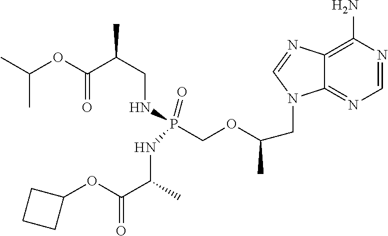 [SMILES: CC(C)OC(=O)[C@@H](C)CN[P@@](=O)(CO[C@H](C)Cn1cnc2c(N)ncnc21)N[C@H](C)C(=O)OC1CCC1] |
| III-84 | 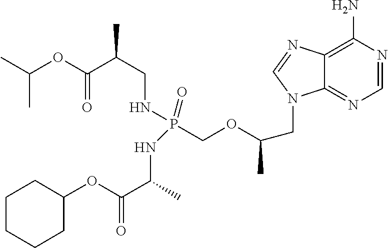 [SMILES: CC(C)OC(=O)[C@@H](C)CNP(=O)(CO[C@H](C)Cn1cnc2c(N)ncnc21)N[C@H](C)C(=O)OC1CCCCC1] |
| III-85 | 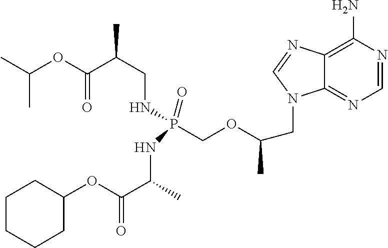 [SMILES: CC(C)OC(=O)[C@@H](C)CN[P@](=O)(CO[C@H](C)Cn1cnc2c(N)ncnc21)N[C@H](C)C(=O)OC1CCCCC1] |
| III-86 | 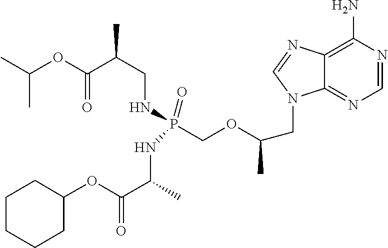 [SMILES: CC(C)OC(=O)[C@@H](C)CN[P@@](=O)(CO[C@H](C)Cn1cnc2c(N)ncnc21)N[C@H](C)C(=O)OC1CCCCC1] |
| III-87 | 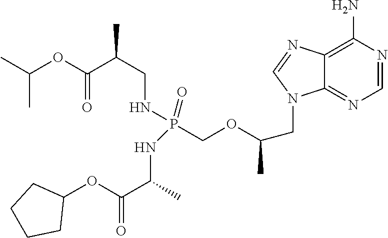 [SMILES: CC(C)OC(=O)[C@@H](C)CNP(=O)(CO[C@H](C)Cn1cnc2c(N)ncnc21)N[C@H](C)C(=O)OC1CCCC1] |
| III-88 | 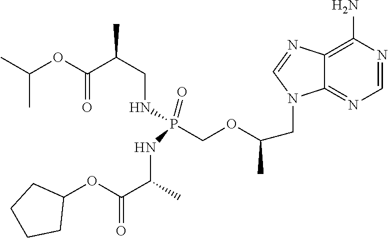 [SMILES: CC(C)OC(=O)[C@@H](C)CN[P@](=O)(CO[C@H](C)Cn1cnc2c(N)ncnc21)N[C@H](C)C(=O)OC1CCCC1] |
| III-89 | 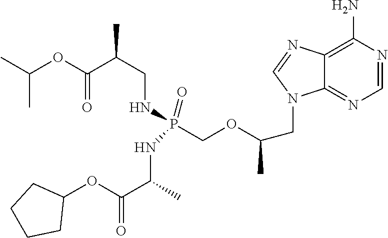 [SMILES: CC(C)OC(=O)[C@@H](C)CN[P@@](=O)(CO[C@H](C)Cn1cnc2c(N)ncnc21)N[C@H](C)C(=O)OC1CCCC1] |
| III-90 | 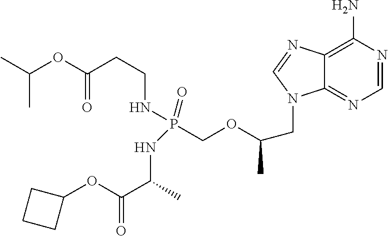 [SMILES: CC(C)OC(=O)CCNP(=O)(CO[C@H](C)Cn1cnc2c(N)ncnc21)N[C@H](C)C(=O)OC1CCC1] |
| III-91 | 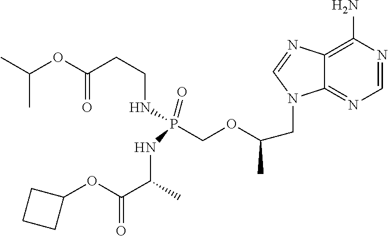 [SMILES: CC(C)OC(=O)CCN[P@](=O)(CO[C@H](C)Cn1cnc2c(N)ncnc21)N[C@H](C)C(=O)OC1CCC1] |
| III-92 | 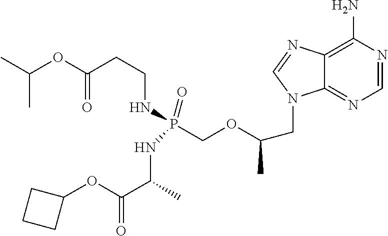 [SMILES: CC(C)OC(=O)CCN[P@@](=O)(CO[C@H](C)Cn1cnc2c(N)ncnc21)N[C@H](C)C(=O)OC1CCC1] |
| III-93 | 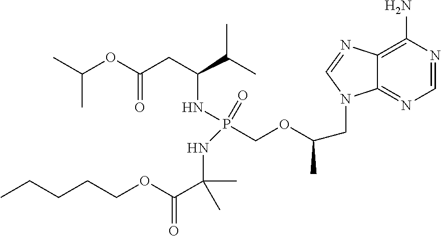 [SMILES: CCCCCOC(=O)C(C)(C)NP(=O)(CO[C@H](C)Cn1cnc2c(N)ncnc21)N[C@@H](CC(=O)OC(C)C)C(C)C] |
| III-94 | 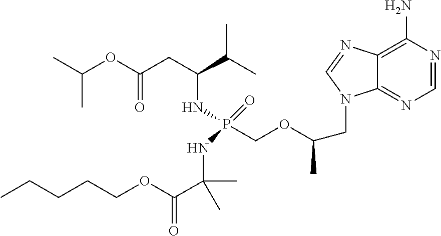 [SMILES: CCCCCOC(=O)C(C)(C)N[P@@](=O)(CO[C@H](C)Cn1cnc2c(N)ncnc21)N[C@@H](CC(=O)OC(C)C)C(C)C] |
| III-95 | 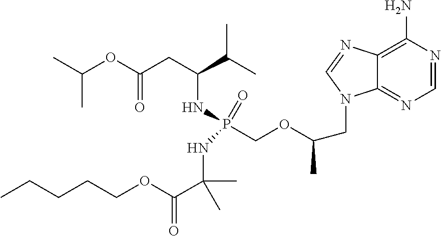 [SMILES: CCCCCOC(=O)C(C)(C)N[P@](=O)(CO[C@H](C)Cn1cnc2c(N)ncnc21)N[C@@H](CC(=O)OC(C)C)C(C)C] |
| III-96 | 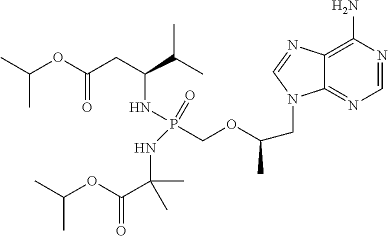 [SMILES: CC(C)OC(=O)C[C@H](NP(=O)(CO[C@H](C)Cn1cnc2c(N)ncnc21)NC(C)(C)C(=O)OC(C)C)C(C)C] |
| III-97 | 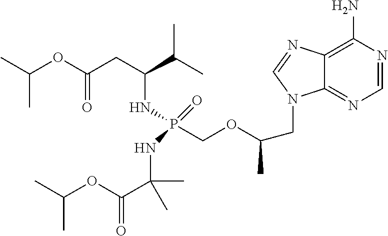 [SMILES: CC(C)OC(=O)C[C@H](N[P@](=O)(CO[C@H](C)Cn1cnc2c(N)ncnc21)NC(C)(C)C(=O)OC(C)C)C(C)C] |
| III-98 | 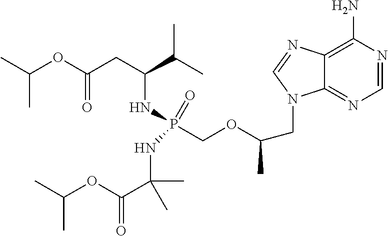 [SMILES: CC(C)OC(=O)C[C@H](N[P@@](=O)(CO[C@H](C)Cn1cnc2c(N)ncnc21)NC(C)(C)C(=O)OC(C)C)C(C)C] |
| III-99 | 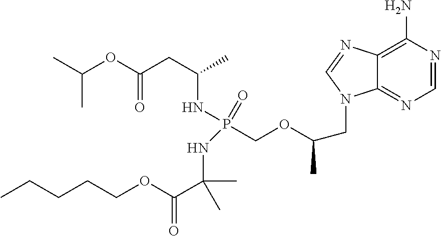 [SMILES: CCCCCOC(=O)C(C)(C)NP(=O)(CO[C@H](C)Cn1cnc2c(N)ncnc21)N[C@@H](C)CC(=O)OC(C)C] |
| III-100 | 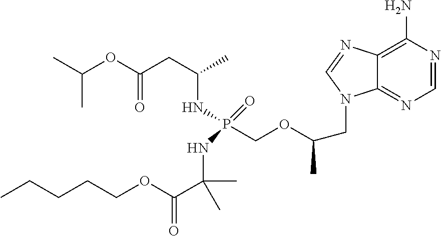 [SMILES: CCCCCOC(=O)C(C)(C)N[P@@](=O)(CO[C@H](C)Cn1cnc2c(N)ncnc21)N[C@@H](C)CC(=O)OC(C)C] |
| III-101 | 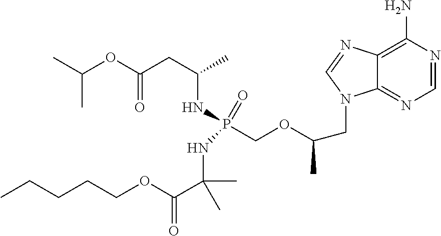 [SMILES: CCCCCOC(=O)C(C)(C)N[P@](=O)(CO[C@H](C)Cn1cnc2c(N)ncnc21)N[C@@H](C)CC(=O)OC(C)C] |
| III-102 | 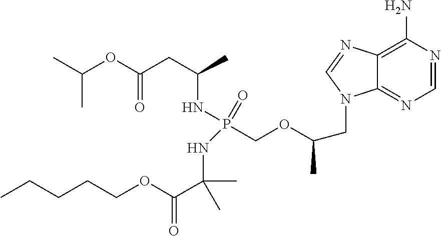 [SMILES: CCCCCOC(=O)C(C)(C)NP(=O)(CO[C@H](C)Cn1cnc2c(N)ncnc21)N[C@H](C)CC(=O)OC(C)C] |
| III-103 | 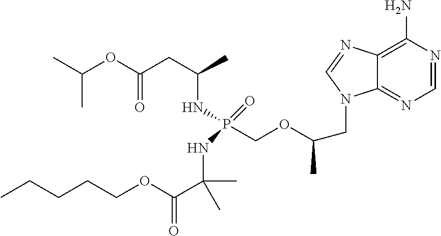 [SMILES: CCCCCOC(=O)C(C)(C)N[P@@](=O)(CO[C@H](C)Cn1cnc2c(N)ncnc21)N[C@H](C)CC(=O)OC(C)C] |
| III-104 | 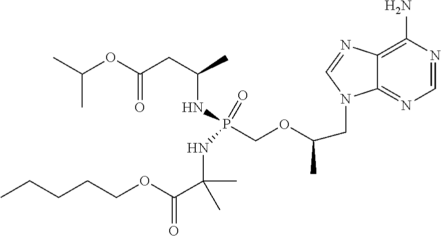 [SMILES: CCCCCOC(=O)C(C)(C)N[P@](=O)(CO[C@H](C)Cn1cnc2c(N)ncnc21)N[C@H](C)CC(=O)OC(C)C] |
| III-105 | 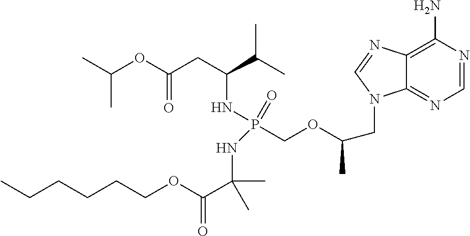 [SMILES: CCCCCCOC(=O)C(C)(C)NP(=O)(CO[C@H](C)Cn1cnc2c(N)ncnc21)N[C@@H](CC(=O)OC(C)C)C(C)C] |
| III-106 | 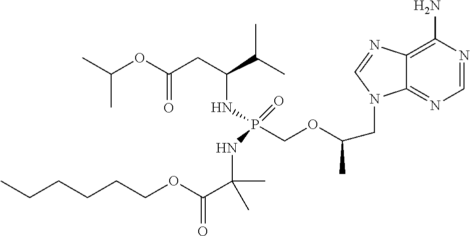 [SMILES: CCCCCCOC(=O)C(C)(C)N[P@@](=O)(CO[C@H](C)Cn1cnc2c(N)ncnc21)N[C@@H](CC(=O)OC(C)C)C(C)C] |
| III-107 | 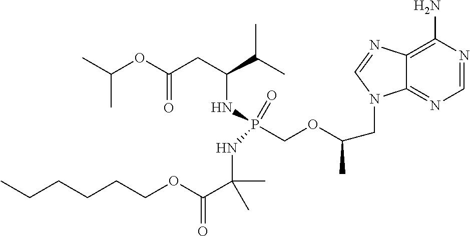 [SMILES: CCCCCCOC(=O)C(C)(C)N[P@](=O)(CO[C@H](C)Cn1cnc2c(N)ncnc21)N[C@@H](CC(=O)OC(C)C)C(C)C] |
| III-108 | 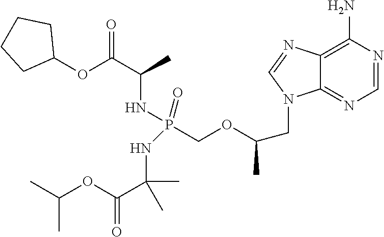 [SMILES: CC(C)OC(=O)C(C)(C)NP(=O)(CO[C@H](C)Cn1cnc2c(N)ncnc21)N[C@H](C)C(=O)OC1CCCC1] |
| III-109 | 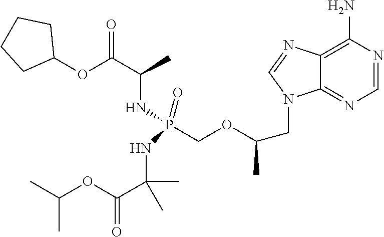 [SMILES: CC(C)OC(=O)C(C)(C)N[P@@](=O)(CO[C@H](C)Cn1cnc2c(N)ncnc21)N[C@H](C)C(=O)OC1CCCC1] |
| III-110 | 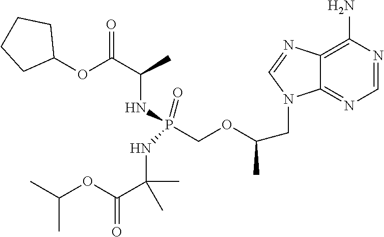 [SMILES: CC(C)OC(=O)C(C)(C)N[P@](=O)(CO[C@H](C)Cn1cnc2c(N)ncnc21)N[C@H](C)C(=O)OC1CCCC1] |
| III-111 | 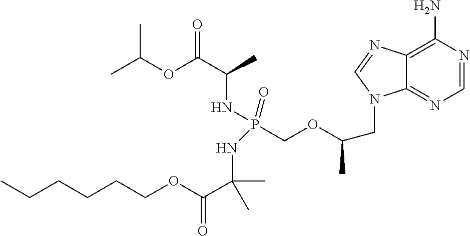 [SMILES: CCCCCCOC(=O)C(C)(C)NP(=O)(CO[C@H](C)Cn1cnc2c(N)ncnc21)N[C@H](C)C(=O)OC(C)C] |
| III-112 | 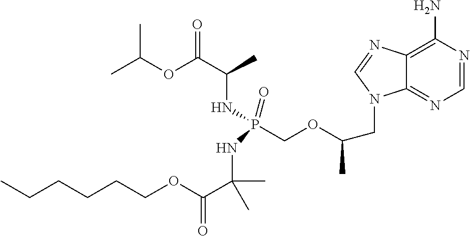 [SMILES: CCCCCCOC(=O)C(C)(C)N[P@@](=O)(CO[C@H](C)Cn1cnc2c(N)ncnc21)N[C@H](C)C(=O)OC(C)C] |
| III-113 | 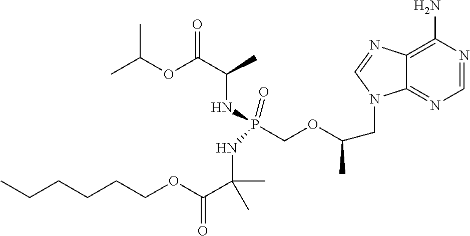 [SMILES: CCCCCCOC(=O)C(C)(C)N[P@](=O)(CO[C@H](C)Cn1cnc2c(N)ncnc21)N[C@H](C)C(=O)OC(C)C] |
| III-114 | 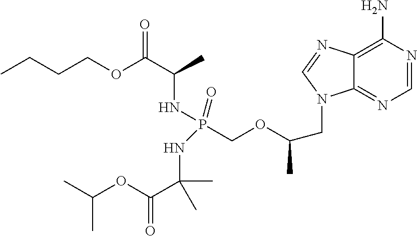 [SMILES: CCCCOC(=O)[C@@H](C)NP(=O)(CO[C@H](C)Cn1cnc2c(N)ncnc21)NC(C)(C)C(=O)OC(C)C] |
| III-115 | 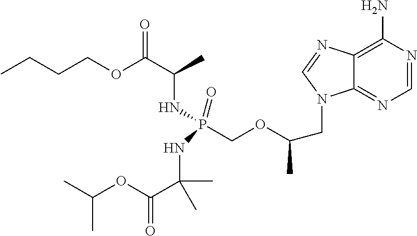 [SMILES: CCCCOC(=O)[C@@H](C)N[P@](=O)(CO[C@H](C)Cn1cnc2c(N)ncnc21)NC(C)(C)C(=O)OC(C)C] |
| III-116 | 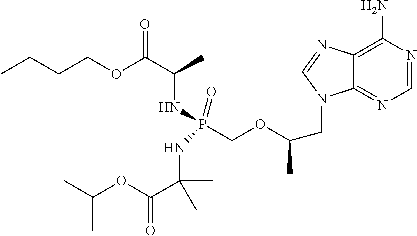 [SMILES: CCCCOC(=O)[C@@H](C)N[P@@](=O)(CO[C@H](C)Cn1cnc2c(N)ncnc21)NC(C)(C)C(=O)OC(C)C] |
| III-117 | 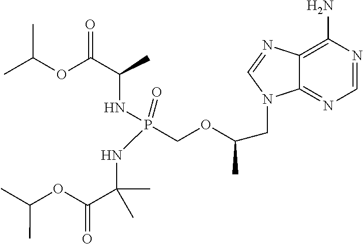 [SMILES: CC(C)OC(=O)[C@@H](C)NP(=O)(CO[C@H](C)Cn1cnc2c(N)ncnc21)NC(C)(C)C(=O)OC(C)C] |
| III-118 | 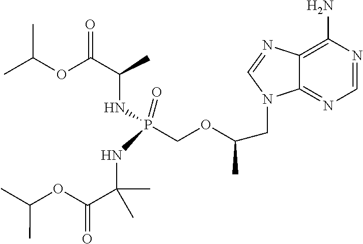 [SMILES: CC(C)OC(=O)[C@@H](C)N[P@](=O)(CO[C@H](C)Cn1cnc2c(N)ncnc21)NC(C)(C)C(=O)OC(C)C] |
| III-119 | 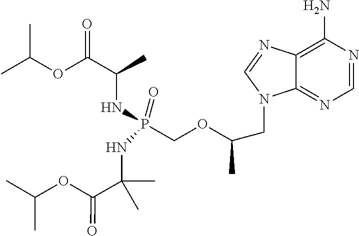 [SMILES: CC(C)OC(=O)[C@@H](C)N[P@@](=O)(CO[C@H](C)Cn1cnc2c(N)ncnc21)NC(C)(C)C(=O)OC(C)C] |
| III-120 | 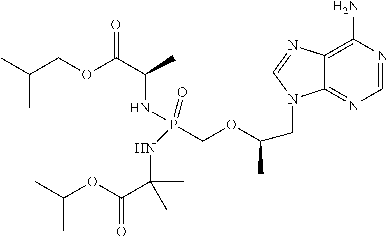 [SMILES: CC(C)COC(=O)[C@@H](C)NP(=O)(CO[C@H](C)Cn1cnc2c(N)ncnc21)NC(C)(C)C(=O)OC(C)C] |
| III-121 | 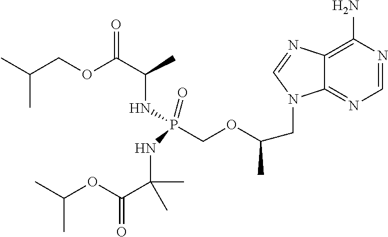 [SMILES: CC(C)COC(=O)[C@@H](C)N[P@](=O)(CO[C@H](C)Cn1cnc2c(N)ncnc21)NC(C)(C)C(=O)OC(C)C] |
| III-122 | 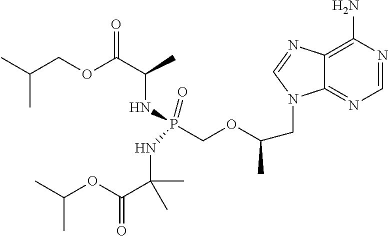 [SMILES: CC(C)COC(=O)[C@@H](C)N[P@@](=O)(CO[C@H](C)Cn1cnc2c(N)ncnc21)NC(C)(C)C(=O)OC(C)C] |
| III-123 | 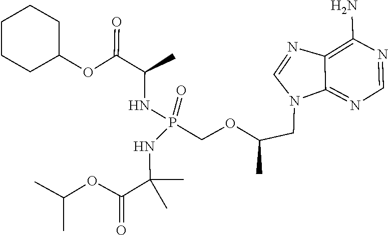 [SMILES: CC(C)OC(=O)C(C)(C)NP(=O)(CO[C@H](C)Cn1cnc2c(N)ncnc21)N[C@H](C)C(=O)OC1CCCCC1] |
| III-124 | 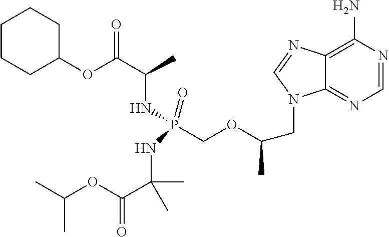 [SMILES: CC(C)OC(=O)C(C)(C)N[P@@](=O)(CO[C@H](C)Cn1cnc2c(N)ncnc21)N[C@H](C)C(=O)OC1CCCCC1] |
| III-125 | 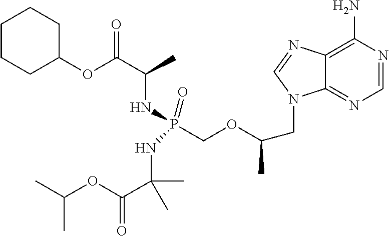 [SMILES: CC(C)OC(=O)C(C)(C)N[P@](=O)(CO[C@H](C)Cn1cnc2c(N)ncnc21)N[C@H](C)C(=O)OC1CCCCC1] |
| III-126 | 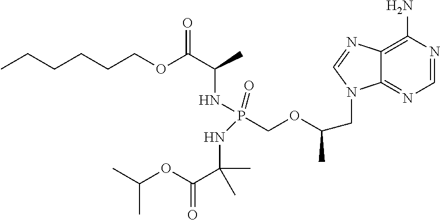 [SMILES: CCCCCCOC(=O)[C@@H](C)NP(=O)(CO[C@H](C)Cn1cnc2c(N)ncnc21)NC(C)(C)C(=O)OC(C)C] |
| III-127 | 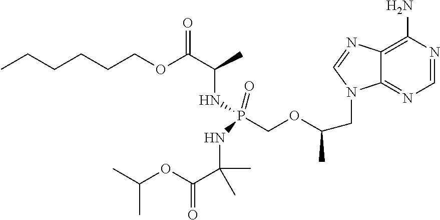 [SMILES: CCCCCCOC(=O)[C@@H](C)N[P@](=O)(CO[C@H](C)Cn1cnc2c(N)ncnc21)NC(C)(C)C(=O)OC(C)C] |
| III-128 | 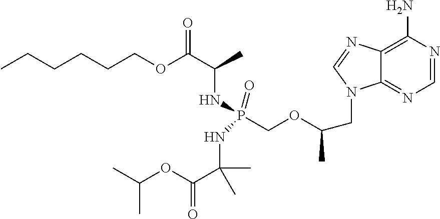 [SMILES: CCCCCCOC(=O)[C@@H](C)N[P@@](=O)(CO[C@H](C)Cn1cnc2c(N)ncnc21)NC(C)(C)C(=O)OC(C)C] |
| III-129 | 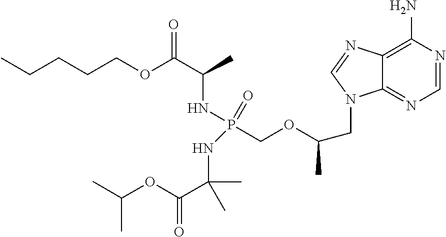 [SMILES: CCCCCOC(=O)[C@@H](C)NP(=O)(CO[C@H](C)Cn1cnc2c(N)ncnc21)NC(C)(C)C(=O)OC(C)C] |
| III-130 | 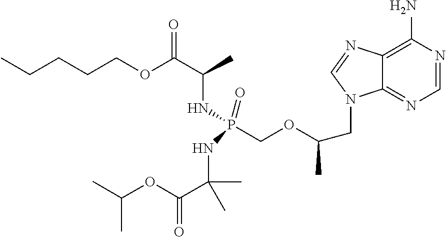 [SMILES: CCCCCOC(=O)[C@@H](C)N[P@](=O)(CO[C@H](C)Cn1cnc2c(N)ncnc21)NC(C)(C)C(=O)OC(C)C] |
| III-131 | 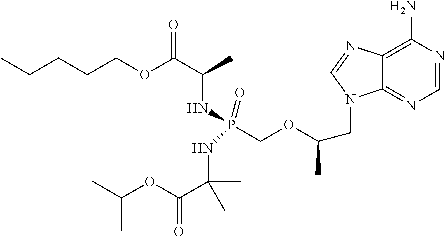 [SMILES: CCCCCOC(=O)[C@@H](C)N[P@@](=O)(CO[C@H](C)Cn1cnc2c(N)ncnc21)NC(C)(C)C(=O)OC(C)C] |
| III-132 | 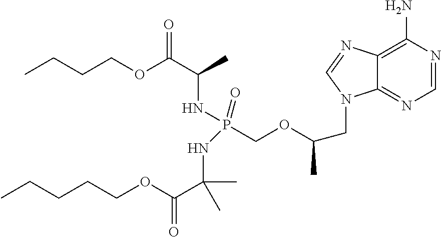 [SMILES: CCCCCOC(=O)C(C)(C)NP(=O)(CO[C@H](C)Cn1cnc2c(N)ncnc21)N[C@H](C)C(=O)OCCCC] |
| III-133 | 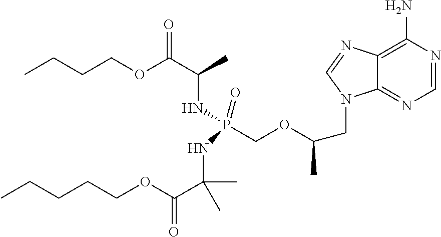 [SMILES: CCCCCOC(=O)C(C)(C)N[P@@](=O)(CO[C@H](C)Cn1cnc2c(N)ncnc21)N[C@H](C)C(=O)OCCCC] |
| III-134 | 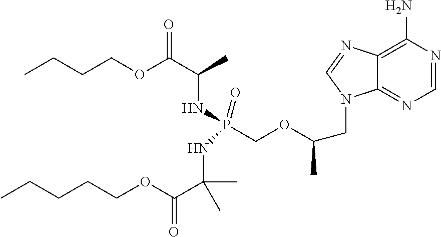 [SMILES: CCCCCOC(=O)C(C)(C)N[P@](=O)(CO[C@H](C)Cn1cnc2c(N)ncnc21)N[C@H](C)C(=O)OCCCC] |
| III-135 | 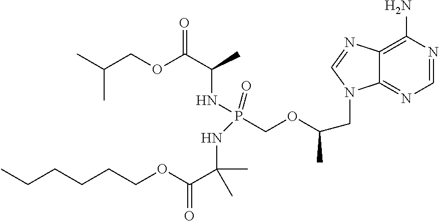 [SMILES: CCCCCCOC(=O)C(C)(C)NP(=O)(CO[C@H](C)Cn1cnc2c(N)ncnc21)N[C@H](C)C(=O)OCC(C)C] |
| III-136 | 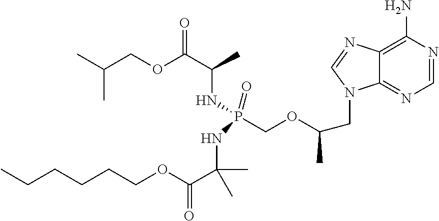 [SMILES: CCCCCCOC(=O)C(C)(C)N[P@@](=O)(CO[C@H](C)Cn1cnc2c(N)ncnc21)N[C@H](C)C(=O)OCC(C)C] |
| III-137 | 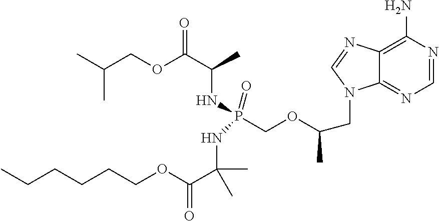 [SMILES: CCCCCCOC(=O)C(C)(C)N[P@](=O)(CO[C@H](C)Cn1cnc2c(N)ncnc21)N[C@H](C)C(=O)OCC(C)C] |
| III-138 | 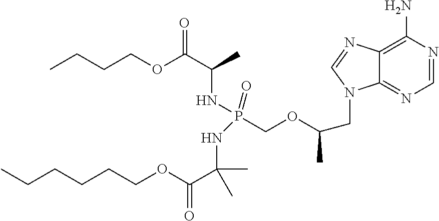 [SMILES: CCCCCCOC(=O)C(C)(C)NP(=O)(CO[C@H](C)Cn1cnc2c(N)ncnc21)N[C@H](C)C(=O)OCCCC] |
| III-139 | 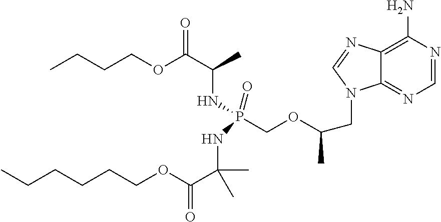 [SMILES: CCCCCCOC(=O)C(C)(C)N[P@@](=O)(CO[C@H](C)Cn1cnc2c(N)ncnc21)N[C@H](C)C(=O)OCCCC] |
| III-140 | 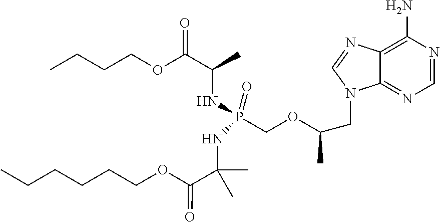 [SMILES: CCCCCCOC(=O)C(C)(C)N[P@](=O)(CO[C@H](C)Cn1cnc2c(N)ncnc21)N[C@H](C)C(=O)OCCCC] |
| III-141 | 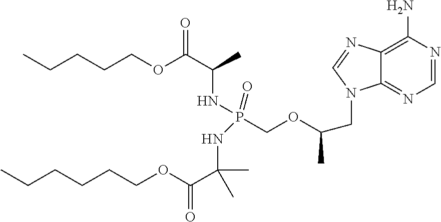 [SMILES: CCCCCCOC(=O)C(C)(C)NP(=O)(CO[C@H](C)Cn1cnc2c(N)ncnc21)N[C@H](C)C(=O)OCCCCC] |
| III-142 | 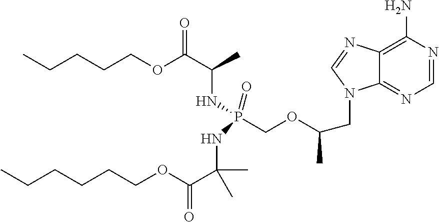 [SMILES: CCCCCCOC(=O)C(C)(C)N[P@@](=O)(CO[C@H](C)Cn1cnc2c(N)ncnc21)N[C@H](C)C(=O)OCCCCC] |
| III-143 | 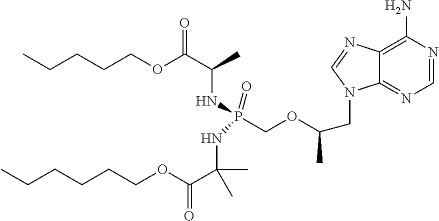 [SMILES: CCCCCCOC(=O)C(C)(C)N[P@](=O)(CO[C@H](C)Cn1cnc2c(N)ncnc21)N[C@H](C)C(=O)OCCCCC] |
| III-144 | 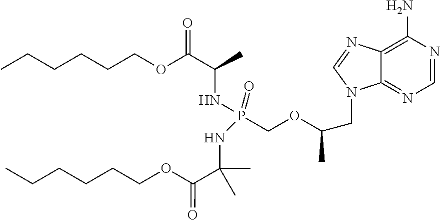 [SMILES: CCCCCCOC(=O)[C@@H](C)NP(=O)(CO[C@H](C)Cn1cnc2c(N)ncnc21)NC(C)(C)C(=O)OCCCCCC] |
| III-145 | 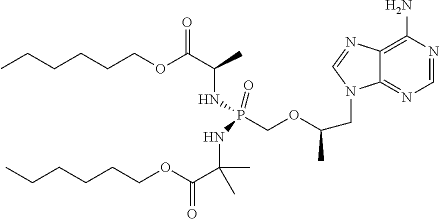 [SMILES: CCCCCCOC(=O)[C@@H](C)N[P@](=O)(CO[C@H](C)Cn1cnc2c(N)ncnc21)NC(C)(C)C(=O)OCCCCCC] |
| III-146 | 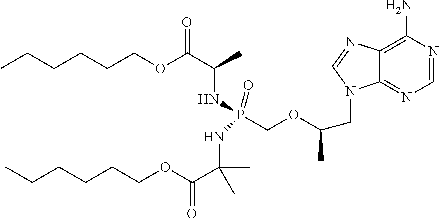 [SMILES: CCCCCCOC(=O)[C@@H](C)N[P@@](=O)(CO[C@H](C)Cn1cnc2c(N)ncnc21)NC(C)(C)C(=O)OCCCCCC] |
| III-147 | 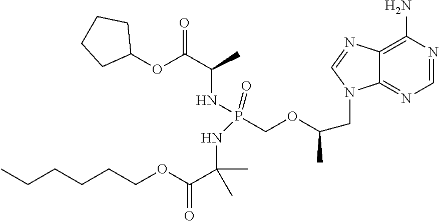 [SMILES: CCCCCCOC(=O)C(C)(C)NP(=O)(CO[C@H](C)Cn1cnc2c(N)ncnc21)N[C@H](C)C(=O)OC1CCCC1] |
| III-148 | 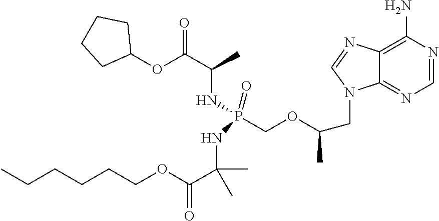 [SMILES: CCCCCCOC(=O)C(C)(C)N[P@@](=O)(CO[C@H](C)Cn1cnc2c(N)ncnc21)N[C@H](C)C(=O)OC1CCCC1] |
| III-149 | 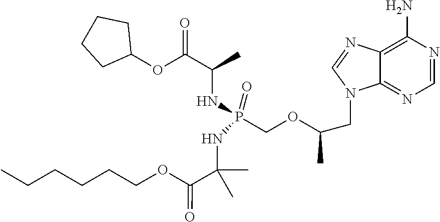 [SMILES: CCCCCCOC(=O)C(C)(C)N[P@](=O)(CO[C@H](C)Cn1cnc2c(N)ncnc21)N[C@H](C)C(=O)OC1CCCC1] |
| III-150 | 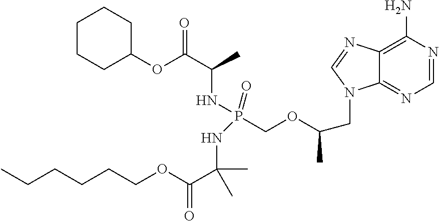 [SMILES: CCCCCCOC(=O)C(C)(C)NP(=O)(CO[C@H](C)Cn1cnc2c(N)ncnc21)N[C@H](C)C(=O)OC1CCCCC1] |
| III-151 | 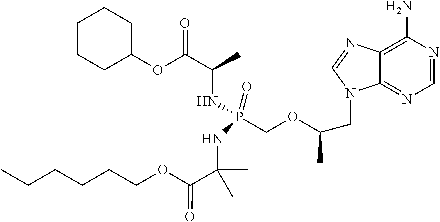 [SMILES: CCCCCCOC(=O)C(C)(C)N[P@@](=O)(CO[C@H](C)Cn1cnc2c(N)ncnc21)N[C@H](C)C(=O)OC1CCCCC1] |
| III-152 | 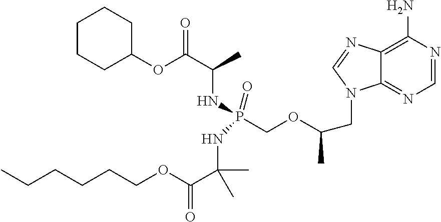 [SMILES: CCCCCCOC(=O)C(C)(C)N[P@](=O)(CO[C@H](C)Cn1cnc2c(N)ncnc21)N[C@H](C)C(=O)OC1CCCCC1] |
| III-153 | 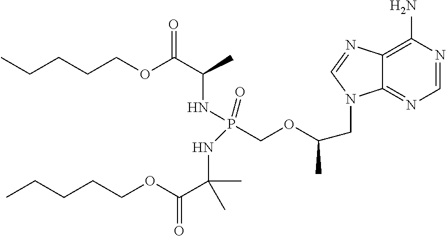 [SMILES: CCCCCOC(=O)[C@@H](C)NP(=O)(CO[C@H](C)Cn1cnc2c(N)ncnc21)NC(C)(C)C(=O)OCCCCC] |
| III-154 | 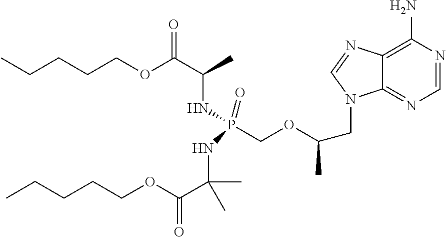 [SMILES: CCCCCOC(=O)[C@@H](C)N[P@](=O)(CO[C@H](C)Cn1cnc2c(N)ncnc21)NC(C)(C)C(=O)OCCCCC] |
| III-155 | 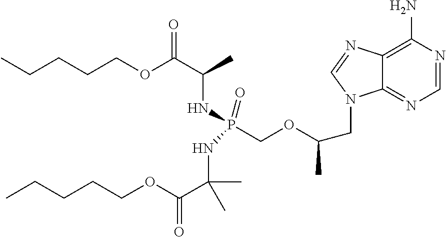 [SMILES: CCCCCOC(=O)[C@@H](C)N[P@@](=O)(CO[C@H](C)Cn1cnc2c(N)ncnc21)NC(C)(C)C(=O)OCCCCC] |
| III-156 | 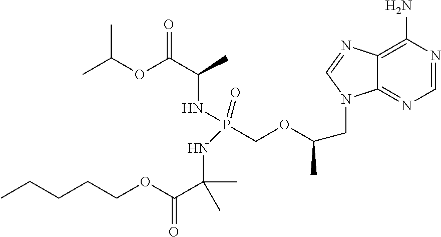 [SMILES: CCCCCOC(=O)C(C)(C)NP(=O)(CO[C@H](C)Cn1cnc2c(N)ncnc21)N[C@H](C)C(=O)OC(C)C] |
| III-157 | 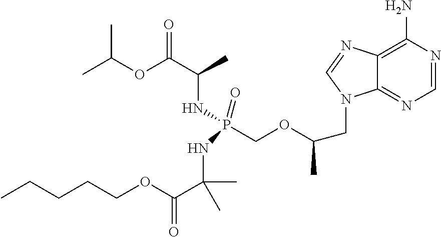 [SMILES: CCCCCOC(=O)C(C)(C)N[P@@](=O)(CO[C@H](C)Cn1cnc2c(N)ncnc21)N[C@H](C)C(=O)OC(C)C] |
| III-158 | 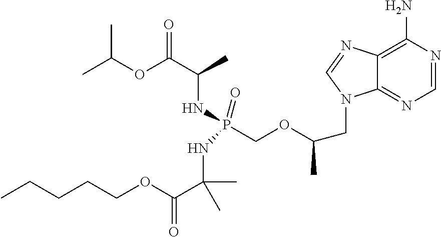 [SMILES: CCCCCOC(=O)C(C)(C)N[P@](=O)(CO[C@H](C)Cn1cnc2c(N)ncnc21)N[C@H](C)C(=O)OC(C)C] |
| III-159 | 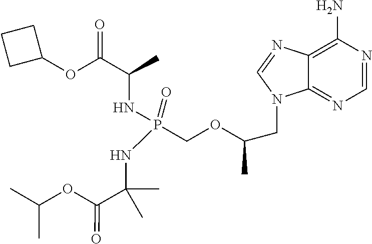 [SMILES: CC(C)OC(=O)C(C)(C)NP(=O)(CO[C@H](C)Cn1cnc2c(N)ncnc21)N[C@H](C)C(=O)OC1CCC1] |
| III-160 | 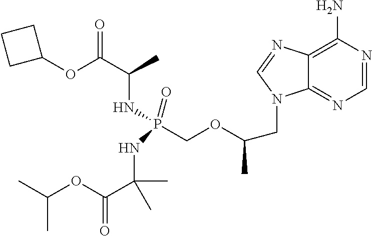 [SMILES: CC(C)OC(=O)C(C)(C)N[P@@](=O)(CO[C@H](C)Cn1cnc2c(N)ncnc21)N[C@H](C)C(=O)OC1CCC1] |
| III-161 | 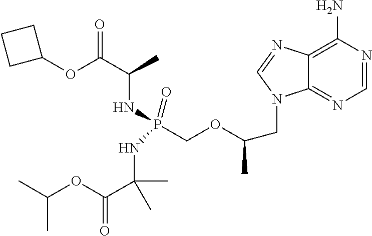 [SMILES: CC(C)OC(=O)C(C)(C)N[P@](=O)(CO[C@H](C)Cn1cnc2c(N)ncnc21)N[C@H](C)C(=O)OC1CCC1] |
| III-162 | 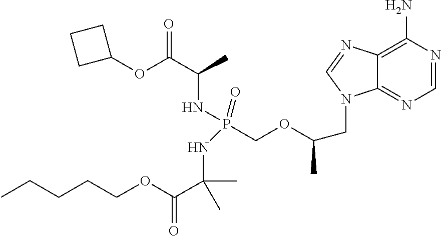 [SMILES: CCCCCOC(=O)C(C)(C)NP(=O)(CO[C@H](C)Cn1cnc2c(N)ncnc21)N[C@H](C)C(=O)OC1CCC1] |
| III-163 | 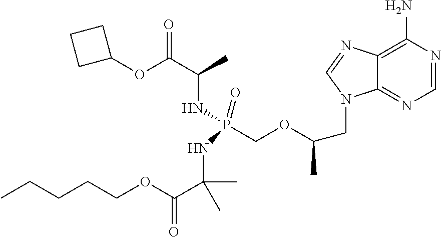 [SMILES: CCCCCOC(=O)C(C)(C)N[P@@](=O)(CO[C@H](C)Cn1cnc2c(N)ncnc21)N[C@H](C)C(=O)OC1CCC1] |
| III-164 | 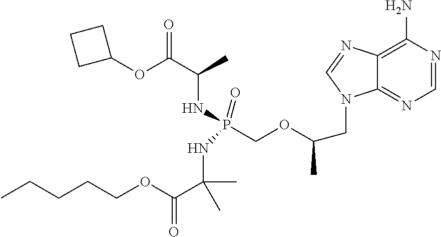 [SMILES: CCCCCOC(=O)C(C)(C)N[P@](=O)(CO[C@H](C)Cn1cnc2c(N)ncnc21)N[C@H](C)C(=O)OC1CCC1] |
| III-165 | 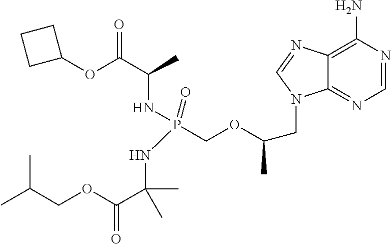 [SMILES: CC(C)COC(=O)C(C)(C)NP(=O)(CO[C@H](C)Cn1cnc2c(N)ncnc21)N[C@H](C)C(=O)OC1CCC1] |
| III-166 | 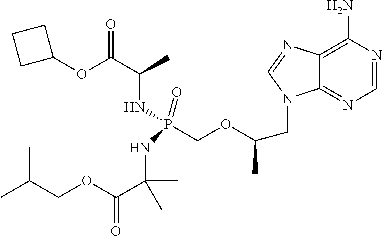 [SMILES: CC(C)COC(=O)C(C)(C)N[P@@](=O)(CO[C@H](C)Cn1cnc2c(N)ncnc21)N[C@H](C)C(=O)OC1CCC1] |
| III-167 | 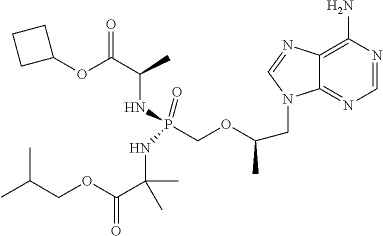 [SMILES: CC(C)COC(=O)C(C)(C)N[P@](=O)(CO[C@H](C)Cn1cnc2c(N)ncnc21)N[C@H](C)C(=O)OC1CCC1] |
| III-168 | 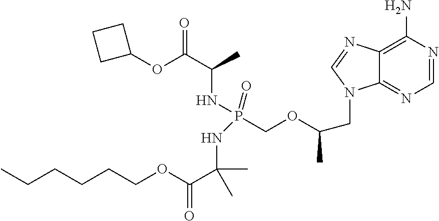 [SMILES: CCCCCCOC(=O)C(C)(C)NP(=O)(CO[C@H](C)Cn1cnc2c(N)ncnc21)N[C@H](C)C(=O)OC1CCC1] |
| III-169 | 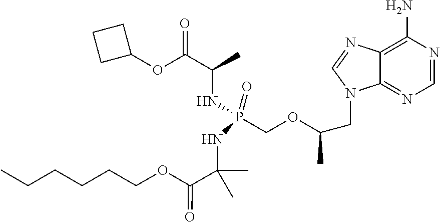 [SMILES: CCCCCCOC(=O)C(C)(C)N[P@@](=O)(CO[C@H](C)Cn1cnc2c(N)ncnc21)N[C@H](C)C(=O)OC1CCC1] |
| III-170 | 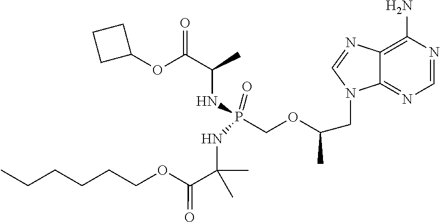 [SMILES: CCCCCCOC(=O)C(C)(C)N[P@](=O)(CO[C@H](C)Cn1cnc2c(N)ncnc21)N[C@H](C)C(=O)OC1CCC1] |
| III-171 | 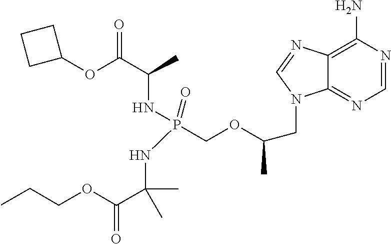 [SMILES: CCCOC(=O)C(C)(C)NP(=O)(CO[C@H](C)Cn1cnc2c(N)ncnc21)N[C@H](C)C(=O)OC1CCC1] |
| III-172 | 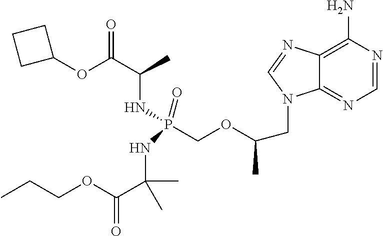 [SMILES: CCCOC(=O)C(C)(C)N[P@@](=O)(CO[C@H](C)Cn1cnc2c(N)ncnc21)N[C@H](C)C(=O)OC1CCC1] |
| III-173 | 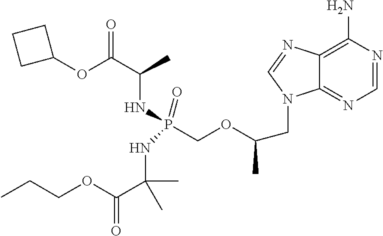 [SMILES: CCCOC(=O)C(C)(C)N[P@](=O)(CO[C@H](C)Cn1cnc2c(N)ncnc21)N[C@H](C)C(=O)OC1CCC1] |
| III-174 | 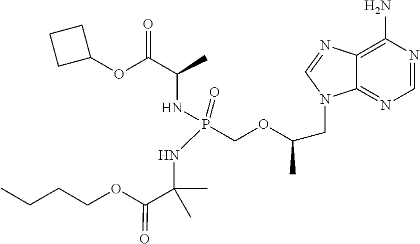 [SMILES: CCCCOC(=O)C(C)(C)NP(=O)(CO[C@H](C)Cn1cnc2c(N)ncnc21)N[C@H](C)C(=O)OC1CCC1] |
| III-175 | 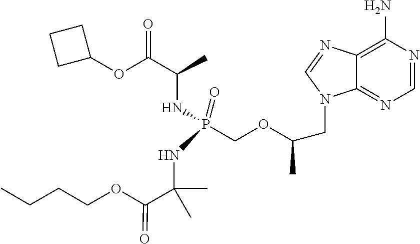 [SMILES: CCCCOC(=O)C(C)(C)N[P@@](=O)(CO[C@H](C)Cn1cnc2c(N)ncnc21)N[C@H](C)C(=O)OC1CCC1] |
| III-176 | 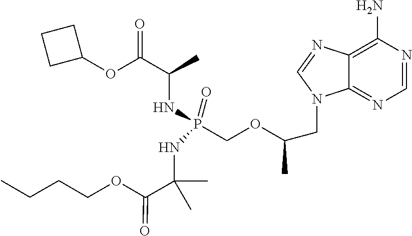 [SMILES: CCCCOC(=O)C(C)(C)N[P@](=O)(CO[C@H](C)Cn1cnc2c(N)ncnc21)N[C@H](C)C(=O)OC1CCC1] |
| III-177 | 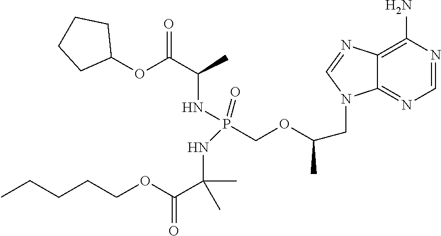 [SMILES: CCCCCOC(=O)C(C)(C)NP(=O)(CO[C@H](C)Cn1cnc2c(N)ncnc21)N[C@H](C)C(=O)OC1CCCC1] |
| III-178 | 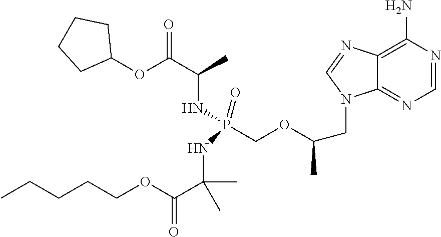 [SMILES: CCCCCOC(=O)C(C)(C)N[P@@](=O)(CO[C@H](C)Cn1cnc2c(N)ncnc21)N[C@H](C)C(=O)OC1CCCC1] |
| III-179 | 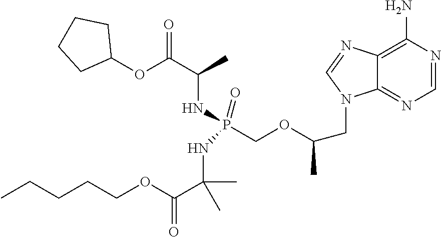 [SMILES: CCCCCOC(=O)C(C)(C)N[P@](=O)(CO[C@H](C)Cn1cnc2c(N)ncnc21)N[C@H](C)C(=O)OC1CCCC1] |
| III-180 | 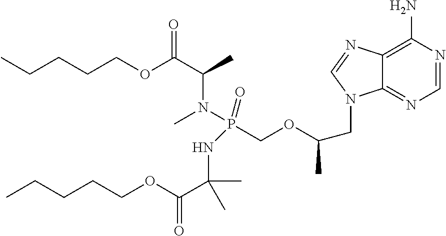 [SMILES: CCCCCOC(=O)[C@@H](C)N(C)P(=O)(CO[C@H](C)Cn1cnc2c(N)ncnc21)NC(C)(C)C(=O)OCCCCC] |
| III-181 | 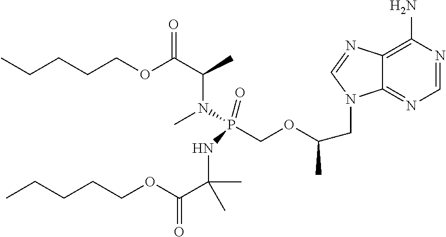 [SMILES: CCCCCOC(=O)[C@@H](C)N(C)[P@](=O)(CO[C@H](C)Cn1cnc2c(N)ncnc21)NC(C)(C)C(=O)OCCCCC] |
| III-182 | 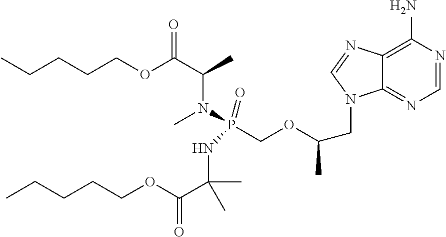 [SMILES: CCCCCOC(=O)[C@@H](C)N(C)[P@@](=O)(CO[C@H](C)Cn1cnc2c(N)ncnc21)NC(C)(C)C(=O)OCCCCC] |
| III-183 | 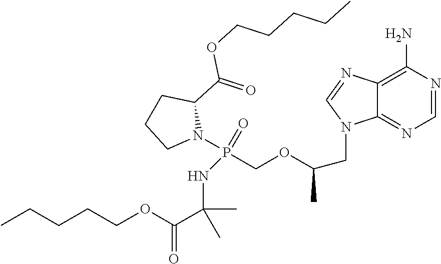 [SMILES: CCCCCOC(=O)[C@H]1CCCN1P(=O)(CO[C@H](C)Cn1cnc2c(N)ncnc21)NC(C)(C)C(=O)OCCCCC] |
| III-184 | 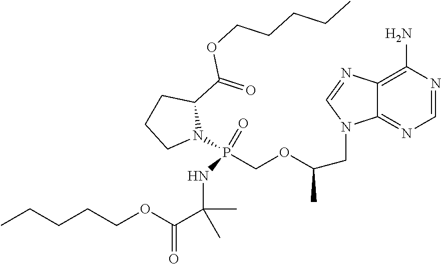 [SMILES: CCCCCOC(=O)[C@H]1CCCN1[P@](=O)(CO[C@H](C)Cn1cnc2c(N)ncnc21)NC(C)(C)C(=O)OCCCCC] |
| III-185 | 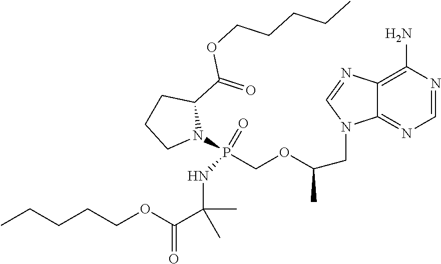 [SMILES: CCCCCOC(=O)[C@H]1CCCN1[P@@](=O)(CO[C@H](C)Cn1cnc2c(N)ncnc21)NC(C)(C)C(=O)OCCCCC] |
| III-186 | 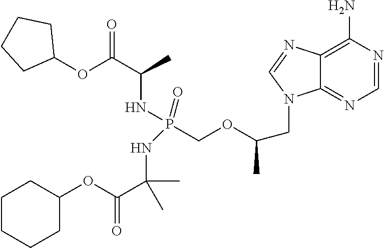 [SMILES: C[C@H](Cn1cnc2c(N)ncnc21)OCP(=O)(N[C@H](C)C(=O)OC1CCCC1)NC(C)(C)C(=O)OC1CCCCC1] |
| III-187 | 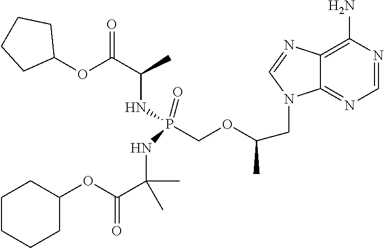 [SMILES: C[C@H](Cn1cnc2c(N)ncnc21)OC[P@@](=O)(N[C@H](C)C(=O)OC1CCCC1)NC(C)(C)C(=O)OC1CCCCC1] |
| III-188 | 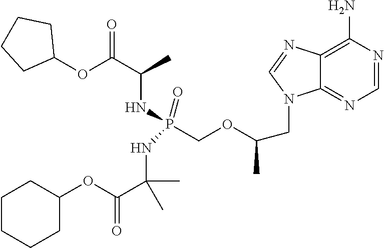 [SMILES: C[C@H](Cn1cnc2c(N)ncnc21)OC[P@](=O)(N[C@H](C)C(=O)OC1CCCC1)NC(C)(C)C(=O)OC1CCCCC1] |
| III-189 | 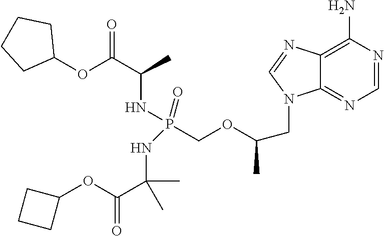 [SMILES: C[C@H](Cn1cnc2c(N)ncnc21)OCP(=O)(N[C@H](C)C(=O)OC1CCCC1)NC(C)(C)C(=O)OC1CCC1] |
| III-190 | 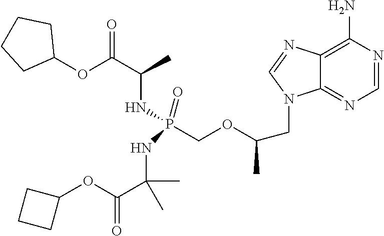 [SMILES: C[C@H](Cn1cnc2c(N)ncnc21)OC[P@@](=O)(N[C@H](C)C(=O)OC1CCCC1)NC(C)(C)C(=O)OC1CCC1] |
| III-191 | 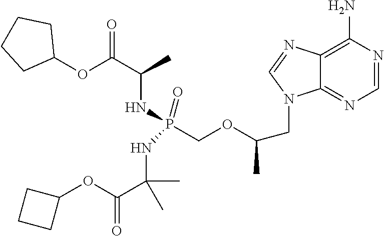 [SMILES: C[C@H](Cn1cnc2c(N)ncnc21)OC[P@](=O)(N[C@H](C)C(=O)OC1CCCC1)NC(C)(C)C(=O)OC1CCC1] |

In certain embodiments, the compound is a compound in Table 4 below, or a pharmaceutically acceptable salt thereof. In certain embodiments, the compound is a compound in Table 4 below. Compounds in Table 4 below can be prepared based on procedures described in, for example, WO 2018/080903 and WO 2018/080903; and related patents and applications, such as U.S. Pat. No. 10,736,908 and U.S. 2019/0321380; each of which is hereby incorporated by reference.

TABLE 4

| Compound No. | Chemical Structure |
|---|---|
| IV-1 | 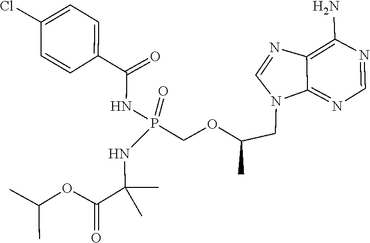 [SMILES: CC(C)OC(=O)C(C)(C)NP(=O)(CO[C@H](C)Cn1cnc2c(N)ncnc21)NC(=O)c1ccc(Cl)cc1] |
| IV-2 | 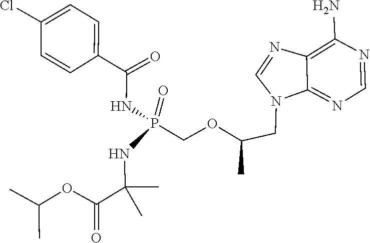 [SMILES: CC(C)OC(=O)C(C)(C)N[P@@](=O)(CO[C@H](C)Cn1cnc2c(N)ncnc21)NC(=O)c1ccc(Cl)cc1] |
| IV-3 | 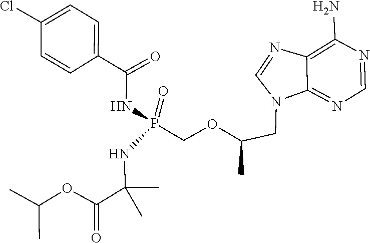 [SMILES: CC(C)OC(=O)C(C)(C)N[P@](=O)(CO[C@H](C)Cn1cnc2c(N)ncnc21)NC(=O)c1ccc(Cl)cc1] |
| IV-4 | 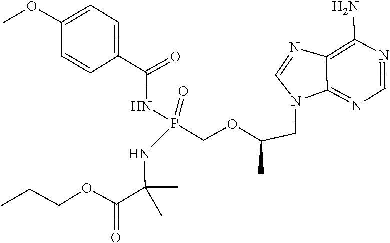 [SMILES: CCCOC(=O)C(C)(C)NP(=O)(CO[C@H](C)Cn1cnc2c(N)ncnc21)NC(=O)c1ccc(OC)cc1] |
| IV-5 | 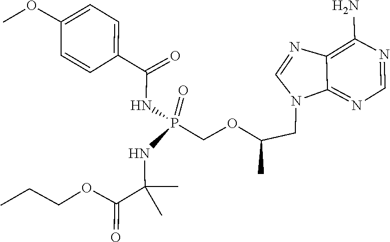 [SMILES: CCCOC(=O)C(C)(C)N[P@@](=O)(CO[C@H](C)Cn1cnc2c(N)ncnc21)NC(=O)c1ccc(OC)cc1] |
| IV-6 | 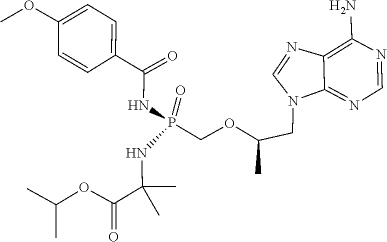 [SMILES: COc1ccc(C(=O)N[P@@](=O)(CO[C@H](C)Cn2cnc3c(N)ncnc32)NC(C)(C)C(=O)OC(C)C)cc1] |
| IV-7 | 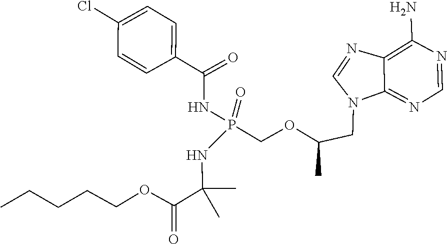 [SMILES: CCCCCOC(=O)C(C)(C)NP(=O)(CO[C@H](C)Cn1cnc2c(N)ncnc21)NC(=O)c1ccc(Cl)cc1] |
| IV-8 | 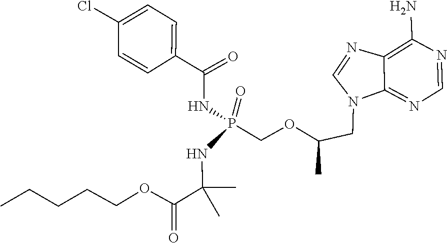 [SMILES: CCCCCOC(=O)C(C)(C)N[P@@](=O)(CO[C@H](C)Cn1cnc2c(N)ncnc21)NC(=O)c1ccc(Cl)cc1] |
| IV-9 | 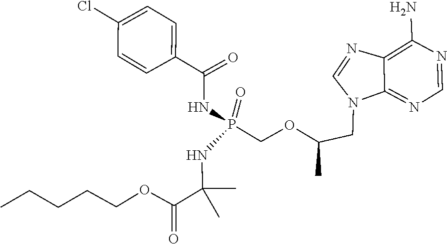 [SMILES: CCCCCOC(=O)C(C)(C)N[P@](=O)(CO[C@H](C)Cn1cnc2c(N)ncnc21)NC(=O)c1ccc(Cl)cc1] |
| IV-10 | 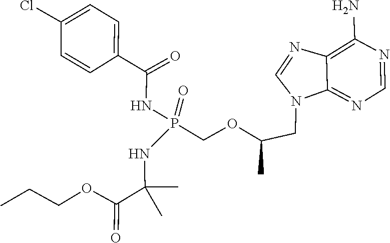 [SMILES: CCCOC(=O)C(C)(C)NP(=O)(CO[C@H](C)Cn1cnc2c(N)ncnc21)NC(=O)c1ccc(Cl)cc1] |
| IV-11 | 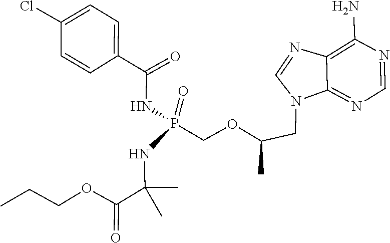 [SMILES: CCCOC(=O)C(C)(C)N[P@@](=O)(CO[C@H](C)Cn1cnc2c(N)ncnc21)NC(=O)c1ccc(Cl)cc1] |
| IV-12 | 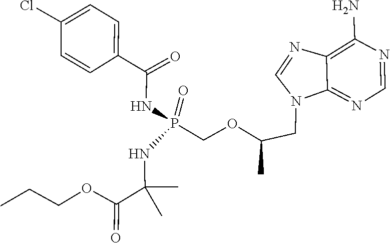 [SMILES: CCCOC(=O)C(C)(C)N[P@](=O)(CO[C@H](C)Cn1cnc2c(N)ncnc21)NC(=O)c1ccc(Cl)cc1] |
| IV-13 | 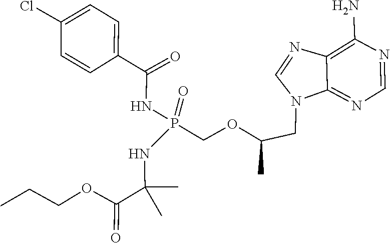 [SMILES: CCCOC(=O)C(C)(C)NP(=O)(CO[C@H](C)Cn1cnc2c(N)ncnc21)NC(=O)c1ccc(Cl)cc1] |
| IV-14 | 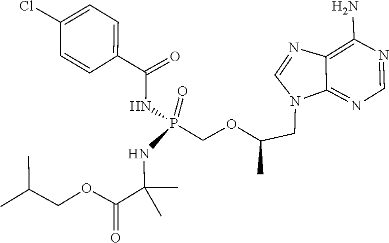 [SMILES: CC(C)COC(=O)C(C)(C)N[P@@](=O)(CO[C@H](C)Cn1cnc2c(N)ncnc21)NC(=O)c1ccc(Cl)cc1] |
| IV-15 | 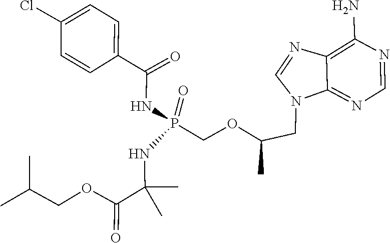 [SMILES: CC(C)COC(=O)C(C)(C)N[P@](=O)(CO[C@H](C)Cn1cnc2c(N)ncnc21)NC(=O)c1ccc(Cl)cc1] |
| IV-16 | 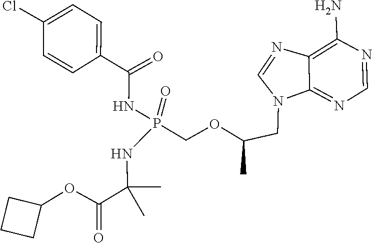 [SMILES: C[C@H](Cn1cnc2c(N)ncnc21)OCP(=O)(NC(=O)c1ccc(Cl)cc1)NC(C)(C)C(=O)OC1CCC1] |
| IV-17 | 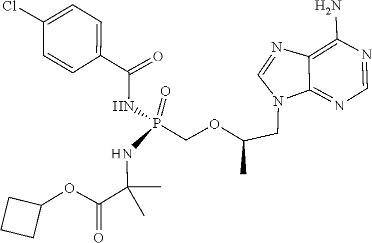 [SMILES: C[C@H](Cn1cnc2c(N)ncnc21)OC[P@@](=O)(NC(=O)c1ccc(Cl)cc1)NC(C)(C)C(=O)OC1CCC1] |
| IV-18 | 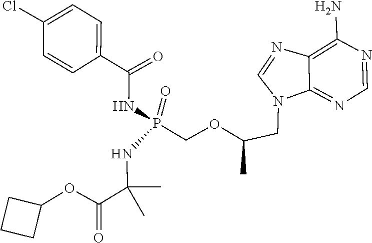 [SMILES: C[C@H](Cn1cnc2c(N)ncnc21)OC[P@](=O)(NC(=O)c1ccc(Cl)cc1)NC(C)(C)C(=O)OC1CCC1] |
| IV-19 | 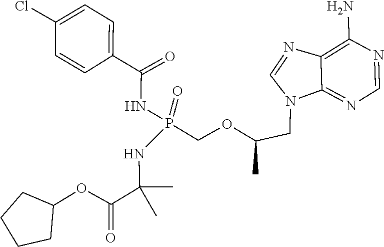 [SMILES: C[C@H](Cn1cnc2c(N)ncnc21)OCP(=O)(NC(=O)c1ccc(Cl)cc1)NC(C)(C)C(=O)OC1CCCC1] |
| IV-20 | 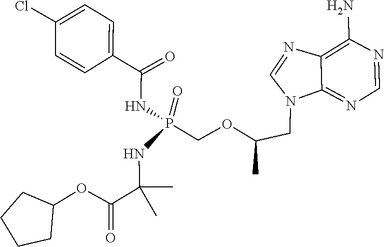 [SMILES: C[C@H](Cn1cnc2c(N)ncnc21)OC[P@@](=O)(NC(=O)c1ccc(Cl)cc1)NC(C)(C)C(=O)OC1CCCC1] |
| IV-21 | 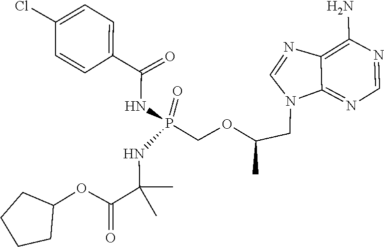 [SMILES: C[C@H](Cn1cnc2c(N)ncnc21)OC[P@](=O)(NC(=O)c1ccc(Cl)cc1)NC(C)(C)C(=O)OC1CCCC1] |
| IV-22 | 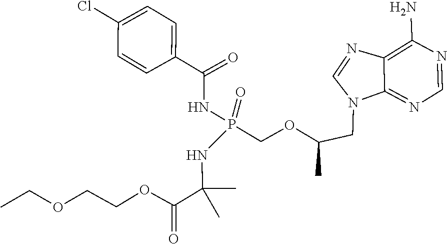 [SMILES: CCOCCOC(=O)C(C)(C)NP(=O)(CO[C@H](C)Cn1cnc2c(N)ncnc21)NC(=O)c1ccc(Cl)cc1] |
| IV-23 | 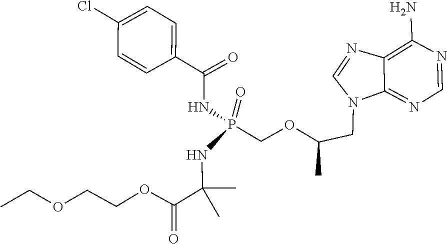 [SMILES: CCOCCOC(=O)C(C)(C)N[P@@](=O)(CO[C@H](C)Cn1cnc2c(N)ncnc21)NC(=O)c1ccc(Cl)cc1] |
| IV-24 | 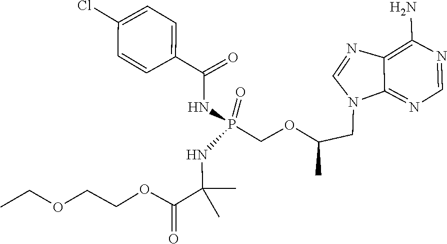 [SMILES: CCOCCOC(=O)C(C)(C)N[P@](=O)(CO[C@H](C)Cn1cnc2c(N)ncnc21)NC(=O)c1ccc(Cl)cc1] |
| IV-25 | 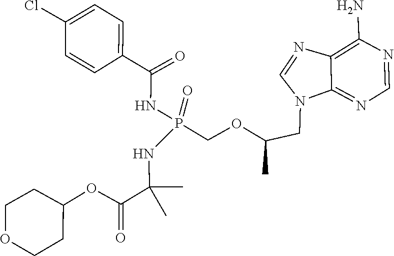 [SMILES: C[C@H](Cn1cnc2c(N)ncnc21)OCP(=O)(NC(=O)c1ccc(Cl)cc1)NC(C)(C)C(=O)OC1CCOCC1] |
| IV-26 | 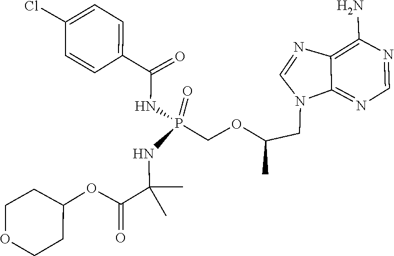 [SMILES: C[C@H](Cn1cnc2c(N)ncnc21)OC[P@@](=O)(NC(=O)c1ccc(Cl)cc1)NC(C)(C)C(=O)OC1CCOCC1] |
| IV-27 | 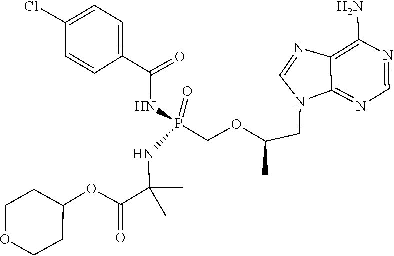 [SMILES: C[C@H](Cn1cnc2c(N)ncnc21)OC[P@](=O)(NC(=O)c1ccc(Cl)cc1)NC(C)(C)C(=O)OC1CCOCC1] |
| IV-28 | 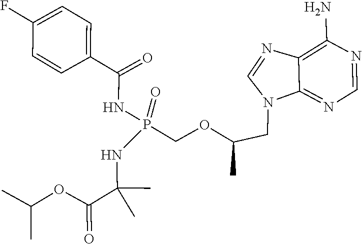 [SMILES: CC(C)OC(=O)C(C)(C)NP(=O)(CO[C@H](C)Cn1cnc2c(N)ncnc21)NC(=O)c1ccc(F)cc1] |
| IV-29 | 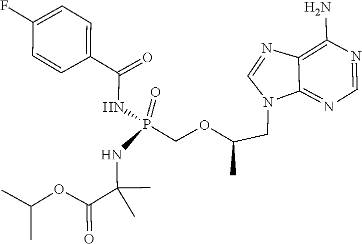 [SMILES: CC(C)OC(=O)C(C)(C)N[P@@](=O)(CO[C@H](C)Cn1cnc2c(N)ncnc21)NC(=O)c1ccc(F)cc1] |
| IV-30 | 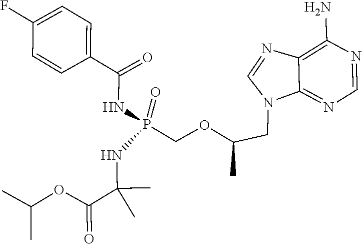 [SMILES: CC(C)OC(=O)C(C)(C)N[P@](=O)(CO[C@H](C)Cn1cnc2c(N)ncnc21)NC(=O)c1ccc(F)cc1] |
| IV-31 | 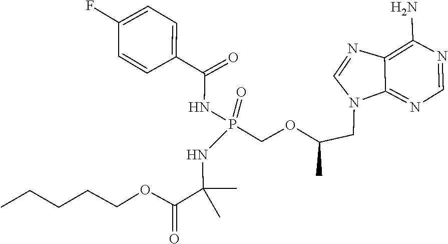 [SMILES: CCCCCOC(=O)C(C)(C)NP(=O)(CO[C@H](C)Cn1cnc2c(N)ncnc21)NC(=O)c1ccc(F)cc1] |
| IV-32 | 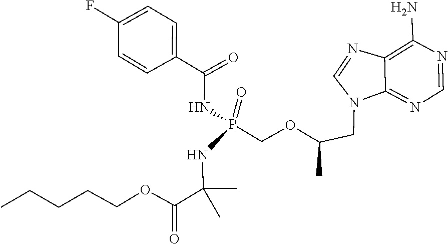 [SMILES: CCCCCOC(=O)C(C)(C)N[P@@](=O)(CO[C@H](C)Cn1cnc2c(N)ncnc21)NC(=O)c1ccc(F)cc1] |
| IV-33 | 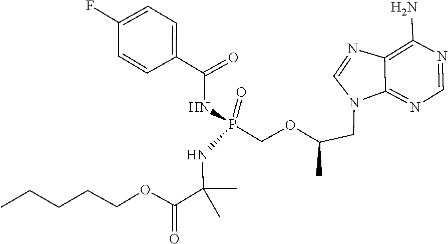 [SMILES: CCCCCOC(=O)C(C)(C)N[P@](=O)(CO[C@H](C)Cn1cnc2c(N)ncnc21)NC(=O)c1ccc(F)cc1] |
| IV-34 | 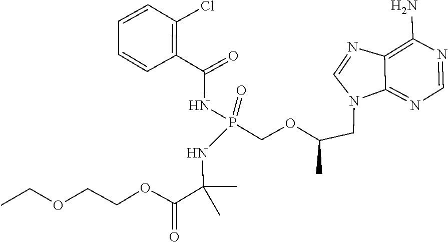 [SMILES: CCOCCOC(=O)C(C)(C)NP(=O)(CO[C@H](C)Cn1cnc2c(N)ncnc21)NC(=O)c1ccccc1Cl] |
| IV-35 | 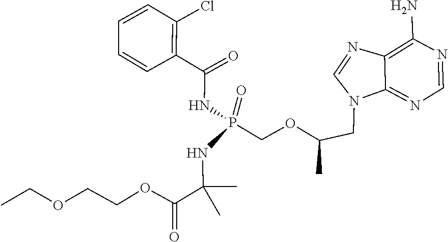 [SMILES: CCOCCOC(=O)C(C)(C)N[P@@](=O)(CO[C@H](C)Cn1cnc2c(N)ncnc21)NC(=O)c1ccccc1Cl] |
| IV-36 | 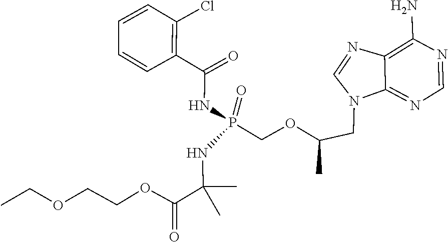 [SMILES: CCOCCOC(=O)C(C)(C)N[P@](=O)(CO[C@H](C)Cn1cnc2c(N)ncnc21)NC(=O)c1ccccc1Cl] |
| IV-37 | 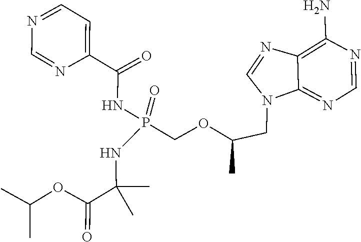 [SMILES: CC(C)OC(=O)C(C)(C)NP(=O)(CO[C@H](C)Cn1cnc2c(N)ncnc21)NC(=O)c1ccncn1] |
| IV-38 | 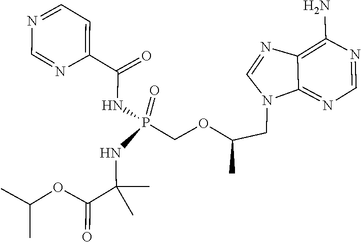 [SMILES: CC(C)OC(=O)C(C)(C)N[P@@](=O)(CO[C@H](C)Cn1cnc2c(N)ncnc21)NC(=O)c1ccncn1] |
| IV-39 | 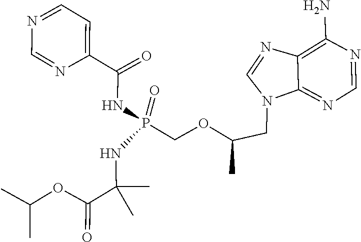 [SMILES: CC(C)OC(=O)C(C)(C)N[P@](=O)(CO[C@H](C)Cn1cnc2c(N)ncnc21)NC(=O)c1ccncn1] |
| IV-40 | 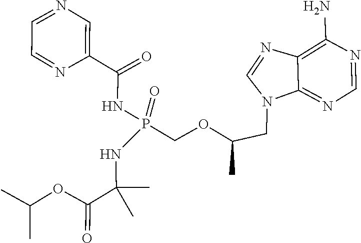 [SMILES: CC(C)OC(=O)C(C)(C)NP(=O)(CO[C@H](C)Cn1cnc2c(N)ncnc21)NC(=O)c1cnccn1] |
| IV-41 | 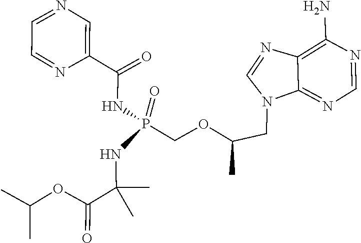 [SMILES: CC(C)OC(=O)C(C)(C)N[P@@](=O)(CO[C@H](C)Cn1cnc2c(N)ncnc21)NC(=O)c1cnccn1] |
| IV-42 | 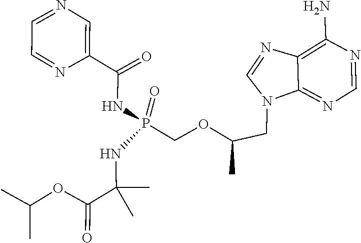 [SMILES: CC(C)OC(=O)C(C)(C)N[P@](=O)(CO[C@H](C)Cn1cnc2c(N)ncnc21)NC(=O)c1cnccn1] |
| IV-43 | 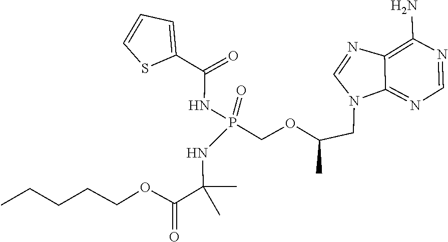 [SMILES: CCCCCOC(=O)C(C)(C)NP(=O)(CO[C@H](C)Cn1cnc2c(N)ncnc21)NC(=O)c1cccs1] |
| IV-44 | 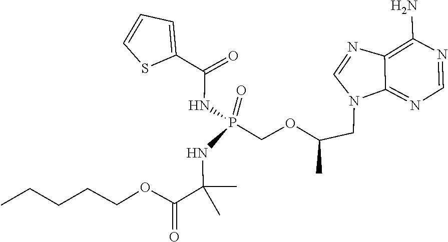 [SMILES: CCCCCOC(=O)C(C)(C)N[P@@](=O)(CO[C@H](C)Cn1cnc2c(N)ncnc21)NC(=O)c1cccs1] |
| IV-45 | 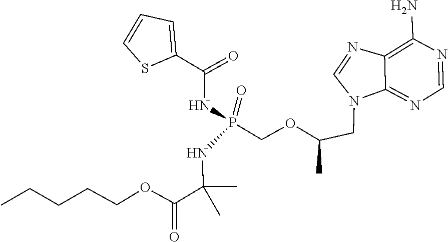 [SMILES: CCCCCOC(=O)C(C)(C)N[P@](=O)(CO[C@H](C)Cn1cnc2c(N)ncnc21)NC(=O)c1cccs1] |
| IV-46 | 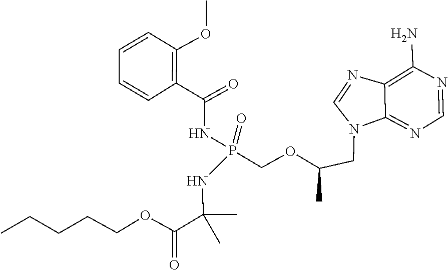 [SMILES: CCCCCOC(=O)C(C)(C)NP(=O)(CO[C@H](C)Cn1cnc2c(N)ncnc21)NC(=O)c1ccccc1OC] |
| IV-47 | 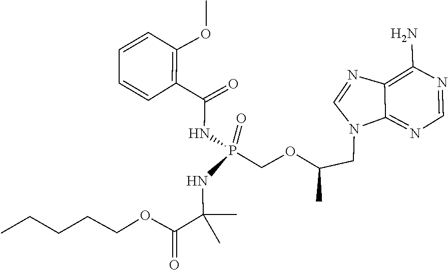 [SMILES: CCCCCOC(=O)C(C)(C)N[P@@](=O)(CO[C@H](C)Cn1cnc2c(N)ncnc21)NC(=O)c1ccccc1OC] |
| IV-48 | 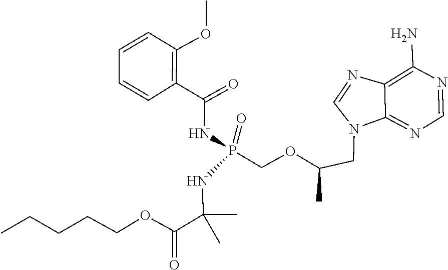 [SMILES: CCCCCOC(=O)C(C)(C)N[P@](=O)(CO[C@H](C)Cn1cnc2c(N)ncnc21)NC(=O)c1ccccc1OC] |
| IV-49 | 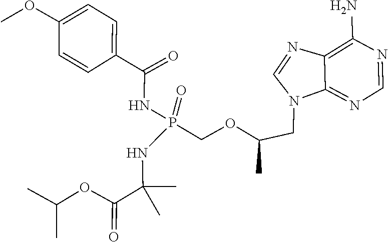 [SMILES: COc1ccc(C(=O)NP(=O)(CO[C@H](C)Cn2cnc3c(N)ncnc32)NC(C)(C)C(=O)OC(C)C)cc1] |
| IV-50 | 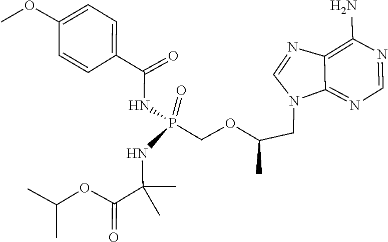 [SMILES: COc1ccc(C(=O)N[P@](=O)(CO[C@H](C)Cn2cnc3c(N)ncnc32)NC(C)(C)C(=O)OC(C)C)cc1] |
| IV-51 | 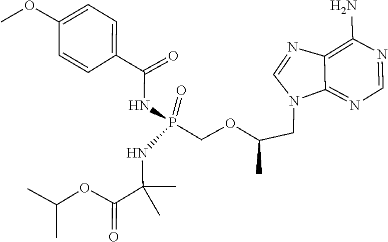 [SMILES: COc1ccc(C(=O)N[P@@](=O)(CO[C@H](C)Cn2cnc3c(N)ncnc32)NC(C)(C)C(=O)OC(C)C)cc1] |
| IV-52 | 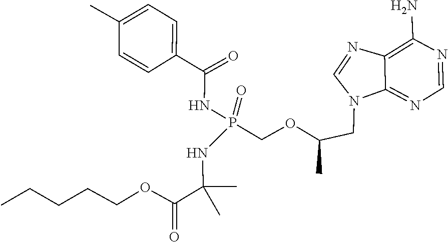 [SMILES: CCCCCOC(=O)C(C)(C)NP(=O)(CO[C@H](C)Cn1cnc2c(N)ncnc21)NC(=O)c1ccc(C)cc1] |
| IV-53 | 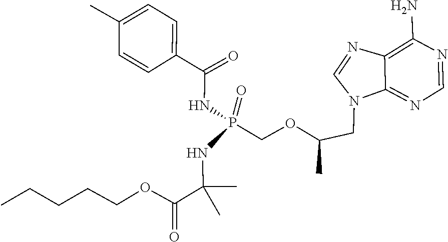 [SMILES: CCCCCOC(=O)C(C)(C)N[P@@](=O)(CO[C@H](C)Cn1cnc2c(N)ncnc21)NC(=O)c1ccc(C)cc1] |
| IV-54 | 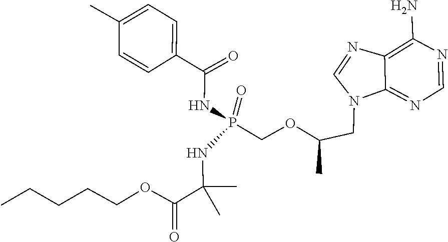 [SMILES: CCCCCOC(=O)C(C)(C)N[P@](=O)(CO[C@H](C)Cn1cnc2c(N)ncnc21)NC(=O)c1ccc(C)cc1] |
| IV-55 | 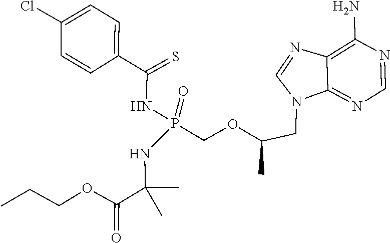 [SMILES: CCCOC(=O)C(C)(C)NP(=O)(CO[C@H](C)Cn1cnc2c(N)ncnc21)NC(=S)c1ccc(Cl)cc1] |
| IV-56 | 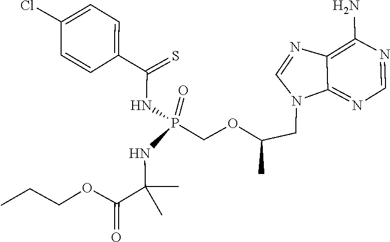 [SMILES: CCCOC(=O)C(C)(C)N[P@@](=O)(CO[C@H](C)Cn1cnc2c(N)ncnc21)NC(=S)c1ccc(Cl)cc1] |
| IV-57 | 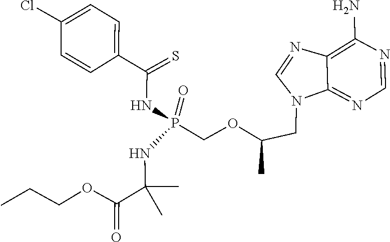 [SMILES: CCCOC(=O)C(C)(C)N[P@](=O)(CO[C@H](C)Cn1cnc2c(N)ncnc21)NC(=S)c1ccc(Cl)cc1] |
| IV-58 | 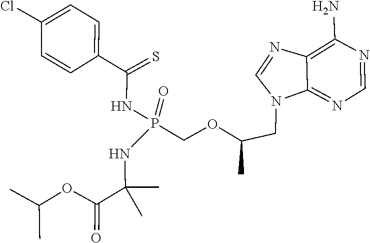 [SMILES: CC(C)OC(=O)C(C)(C)NP(=O)(CO[C@H](C)Cn1cnc2c(N)ncnc21)NC(=S)c1ccc(Cl)cc1] |
| IV-59 | 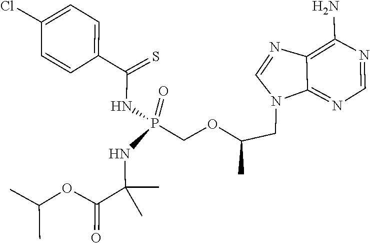 [SMILES: CC(C)OC(=O)C(C)(C)N[P@@](=O)(CO[C@H](C)Cn1cnc2c(N)ncnc21)NC(=S)c1ccc(Cl)cc1] |
| IV-60 | 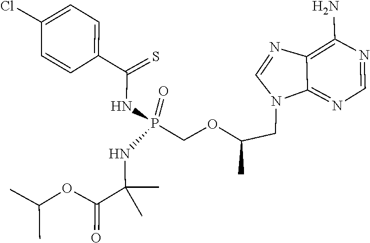 [SMILES: CC(C)OC(=O)C(C)(C)N[P@](=O)(CO[C@H](C)Cn1cnc2c(N)ncnc21)NC(=S)c1ccc(Cl)cc1] |
| IV-61 | 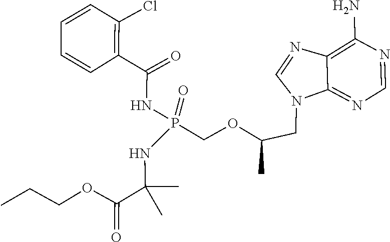 [SMILES: CCCOC(=O)C(C)(C)NP(=O)(CO[C@H](C)Cn1cnc2c(N)ncnc21)NC(=O)c1ccccc1Cl] |
| IV-62 | 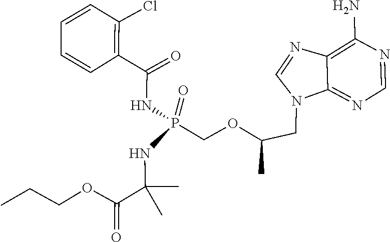 [SMILES: CCCOC(=O)C(C)(C)N[P@@](=O)(CO[C@H](C)Cn1cnc2c(N)ncnc21)NC(=O)c1ccccc1Cl] |
| IV-63 | 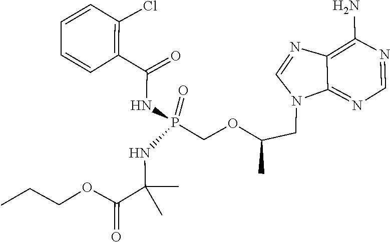 [SMILES: CCCOC(=O)C(C)(C)N[P@](=O)(CO[C@H](C)Cn1cnc2c(N)ncnc21)NC(=O)c1ccccc1Cl] |
| IV-64 | 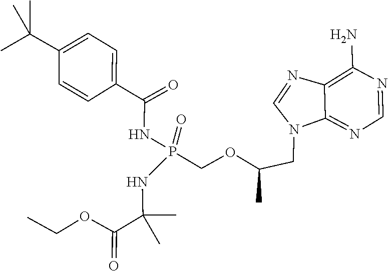 [SMILES: CCOC(=O)C(C)(C)NP(=O)(CO[C@H](C)Cn1cnc2c(N)ncnc21)NC(=O)c1ccc(C(C)(C)C)cc1] |
| IV-65 | 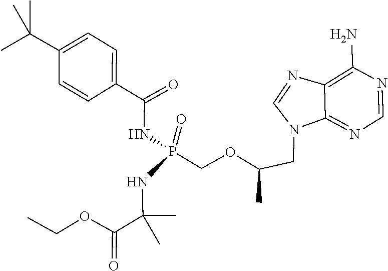 [SMILES: CCOC(=O)C(C)(C)N[P@@](=O)(CO[C@H](C)Cn1cnc2c(N)ncnc21)NC(=O)c1ccc(C(C)(C)C)cc1] |
| IV-66 | 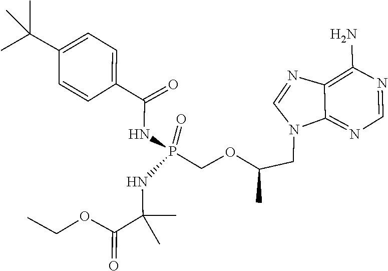 [SMILES: CCOC(=O)C(C)(C)N[P@](=O)(CO[C@H](C)Cn1cnc2c(N)ncnc21)NC(=O)c1ccc(C(C)(C)C)cc1] |
| IV-67 | 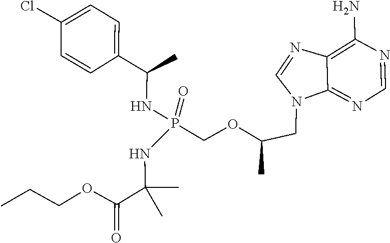 [SMILES: CCCOC(=O)C(C)(C)NP(=O)(CO[C@H](C)Cn1cnc2c(N)ncnc21)N[C@H](C)c1ccc(Cl)cc1] |
| IV-68 | 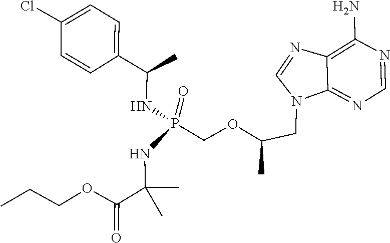 [SMILES: CCCOC(=O)C(C)(C)N[P@@](=O)(CO[C@H](C)Cn1cnc2c(N)ncnc21)N[C@H](C)c1ccc(Cl)cc1] |
| IV-69 | 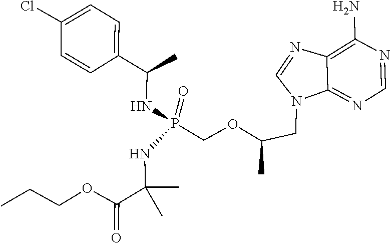 [SMILES: CCCOC(=O)C(C)(C)N[P@](=O)(CO[C@H](C)Cn1cnc2c(N)ncnc21)N[C@H](C)c1ccc(Cl)cc1] |
| IV-70 | 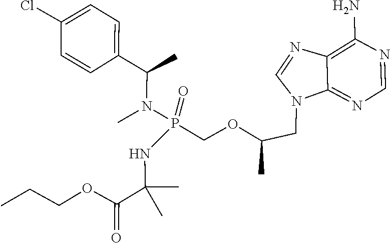 [SMILES: CCCOC(=O)C(C)(C)NP(=O)(CO[C@H](C)Cn1cnc2c(N)ncnc21)N(C)[C@H](C)c1ccc(Cl)cc1] |
| IV-71 | 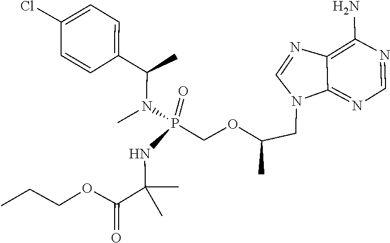 [SMILES: CCCOC(=O)C(C)(C)N[P@@](=O)(CO[C@H](C)Cn1cnc2c(N)ncnc21)N(C)[C@H](C)c1ccc(Cl)cc1] |
| IV-72 | 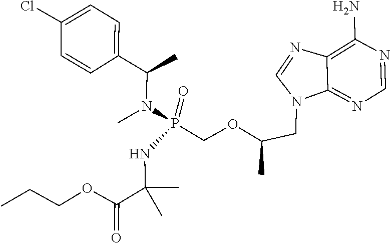 [SMILES: CCCOC(=O)C(C)(C)N[P@](=O)(CO[C@H](C)Cn1cnc2c(N)ncnc21)N(C)[C@H](C)c1ccc(Cl)cc1] |
| IV-73 | 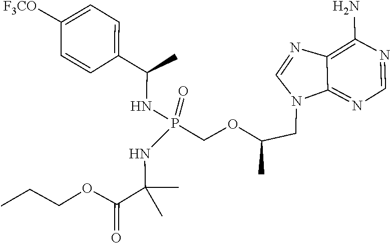 [SMILES: CCCOC(=O)C(C)(C)NP(=O)(CO[C@H](C)Cn1cnc2c(N)ncnc21)N[C@H](C)c1ccc(OC(F)(F)F)cc1] |
| IV-74 | 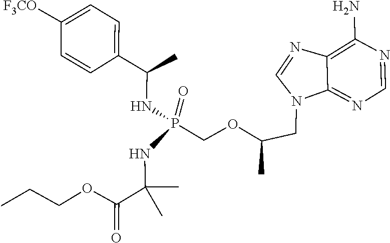 [SMILES: CCCOC(=O)C(C)(C)N[P@@](=O)(CO[C@H](C)Cn1cnc2c(N)ncnc21)N[C@H](C)c1ccc(OC(F)(F)F)cc1] |
| IV-75 | 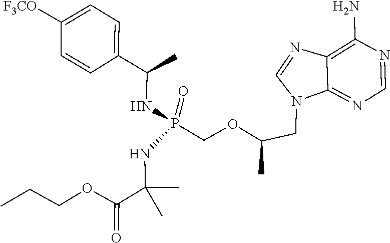 [SMILES: CCCOC(=O)C(C)(C)N[P@](=O)(CO[C@H](C)Cn1cnc2c(N)ncnc21)N[C@H](C)c1ccc(OC(F)(F)F)cc1] |
| IV-76 | 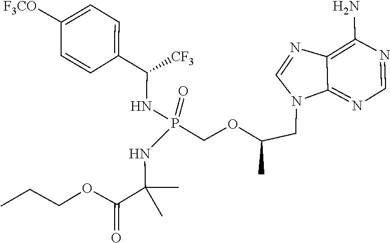 [SMILES: CCCOC(=O)C(C)(C)NP(=O)(CO[C@H](C)Cn1cnc2c(N)ncnc21)N[C@H](c1ccc(OC(F)(F)F)cc1)C(F)(F)F] |
| IV-77 | 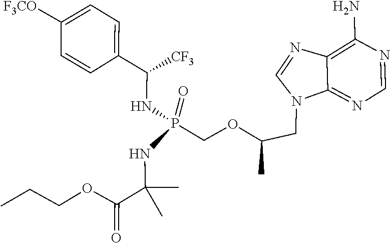 [SMILES: CCCOC(=O)C(C)(C)N[P@@](=O)(CO[C@H](C)Cn1cnc2c(N)ncnc21)N[C@H](c1ccc(OC(F)(F)F)cc1)C(F)(F)F] |
| IV-78 | 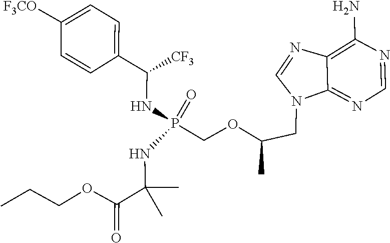 [SMILES: CCCOC(=O)C(C)(C)N[P@](=O)(CO[C@H](C)Cn1cnc2c(N)ncnc21)N[C@H](c1ccc(OC(F)(F)F)cc1)C(F)(F)F] |
| IV-79 | 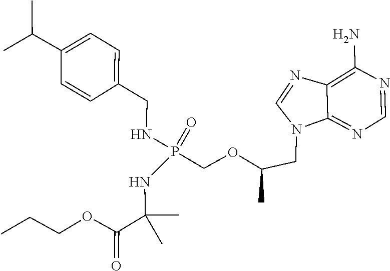 [SMILES: CCCOC(=O)C(C)(C)NP(=O)(CO[C@H](C)Cn1cnc2c(N)ncnc21)NCc1ccc(C(C)C)cc1] |
| IV-80 | 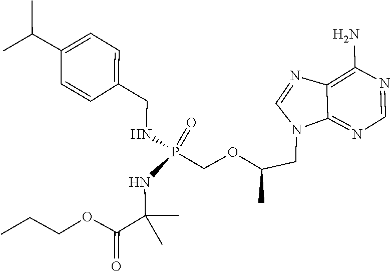 [SMILES: CCCOC(=O)C(C)(C)N[P@@](=O)(CO[C@H](C)Cn1cnc2c(N)ncnc21)NCc1ccc(C(C)C)cc1] |
| IV-81 | 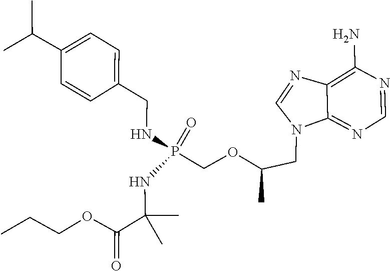 [SMILES: CCCOC(=O)C(C)(C)N[P@](=O)(CO[C@H](C)Cn1cnc2c(N)ncnc21)NCc1ccc(C(C)C)cc1] |
| IV-82 | 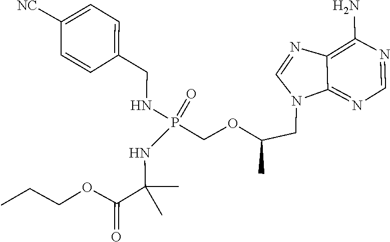 [SMILES: CCCOC(=O)C(C)(C)NP(=O)(CO[C@H](C)Cn1cnc2c(N)ncnc21)NCc1ccc(C#N)cc1] |
| IV-83 | 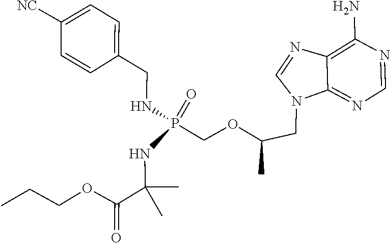 [SMILES: CCCOC(=O)C(C)(C)N[P@@](=O)(CO[C@H](C)Cn1cnc2c(N)ncnc21)NCc1ccc(C#N)cc1] |
| IV-84 | 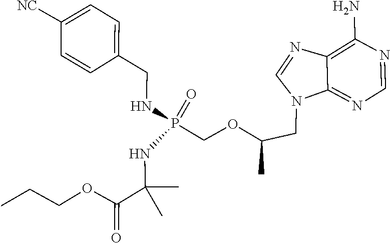 [SMILES: CCCOC(=O)C(C)(C)N[P@](=O)(CO[C@H](C)Cn1cnc2c(N)ncnc21)NCc1ccc(C#N)cc1] |
| IV-85 | 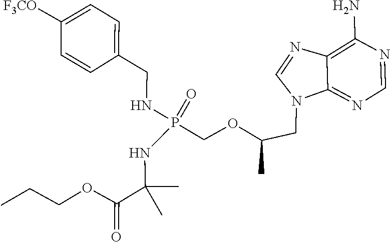 [SMILES: CCCOC(=O)C(C)(C)NP(=O)(CO[C@H](C)Cn1cnc2c(N)ncnc21)NCc1ccc(OC(F)(F)F)cc1] |
| IV-86 | 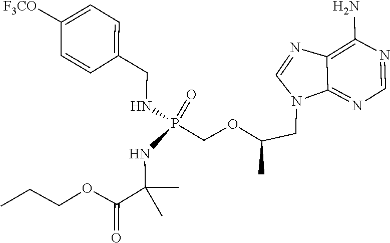 [SMILES: CCCOC(=O)C(C)(C)N[P@@](=O)(CO[C@H](C)Cn1cnc2c(N)ncnc21)NCc1ccc(OC(F)(F)F)cc1] |
| IV-87 | 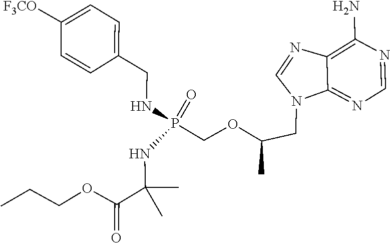 [SMILES: CCCOC(=O)C(C)(C)N[P@](=O)(CO[C@H](C)Cn1cnc2c(N)ncnc21)NCc1ccc(OC(F)(F)F)cc1] |
| IV-88 | 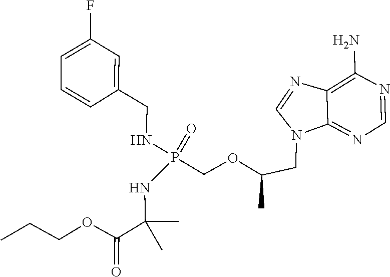 [SMILES: CCCOC(=O)C(C)(C)NP(=O)(CO[C@H](C)Cn1cnc2c(N)ncnc21)NCc1cccc(F)c1] |
| IV-89 | 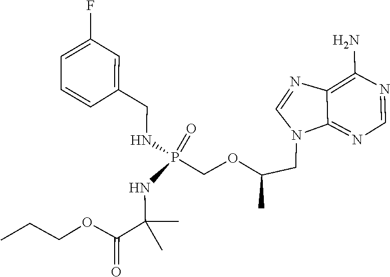 [SMILES: CCCOC(=O)C(C)(C)N[P@@](=O)(CO[C@H](C)Cn1cnc2c(N)ncnc21)NCc1cccc(F)c1] |
| IV-90 | 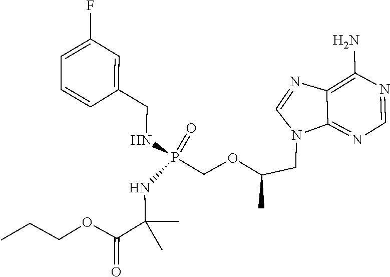 [SMILES: CCCOC(=O)C(C)(C)N[P@](=O)(CO[C@H](C)Cn1cnc2c(N)ncnc21)NCc1cccc(F)c1] |
| IV-91 | 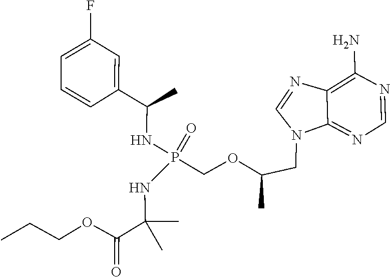 [SMILES: CCCOC(=O)C(C)(C)NP(=O)(CO[C@H](C)Cn1cnc2c(N)ncnc21)N[C@H](C)c1cccc(F)c1] |
| IV-92 | 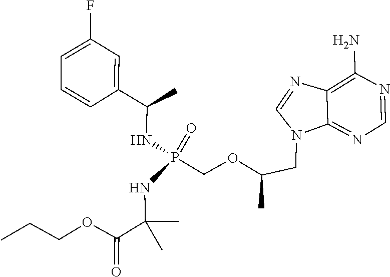 [SMILES: CCCOC(=O)C(C)(C)N[P@@](=O)(CO[C@H](C)Cn1cnc2c(N)ncnc21)N[C@H](C)c1cccc(F)c1] |
| IV-93 | 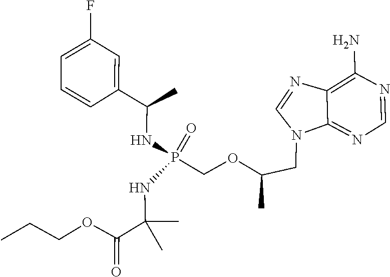 [SMILES: CCCOC(=O)C(C)(C)N[P@](=O)(CO[C@H](C)Cn1cnc2c(N)ncnc21)N[C@H](C)c1cccc(F)c1] |
| IV-94 | 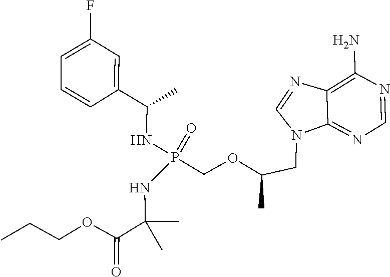 [SMILES: CCCOC(=O)C(C)(C)NP(=O)(CO[C@H](C)Cn1cnc2c(N)ncnc21)N[C@@H](C)c1cccc(F)c1] |
| IV-95 | 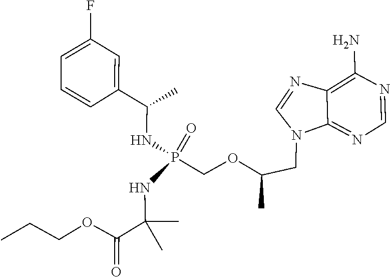 [SMILES: CCCOC(=O)C(C)(C)N[P@@](=O)(CO[C@H](C)Cn1cnc2c(N)ncnc21)N[C@@H](C)c1cccc(F)c1] |
| IV-96 | 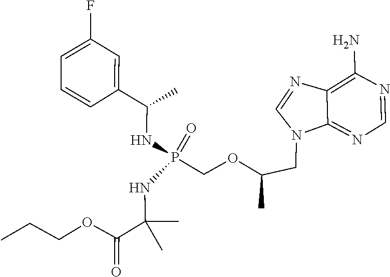 [SMILES: CCCOC(=O)C(C)(C)N[P@](=O)(CO[C@H](C)Cn1cnc2c(N)ncnc21)N[C@@H](C)c1cccc(F)c1] |
| IV-97 | 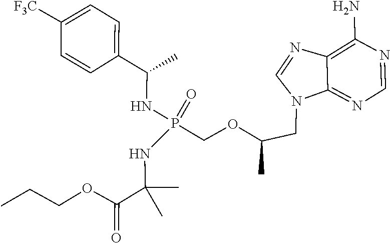 [SMILES: CCCOC(=O)C(C)(C)NP(=O)(CO[C@H](C)Cn1cnc2c(N)ncnc21)N[C@@H](C)c1ccc(C(F)(F)F)cc1] |
| IV-98 | 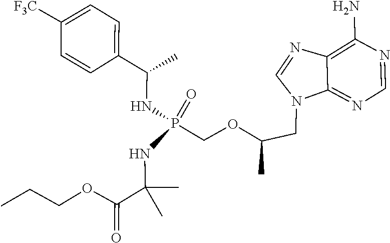 [SMILES: CCCOC(=O)C(C)(C)N[P@@](=O)(CO[C@H](C)Cn1cnc2c(N)ncnc21)N[C@@H](C)c1ccc(C(F)(F)F)cc1] |
| IV-99 | 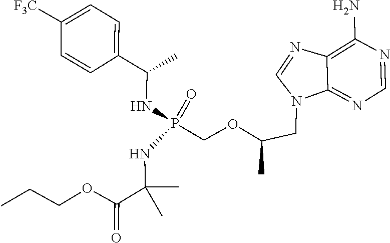 [SMILES: CCCOC(=O)C(C)(C)N[P@](=O)(CO[C@H](C)Cn1cnc2c(N)ncnc21)N[C@@H](C)c1ccc(C(F)(F)F)cc1] |
| IV-100 | 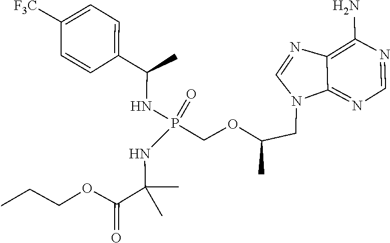 [SMILES: CCCOC(=O)C(C)(C)NP(=O)(CO[C@H](C)Cn1cnc2c(N)ncnc21)N[C@H](C)c1ccc(C(F)(F)F)cc1] |
| IV-101 | 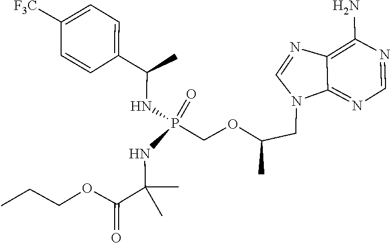 [SMILES: CCCOC(=O)C(C)(C)N[P@@](=O)(CO[C@H](C)Cn1cnc2c(N)ncnc21)N[C@H](C)c1ccc(C(F)(F)F)cc1] |
| IV-102 | 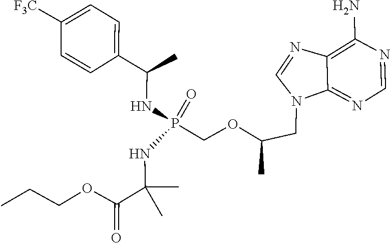 [SMILES: CCCOC(=O)C(C)(C)N[P@](=O)(CO[C@H](C)Cn1cnc2c(N)ncnc21)N[C@H](C)c1ccc(C(F)(F)F)cc1] |
| IV-103 | 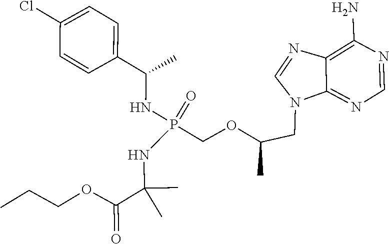 [SMILES: CCCOC(=O)C(C)(C)NP(=O)(CO[C@H](C)Cn1cnc2c(N)ncnc21)N[C@@H](C)c1ccc(Cl)cc1] |
| IV-104 | 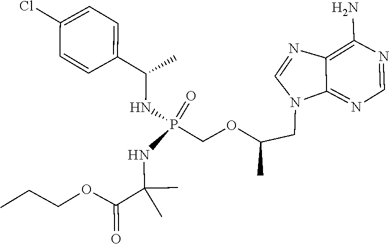 [SMILES: CCCOC(=O)C(C)(C)N[P@@](=O)(CO[C@H](C)Cn1cnc2c(N)ncnc21)N[C@@H](C)c1ccc(Cl)cc1] |
| IV-105 | 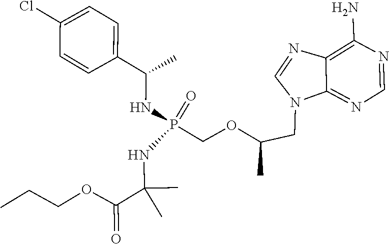 [SMILES: CCCOC(=O)C(C)(C)N[P@](=O)(CO[C@H](C)Cn1cnc2c(N)ncnc21)N[C@@H](C)c1ccc(Cl)cc1] |
| IV-106 | 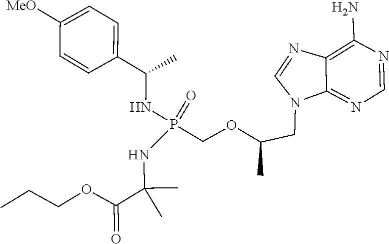 [SMILES: CCCOC(=O)C(C)(C)NP(=O)(CO[C@H](C)Cn1cnc2c(N)ncnc21)N[C@@H](C)c1ccc(OC)cc1] |
| IV-107 | 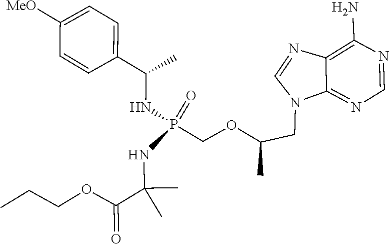 [SMILES: CCCOC(=O)C(C)(C)N[P@@](=O)(CO[C@H](C)Cn1cnc2c(N)ncnc21)N[C@@H](C)c1ccc(OC)cc1] |
| IV-108 | 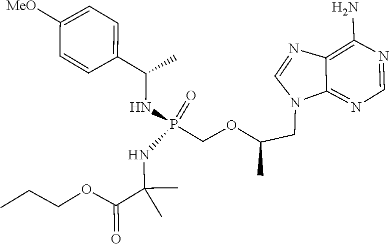 [SMILES: CCCOC(=O)C(C)(C)N[P@](=O)(CO[C@H](C)Cn1cnc2c(N)ncnc21)N[C@@H](C)c1ccc(OC)cc1] |
| IV-109 | 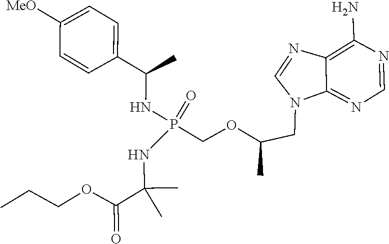 [SMILES: CCCOC(=O)C(C)(C)NP(=O)(CO[C@H](C)Cn1cnc2c(N)ncnc21)N[C@H](C)c1ccc(OC)cc1] |
| IV-110 | 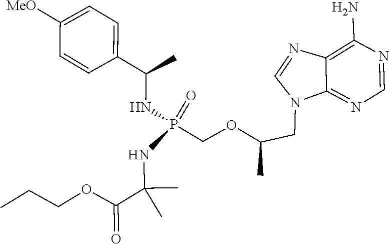 [SMILES: CCCOC(=O)C(C)(C)N[P@@](=O)(CO[C@H](C)Cn1cnc2c(N)ncnc21)N[C@H](C)c1ccc(OC)cc1] |
| IV-111 | 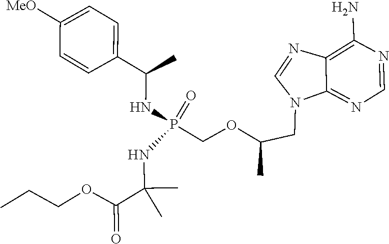 [SMILES: CCCOC(=O)C(C)(C)N[P@](=O)(CO[C@H](C)Cn1cnc2c(N)ncnc21)N[C@H](C)c1ccc(OC)cc1] |
| IV-112 | 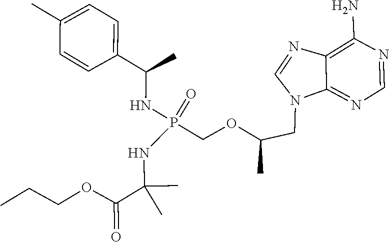 [SMILES: CCCOC(=O)C(C)(C)NP(=O)(CO[C@H](C)Cn1cnc2c(N)ncnc21)N[C@H](C)c1ccc(C)cc1] |
| IV-113 | 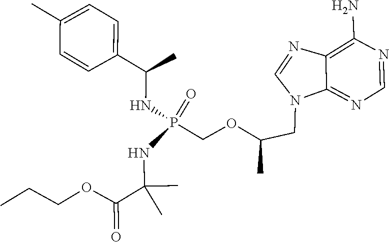 [SMILES: CCCOC(=O)C(C)(C)N[P@@](=O)(CO[C@H](C)Cn1cnc2c(N)ncnc21)N[C@H](C)c1ccc(C)cc1] |
| IV-114 | 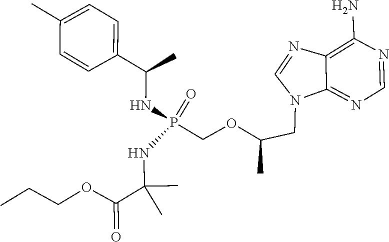 [SMILES: CCCOC(=O)C(C)(C)N[P@](=O)(CO[C@H](C)Cn1cnc2c(N)ncnc21)N[C@H](C)c1ccc(C)cc1] |
| IV-115 | 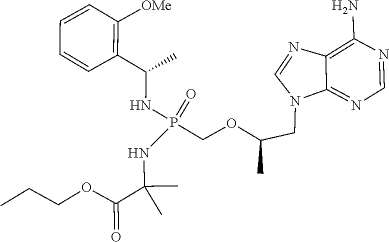 [SMILES: CCCOC(=O)C(C)(C)NP(=O)(CO[C@H](C)Cn1cnc2c(N)ncnc21)N[C@@H](C)c1ccccc1OC] |
| IV-116 | 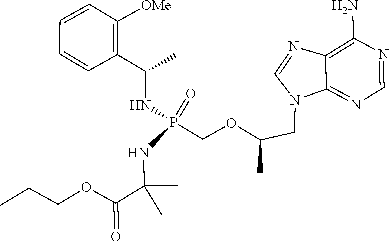 [SMILES: CCCOC(=O)C(C)(C)N[P@@](=O)(CO[C@H](C)Cn1cnc2c(N)ncnc21)N[C@@H](C)c1ccccc1OC] |
| IV-117 | 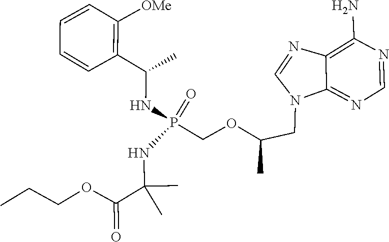 [SMILES: CCCOC(=O)C(C)(C)N[P@](=O)(CO[C@H](C)Cn1cnc2c(N)ncnc21)N[C@@H](C)c1ccccc1OC] |
| IV-118 | 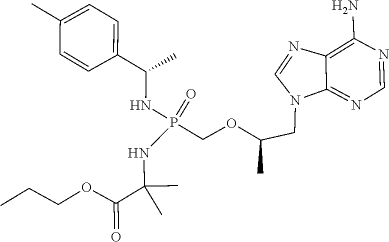 [SMILES: CCCOC(=O)C(C)(C)NP(=O)(CO[C@H](C)Cn1cnc2c(N)ncnc21)N[C@@H](C)c1ccc(C)cc1] |
| IV-119 | 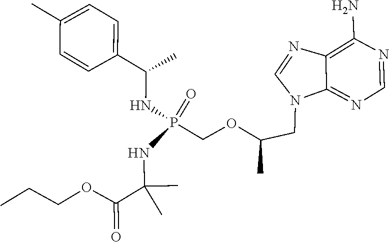 [SMILES: CCCOC(=O)C(C)(C)N[P@@](=O)(CO[C@H](C)Cn1cnc2c(N)ncnc21)N[C@@H](C)c1ccc(C)cc1] |
| IV-120 | 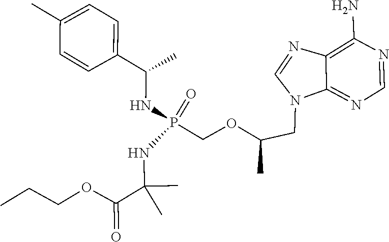 [SMILES: CCCOC(=O)C(C)(C)N[P@](=O)(CO[C@H](C)Cn1cnc2c(N)ncnc21)N[C@@H](C)c1ccc(C)cc1] |
| IV-121 | 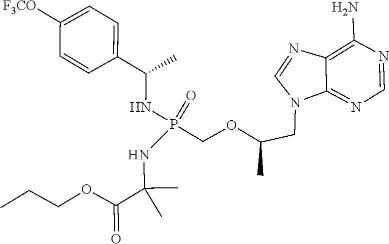 [SMILES: CCCOC(=O)C(C)(C)NP(=O)(CO[C@H](C)Cn1cnc2c(N)ncnc21)N[C@@H](C)c1ccc(OC(F)(F)F)cc1] |
| IV-122 | 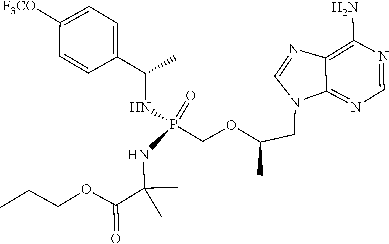 [SMILES: CCCOC(=O)C(C)(C)N[P@@](=O)(CO[C@H](C)Cn1cnc2c(N)ncnc21)N[C@@H](C)c1ccc(OC(F)(F)F)cc1] |
| IV-123 | 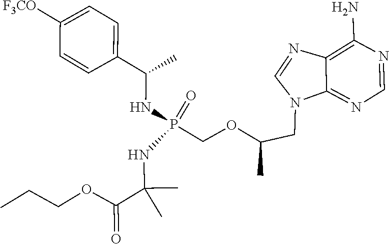 [SMILES: CCCOC(=O)C(C)(C)N[P@](=O)(CO[C@H](C)Cn1cnc2c(N)ncnc21)N[C@@H](C)c1ccc(OC(F)(F)F)cc1] |
| IV-124 | 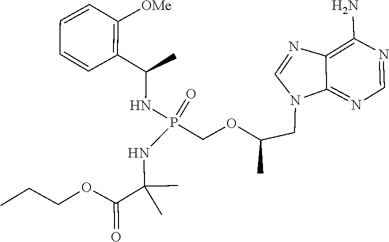 [SMILES: CCCOC(=O)C(C)(C)NP(=O)(CO[C@H](C)Cn1cnc2c(N)ncnc21)N[C@H](C)c1ccccc1OC] |
| IV-125 | 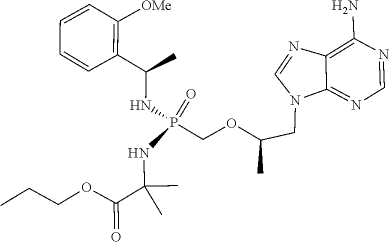 [SMILES: CCCOC(=O)C(C)(C)N[P@@](=O)(CO[C@H](C)Cn1cnc2c(N)ncnc21)N[C@H](C)c1ccccc1OC] |
| IV-126 | 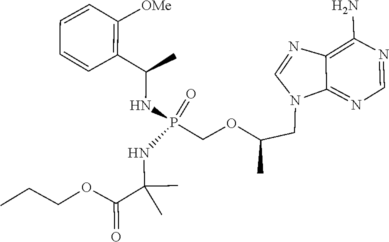 [SMILES: CCCOC(=O)C(C)(C)N[P@](=O)(CO[C@H](C)Cn1cnc2c(N)ncnc21)N[C@H](C)c1ccccc1OC] |
| IV-127 | 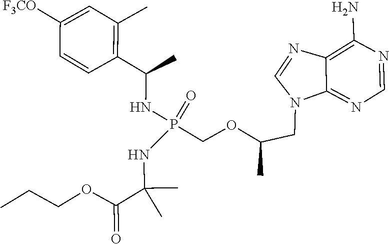 [SMILES: CCCOC(=O)C(C)(C)NP(=O)(CO[C@H](C)Cn1cnc2c(N)ncnc21)N[C@H](C)c1ccc(OC(F)(F)F)cc1C] |
| IV-128 | 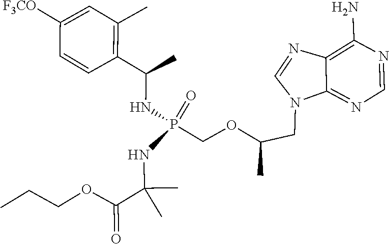 [SMILES: CCCOC(=O)C(C)(C)N[P@@](=O)(CO[C@H](C)Cn1cnc2c(N)ncnc21)N[C@H](C)c1ccc(OC(F)(F)F)cc1C] |
| IV-129 | 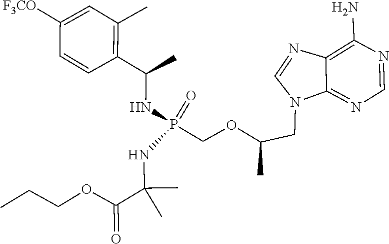 [SMILES: CCCOC(=O)C(C)(C)N[P@](=O)(CO[C@H](C)Cn1cnc2c(N)ncnc21)N[C@H](C)c1ccc(OC(F)(F)F)cc1C] |
| IV-130 | 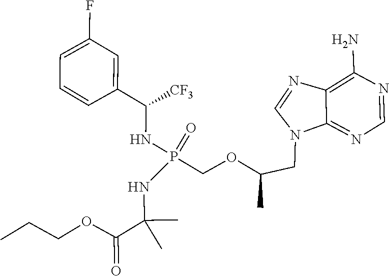 [SMILES: CCCOC(=O)C(C)(C)NP(=O)(CO[C@H](C)Cn1cnc2c(N)ncnc21)N[C@H](c1cccc(F)c1)C(F)(F)F] |
| IV-131 | 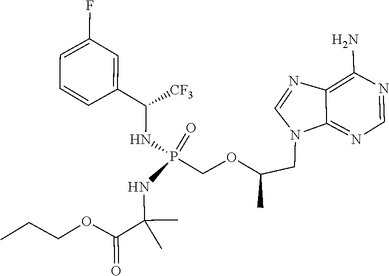 [SMILES: CCCOC(=O)C(C)(C)N[P@@](=O)(CO[C@H](C)Cn1cnc2c(N)ncnc21)N[C@H](c1cccc(F)c1)C(F)(F)F] |
| IV-132 | 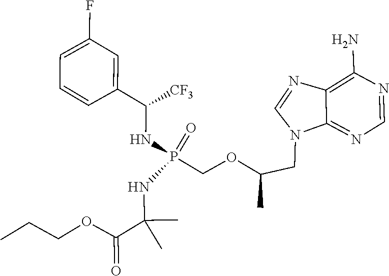 [SMILES: CCCOC(=O)C(C)(C)N[P@](=O)(CO[C@H](C)Cn1cnc2c(N)ncnc21)N[C@H](c1cccc(F)c1)C(F)(F)F] |
| IV-133 | 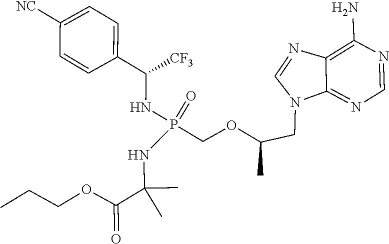 [SMILES: CCCOC(=O)C(C)(C)NP(=O)(CO[C@H](C)Cn1cnc2c(N)ncnc21)N[C@H](c1ccc(C#N)cc1)C(F)(F)F] |
| IV-134 | 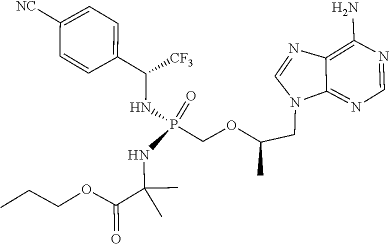 [SMILES: CCCOC(=O)C(C)(C)N[P@@](=O)(CO[C@H](C)Cn1cnc2c(N)ncnc21)N[C@H](c1ccc(C#N)cc1)C(F)(F)F] |
| IV-135 | 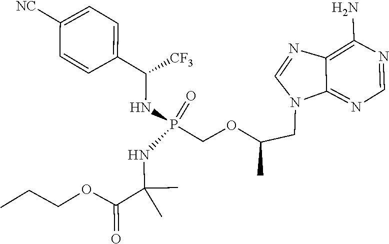 [SMILES: CCCOC(=O)C(C)(C)N[P@](=O)(CO[C@H](C)Cn1cnc2c(N)ncnc21)N[C@H](c1ccc(C#N)cc1)C(F)(F)F] |
| IV-136 | 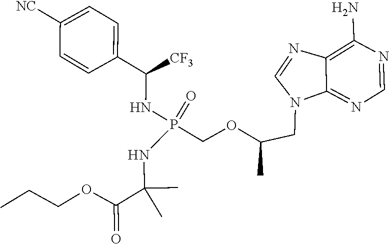 [SMILES: CCCOC(=O)C(C)(C)NP(=O)(CO[C@H](C)Cn1cnc2c(N)ncnc21)N[C@@H](c1ccc(C#N)cc1)C(F)(F)F] |
| IV-137 | 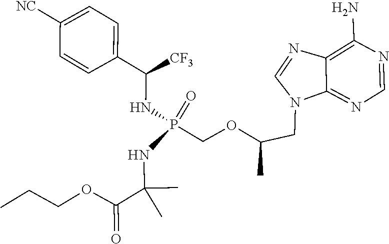 [SMILES: CCCOC(=O)C(C)(C)N[P@@](=O)(CO[C@H](C)Cn1cnc2c(N)ncnc21)N[C@@H](c1ccc(C#N)cc1)C(F)(F)F] |
| IV-138 | 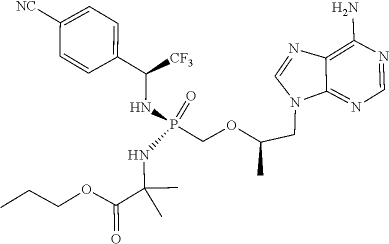 [SMILES: CCCOC(=O)C(C)(C)N[P@](=O)(CO[C@H](C)Cn1cnc2c(N)ncnc21)N[C@@H](c1ccc(C#N)cc1)C(F)(F)F] |
| IV-139 | 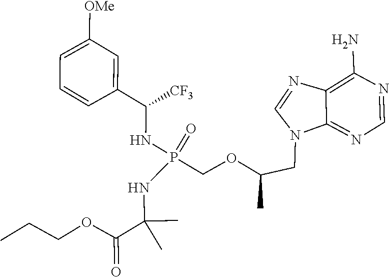 [SMILES: CCCOC(=O)C(C)(C)NP(=O)(CO[C@H](C)Cn1cnc2c(N)ncnc21)N[C@H](c1cccc(OC)c1)C(F)(F)F] |
| IV-140 | 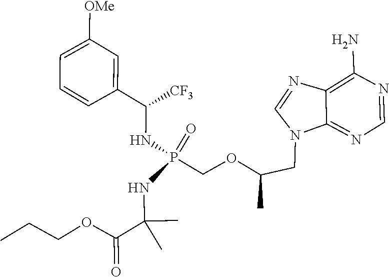 [SMILES: CCCOC(=O)C(C)(C)N[P@@](=O)(CO[C@H](C)Cn1cnc2c(N)ncnc21)N[C@H](c1cccc(OC)c1)C(F)(F)F] |
| IV-141 | 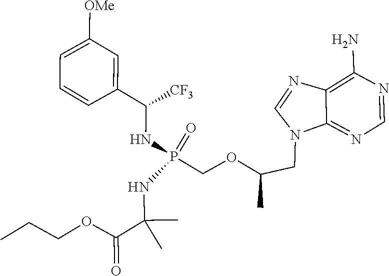 [SMILES: CCCOC(=O)C(C)(C)N[P@](=O)(CO[C@H](C)Cn1cnc2c(N)ncnc21)N[C@H](c1cccc(OC)c1)C(F)(F)F] |
| IV-142 | 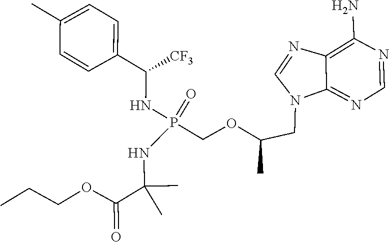 [SMILES: CCCOC(=O)C(C)(C)NP(=O)(CO[C@H](C)Cn1cnc2c(N)ncnc21)N[C@H](c1ccc(C)cc1)C(F)(F)F] |
| IV-143 | 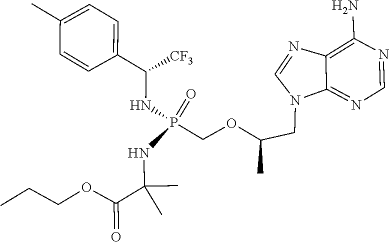 [SMILES: CCCOC(=O)C(C)(C)N[P@@](=O)(CO[C@H](C)Cn1cnc2c(N)ncnc21)N[C@H](c1ccc(C)cc1)C(F)(F)F] |
| IV-144 | 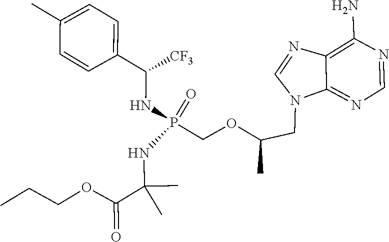 [SMILES: CCCOC(=O)C(C)(C)N[P@](=O)(CO[C@H](C)Cn1cnc2c(N)ncnc21)N[C@H](c1ccc(C)cc1)C(F)(F)F] |
| IV-145 | 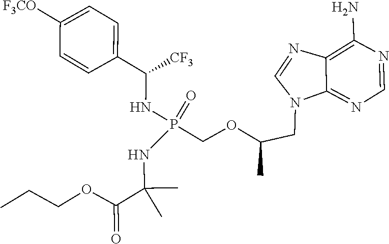 [SMILES: CCCOC(=O)C(C)(C)NP(=O)(CO[C@H](C)Cn1cnc2c(N)ncnc21)N[C@H](c1ccc(OC(F)(F)F)cc1)C(F)(F)F] |
| IV-146 | 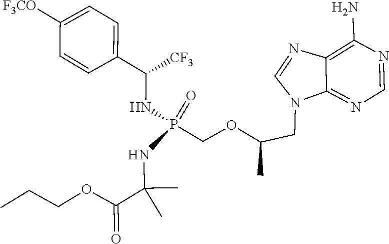 [SMILES: CCCOC(=O)C(C)(C)N[P@@](=O)(CO[C@H](C)Cn1cnc2c(N)ncnc21)N[C@H](c1ccc(OC(F)(F)F)cc1)C(F)(F)F] |
| IV-147 | 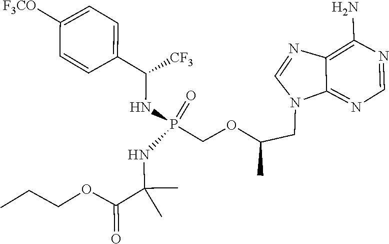 [SMILES: CCCOC(=O)C(C)(C)N[P@](=O)(CO[C@H](C)Cn1cnc2c(N)ncnc21)N[C@H](c1ccc(OC(F)(F)F)cc1)C(F)(F)F] |
| IV-148 | 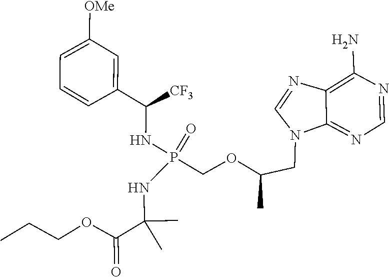 [SMILES: CCCOC(=O)C(C)(C)NP(=O)(CO[C@H](C)Cn1cnc2c(N)ncnc21)N[C@@H](c1cccc(OC)c1)C(F)(F)F] |
| IV-149 | 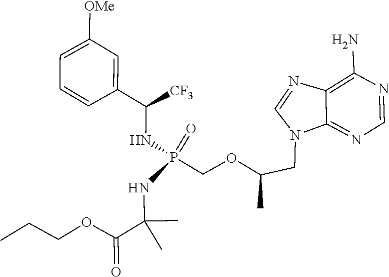 [SMILES: CCCOC(=O)C(C)(C)N[P@@](=O)(CO[C@H](C)Cn1cnc2c(N)ncnc21)N[C@@H](c1cccc(OC)c1)C(F)(F)F] |
| IV-150 | 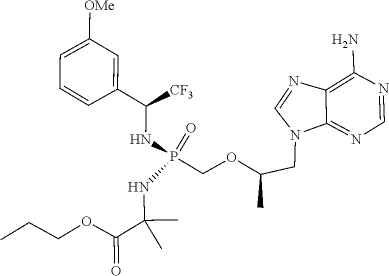 [SMILES: CCCOC(=O)C(C)(C)N[P@](=O)(CO[C@H](C)Cn1cnc2c(N)ncnc21)N[C@@H](c1cccc(OC)c1)C(F)(F)F] |
| IV-151 | 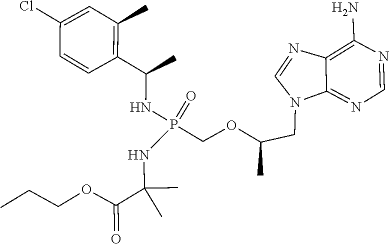 |
| IV-152 | 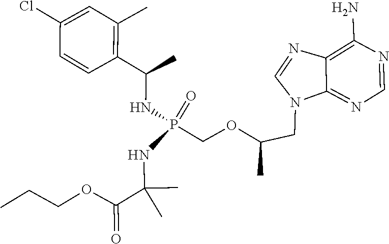 [SMILES: CCCOC(=O)C(C)(C)N[P@@](=O)(CO[C@H](C)Cn1cnc2c(N)ncnc21)N[C@H](C)c1ccc(Cl)cc1C] |
| IV-153 | 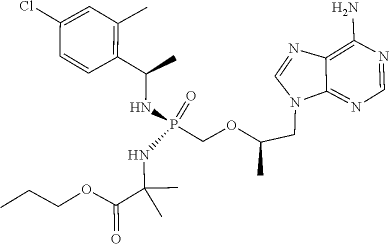 [SMILES: CCCOC(=O)C(C)(C)N[P@](=O)(CO[C@H](C)Cn1cnc2c(N)ncnc21)N[C@H](C)c1ccc(Cl)cc1C] |
| IV-154 | 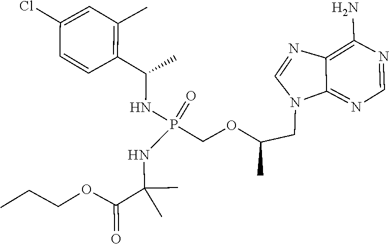 [SMILES: CCCOC(=O)C(C)(C)NP(=O)(CO[C@H](C)Cn1cnc2c(N)ncnc21)N[C@@H](C)c1ccc(Cl)cc1C] |
| IV-155 | 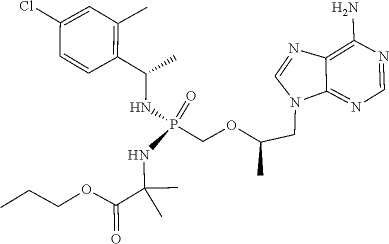 [SMILES: CCCOC(=O)C(C)(C)N[P@@](=O)(CO[C@H](C)Cn1cnc2c(N)ncnc21)N[C@@H](C)c1ccc(Cl)cc1C] |
| IV-156 | 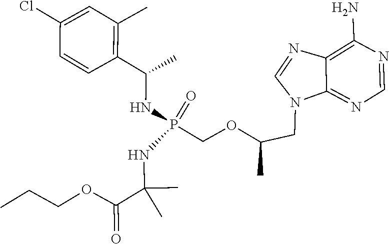 [SMILES: CCCOC(=O)C(C)(C)N[P@](=O)(CO[C@H](C)Cn1cnc2c(N)ncnc21)N[C@@H](C)c1ccc(Cl)cc1C] |
| IV-157 | 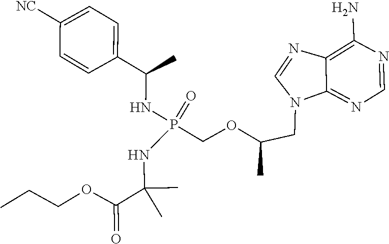 [SMILES: CCCOC(=O)C(C)(C)NP(=O)(CO[C@H](C)Cn1cnc2c(N)ncnc21)N[C@H](C)c1ccc(C#N)cc1] |
| IV-158 | 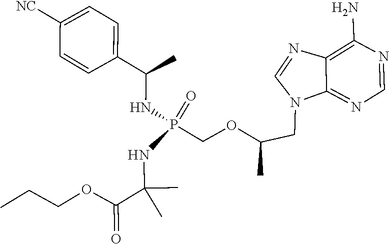 [SMILES: CCCOC(=O)C(C)(C)N[P@@](=O)(CO[C@H](C)Cn1cnc2c(N)ncnc21)N[C@H](C)c1ccc(C#N)cc1] |
| IV-159 | 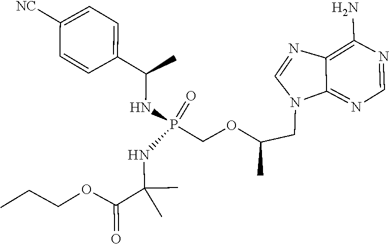 [SMILES: CCCOC(=O)C(C)(C)N[P@](=O)(CO[C@H](C)Cn1cnc2c(N)ncnc21)N[C@H](C)c1ccc(C#N)cc1] |
| IV-160 | 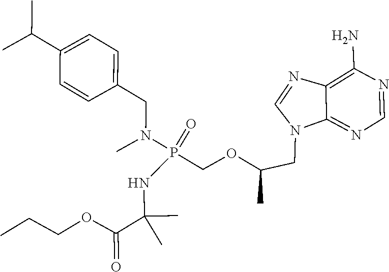 [SMILES: CCCOC(=O)C(C)(C)NP(=O)(CO[C@H](C)Cn1cnc2c(N)ncnc21)N(C)Cc1ccc(C(C)C)cc1] |
| IV-161 | 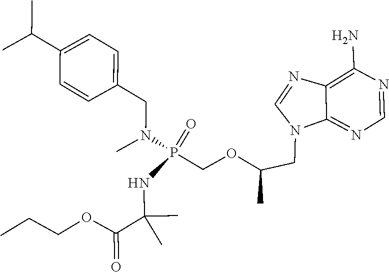 [SMILES: CCCOC(=O)C(C)(C)N[P@@](=O)(CO[C@H](C)Cn1cnc2c(N)ncnc21)N(C)Cc1ccc(C(C)C)cc1] |
| IV-162 | 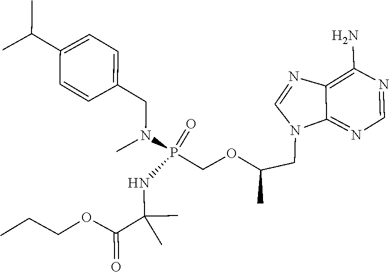 [SMILES: CCCOC(=O)C(C)(C)N[P@](=O)(CO[C@H](C)Cn1cnc2c(N)ncnc21)N(C)Cc1ccc(C(C)C)cc1] |
| IV-163 | 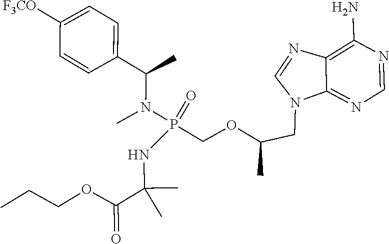 [SMILES: CCCOC(=O)C(C)(C)NP(=O)(CO[C@H](C)Cn1cnc2c(N)ncnc21)N(C)[C@H](C)c1ccc(OC(F)(F)F)cc1] |
| IV-164 | 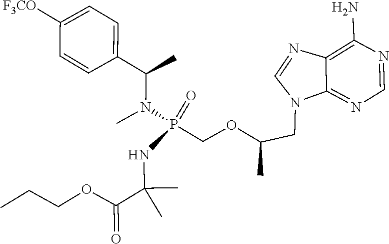 [SMILES: CCCOC(=O)C(C)(C)N[P@@](=O)(CO[C@H](C)Cn1cnc2c(N)ncnc21)N(C)[C@H](C)c1ccc(OC(F)(F)F)cc1] |
| IV-165 | 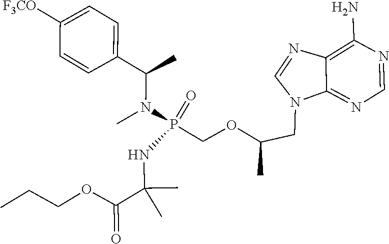 [SMILES: CCCOC(=O)C(C)(C)N[P@](=O)(CO[C@H](C)Cn1cnc2c(N)ncnc21)N(C)[C@H](C)c1ccc(OC(F)(F)F)cc1] |
| IV-166 | 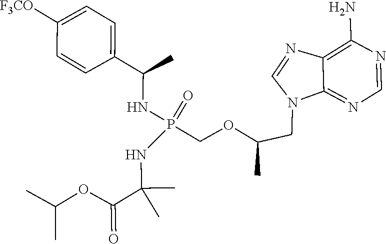 [SMILES: CC(C)OC(=O)C(C)(C)NP(=O)(CO[C@H](C)Cn1cnc2c(N)ncnc21)N[C@H](C)c1ccc(OC(F)(F)F)cc1] |
| IV-167 | 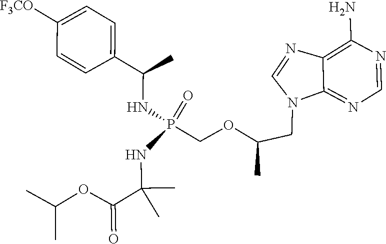 [SMILES: CC(C)OC(=O)C(C)(C)N[P@@](=O)(CO[C@H](C)Cn1cnc2c(N)ncnc21)N[C@H](C)c1ccc(OC(F)(F)F)cc1] |
| IV-168 | 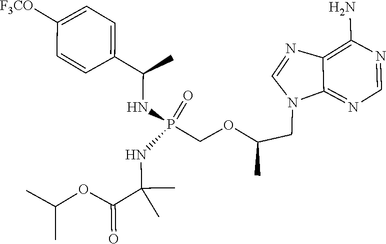 [SMILES: CC(C)OC(=O)C(C)(C)N[P@](=O)(CO[C@H](C)Cn1cnc2c(N)ncnc21)N[C@H](C)c1ccc(OC(F)(F)F)cc1] |
| IV-169 | 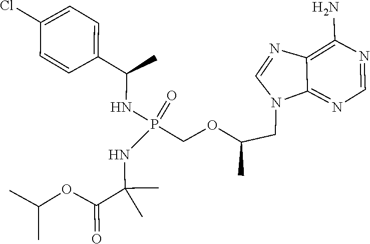 [SMILES: CC(C)OC(=O)C(C)(C)NP(=O)(CO[C@H](C)Cn1cnc2c(N)ncnc21)N[C@H](C)c1ccc(Cl)cc1] |
| IV-170 | 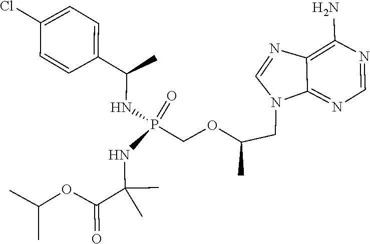 [SMILES: CC(C)OC(=O)C(C)(C)N[P@@](=O)(CO[C@H](C)Cn1cnc2c(N)ncnc21)N[C@H](C)c1ccc(Cl)cc1] |
| IV-171 | 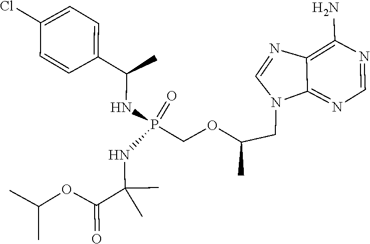 [SMILES: CC(C)OC(=O)C(C)(C)N[P@](=O)(CO[C@H](C)Cn1cnc2c(N)ncnc21)N[C@H](C)c1ccc(Cl)cc1] |
| IV-172 | 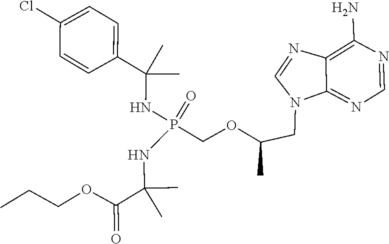 [SMILES: CCCOC(=O)C(C)(C)NP(=O)(CO[C@H](C)Cn1cnc2c(N)ncnc21)NC(C)(C)c1ccc(Cl)cc1] |
| IV-173 | 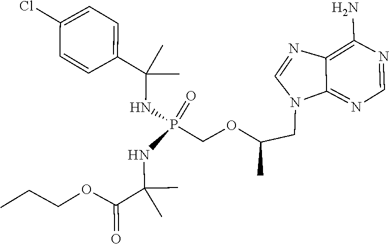 [SMILES: CCCOC(=O)C(C)(C)N[P@@](=O)(CO[C@H](C)Cn1cnc2c(N)ncnc21)NC(C)(C)c1ccc(Cl)cc1] |
| IV-174 | 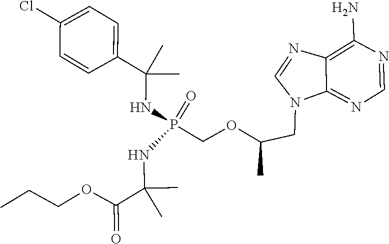 [SMILES: CCCOC(=O)C(C)(C)N[P@](=O)(CO[C@H](C)Cn1cnc2c(N)ncnc21)NC(C)(C)c1ccc(Cl)cc1] |
| IV-175 | 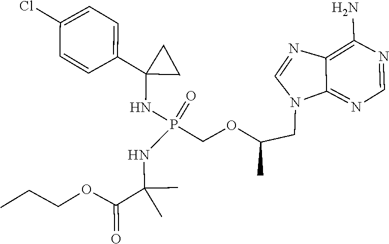 [SMILES: CCCOC(=O)C(C)(C)NP(=O)(CO[C@H](C)Cn1cnc2c(N)ncnc21)NC1(c2ccc(Cl)cc2)CC1] |
| IV-176 | 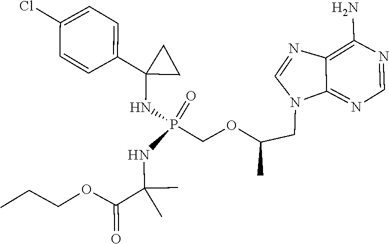 [SMILES: CCCOC(=O)C(C)(C)N[P@@](=O)(CO[C@H](C)Cn1cnc2c(N)ncnc21)NC1(c2ccc(Cl)cc2)CC1] |
| IV-177 | 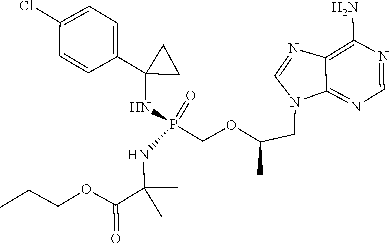 [SMILES: CCCOC(=O)C(C)(C)N[P@](=O)(CO[C@H](C)Cn1cnc2c(N)ncnc21)NC1(c2ccc(Cl)cc2)CC1] |

In certain embodiments, the compound is a compound in Table 5 below, or a pharmaceutically acceptable salt thereof. In certain embodiments, the compound is a compound in Table 5 below. Compounds in Table 5 below can be prepared based on procedures described in, for example, WO 94/03467, WO 98/04569, WO 2019/120071, WO 2014/068265, U.S. 2019/0015432, WO 2021/074443, and WO 2017/156262; and related patents and applications, such as U.S. Pat. Nos. 6,653,296; 5,922,695; EP 3719027 A1; U.S. Pat. Nos. 9,227,990; 10,688,112; and 10,745,427.

TABLE 5

| Compound No. | Chemical Structure |
|---|---|
| V-1 | 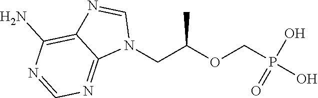 [SMILES: C[C@H](Cn1cnc2c(N)ncnc21)OCP(=O)(O)O] |
| V-2 | 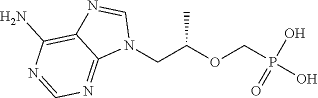 [SMILES: C[C@@H](Cn1cnc2c(N)ncnc21)OCP(=O)(O)O] |
| V-3 | 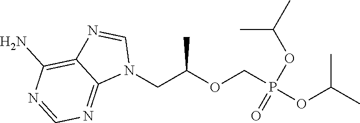 [SMILES: CC(C)OP(=O)(CO[C@H](C)Cn1cnc2c(N)ncnc21)OC(C)C] |
| V-4 | 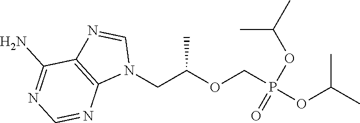 [SMILES: CC(C)OP(=O)(CO[C@@H](C)Cn1cnc2c(N)ncnc21)OC(C)C] |
| V-5 | 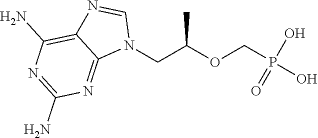 [SMILES: C[C@H](Cn1cnc2c(N)nc(N)nc21)OCP(=O)(O)O] |
| V-6 | 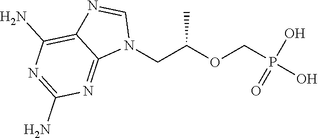 [SMILES: C[C@@H](Cn1cnc2c(N)nc(N)nc21)OCP(=O)(O)O] |
| V-7 | 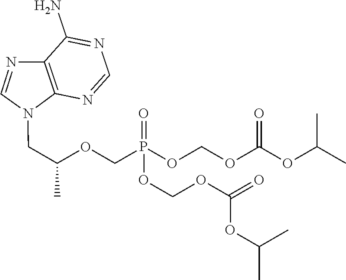 [SMILES: CC(C)OC(=O)OCOP(=O)(CO[C@H](C)Cn1cnc2c(N)ncnc21)OCOC(=O)OC(C)C] |
| V-8 | 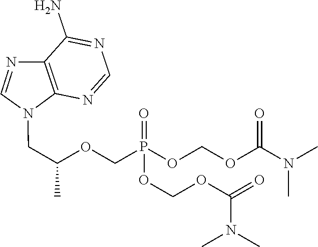 [SMILES: C[C@H](Cn1cnc2c(N)ncnc21)OCP(=O)(OCOC(=O)N(C)C)OCOC(=O)N(C)C] |
| V-9 | 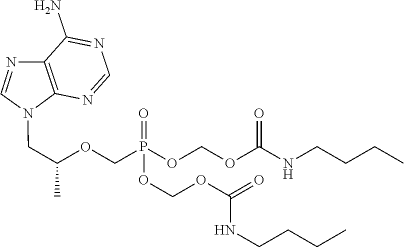 [SMILES: CCCCNC(=O)OCOP(=O)(CO[C@H](C)Cn1cnc2c(N)ncnc21)OCOC(=O)NCCCC] |
| V-10 | 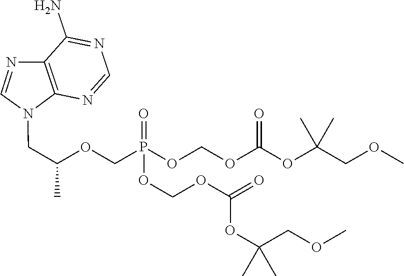 [SMILES: COCC(C)(C)OC(=O)OCOP(=O)(CO[C@H](C)Cn1cnc2c(N)ncnc21)OCOC(=O)OC(C)(C)COC] |
| V-11 | 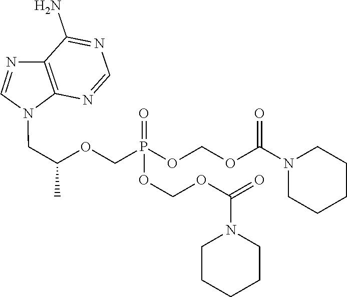 [SMILES: C[C@H](Cn1cnc2c(N)ncnc21)OCP(=O)(OCOC(=O)N1CCCCC1)OCOC(=O)N1CCCCC1] |
| V-12 | 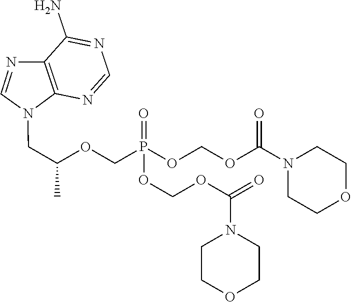 [SMILES: C[C@H](Cn1cnc2c(N)ncnc21)OCP(=O)(OCOC(=O)N1CCOCC1)OCOC(=O)N1CCOCC1] |
| V-13 | 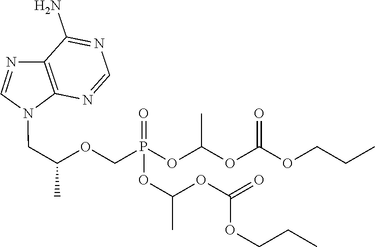 [SMILES: CCCOC(=O)OC(C)OP(=O)(CO[C@H](C)Cn1cnc2c(N)ncnc21)OC(C)OC(=O)OCCC] |
| V-14 | 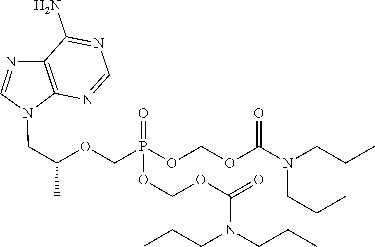 [SMILES: CCCN(CCC)C(=O)OCOP(=O)(CO[C@H](C)Cn1cnc2c(N)ncnc21)OCOC(=O)N(CCC)CCC] |
| V-15 | 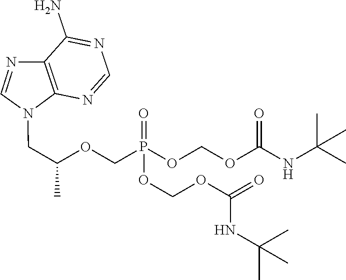 [SMILES: C[C@H](Cn1cnc2c(N)ncnc21)OCP(=O)(OCOC(=O)NC(C)(C)C)OCOC(=O)NC(C)(C)C] |
| V-16 | 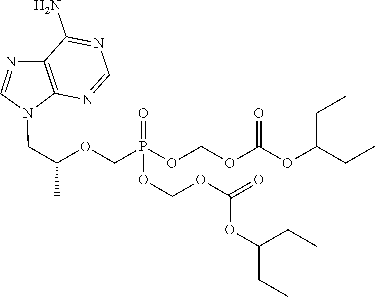 [SMILES: CCC(CC)OC(=O)OCOP(=O)(CO[C@H](C)Cn1cnc2c(N)ncnc21)OCOC(=O)OC(CC)CC] |
| V-17 | 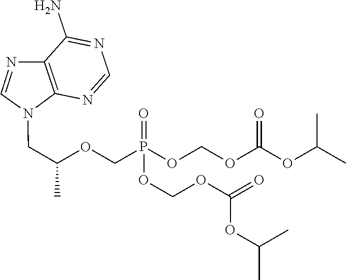 [SMILES: CC(C)OC(=O)OCOP(=O)(CO[C@H](C)Cn1cnc2c(N)ncnc21)OCOC(=O)OC(C)C] |
| V-18 | 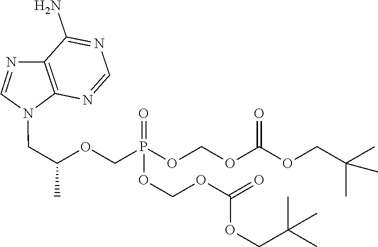 [SMILES: C[C@H](Cn1cnc2c(N)ncnc21)OCP(=O)(OCOC(=O)OCC(C)(C)C)OCOC(=O)OCC(C)(C)C] |
| V-19 | 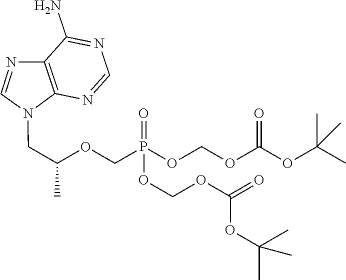 [SMILES: C[C@H](Cn1cnc2c(N)ncnc21)OCP(=O)(OCOC(=O)OC(C)(C)C)OCOC(=O)OC(C)(C)C] |
| V-20 | 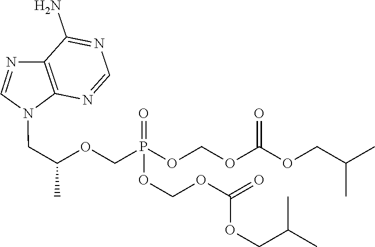 [SMILES: CC(C)COC(=O)OCOP(=O)(CO[C@H](C)Cn1cnc2c(N)ncnc21)OCOC(=O)OCC(C)C] |
| V-21 | 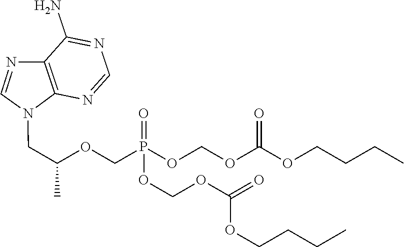 [SMILES: CCCCOC(=O)OCOP(=O)(CO[C@H](C)Cn1cnc2c(N)ncnc21)OCOC(=O)OCCCC] |
| V-22 | 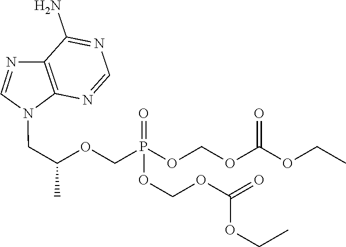 [SMILES: CCOC(=O)OCOP(=O)(CO[C@H](C)Cn1cnc2c(N)ncnc21)OCOC(=O)OCC] |
| V-23 | 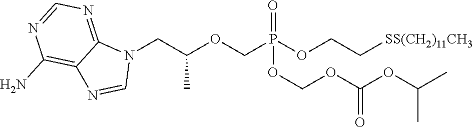 |
| V-24 | 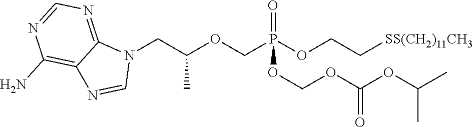 |
| V-25 | 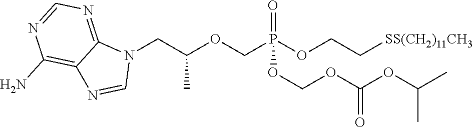 |
| V-26 | 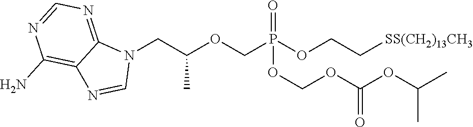 |
| V-27 | 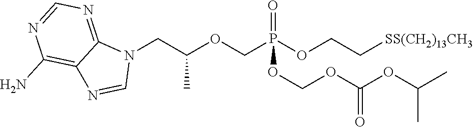 |
| V-28 | 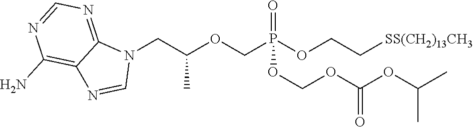 |
| V-29 | 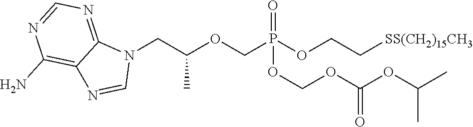 |
| V-30 | 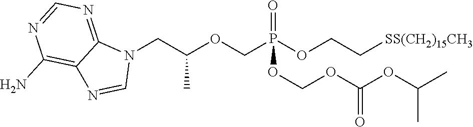 |
| V-31 | 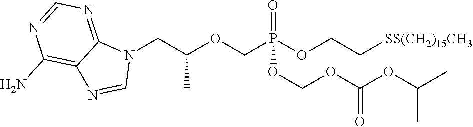 |
| V-32 | 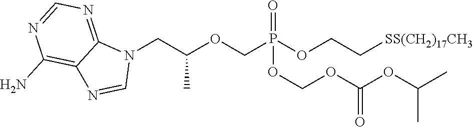 |
| V-33 | 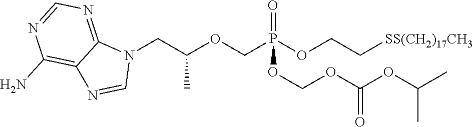 |
| V-34 | 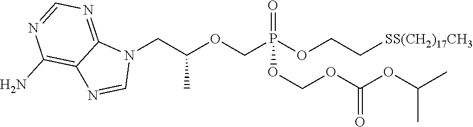 |
| V-35 | 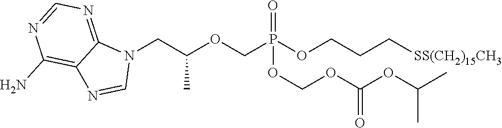 |
| V-36 | 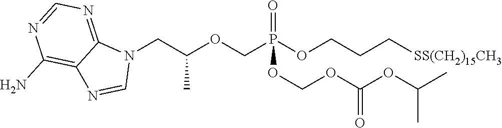 |
| V-37 | 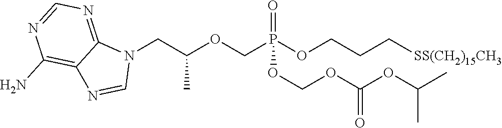 |
| V-38 | 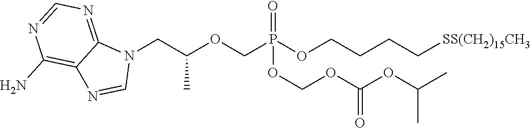 |
| V-39 | 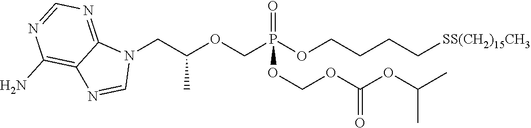 |
| V-40 | 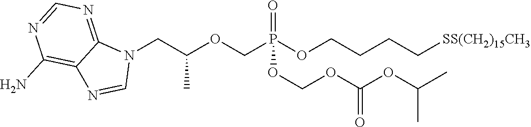 |
| V-41 | 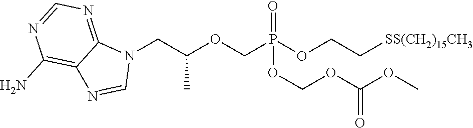 |
| V-42 | 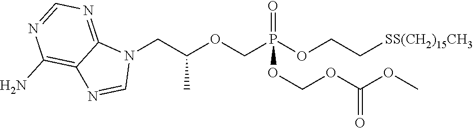 |
| V-43 | 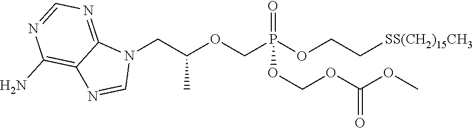 |
| V-44 | 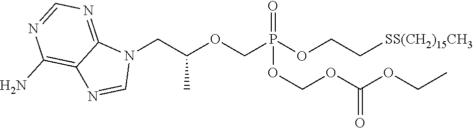 |
| V-45 | 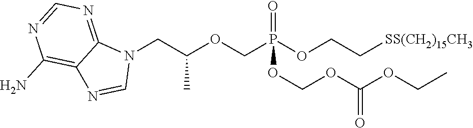 |
| V-46 | 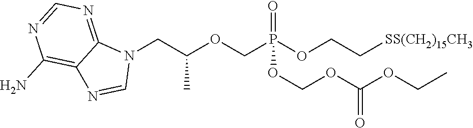 |
| V-47 | 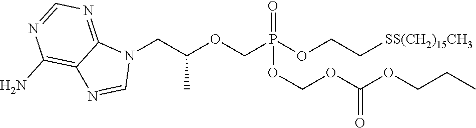 |
| V-48 | 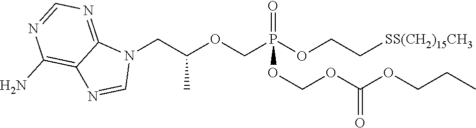 |
| V-49 | 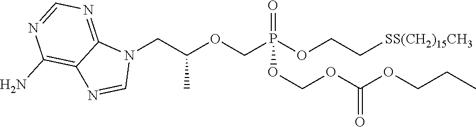 |
| V-50 | 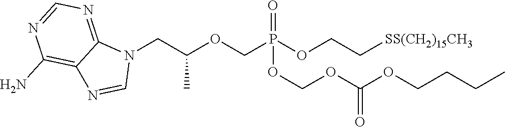 |
| V-51 | 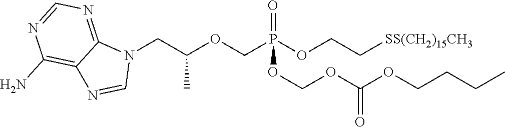 |
| V-52 | 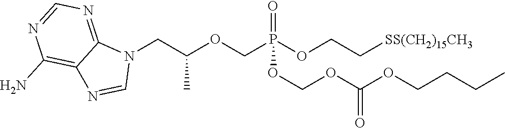 |
| V-53 | 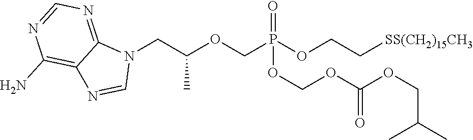 |
| V-54 | 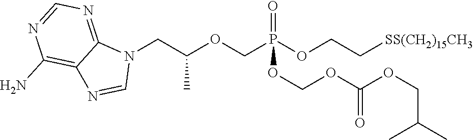 |
| V-55 | 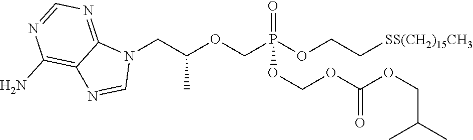 |
| V-56 | 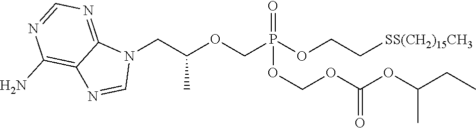 |
| V-57 | 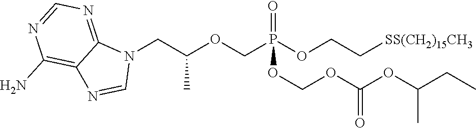 |
| V-58 | 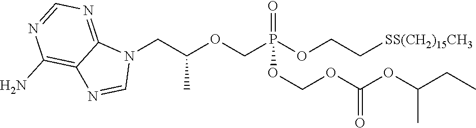 |
| V-59 | 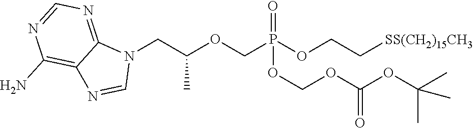 |
| V-60 | 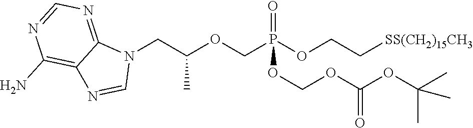 |
| V-61 | 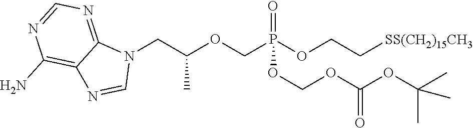 |
| V-62 | 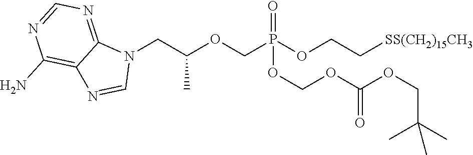 |
| V-63 | 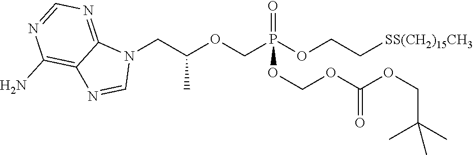 |
| V-64 | 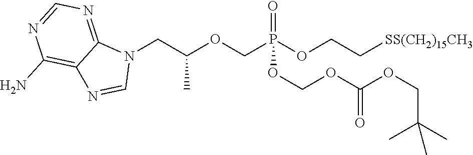 |
| V-65 | 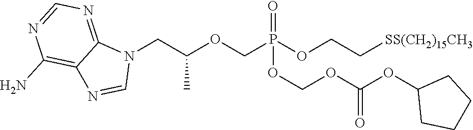 |
| V-66 | 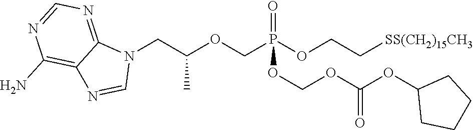 |
| V-67 | 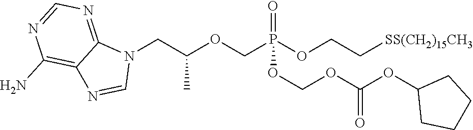 |
| V-68 | 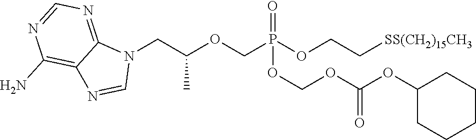 |
| V-69 | 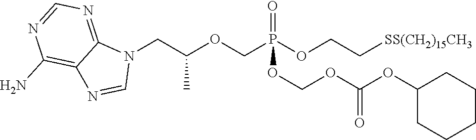 |
| V-70 | 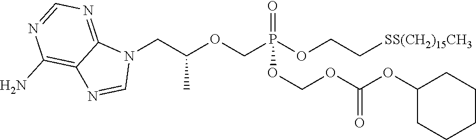 |
| V-71 | 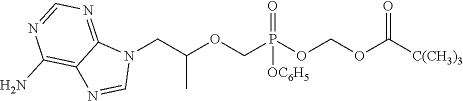 |
| V-72 | 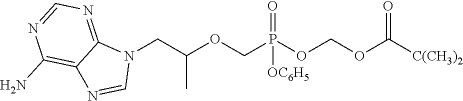 |
| V-73 | 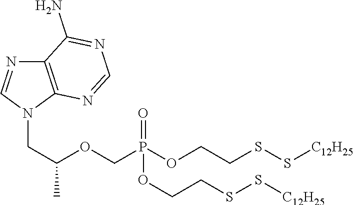 |
| V-74 | 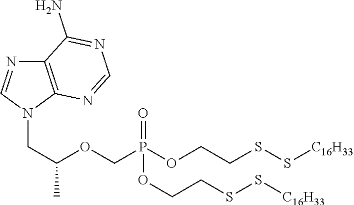 |
| V-75 | 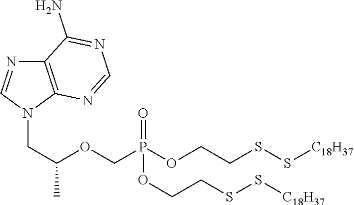 |
| V-76 | 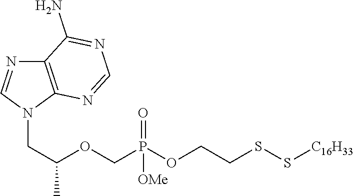 |
| V-77 | 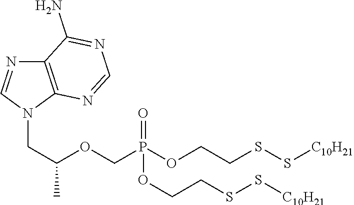 |
| V-78 | 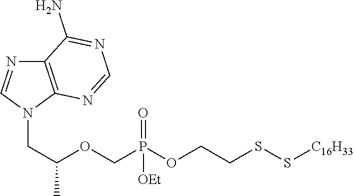 |
| V-79 | 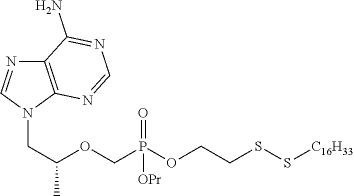 |
| V-80 | 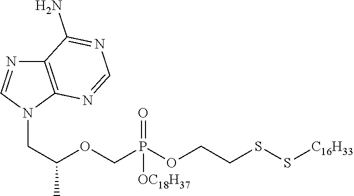 |
| V-81 | 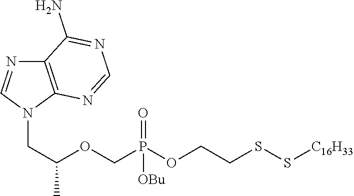 |
| V-82 | 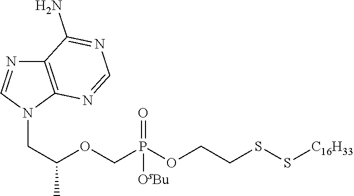 |
| V-83 | 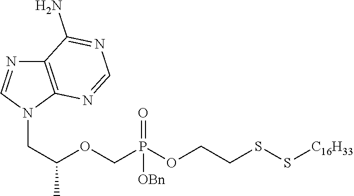 |
| V-84 | 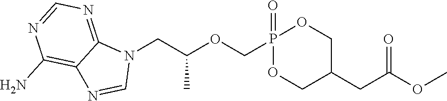 [SMILES: COC(=O)CC1COP(=O)(CO[C@H](C)Cn2cnc3c(N)ncnc32)OC1] |
| V-85 | 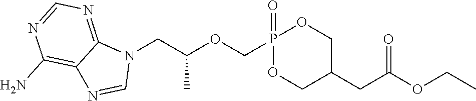 [SMILES: CCOC(=O)CC1COP(=O)(CO[C@H](C)Cn2cnc3c(N)ncnc32)OC1] |
| V-86 | 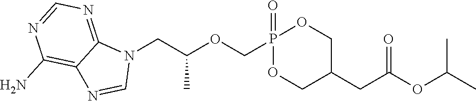 [SMILES: CC(C)OC(=O)CC1COP(=O)(CO[C@H](C)Cn2cnc3c(N)ncnc32)OC1] |
| V-87 | 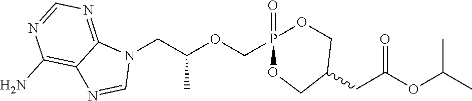 |
| V-88 | 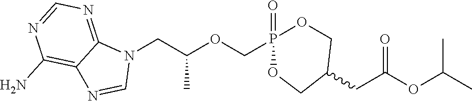 |
| V-89 | 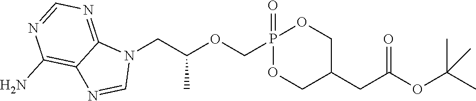 [SMILES: C[C@H](Cn1cnc2c(N)ncnc21)OCP1(=O)OCC(CC(=O)OC(C)(C)C)CO1] |
| V-90 | 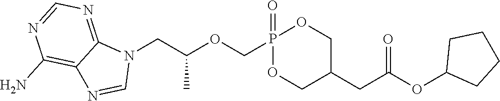 [SMILES: C[C@H](Cn1cnc2c(N)ncnc21)OCP1(=O)OCC(CC(=O)OC2CCCC2)CO1] |
| V-91 | 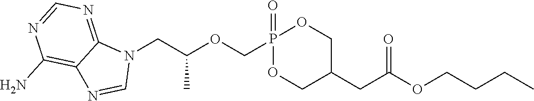 [SMILES: CCCCOC(=O)CC1COP(=O)(CO[C@H](C)Cn2cnc3c(N)ncnc32)OC1] |
| V-92 | 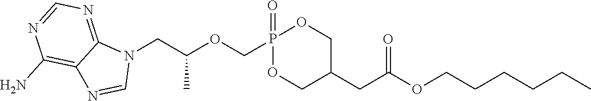 [SMILES: CCCCCCOC(=O)CC1COP(=O)(CO[C@H](C)Cn2cnc3c(N)ncnc32)OC1] |
| V-93 | 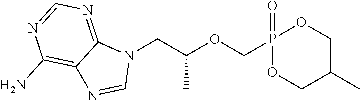 [SMILES: CC1COP(=O)(CO[C@H](C)Cn2cnc3c(N)ncnc32)OC1] |
| V-94 | 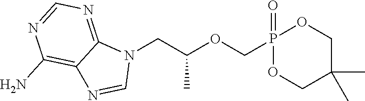 [SMILES: C[C@H](Cn1cnc2c(N)ncnc21)OCP1(=O)OCC(C)(C)CO1] |
| V-95 | 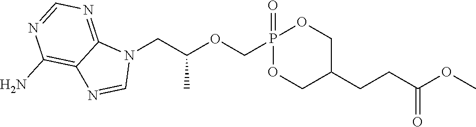 [SMILES: COC(=O)CCC1COP(=O)(CO[C@H](C)Cn2cnc3c(N)ncnc32)OC1] |
| V-96 | 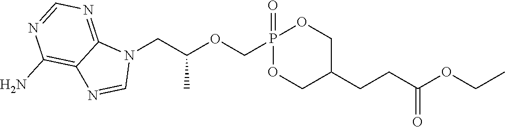 [SMILES: CCOC(=O)CCC1COP(=O)(CO[C@H](C)Cn2cnc3c(N)ncnc32)OC1] |
| V-97 | 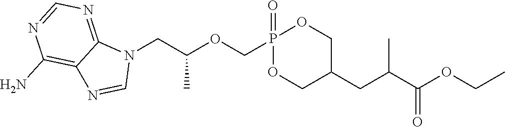 [SMILES: CCOC(=O)C(C)CC1COP(=O)(CO[C@H](C)Cn2cnc3c(N)ncnc32)OC1] |
| V-98 | 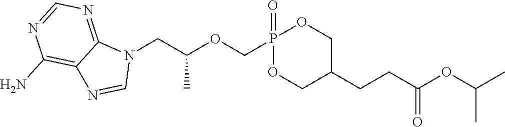 [SMILES: CC(C)OC(=O)CCC1COP(=O)(CO[C@H](C)Cn2cnc3c(N)ncnc32)OC1] |
| V-99 | 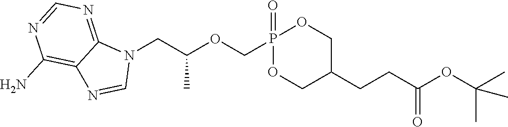 [SMILES: C[C@H](Cn1cnc2c(N)ncnc21)OCP1(=O)OCC(CCC(=O)OC(C)(C)C)CO1] |
| V-100 | 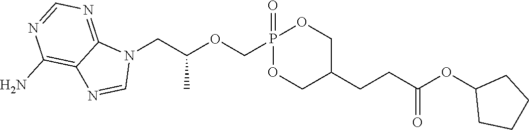 [SMILES: C[C@H](Cn1cnc2c(N)ncnc21)OCP1(=O)OCC(CCC(=O)OC2CCCC2)CO1] |
| V-101 | 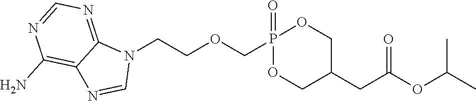 [SMILES: CC(C)OC(=O)CC1COP(=O)(COCCn2cnc3c(N)ncnc32)OC1] |
| V-102 | 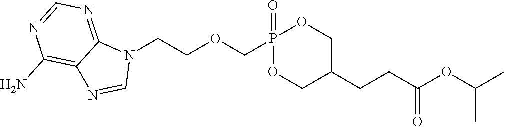 [SMILES: CC(C)OC(=O)CCC1COP(=O)(COCCn2cnc3c(N)ncnc32)OC1] |
| V-103 | 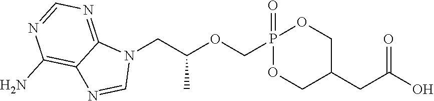 [SMILES: C[C@H](Cn1cnc2c(N)ncnc21)OCP1(=O)OCC(CC(=O)O)CO1] |
| V-104 | 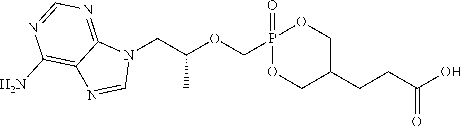 [SMILES: C[C@H](Cn1cnc2c(N)ncnc21)OCP1(=O)OCC(CCC(=O)O)CO1] |
| V-105 | 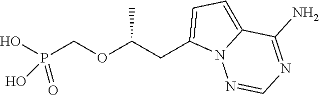 [SMILES: C[C@H](Cc1ccc2c(N)ncnn12)OCP(=O)(O)O] |
| V-106 | 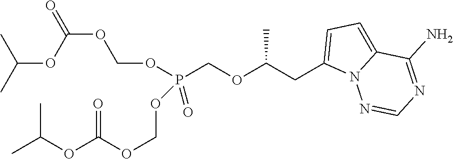 [SMILES: CC(C)OC(=O)OCOP(=O)(CO[C@H](C)Cc1ccc2c(N)ncnn12)OCOC(=O)OC(C)C] |
| V-107 | 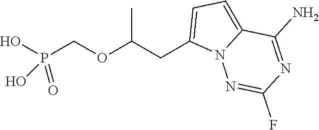 [SMILES: CC(Cc1ccc2c(N)nc(F)nn12)OCP(=O)(O)O] |
| V-108 | 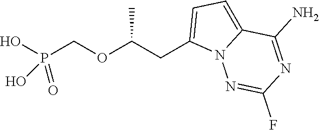 [SMILES: C[C@H](Cc1ccc2c(N)nc(F)nn12)OCP(=O)(O)O] |
| V-109 | 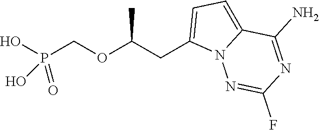 [SMILES: C[C@@H](Cc1ccc2c(N)nc(F)nn12)OCP(=O)(O)O] |
| V-110 | 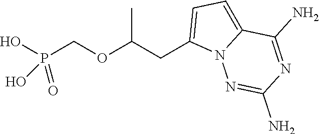 [SMILES: CC(Cc1ccc2c(N)nc(N)nn12)OCP(=O)(O)O] |
| V-111 | 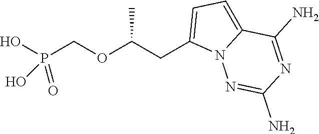 [SMILES: C[C@H](Cc1ccc2c(N)nc(N)nn12)OCP(=O)(O)O] |
| V-112 | 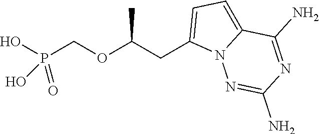 [SMILES: C[C@@H](Cc1ccc2c(N)nc(N)nn12)OCP(=O)(O)O] |
| V-113 | 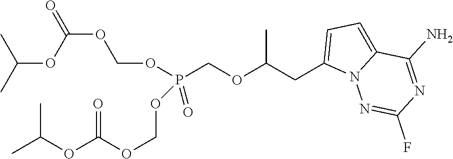 [SMILES: CC(C)OC(=O)OCOP(=O)(COC(C)Cc1ccc2c(N)nc(F)nn12)OCOC(=O)OC(C)C] |
| V-114 | 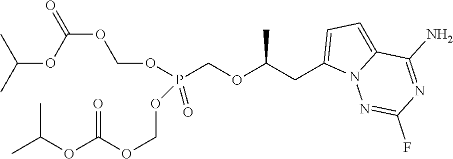 [SMILES: CC(C)OC(=O)OCOP(=O)(CO[C@@H](C)Cc1ccc2c(N)nc(F)nn12)OCOC(=O)OC(C)C] |
| V-115 | 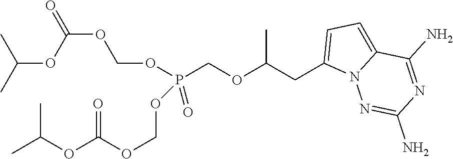 [SMILES: CC(C)OC(=O)OCOP(=O)(COC(C)Cc1ccc2c(N)nc(N)nn12)OCOC(=O)OC(C)C] |
| V-116 | 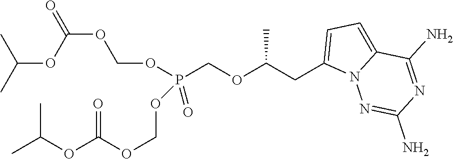 [SMILES: CC(C)OC(=O)OCOP(=O)(CO[C@H](C)Cc1ccc2c(N)nc(N)nn12)OCOC(=O)OC(C)C] |
| V-117 | 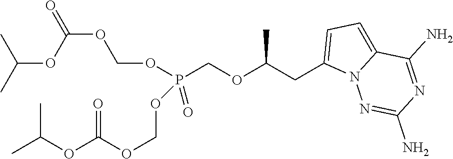 [SMILES: CC(C)OC(=O)OCOP(=O)(CO[C@@H](C)Cc1ccc2c(N)nc(N)nn12)OCOC(=O)OC(C)C] |
| V-118 | 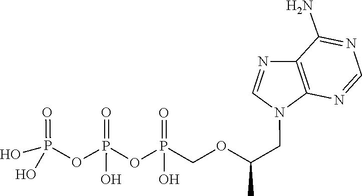 [SMILES: C[C@H](Cn1cnc2c(N)ncnc21)OCP(=O)(O)OP(=O)(O)OP(=O)(O)O] |
| V-119 | 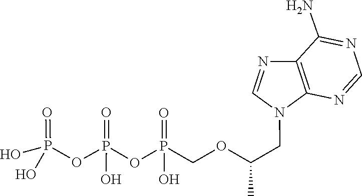 [SMILES: C[C@@H](Cn1cnc2c(N)ncnc21)OCP(=O)(O)OP(=O)(O)OP(=O)(O)O] |
| V-120 | 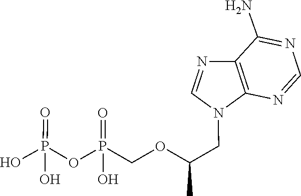 [SMILES: C[C@H](Cn1cnc2c(N)ncnc21)OCP(=O)(O)OP(=O)(O)O] |
| V-121 | 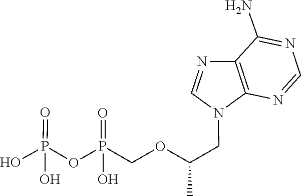 [SMILES: C[C@@H](Cn1cnc2c(N)ncnc21)OCP(=O)(O)OP(=O)(O)O] |
| V-122 | 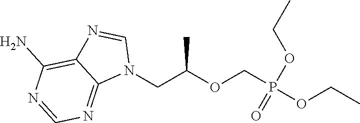 [SMILES: CCOP(=O)(CO[C@H](C)Cn1cnc2c(N)ncnc21)OCC] |
| V-123 | 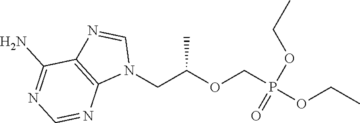 [SMILES: CCOP(=O)(CO[C@@H](C)Cn1cnc2c(N)ncnc21)OCC] |

In certain embodiments, the compound is a compound in Table 6 below, or a pharmaceutically acceptable salt thereof. In certain embodiments, the compound is a compound in Table 6 below. Compounds in Table 6 below can be prepared based on procedures described in, for example, WO 2011/053812, WO 2011/100698, WO 02/08241, and U.S. 2019/0015432; and related patents and applications, such as U.S. Pat. Nos. 9,006,218; 7,390,791; and 10,688,112.

TABLE 6

| Cmpnd No. | Chemical Structure |
|---|---|
| VI-1 | 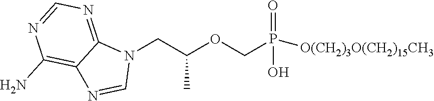 |
| VI-2 | 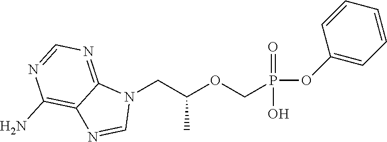 [SMILES: C[C@H](Cn1cnc2c(N)ncnc21)OCP(=O)(O)Oc1ccccc1] |
| VI-3 | 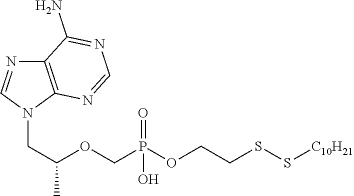 |
| VI-4 | 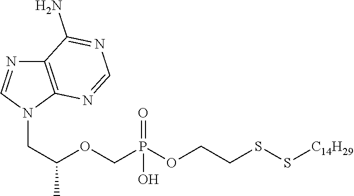 |
| VI-5 | 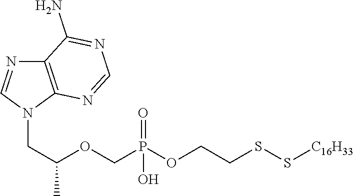 |
| VI-6 | 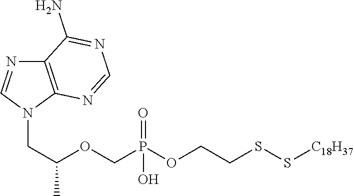 |
| VI-7 | 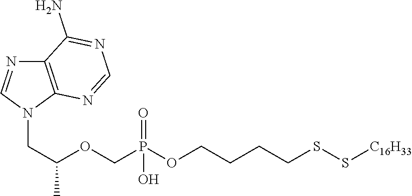 |
| VI-8 | 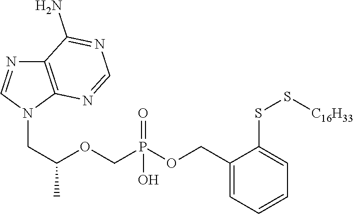 |
| VI-9 | 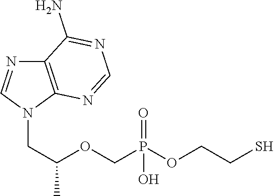 [SMILES: C[C@H](Cn1cnc2c(N)ncnc21)OCP(=O)(O)OCCS] |
| VI-10 | 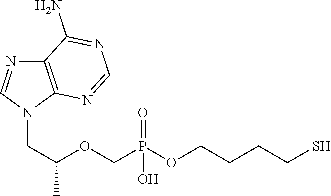 [SMILES: C[C@H](Cn1cnc2c(N)ncnc21)OCP(=O)(O)OCCCCS] |
| VI-11 | 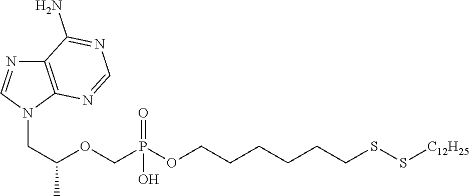 |
| VI-12 | 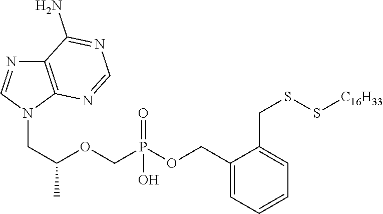 |
| VI-13 | 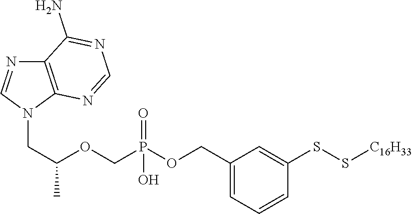 |
| VI-14 | 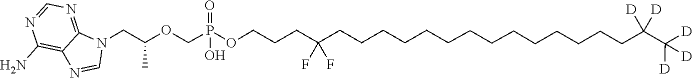 [SMILES: [2H]C([2H])([2H])C([2H])([2H])CCCCCCCCCCCCCCC(F)(F)CCCOP(=O)(O)CO[C@H](C)Cn1cnc2c(N)ncnc21] |
| VI-15 | 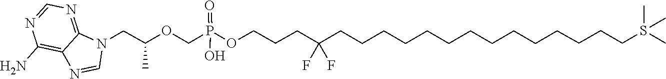 [SMILES: C[C@H](Cn1cnc2c(N)ncnc21)OCP(=O)(O)OCCCC(F)(F)CCCCCCCCCCCCCCS(C)(C)C] |
| VI-16 | 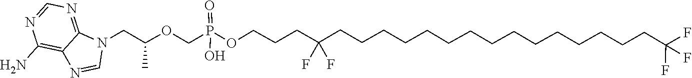 [SMILES: C[C@H](Cn1cnc2c(N)ncnc21)OCP(=O)(O)OCCCC(F)(F)CCCCCCCCCCCCCCCC(F)(F)F] |
| VI-17 | 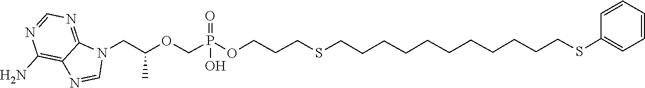 [SMILES: C[C@H](Cn1cnc2c(N)ncnc21)OCP(=O)(O)OCCCSCCCCCCCCCCCSc1ccccc1] |
| VI-18 | 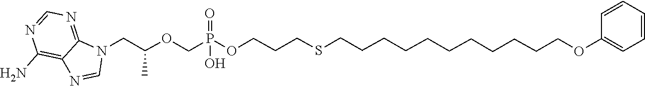 [SMILES: C[C@H](Cn1cnc2c(N)ncnc21)OCP(=O)(O)OCCCSCCCCCCCCCCCOc1ccccc1] |
| VI-19 | 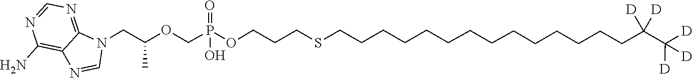 [SMILES: [2H]C([2H])([2H])C([2H])([2H])CCCCCCCCCCCCCCSCCCOP(=O)(O)CO[C@H](C)Cn1cnc2c(N)ncnc21] |
| VI-20 | 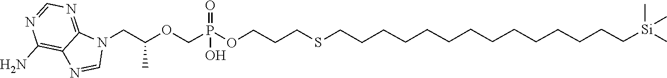 [SMILES: C[C@H](Cn1cnc2c(N)ncnc21)OCP(=O)(O)OCCCSCCCCCCCCCCCCCC[Si](C)(C)C] |
| VI-21 | 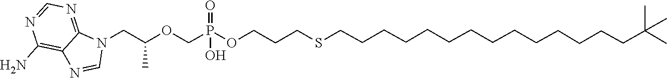 [SMILES: C[C@H](Cn1cnc2c(N)ncnc21)OCP(=O)(O)OCCCSCCCCCCCCCCCCCCC(C)(C)C] |
| VI-22 | 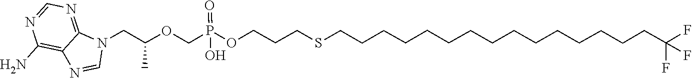 [SMILES: C[C@H](Cn1cnc2c(N)ncnc21)OCP(=O)(O)OCCCSCCCCCCCCCCCCCCCC(F)(F)F] |
| VI-23 | 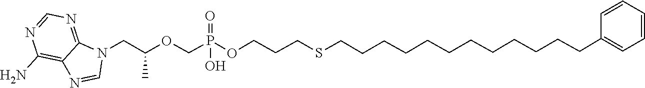 [SMILES: C[C@H](Cn1cnc2c(N)ncnc21)OCP(=O)(O)OCCCSCCCCCCCCCCCCc1ccccc1] |
| VI-24 | 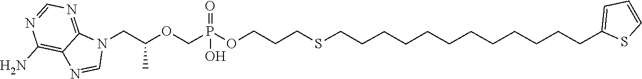 [SMILES: C[C@H](Cn1cnc2c(N)ncnc21)OCP(=O)(O)OCCCSCCCCCCCCCCCCc1cccs1] |
| VI-25 | 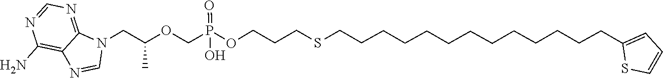 [SMILES: C[C@H](Cn1cnc2c(N)ncnc21)OCP(=O)(O)OCCCSCCCCCCCCCCCCCc1cccs1] |
| VI-26 | 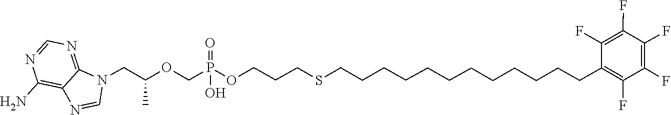 [SMILES: C[C@H](Cn1cnc2c(N)ncnc21)OCP(=O)(O)OCCCSCCCCCCCCCCCCc1c(F)c(F)c(F)c(F)c1F] |
| VI-27 | 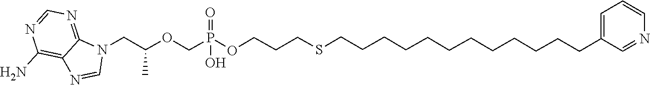 [SMILES: C[C@H](Cn1cnc2c(N)ncnc21)OCP(=O)(O)OCCCSCCCCCCCCCCCCc1cccnc1] |
| VI-28 | 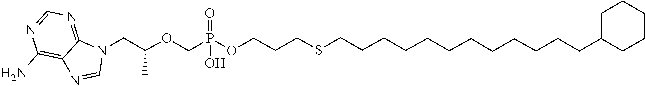 [SMILES: C[C@H](Cn1cnc2c(N)ncnc21)OCP(=O)(O)OCCCSCCCCCCCCCCCCC1CCCCC1] |
| VI-29 | 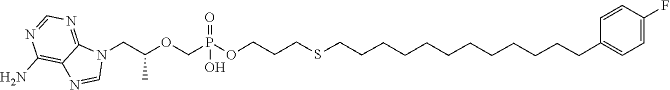 [SMILES: C[C@H](Cn1cnc2c(N)ncnc21)OCP(=O)(O)OCCCSCCCCCCCCCCCCc1ccc(F)cc1] |
| VI-30 | 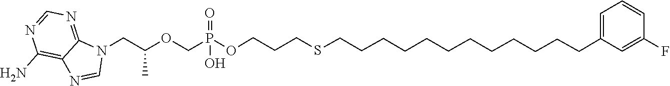 [SMILES: C[C@H](Cn1cnc2c(N)ncnc21)OCP(=O)(O)OCCCSCCCCCCCCCCCCc1cccc(F)c1] |
| VI-31 | 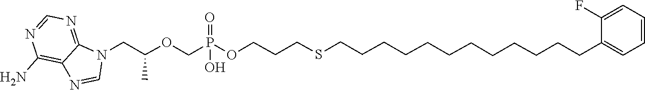 [SMILES: C[C@H](Cn1cnc2c(N)ncnc21)OCP(=O)(O)OCCCSCCCCCCCCCCCCc1ccccc1F] |
| VI-32 | 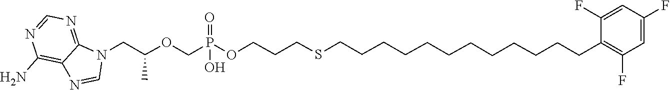 [SMILES: C[C@H](Cn1cnc2c(N)ncnc21)OCP(=O)(O)OCCCSCCCCCCCCCCCCc1c(F)cc(F)cc1F] |
| VI-33 | 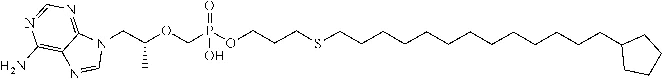 [SMILES: C[C@H](Cn1cnc2c(N)ncnc21)OCP(=O)(O)OCCCSCCCCCCCCCCCCCC1CCCC1] |
| VI-34 | 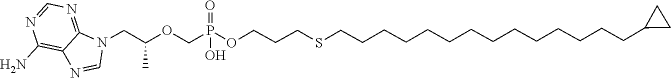 [SMILES: C[C@H](Cn1cnc2c(N)ncnc21)OCP(=O)(O)OCCCSCCCCCCCCCCCCCCC1CC1] |
| VI-35 | 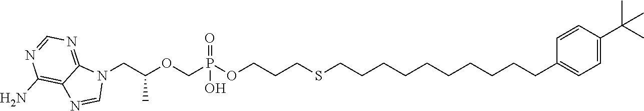 [SMILES: C[C@H](Cn1cnc2c(N)ncnc21)OCP(=O)(O)OCCCSCCCCCCCCCCc1ccc(C(C)(C)C)cc1] |
| VI-36 | 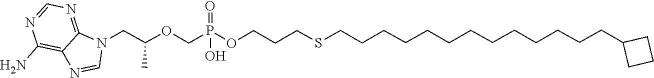 [SMILES: C[C@H](Cn1cnc2c(N)ncnc21)OCP(=O)(O)OCCCSCCCCCCCCCCCCCC1CCC1] |
| VI-37 | 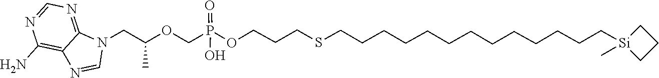 [SMILES: C[C@H](Cn1cnc2c(N)ncnc21)OCP(=O)(O)OCCCSCCCCCCCCCCCCC[Si]1(C)CCC1] |
| VI-38 | 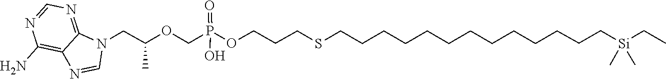 [SMILES: CC[Si](C)(C)CCCCCCCCCCCCCSCCCOP(=O)(O)CO[C@H](C)Cn1cnc2c(N)ncnc21] |
| VI-39 | 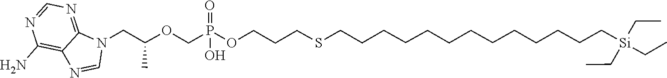 [SMILES: CC[Si](CC)(CC)CCCCCCCCCCCCCSCCCOP(=O)(O)CO[C@H](C)Cn1cnc2c(N)ncnc21] |
| VI-40 | 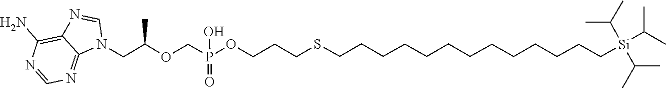 [SMILES: CC(C)[Si](CCCCCCCCCCCCCSCCCOP(=O)(O)CO[C@H](C)Cn1cnc2c(N)ncnc21)(C(C)C)C(C)C] |
| VI-41 | 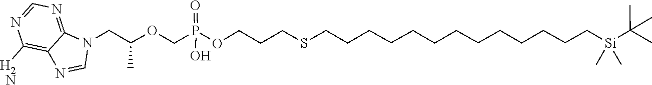 [SMILES: C[C@H](Cn1cnc2c(N)ncnc21)OCP(=O)(O)OCCCSCCCCCCCCCCCCC[Si](C)(C)C(C)(C)C] |
| VI-42 | 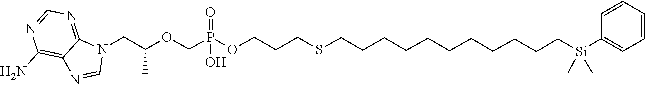 [SMILES: C[C@H](Cn1cnc2c(N)ncnc21)OCP(=O)(O)OCCCSCCCCCCCCCCC[Si](C)(C)c1ccccc1] |
| VI-43 | 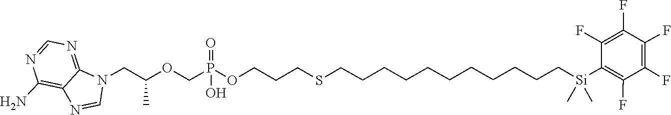 [SMILES: C[C@H](Cn1cnc2c(N)ncnc21)OCP(=O)(O)OCCCSCCCCCCCCCCC[Si](C)(C)c1c(F)c(F)c(F)c(F)c1F] |
| VI-44 | 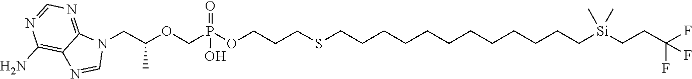 [SMILES: C[C@H](Cn1cnc2c(N)ncnc21)OCP(=O)(O)OCCCSCCCCCCCCCCCC[Si](C)(C)CCC(F)(F)F] |
| VI-45 | 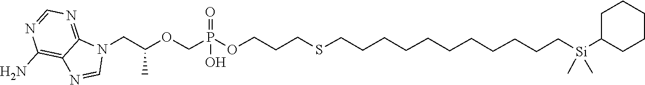 [SMILES: C[C@H](Cn1cnc2c(N)ncnc21)OCP(=O)(O)OCCCSCCCCCCCCCCC[Si](C)(C)C1CCCCC1] |
| VI-46 | 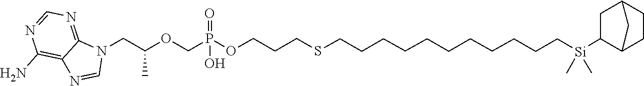 [SMILES: C[C@H](Cn1cnc2c(N)ncnc21)OCP(=O)(O)OCCCSCCCCCCCCCCC[Si](C)(C)C1CC2CCC1C2] |
| VI-47 | 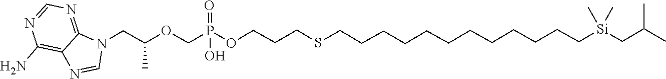 [SMILES: CC(C)C[Si](C)(C)CCCCCCCCCCCCSCCCOP(=O)(O)CO[C@H](C)Cn1cnc2c(N)ncnc21] |
| VI-48 | 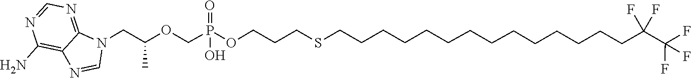 [SMILES: C[C@H](Cn1cnc2c(N)ncnc21)OCP(=O)(O)OCCCSCCCCCCCCCCCCCCC(F)(F)C(F)(F)F] |
| VI-49 | 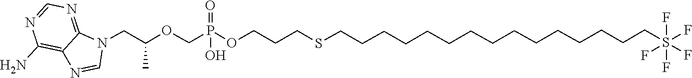 [SMILES: C[C@H](Cn1cnc2c(N)ncnc21)OCP(=O)(O)OCCCSCCCCCCCCCCCCCCCS(F)(F)(F)(F)F] |
| VI-50 | 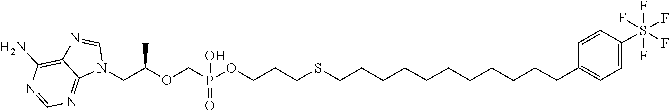 [SMILES: C[C@H](Cn1cnc2c(N)ncnc21)OCP(=O)(O)OCCCSCCCCCCCCCCCc1ccc(S(F)(F)(F)(F)F)cc1] |
| VI-51 | 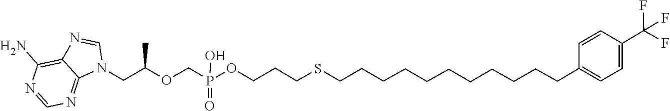 [SMILES: C[C@H](Cn1cnc2c(N)ncnc21)OCP(=O)(O)OCCCSCCCCCCCCCCCc1ccc(C(F)(F)F)cc1] |
| VI-52 | 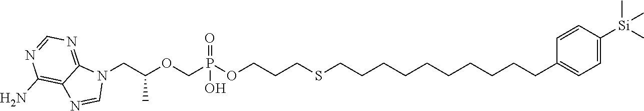 [SMILES: C[C@H](Cn1cnc2c(N)ncnc21)OCP(=O)(O)OCCCSCCCCCCCCCCc1ccc([Si](C)(C)C)cc1] |
| VI-53 | 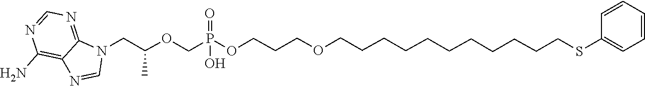 [SMILES: C[C@H](Cn1cnc2c(N)ncnc21)OCP(=O)(O)OCCCOCCCCCCCCCCCSc1ccccc1] |
| VI-54 | 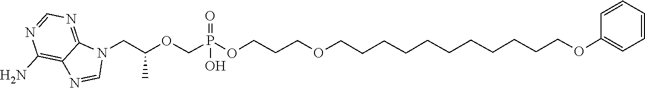 [SMILES: C[C@H](Cn1cnc2c(N)ncnc21)OCP(=O)(O)OCCCOCCCCCCCCCCCOc1ccccc1] |
| VI-55 | 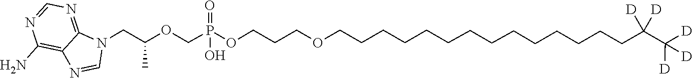 [SMILES: [2H]C([2H])([2H])C([2H])([2H])CCCCCCCCCCCCCCOCCCOP(=O)(O)CO[C@H](C)Cn1cnc2c(N)ncnc21] |
| VI-56 | 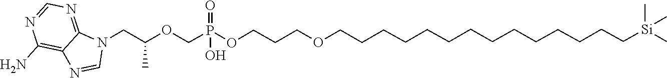 [SMILES: C[C@H](Cn1cnc2c(N)ncnc21)OCP(=O)(O)OCCCOCCCCCCCCCCCCCC[Si](C)(C)C] |
| VI-57 | 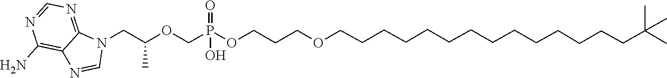 [SMILES: C[C@H](Cn1cnc2c(N)ncnc21)OCP(=O)(O)OCCCOCCCCCCCCCCCCCCC(C)(C)C] |
| VI-58 | 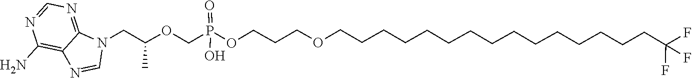 [SMILES: C[C@H](Cn1cnc2c(N)ncnc21)OCP(=O)(O)OCCCOCCCCCCCCCCCCCCCC(F)(F)F] |
| VI-59 | 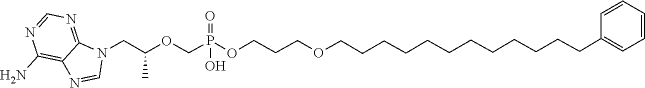 [SMILES: C[C@H](Cn1cnc2c(N)ncnc21)OCP(=O)(O)OCCCOCCCCCCCCCCCCc1ccccc1] |
| VI-60 | 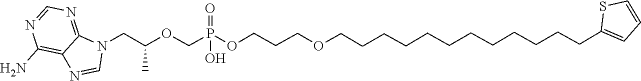 [SMILES: C[C@H](Cn1cnc2c(N)ncnc21)OCP(=O)(O)OCCCOCCCCCCCCCCCCc1cccs1] |
| VI-61 | 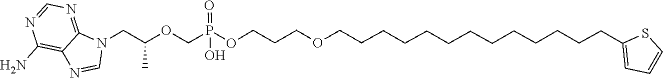 [SMILES: C[C@H](Cn1cnc2c(N)ncnc21)OCP(=O)(O)OCCCOCCCCCCCCCCCCCc1cccs1] |
| VI-62 | 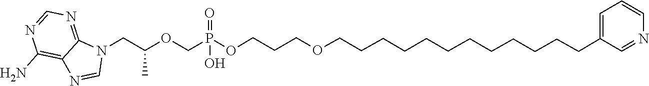 [SMILES: C[C@H](Cn1cnc2c(N)ncnc21)OCP(=O)(O)OCCCOCCCCCCCCCCCCc1cccnc1] |
| VI-63 | 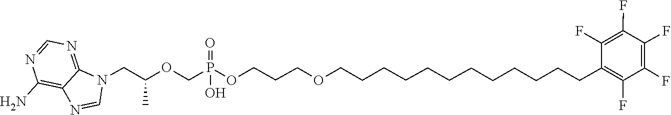 [SMILES: C[C@H](Cn1cnc2c(N)ncnc21)OCP(=O)(O)OCCCOCCCCCCCCCCCCc1c(F)c(F)c(F)c(F)c1F] |
| VI-64 | 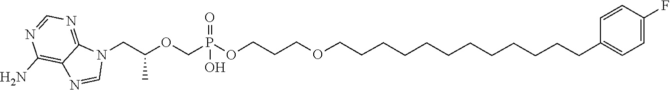 [SMILES: C[C@H](Cn1cnc2c(N)ncnc21)OCP(=O)(O)OCCCOCCCCCCCCCCCCc1ccc(F)cc1] |
| VI-65 | 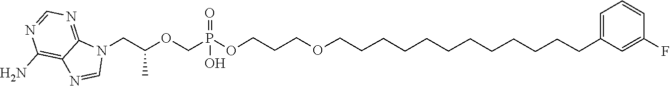 [SMILES: C[C@H](Cn1cnc2c(N)ncnc21)OCP(=O)(O)OCCCOCCCCCCCCCCCCc1cccc(F)c1] |
| VI-66 | 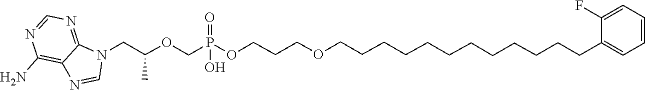 [SMILES: C[C@H](Cn1cnc2c(N)ncnc21)OCP(=O)(O)OCCCOCCCCCCCCCCCCc1ccccc1F] |
| VI-67 | 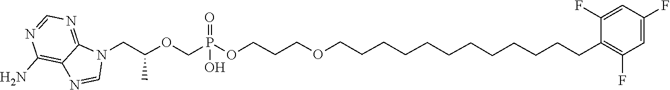 [SMILES: C[C@H](Cn1cnc2c(N)ncnc21)OCP(=O)(O)OCCCOCCCCCCCCCCCCc1c(F)cc(F)cc1F] |
| VI-68 | 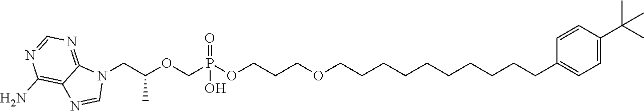 [SMILES: C[C@H](Cn1cnc2c(N)ncnc21)OCP(=O)(O)OCCCOCCCCCCCCCCc1ccc(C(C)(C)C)cc1] |
| VI-69 | 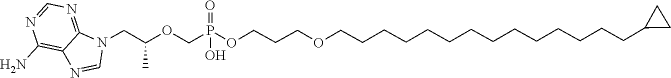 [SMILES: C[C@H](Cn1cnc2c(N)ncnc21)OCP(=O)(O)OCCCOCCCCCCCCCCCCCCC1CC1] |
| VI-70 | 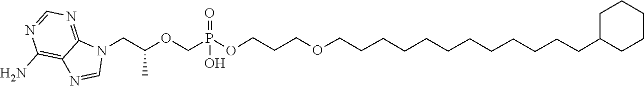 [SMILES: C[C@H](Cn1cnc2c(N)ncnc21)OCP(=O)(O)OCCCOCCCCCCCCCCCCC1CCCCC1] |
| VI-71 | 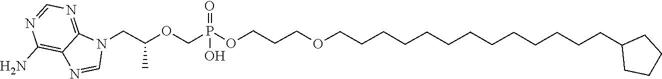 [SMILES: C[C@H](Cn1cnc2c(N)ncnc21)OCP(=O)(O)OCCCOCCCCCCCCCCCCCC1CCCC1] |
| VI-72 | 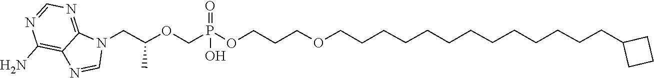 [SMILES: C[C@H](Cn1cnc2c(N)ncnc21)OCP(=O)(O)OCCCOCCCCCCCCCCCCCC1CCC1] |
| VI-73 | 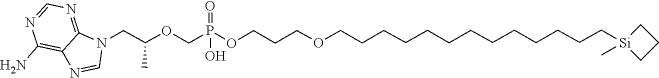 [SMILES: C[C@H](Cn1cnc2c(N)ncnc21)OCP(=O)(O)OCCCOCCCCCCCCCCCCC[Si]1(C)CCC1] |
| VI-74 | 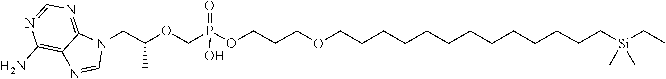 [SMILES: CC[Si](C)(C)CCCCCCCCCCCCCOCCCOP(=O)(O)CO[C@H](C)Cn1cnc2c(N)ncnc21] |
| VI-75 | 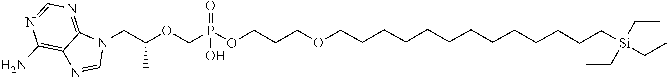 [SMILES: CC[Si](CC)(CC)CCCCCCCCCCCCCOCCCOP(=O)(O)CO[C@H](C)Cn1cnc2c(N)ncnc21] |
| VI-76 | 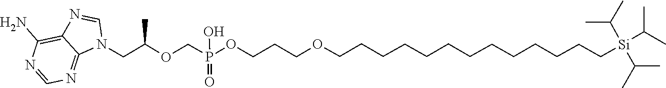 [SMILES: CC(C)[Si](CCCCCCCCCCCCCOCCCOP(=O)(O)CO[C@H](C)Cn1cnc2c(N)ncnc21)(C(C)C)C(C)C] |
| VI-77 | 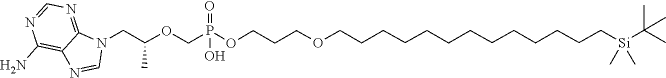 [SMILES: C[C@H](Cn1cnc2c(N)ncnc21)OCP(=O)(O)OCCCOCCCCCCCCCCCCC[Si](C)(C)C(C)(C)C] |
| VI-78 | 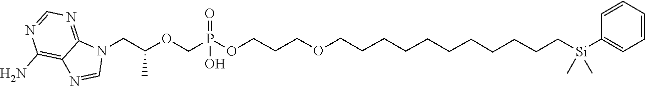 [SMILES: C[C@H](Cn1cnc2c(N)ncnc21)OCP(=O)(O)OCCCOCCCCCCCCCCC[Si](C)(C)c1ccccc1] |
| VI-79 | 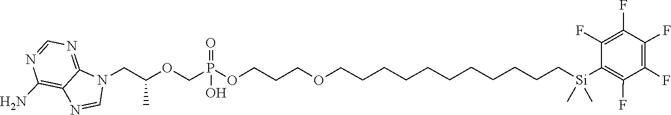 [SMILES: C[C@H](Cn1cnc2c(N)ncnc21)OCP(=O)(O)OCCCOCCCCCCCCCCC[Si](C)(C)c1c(F)c(F)c(F)c(F)c1F] |
| VI-80 | 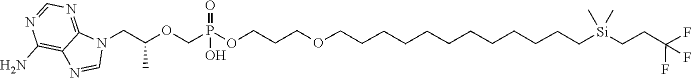 [SMILES: C[C@H](Cn1cnc2c(N)ncnc21)OCP(=O)(O)OCCCOCCCCCCCCCCCC[Si](C)(C)CCC(F)(F)F] |
| VI-81 | 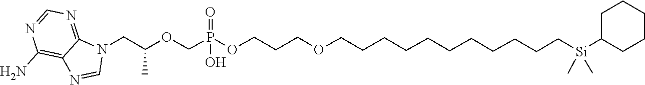 [SMILES: C[C@H](Cn1cnc2c(N)ncnc21)OCP(=O)(O)OCCCOCCCCCCCCCCC[Si](C)(C)C1CCCCC1] |
| VI-82 | 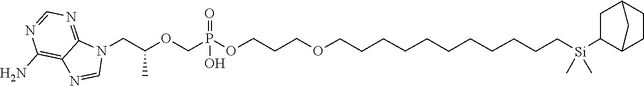 [SMILES: C[C@H](Cn1cnc2c(N)ncnc21)OCP(=O)(O)OCCCOCCCCCCCCCCC[Si](C)(C)C1CC2CCC1C2] |
| VI-83 | 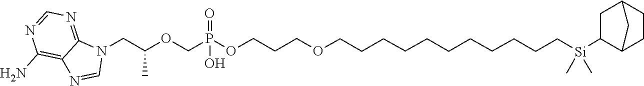 [SMILES: C[C@H](Cn1cnc2c(N)ncnc21)OCP(=O)(O)OCCCOCCCCCCCCCCC[Si](C)(C)C1CC2CCC1C2] |
| VI-84 | 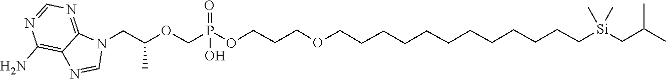 [SMILES: CC(C)C[Si](C)(C)CCCCCCCCCCCCOCCCOP(=O)(O)CO[C@H](C)Cn1cnc2c(N)ncnc21] |
| VI-85 | 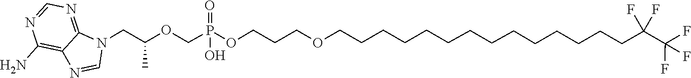 [SMILES: C[C@H](Cn1cnc2c(N)ncnc21)OCP(=O)(O)OCCCOCCCCCCCCCCCCCCC(F)(F)C(F)(F)F] |
| VI-86 | 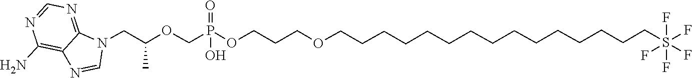 [SMILES: C[C@H](Cn1cnc2c(N)ncnc21)OCP(=O)(O)OCCCOCCCCCCCCCCCCCCCS(F)(F)(F)(F)F] |
| VI-87 | 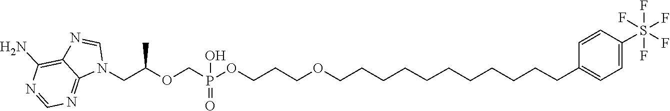 [SMILES: C[C@H](Cn1cnc2c(N)ncnc21)OCP(=O)(O)OCCCOCCCCCCCCCCCc1ccc(S(F)(F)(F)(F)F)cc1] |
| VI-88 | 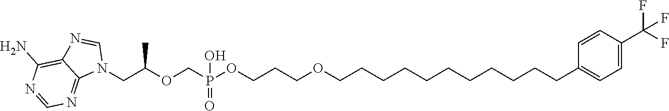 [SMILES: C[C@H](Cn1cnc2c(N)ncnc21)OCP(=O)(O)OCCCOCCCCCCCCCCCc1ccc(C(F)(F)F)cc1] |
| VI-89 | 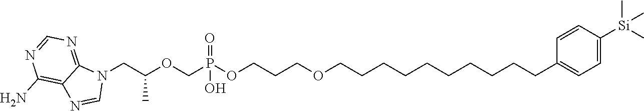 [SMILES: C[C@H](Cn1cnc2c(N)ncnc21)OCP(=O)(O)OCCCOCCCCCCCCCCc1ccc([Si](C)(C)C)cc1] |
| VI-90 | 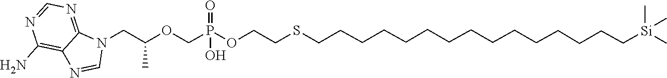 [SMILES: C[C@H](Cn1cnc2c(N)ncnc21)OCP(=O)(O)OCCSCCCCCCCCCCCCCCC[Si](C)(C)C] |
| VI-91 | 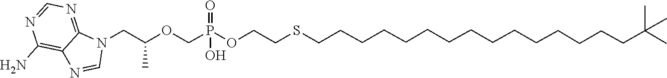 [SMILES: C[C@H](Cn1cnc2c(N)ncnc21)OCP(=O)(O)OCCSCCCCCCCCCCCCCCCC(C)(C)C] |
| VI-92 | 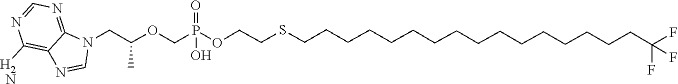 [SMILES: C[C@H](Cn1cnc2c(N)ncnc21)OCP(=O)(O)OCCSCCCCCCCCCCCCCCCCC(F)(F)F] |
| VI-93 | 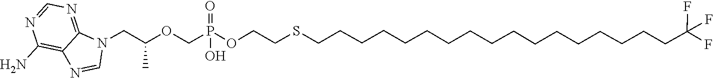 [SMILES: C[C@H](Cn1cnc2c(N)ncnc21)OCP(=O)(O)OCCSCCCCCCCCCCCCCCCCCC(F)(F)F] |
| VI-94 | 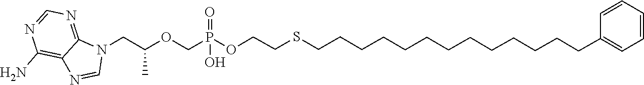 [SMILES: C[C@H](Cn1cnc2c(N)ncnc21)OCP(=O)(O)OCCSCCCCCCCCCCCCCc1ccccc1] |
| VI-95 | 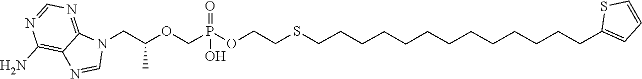 [SMILES: C[C@H](Cn1cnc2c(N)ncnc21)OCP(=O)(O)OCCSCCCCCCCCCCCCCc1cccs1] |
| VI-96 | 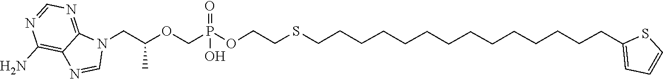 [SMILES: C[C@H](Cn1cnc2c(N)ncnc21)OCP(=O)(O)OCCSCCCCCCCCCCCCCCc1cccs1] |
| VI-97 | 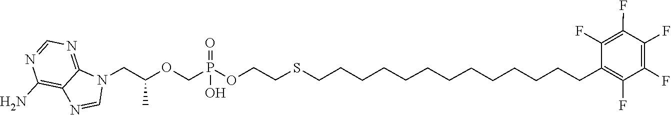 [SMILES: C[C@H](Cn1cnc2c(N)ncnc21)OCP(=O)(O)OCCSCCCCCCCCCCCCCc1c(F)c(F)c(F)c(F)c1F] |
| VI-98 | 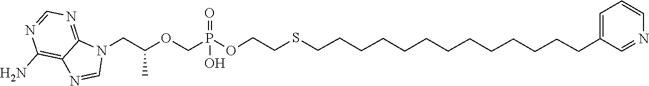 [SMILES: C[C@H](Cn1cnc2c(N)ncnc21)OCP(=O)(O)OCCSCCCCCCCCCCCCCc1cccnc1] |
| VI-99 | 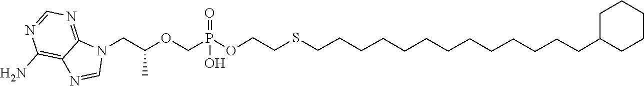 [SMILES: C[C@H](Cn1cnc2c(N)ncnc21)OCP(=O)(O)OCCSCCCCCCCCCCCCCC1CCCCC1] |
| VI-100 | 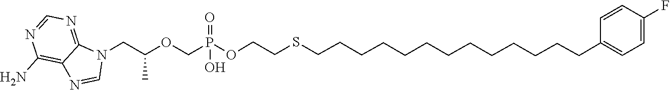 [SMILES: C[C@H](Cn1cnc2c(N)ncnc21)OCP(=O)(O)OCCSCCCCCCCCCCCCCc1ccc(F)cc1] |
| VI-101 | 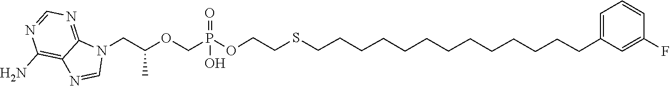 [SMILES: C[C@H](Cn1cnc2c(N)ncnc21)OCP(=O)(O)OCCSCCCCCCCCCCCCCc1cccc(F)c1] |
| VI-102 | 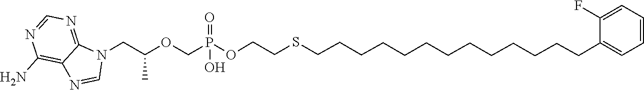 [SMILES: C[C@H](Cn1cnc2c(N)ncnc21)OCP(=O)(O)OCCSCCCCCCCCCCCCCc1ccccc1F] |
| VI-103 | 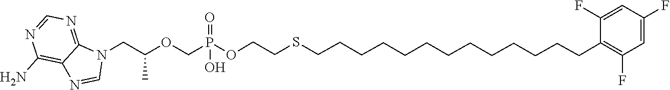 [SMILES: C[C@H](Cn1cnc2c(N)ncnc21)OCP(=O)(O)OCCSCCCCCCCCCCCCCc1c(F)cc(F)cc1F] |
| VI-104 | 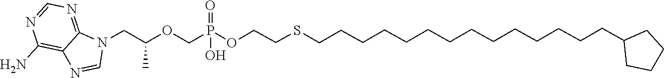 [SMILES: C[C@H](Cn1cnc2c(N)ncnc21)OCP(=O)(O)OCCSCCCCCCCCCCCCCCC1CCCC1] |
| VI-105 | 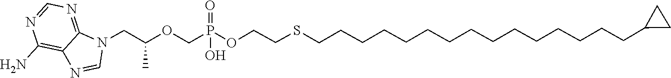 [SMILES: C[C@H](Cn1cnc2c(N)ncnc21)OCP(=O)(O)OCCSCCCCCCCCCCCCCCCC1CC1] |
| VI-106 | 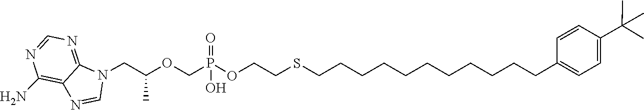 [SMILES: C[C@H](Cn1cnc2c(N)ncnc21)OCP(=O)(O)OCCSCCCCCCCCCCCc1ccc(C(C)(C)C)cc1] |
| VI-107 | 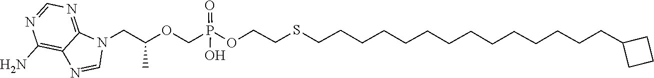 [SMILES: C[C@H](Cn1cnc2c(N)ncnc21)OCP(=O)(O)OCCSCCCCCCCCCCCCCCC1CCC1] |
| VI-108 | 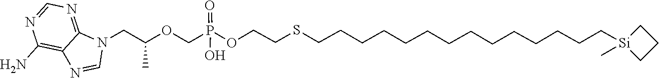 [SMILES: C[C@H](Cn1cnc2c(N)ncnc21)OCP(=O)(O)OCCSCCCCCCCCCCCCCC[Si]1(C)CCC1] |
| VI-109 | 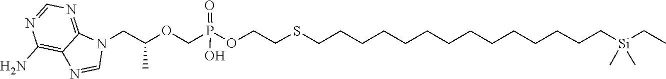 [SMILES: CC[Si](C)(C)CCCCCCCCCCCCCCSCCOP(=O)(O)CO[C@H](C)Cn1cnc2c(N)ncnc21] |
| VI-110 | 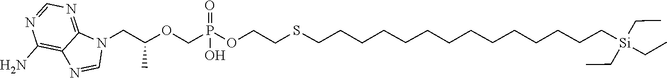 [SMILES: CC[Si](CC)(CC)CCCCCCCCCCCCCCSCCOP(=O)(O)CO[C@H](C)Cn1cnc2c(N)ncnc21] |
| VI-111 | 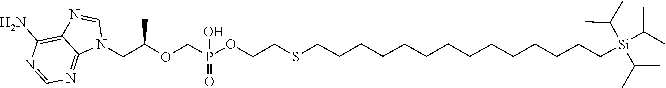 [SMILES: CC(C)[Si](CCCCCCCCCCCCCCSCCOP(=O)(O)CO[C@H](C)Cn1cnc2c(N)ncnc21)(C(C)C)C(C)C] |
| VI-112 | 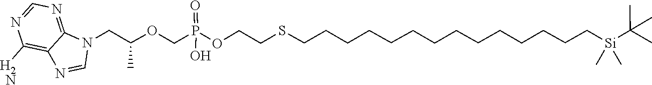 [SMILES: C[C@H](Cn1cnc2c(N)ncnc21)OCP(=O)(O)OCCSCCCCCCCCCCCCCC[Si](C)(C)C(C)(C)C] |
| VI-113 | 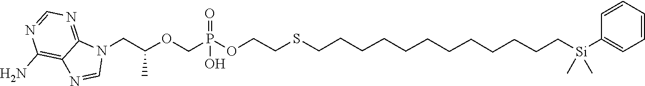 [SMILES: C[C@H](Cn1cnc2c(N)ncnc21)OCP(=O)(O)OCCSCCCCCCCCCCCC[Si](C)(C)c1ccccc1] |
| VI-114 | 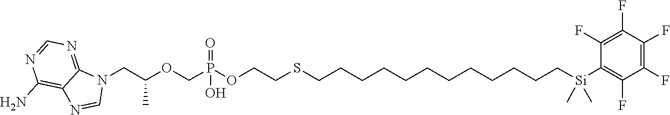 [SMILES: C[C@H](Cn1cnc2c(N)ncnc21)OCP(=O)(O)OCCSCCCCCCCCCCCC[Si](C)(C)c1c(F)c(F)c(F)c(F)c1F] |
| VI-115 | 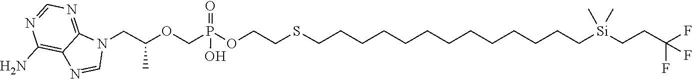 [SMILES: C[C@H](Cn1cnc2c(N)ncnc21)OCP(=O)(O)OCCSCCCCCCCCCCCCC[Si](C)(C)CCC(F)(F)F] |
| VI-116 | 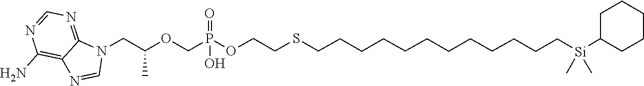 [SMILES: C[C@H](Cn1cnc2c(N)ncnc21)OCP(=O)(O)OCCSCCCCCCCCCCCC[Si](C)(C)C1CCCCC1] |
| VI-117 | 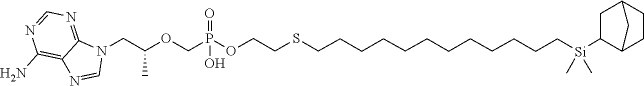 [SMILES: C[C@H](Cn1cnc2c(N)ncnc21)OCP(=O)(O)OCCSCCCCCCCCCCCC[Si](C)(C)C1CC2CCC1C2] |
| VI-118 | 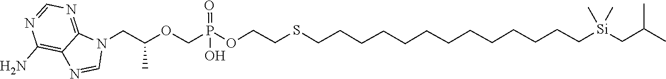 [SMILES: CC(C)C[Si](C)(C)CCCCCCCCCCCCCSCCOP(=O)(O)CO[C@H](C)Cn1cnc2c(N)ncnc21] |
| VI-119 | 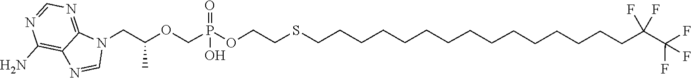 [SMILES: C[C@H](Cn1cnc2c(N)ncnc21)OCP(=O)(O)OCCSCCCCCCCCCCCCCCCC(F)(F)C(F)(F)F] |
| VI-120 | 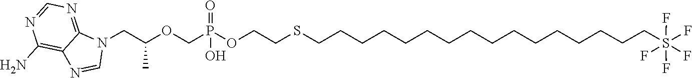 [SMILES: C[C@H](Cn1cnc2c(N)ncnc21)OCP(=O)(O)OCCSCCCCCCCCCCCCCCCCS(F)(F)(F)(F)F] |
| VI-121 | 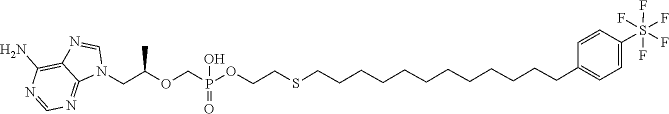 [SMILES: C[C@H](Cn1cnc2c(N)ncnc21)OCP(=O)(O)OCCSCCCCCCCCCCCCc1ccc(S(F)(F)(F)(F)F)cc1] |
| VI-122 | 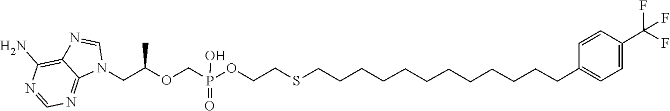 [SMILES: C[C@H](Cn1cnc2c(N)ncnc21)OCP(=O)(O)OCCSCCCCCCCCCCCCc1ccc(C(F)(F)F)cc1] |
| VI-123 | 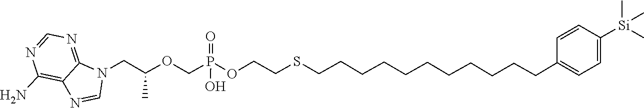 [SMILES: C[C@H](Cn1cnc2c(N)ncnc21)OCP(=O)(O)OCCSCCCCCCCCCCCc1ccc([Si](C)(C)C)cc1] |
| VI-124 | 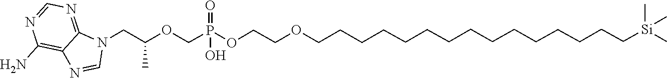 [SMILES: C[C@H](Cn1cnc2c(N)ncnc21)OCP(=O)(O)OCCOCCCCCCCCCCCCCCC[Si](C)(C)C] |
| VI-125 | 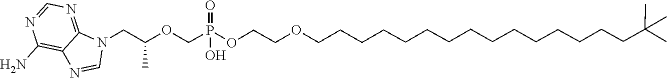 [SMILES: C[C@H](Cn1cnc2c(N)ncnc21)OCP(=O)(O)OCCOCCCCCCCCCCCCCCCC(C)(C)C] |
| VI-126 | 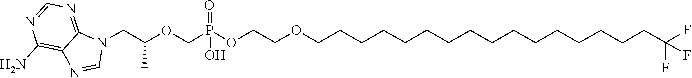 [SMILES: C[C@H](Cn1cnc2c(N)ncnc21)OCP(=O)(O)OCCOCCCCCCCCCCCCCCCCC(F)(F)F] |
| VI-127 | 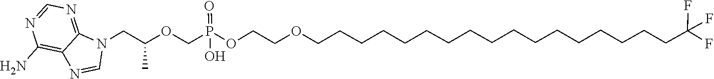 [SMILES: C[C@H](Cn1cnc2c(N)ncnc21)OCP(=O)(O)OCCOCCCCCCCCCCCCCCCCCC(F)(F)F] |
| VI-128 | 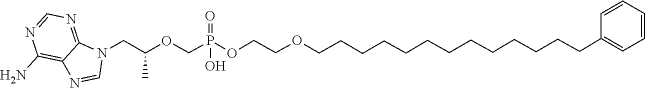 [SMILES: C[C@H](Cn1cnc2c(N)ncnc21)OCP(=O)(O)OCCOCCCCCCCCCCCCCc1ccccc1] |
| VI-129 | 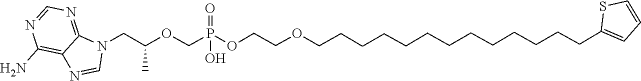 [SMILES: C[C@H](Cn1cnc2c(N)ncnc21)OCP(=O)(O)OCCOCCCCCCCCCCCCCc1cccs1] |
| VI-130 | 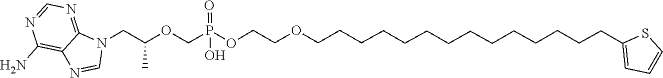 [SMILES: C[C@H](Cn1cnc2c(N)ncnc21)OCP(=O)(O)OCCOCCCCCCCCCCCCCCc1cccs1] |
| VI-131 | 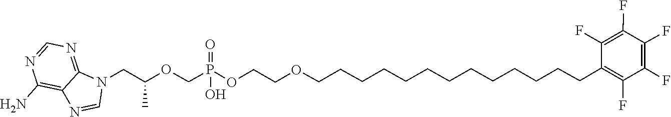 [SMILES: C[C@H](Cn1cnc2c(N)ncnc21)OCP(=O)(O)OCCOCCCCCCCCCCCCCc1c(F)c(F)c(F)c(F)c1F] |
| VI-132 | 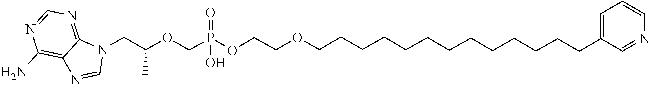 [SMILES: C[C@H](Cn1cnc2c(N)ncnc21)OCP(=O)(O)OCCOCCCCCCCCCCCCCc1cccnc1] |
| VI-133 | 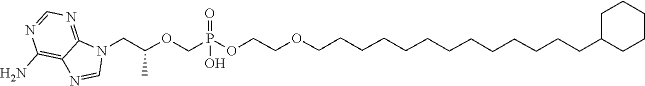 [SMILES: C[C@H](Cn1cnc2c(N)ncnc21)OCP(=O)(O)OCCOCCCCCCCCCCCCCC1CCCCC1] |
| VI-134 | 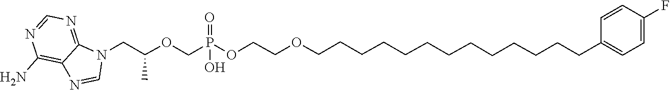 [SMILES: C[C@H](Cn1cnc2c(N)ncnc21)OCP(=O)(O)OCCOCCCCCCCCCCCCCc1ccc(F)cc1] |
| VI-135 | 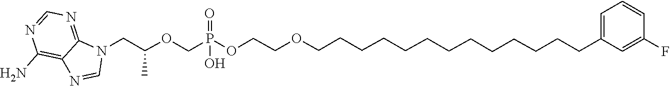 [SMILES: C[C@H](Cn1cnc2c(N)ncnc21)OCP(=O)(O)OCCOCCCCCCCCCCCCCc1cccc(F)c1] |
| VI-136 | 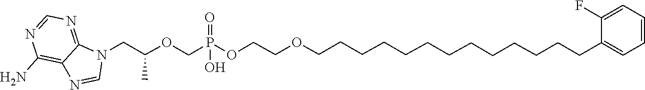 [SMILES: C[C@H](Cn1cnc2c(N)ncnc21)OCP(=O)(O)OCCOCCCCCCCCCCCCCc1ccccc1F] |
| VI-137 | 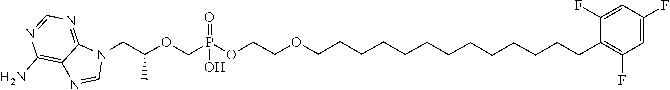 [SMILES: C[C@H](Cn1cnc2c(N)ncnc21)OCP(=O)(O)OCCOCCCCCCCCCCCCCc1c(F)cc(F)cc1F] |
| VI-138 | 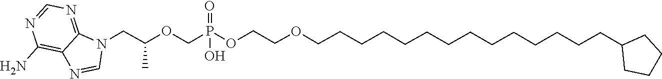 [SMILES: C[C@H](Cn1cnc2c(N)ncnc21)OCP(=O)(O)OCCOCCCCCCCCCCCCCCC1CCCC1] |
| VI-139 | 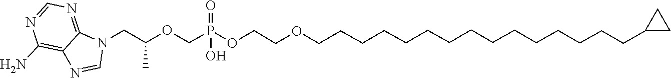 [SMILES: C[C@H](Cn1cnc2c(N)ncnc21)OCP(=O)(O)OCCOCCCCCCCCCCCCCCCC1CC1] |
| VI-140 | 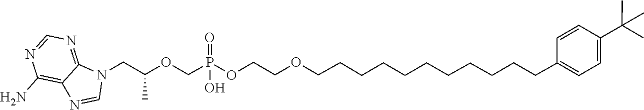 [SMILES: C[C@H](Cn1cnc2c(N)ncnc21)OCP(=O)(O)OCCOCCCCCCCCCCCc1ccc(C(C)(C)C)cc1] |
| VI-141 | 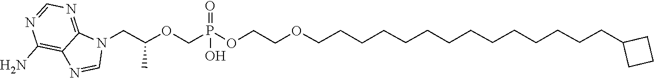 [SMILES: C[C@H](Cn1cnc2c(N)ncnc21)OCP(=O)(O)OCCOCCCCCCCCCCCCCCC1CCC1] |
| VI-142 | 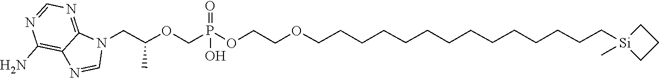 [SMILES: C[C@H](Cn1cnc2c(N)ncnc21)OCP(=O)(O)OCCOCCCCCCCCCCCCCC[Si]1(C)CCC1] |
| VI-143 | 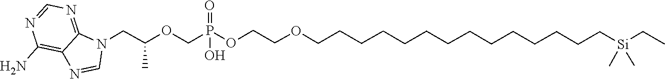 [SMILES: CC[Si](C)(C)CCCCCCCCCCCCCCOCCOP(=O)(O)CO[C@H](C)Cn1cnc2c(N)ncnc21] |
| VI-144 | 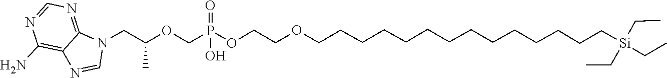 [SMILES: CC[Si](CC)(CC)CCCCCCCCCCCCCCOCCOP(=O)(O)CO[C@H](C)Cn1cnc2c(N)ncnc21] |
| VI-145 | 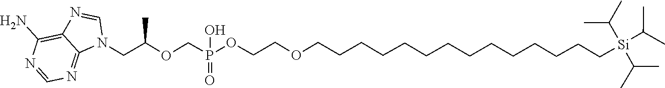 [SMILES: CC(C)[Si](CCCCCCCCCCCCCCOCCOP(=O)(O)CO[C@H](C)Cn1cnc2c(N)ncnc21)(C(C)C)C(C)C] |
| VI-146 | 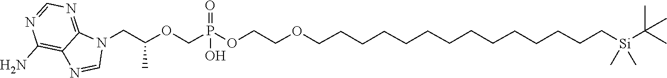 [SMILES: C[C@H](Cn1cnc2c(N)ncnc21)OCP(=O)(O)OCCOCCCCCCCCCCCCCC[Si](C)(C)C(C)(C)C] |
| VI-147 | 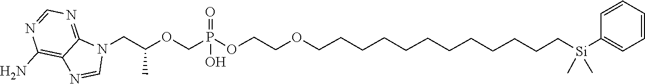 [SMILES: C[C@H](Cn1cnc2c(N)ncnc21)OCP(=O)(O)OCCOCCCCCCCCCCCC[Si](C)(C)c1ccccc1] |
| VI-148 | 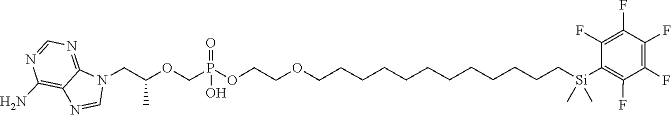 [SMILES: C[C@H](Cn1cnc2c(N)ncnc21)OCP(=O)(O)OCCOCCCCCCCCCCCC[Si](C)(C)c1c(F)c(F)c(F)c(F)c1F] |
| VI-149 | 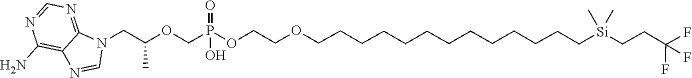 [SMILES: C[C@H](Cn1cnc2c(N)ncnc21)OCP(=O)(O)OCCOCCCCCCCCCCCCC[Si](C)(C)CCC(F)(F)F] |
| VI-150 | 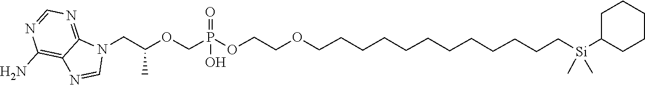 [SMILES: C[C@H](Cn1cnc2c(N)ncnc21)OCP(=O)(O)OCCOCCCCCCCCCCCC[Si](C)(C)C1CCCCC1] |
| VI-151 | 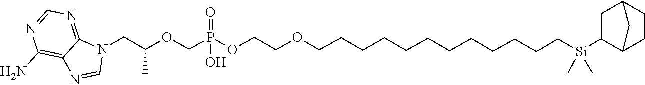 [SMILES: C[C@H](Cn1cnc2c(N)ncnc21)OCP(=O)(O)OCCOCCCCCCCCCCCC[Si](C)(C)C1CC2CCC1C2] |
| VI-152 | 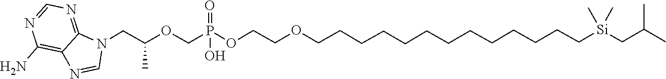 [SMILES: CC(C)C[Si](C)(C)CCCCCCCCCCCCCOCCOP(=O)(O)CO[C@H](C)Cn1cnc2c(N)ncnc21] |
| VI-153 | 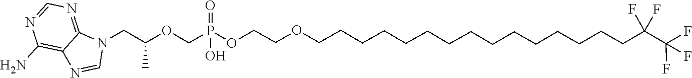 [SMILES: C[C@H](Cn1cnc2c(N)ncnc21)OCP(=O)(O)OCCOCCCCCCCCCCCCCCCC(F)(F)C(F)(F)F] |
| VI-154 | 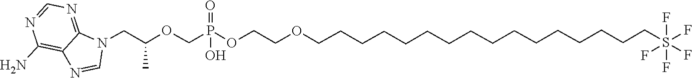 [SMILES: C[C@H](Cn1cnc2c(N)ncnc21)OCP(=O)(O)OCCOCCCCCCCCCCCCCCCCS(F)(F)(F)(F)F] |
| VI-155 | 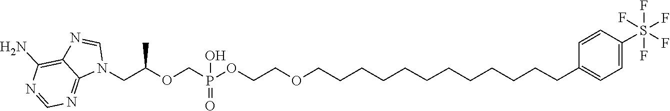 [SMILES: C[C@H](Cn1cnc2c(N)ncnc21)OCP(=O)(O)OCCOCCCCCCCCCCCCc1ccc(S(F)(F)(F)(F)F)cc1] |
| VI-156 | 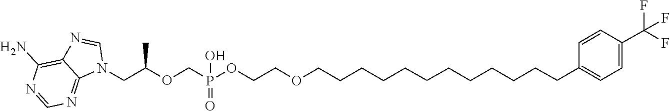 [SMILES: C[C@H](Cn1cnc2c(N)ncnc21)OCP(=O)(O)OCCOCCCCCCCCCCCCc1ccc(C(F)(F)F)cc1] |
| VI-157 | 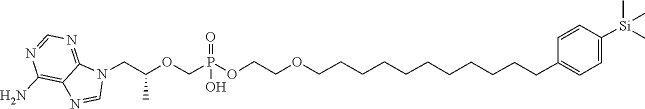 [SMILES: C[C@H](Cn1cnc2c(N)ncnc21)OCP(=O)(O)OCCOCCCCCCCCCCCc1ccc([Si](C)(C)C)cc1] |
| VI-158 | 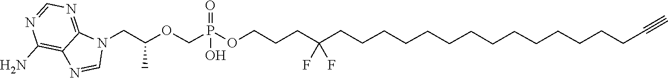 [SMILES: C#CCCCCCCCCCCCCCCC(F)(F)CCCOP(=O)(O)CO[C@H](C)Cn1cnc2c(N)ncnc21] |
| VI-159 | 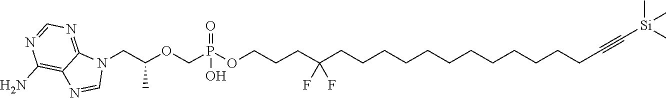 [SMILES: C[C@H](Cn1cnc2c(N)ncnc21)OCP(=O)(O)OCCCC(F)(F)CCCCCCCCCCCCC#C[Si](C)(C)C] |
| VI-160 | 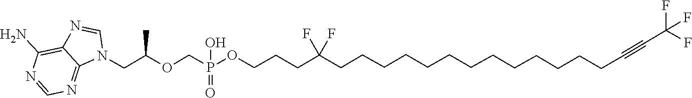 [SMILES: C[C@H](Cn1cnc2c(N)ncnc21)OCP(=O)(O)OCCCC(F)(F)CCCCCCCCCCCCCC#CC(F)(F)F] |
| VI-161 | 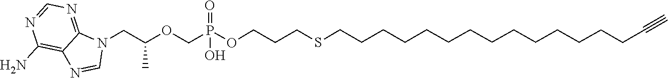 [SMILES: C#CCCCCCCCCCCCCCCSCCCOP(=O)(O)CO[C@H](C)Cn1cnc2c(N)ncnc21] |
| VI-162 | 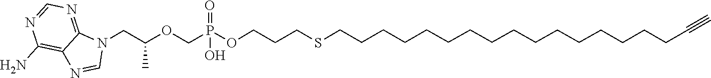 [SMILES: C#CCCCCCCCCCCCCCCCCSCCCOP(=O)(O)CO[C@H](C)Cn1cnc2c(N)ncnc21] |
| VI-163 | 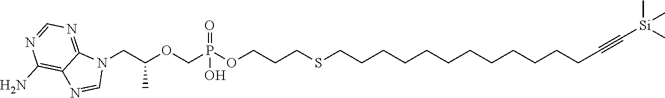 [SMILES: C[C@H](Cn1cnc2c(N)ncnc21)OCP(=O)(O)OCCCSCCCCCCCCCCCCC#C[Si](C)(C)C] |
| VI-164 | 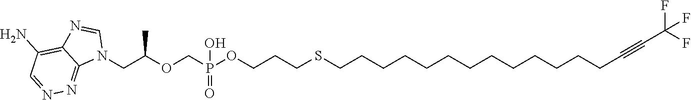 [SMILES: C[C@H](Cn1cnc2c(N)cnnc21)OCP(=O)(O)OCCCSCCCCCCCCCCCCCC#CC(F)(F)F] |
| VI-165 | 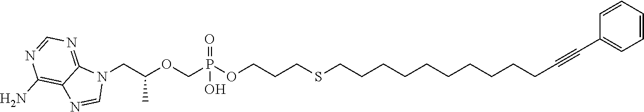 [SMILES: C[C@H](Cn1cnc2c(N)ncnc21)OCP(=O)(O)OCCCSCCCCCCCCCCC#Cc1ccccc1] |
| VI-166 | 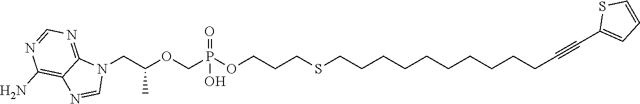 [SMILES: C[C@H](Cn1cnc2c(N)ncnc21)OCP(=O)(O)OCCCSCCCCCCCCCCC#Cc1cccs1] |
| VI-167 | 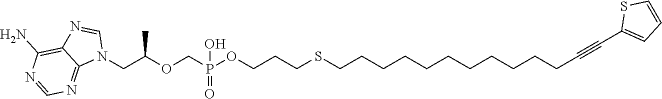 [SMILES: C[C@H](Cn1cnc2c(N)ncnc21)OCP(=O)(O)OCCCSCCCCCCCCCCCC#Cc1cccs1] |
| VI-168 | 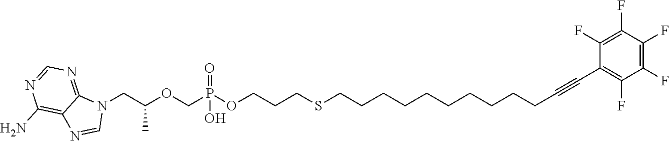 [SMILES: C[C@H](Cn1cnc2c(N)ncnc21)OCP(=O)(O)OCCCSCCCCCCCCCCC#Cc1c(F)c(F)c(F)c(F)c1F] |
| VI-169 | 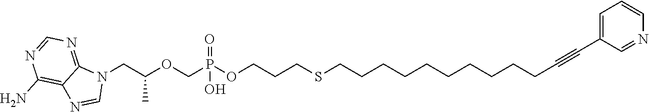 [SMILES: C[C@H](Cn1cnc2c(N)ncnc21)OCP(=O)(O)OCCCSCCCCCCCCCCC#Cc1cccnc1] |
| VI-170 | 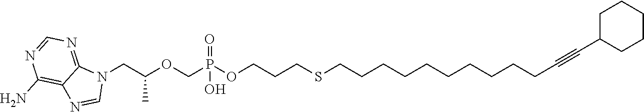 [SMILES: C[C@H](Cn1cnc2c(N)ncnc21)OCP(=O)(O)OCCCSCCCCCCCCCCC#CC1CCCCC1] |
| VI-171 | 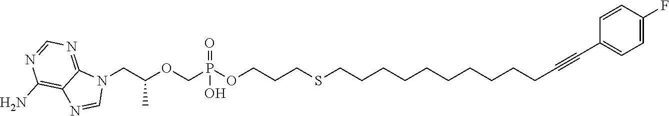 [SMILES: C[C@H](Cn1cnc2c(N)ncnc21)OCP(=O)(O)OCCCSCCCCCCCCCCC#Cc1ccc(F)cc1] |
| VI-172 | 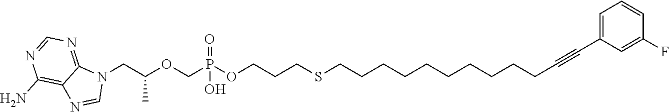 [SMILES: C[C@H](Cn1cnc2c(N)ncnc21)OCP(=O)(O)OCCCSCCCCCCCCCCC#Cc1cccc(F)c1] |
| VI-173 | 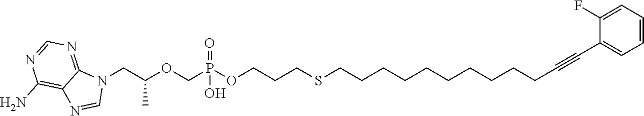 [SMILES: C[C@H](Cn1cnc2c(N)ncnc21)OCP(=O)(O)OCCCSCCCCCCCCCCC#Cc1ccccc1F] |
| VI-174 | 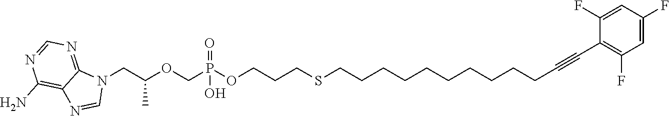 [SMILES: C[C@H](Cn1cnc2c(N)ncnc21)OCP(=O)(O)OCCCSCCCCCCCCCCC#Cc1c(F)cc(F)cc1F] |
| VI-175 | 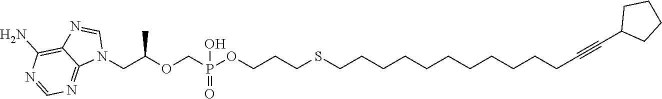 [SMILES: C[C@H](Cn1cnc2c(N)ncnc21)OCP(=O)(O)OCCCSCCCCCCCCCCCC#CC1CCCC1] |
| VI-176 | 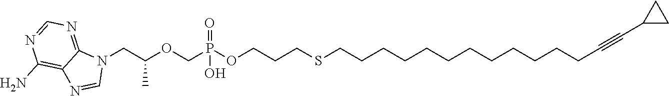 [SMILES: C[C@H](Cn1cnc2c(N)ncnc21)OCP(=O)(O)OCCCSCCCCCCCCCCCCC#CC1CC1] |
| VI-177 | 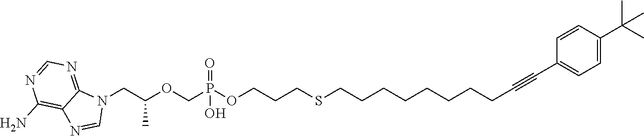 [SMILES: C[C@H](Cn1cnc2c(N)ncnc21)OCP(=O)(O)OCCCSCCCCCCCCC#Cc1ccc(C(C)(C)C)cc1] |
| VI-178 | 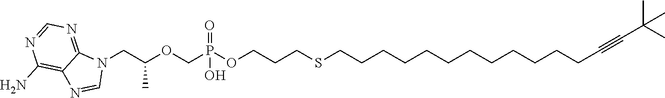 [SMILES: C[C@H](Cn1cnc2c(N)ncnc21)OCP(=O)(O)OCCCSCCCCCCCCCCCCC#CC(C)(C)C] |
| VI-179 | 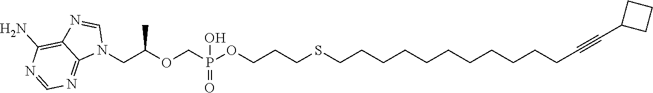 [SMILES: C[C@H](Cn1cnc2c(N)ncnc21)OCP(=O)(O)OCCCSCCCCCCCCCCCC#CC1CCC1] |
| VI-180 | 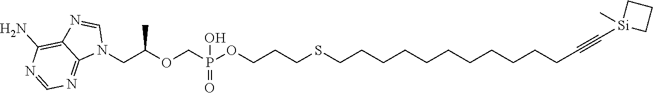 [SMILES: C[C@H](Cn1cnc2c(N)ncnc21)OCP(=O)(O)OCCCSCCCCCCCCCCCC#C[Si]1(C)CCC1] |
| VI-181 | 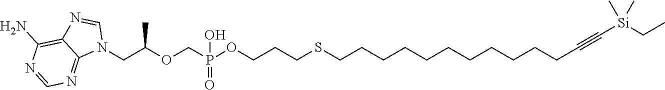 [SMILES: CC[Si](C)(C)C#CCCCCCCCCCCCSCCCOP(=O)(O)CO[C@H](C)Cn1cnc2c(N)ncnc21] |
| VI-182 | 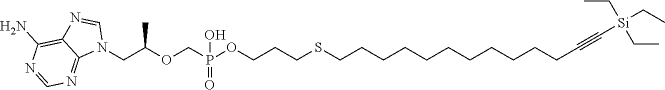 [SMILES: CC[Si](C#CCCCCCCCCCCCSCCCOP(=O)(O)CO[C@H](C)Cn1cnc2c(N)ncnc21)(CC)CC] |
| VI-183 | 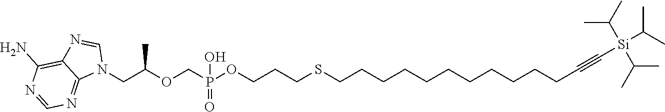 [SMILES: CC(C)[Si](C#CCCCCCCCCCCCSCCCOP(=O)(O)CO[C@H](C)Cn1cnc2c(N)ncnc21)(C(C)C)C(C)C] |
| VI-184 | 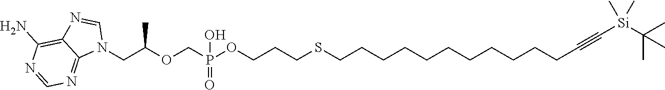 [SMILES: C[C@H](Cn1cnc2c(N)ncnc21)OCP(=O)(O)OCCCSCCCCCCCCCCCC#C[Si](C)(C)C(C)(C)C] |
| VI-185 | 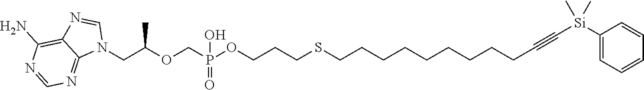 [SMILES: C[C@H](Cn1cnc2c(N)ncnc21)OCP(=O)(O)OCCCSCCCCCCCCCC#C[Si](C)(C)c1ccccc1] |
| VI-186 | 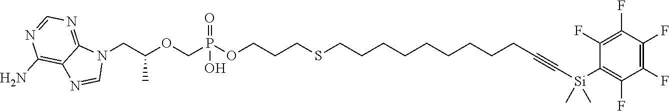 [SMILES: C[C@H](Cn1cnc2c(N)ncnc21)OCP(=O)(O)OCCCSCCCCCCCCCC#C[Si](C)(C)c1c(F)c(F)c(F)c(F)c1F] |
| VI-187 | 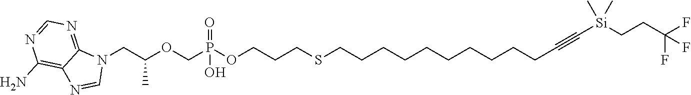 [SMILES: C[C@H](Cn1cnc2c(N)ncnc21)OCP(=O)(O)OCCCSCCCCCCCCCCC#C[Si](C)(C)CCC(F)(F)F] |
| VI-188 | 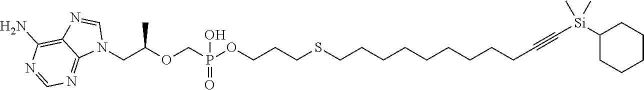 [SMILES: C[C@H](Cn1cnc2c(N)ncnc21)OCP(=O)(O)OCCCSCCCCCCCCCC#C[Si](C)(C)C1CCCCC1] |
| VI-189 | 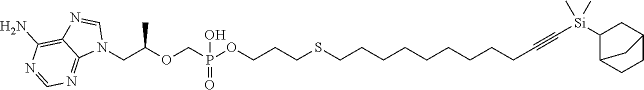 [SMILES: C[C@H](Cn1cnc2c(N)ncnc21)OCP(=O)(O)OCCCSCCCCCCCCCC#C[Si](C)(C)C1CC2CCC1C2] |
| VI-190 | 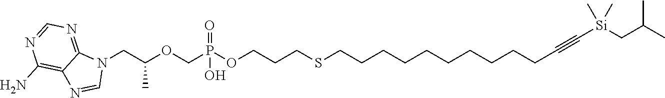 [SMILES: CC(C)C[Si](C)(C)C#CCCCCCCCCCCSCCCOP(=O)(O)CO[C@H](C)Cn1cnc2c(N)ncnc21] |
| VI-191 | 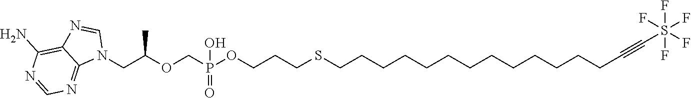 [SMILES: C[C@H](Cn1cnc2c(N)ncnc21)OCP(=O)(O)OCCCSCCCCCCCCCCCCCC#CS(F)(F)(F)(F)F] |
| VI-192 | 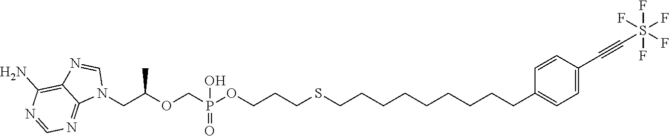 [SMILES: C[C@H](Cn1cnc2c(N)ncnc21)OCP(=O)(O)OCCCSCCCCCCCCCc1ccc(C#CS(F)(F)(F)(F)F)cc1] |
| VI-193 | 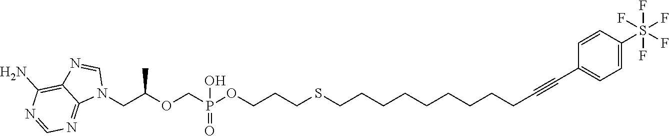 [SMILES: C[C@H](Cn1cnc2c(N)ncnc21)OCP(=O)(O)OCCCSCCCCCCCCCC#Cc1ccc(S(F)(F)(F)(F)F)cc1] |
| VI-194 | 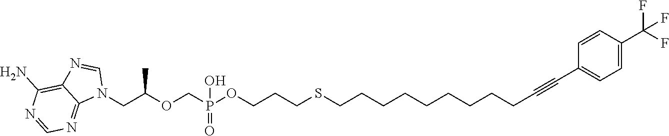 [SMILES: C[C@H](Cn1cnc2c(N)ncnc21)OCP(=O)(O)OCCCSCCCCCCCCCC#Cc1ccc(C(F)(F)F)cc1] |
| VI-195 | 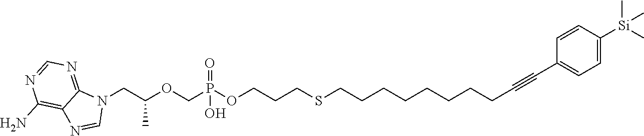 [SMILES: C[C@H](Cn1cnc2c(N)ncnc21)OCP(=O)(O)OCCCSCCCCCCCCC#Cc1ccc([Si](C)(C)C)cc1] |
| VI-196 | 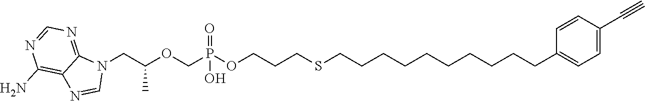 [SMILES: C#Cc1ccc(CCCCCCCCCCSCCCOP(=O)(O)CO[C@H](C)Cn2cnc3c(N)ncnc32)cc1] |
| VI-197 | 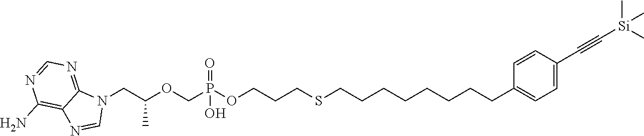 [SMILES: C[C@H](Cn1cnc2c(N)ncnc21)OCP(=O)(O)OCCCSCCCCCCCCc1ccc(C#C[Si](C)(C)C)cc1] |
| VI-198 | 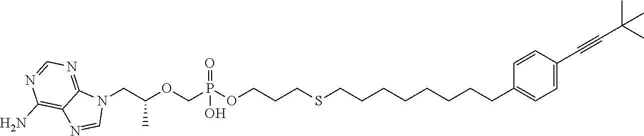 [SMILES: C[C@H](Cn1cnc2c(N)ncnc21)OCP(=O)(O)OCCCSCCCCCCCCc1ccc(C#CC(C)(C)C)cc1] |
| VI-199 | 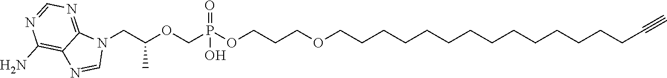 [SMILES: C#CCCCCCCCCCCCCCCOCCCOP(=O)(O)CO[C@H](C)Cn1cnc2c(N)ncnc21] |
| VI-200 | 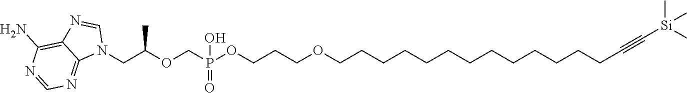 [SMILES: C[C@H](Cn1cnc2c(N)ncnc21)OCP(=O)(O)OCCCOCCCCCCCCCCCCCC#C[Si](C)(C)C] |
| VI-201 | 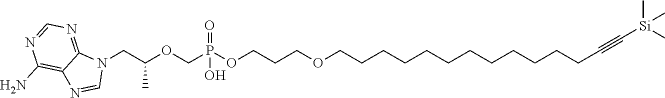 [SMILES: C[C@H](Cn1cnc2c(N)ncnc21)OCP(=O)(O)OCCCOCCCCCCCCCCCCC#C[Si](C)(C)C] |
| VI-202 | 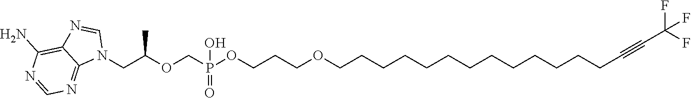 [SMILES: C[C@H](Cn1cnc2c(N)ncnc21)OCP(=O)(O)OCCCOCCCCCCCCCCCCCC#CC(F)(F)F] |
| VI-203 | 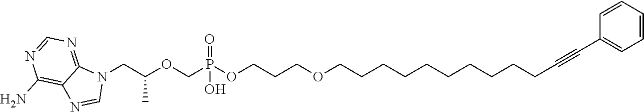 [SMILES: C[C@H](Cn1cnc2c(N)ncnc21)OCP(=O)(O)OCCCOCCCCCCCCCCC#Cc1ccccc1] |
| VI-204 | 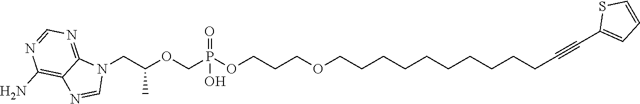 [SMILES: C[C@H](Cn1cnc2c(N)ncnc21)OCP(=O)(O)OCCCOCCCCCCCCCCC#Cc1cccs1] |
| VI-205 | 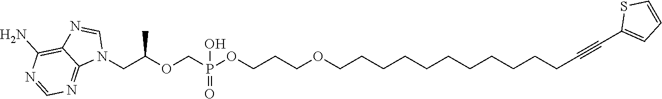 [SMILES: C[C@H](Cn1cnc2c(N)ncnc21)OCP(=O)(O)OCCCOCCCCCCCCCCCC#Cc1cccs1] |
| VI-206 | 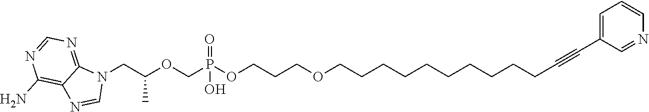 [SMILES: C[C@H](Cn1cnc2c(N)ncnc21)OCP(=O)(O)OCCCOCCCCCCCCCCC#Cc1cccnc1] |
| VI-207 | 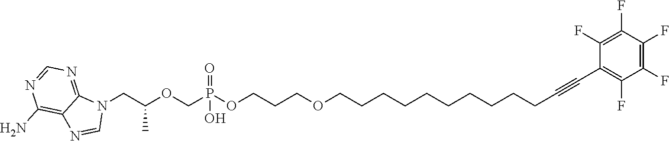 [SMILES: C[C@H](Cn1cnc2c(N)ncnc21)OCP(=O)(O)OCCCOCCCCCCCCCCC#Cc1c(F)c(F)c(F)c(F)c1F] |
| VI-208 | 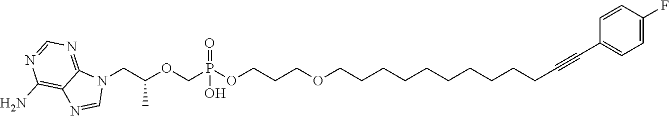 [SMILES: C[C@H](Cn1cnc2c(N)ncnc21)OCP(=O)(O)OCCCOCCCCCCCCCCC#Cc1ccc(F)cc1] |
| VI-209 | 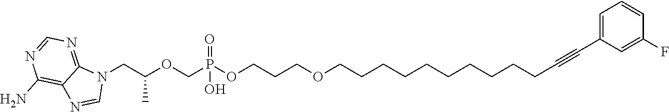 [SMILES: C[C@H](Cn1cnc2c(N)ncnc21)OCP(=O)(O)OCCCOCCCCCCCCCCC#Cc1cccc(F)c1] |
| VI-210 | 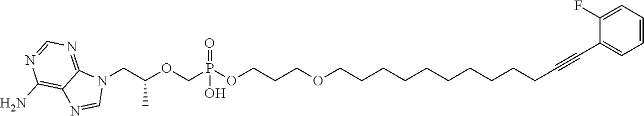 [SMILES: C[C@H](Cn1cnc2c(N)ncnc21)OCP(=O)(O)OCCCOCCCCCCCCCCC#Cc1ccccc1F] |
| VI-211 | 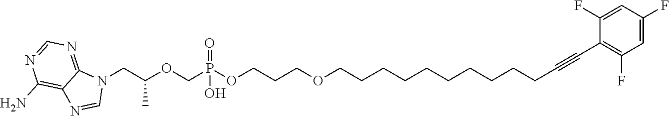 [SMILES: C[C@H](Cn1cnc2c(N)ncnc21)OCP(=O)(O)OCCCOCCCCCCCCCCC#Cc1c(F)cc(F)cc1F] |
| VI-212 | 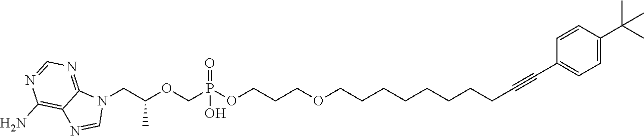 [SMILES: C[C@H](Cn1cnc2c(N)ncnc21)OCP(=O)(O)OCCCOCCCCCCCCC#Cc1ccc(C(C)(C)C)cc1] |
| VI-213 | 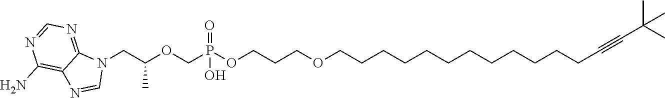 [SMILES: C[C@H](Cn1cnc2c(N)ncnc21)OCP(=O)(O)OCCCOCCCCCCCCCCCCC#CC(C)(C)C] |
| VI-214 | 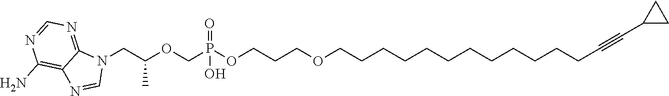 [SMILES: C[C@H](Cn1cnc2c(N)ncnc21)OCP(=O)(O)OCCCOCCCCCCCCCCCCC#CC1CC1] |
| VI-215 | 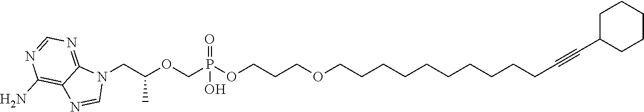 [SMILES: C[C@H](Cn1cnc2c(N)ncnc21)OCP(=O)(O)OCCCOCCCCCCCCCCC#CC1CCCCC1] |
| VI-216 | 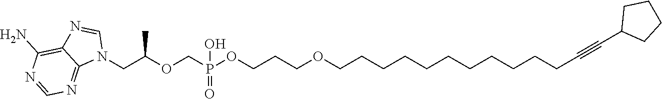 [SMILES: C[C@H](Cn1cnc2c(N)ncnc21)OCP(=O)(O)OCCCOCCCCCCCCCCCC#CC1CCCC1] |
| VI-217 | 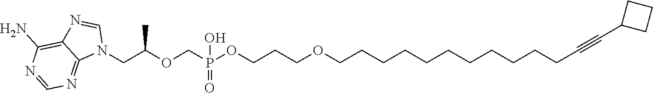 [SMILES: C[C@H](Cn1cnc2c(N)ncnc21)OCP(=O)(O)OCCCOCCCCCCCCCCCC#CC1CCC1] |
| VI-218 | 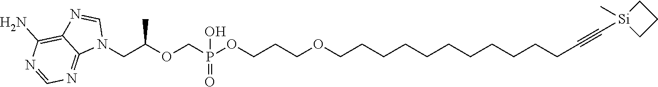 [SMILES: C[C@H](Cn1cnc2c(N)ncnc21)OCP(=O)(O)OCCCOCCCCCCCCCCCC#C[Si]1(C)CCC1] |
| VI-219 | 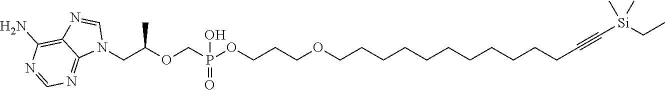 [SMILES: CC[Si](C)(C)C#CCCCCCCCCCCCOCCCOP(=O)(O)CO[C@H](C)Cn1cnc2c(N)ncnc21] |
| VI-220 | 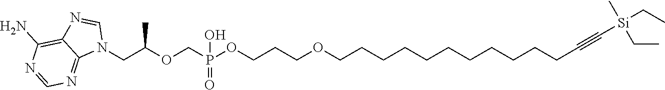 [SMILES: CC[Si](C)(C#CCCCCCCCCCCCOCCCOP(=O)(O)CO[C@H](C)Cn1cnc2c(N)ncnc21)CC] |
| VI-221 | 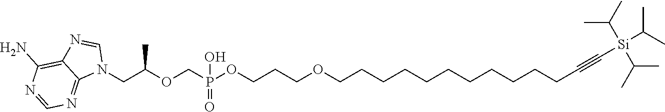 [SMILES: CC(C)[Si](C#CCCCCCCCCCCCOCCCOP(=O)(O)CO[C@H](C)Cn1cnc2c(N)ncnc21)(C(C)C)C(C)C] |
| VI-222 | 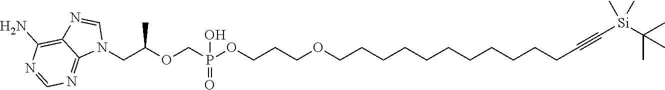 [SMILES: C[C@H](Cn1cnc2c(N)ncnc21)OCP(=O)(O)OCCCOCCCCCCCCCCCC#C[Si](C)(C)C(C)(C)C] |
| VI-223 | 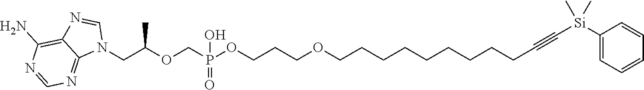 [SMILES: C[C@H](Cn1cnc2c(N)ncnc21)OCP(=O)(O)OCCCOCCCCCCCCCC#C[Si](C)(C)c1ccccc1] |
| VI-224 | 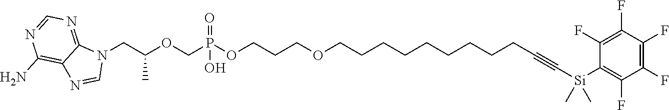 [SMILES: C[C@H](Cn1cnc2c(N)ncnc21)OCP(=O)(O)OCCCOCCCCCCCCCC#C[Si](C)(C)c1c(F)c(F)c(F)c(F)c1F] |
| VI-225 | 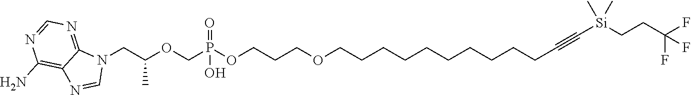 [SMILES: C[C@H](Cn1cnc2c(N)ncnc21)OCP(=O)(O)OCCCOCCCCCCCCCCC#C[Si](C)(C)CCC(F)(F)F] |
| VI-226 | 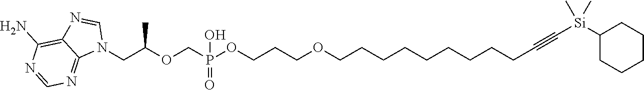 [SMILES: C[C@H](Cn1cnc2c(N)ncnc21)OCP(=O)(O)OCCCOCCCCCCCCCC#C[Si](C)(C)C1CCCCC1] |
| VI-227 | 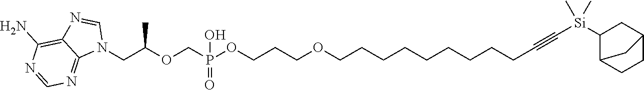 [SMILES: C[C@H](Cn1cnc2c(N)ncnc21)OCP(=O)(O)OCCCOCCCCCCCCCC#C[Si](C)(C)C1CC2CCC1C2] |
| VI-228 | 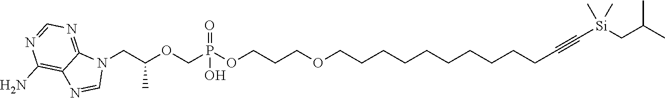 [SMILES: CC(C)C[Si](C)(C)C#CCCCCCCCCCCOCCCOP(=O)(O)CO[C@H](C)Cn1cnc2c(N)ncnc21] |
| VI-229 | 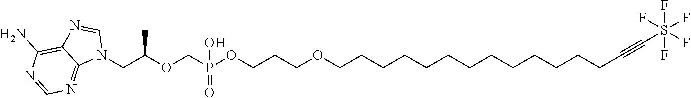 [SMILES: C[C@H](Cn1cnc2c(N)ncnc21)OCP(=O)(O)OCCCOCCCCCCCCCCCCCC#CS(F)(F)(F)(F)F] |
| VI-230 | 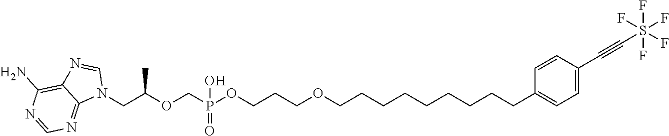 [SMILES: C[C@H](Cn1cnc2c(N)ncnc21)OCP(=O)(O)OCCCOCCCCCCCCCc1ccc(C#CS(F)(F)(F)(F)F)cc1] |
| VI-231 | 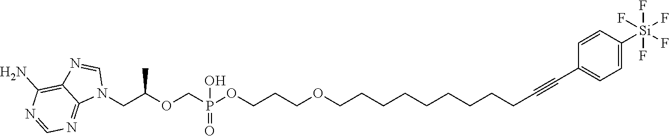 |
| VI-232 | 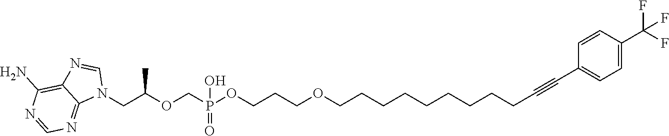 [SMILES: C[C@H](Cn1cnc2c(N)ncnc21)OCP(=O)(O)OCCCOCCCCCCCCCC#Cc1ccc(C(F)(F)F)cc1] |
| VI-233 | 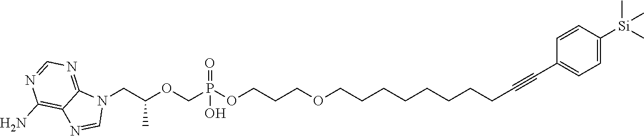 [SMILES: C[C@H](Cn1cnc2c(N)ncnc21)OCP(=O)(O)OCCCOCCCCCCCCC#Cc1ccc([Si](C)(C)C)cc1] |
| VI-234 | 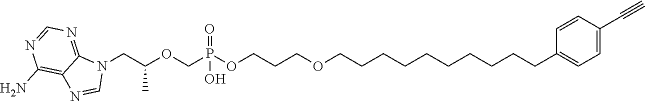 [SMILES: C#Cc1ccc(CCCCCCCCCCOCCCOP(=O)(O)CO[C@H](C)Cn2cnc3c(N)ncnc32)cc1] |
| VI-235 | 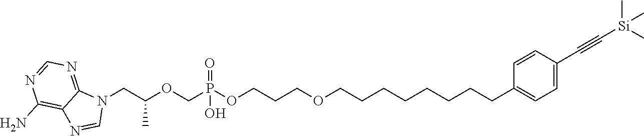 [SMILES: C[C@H](Cn1cnc2c(N)ncnc21)OCP(=O)(O)OCCCOCCCCCCCCc1ccc(C#C[Si](C)(C)C)cc1] |
| VI-236 | 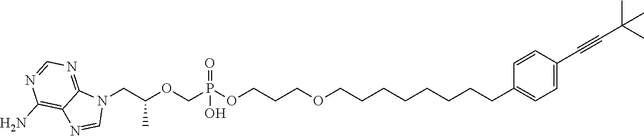 [SMILES: C[C@H](Cn1cnc2c(N)ncnc21)OCP(=O)(O)OCCCOCCCCCCCCc1ccc(C#CC(C)(C)C)cc1] |
| VI-237 | 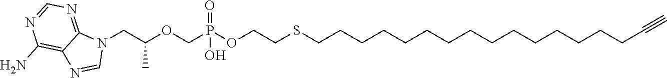 [SMILES: C#CCCCCCCCCCCCCCCCSCCOP(=O)(O)CO[C@H](C)Cn1cnc2c(N)ncnc21] |
| VI-238 | 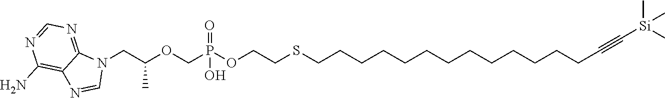 [SMILES: C[C@H](Cn1cnc2c(N)ncnc21)OCP(=O)(O)OCCSCCCCCCCCCCCCCC#C[Si](C)(C)C] |
| VI-239 | 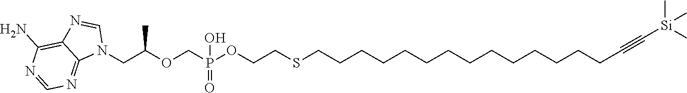 [SMILES: C[C@H](Cn1cnc2c(N)ncnc21)OCP(=O)(O)OCCSCCCCCCCCCCCCCCC#C[Si](C)(C)C] |
| VI-240 | 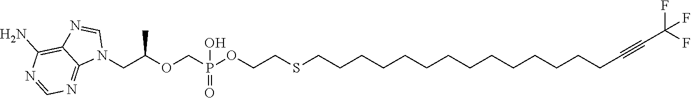 [SMILES: C[C@H](Cn1cnc2c(N)ncnc21)OCP(=O)(O)OCCSCCCCCCCCCCCCCCC#CC(F)(F)F] |
| VI-241 | 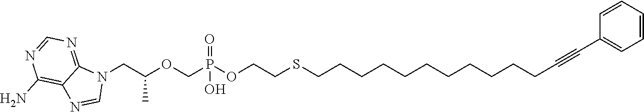 [SMILES: C[C@H](Cn1cnc2c(N)ncnc21)OCP(=O)(O)OCCSCCCCCCCCCCCC#Cc1ccccc1] |
| VI-242 | 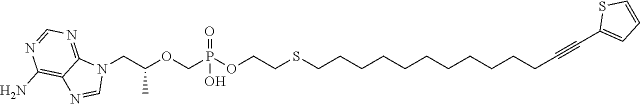 [SMILES: C[C@H](Cn1cnc2c(N)ncnc21)OCP(=O)(O)OCCSCCCCCCCCCCCC#Cc1cccs1] |
| VI-243 | 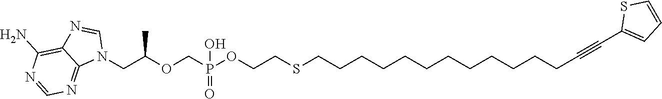 [SMILES: C[C@H](Cn1cnc2c(N)ncnc21)OCP(=O)(O)OCCSCCCCCCCCCCCCC#Cc1cccs1] |
| VI-244 | 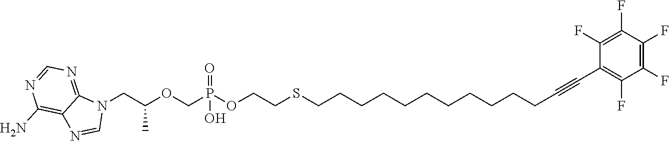 [SMILES: C[C@H](Cn1cnc2c(N)ncnc21)OCP(=O)(O)OCCSCCCCCCCCCCCC#Cc1c(F)c(F)c(F)c(F)c1F] |
| VI-245 | 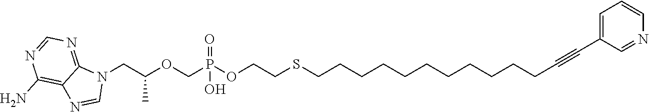 [SMILES: C[C@H](Cn1cnc2c(N)ncnc21)OCP(=O)(O)OCCSCCCCCCCCCCCC#Cc1cccnc1] |
| VI-246 | 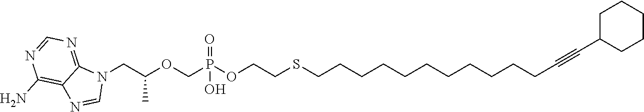 [SMILES: C[C@H](Cn1cnc2c(N)ncnc21)OCP(=O)(O)OCCSCCCCCCCCCCCC#CC1CCCCC1] |
| VI-247 | 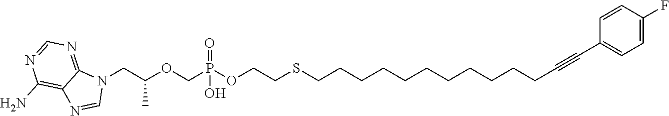 [SMILES: C[C@H](Cn1cnc2c(N)ncnc21)OCP(=O)(O)OCCSCCCCCCCCCCCC#Cc1ccc(F)cc1] |
| VI-248 | 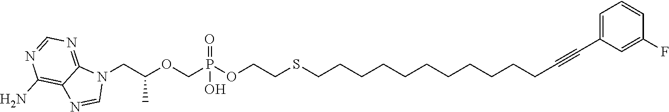 [SMILES: C[C@H](Cn1cnc2c(N)ncnc21)OCP(=O)(O)OCCSCCCCCCCCCCCC#Cc1cccc(F)c1] |
| VI-249 | 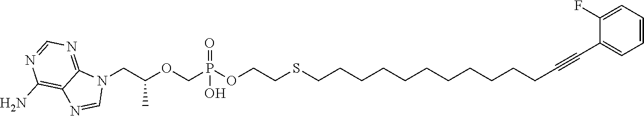 [SMILES: C[C@H](Cn1cnc2c(N)ncnc21)OCP(=O)(O)OCCSCCCCCCCCCCCC#Cc1ccccc1F] |
| VI-250 | 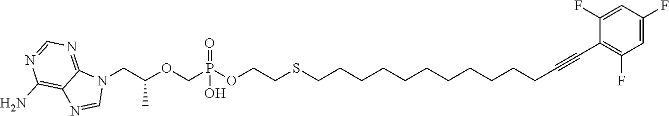 [SMILES: C[C@H](Cn1cnc2c(N)ncnc21)OCP(=O)(O)OCCSCCCCCCCCCCCC#Cc1c(F)cc(F)cc1F] |
| VI-251 | 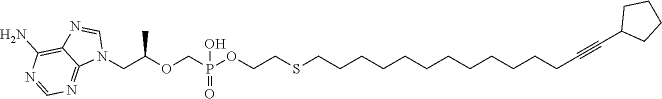 [SMILES: C[C@H](Cn1cnc2c(N)ncnc21)OCP(=O)(O)OCCSCCCCCCCCCCCCC#CC1CCCC1] |
| VI-252 | 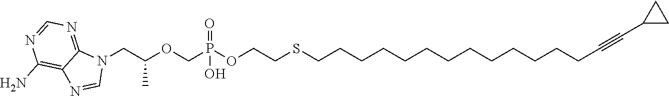 [SMILES: C[C@H](Cn1cnc2c(N)ncnc21)OCP(=O)(O)OCCSCCCCCCCCCCCCCC#CC1CC1] |
| VI-253 | 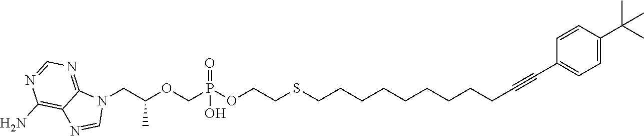 [SMILES: C[C@H](Cn1cnc2c(N)ncnc21)OCP(=O)(O)OCCSCCCCCCCCCC#Cc1ccc(C(C)(C)C)cc1] |
| VI-254 | 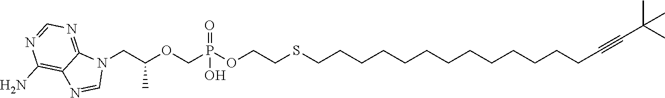 [SMILES: C[C@H](Cn1cnc2c(N)ncnc21)OCP(=O)(O)OCCSCCCCCCCCCCCCCC#CC(C)(C)C] |
| VI-255 | 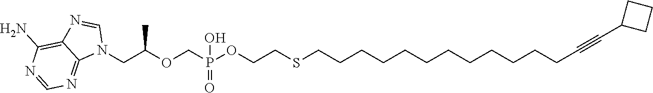 [SMILES: C[C@H](Cn1cnc2c(N)ncnc21)OCP(=O)(O)OCCSCCCCCCCCCCCCC#CC1CCC1] |
| VI-256 | 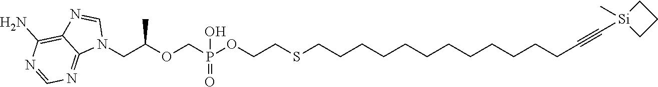 [SMILES: C[C@H](Cn1cnc2c(N)ncnc21)OCP(=O)(O)OCCSCCCCCCCCCCCCC#C[Si]1(C)CCC1] |
| VI-257 | 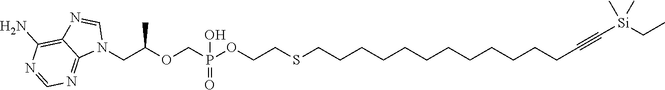 [SMILES: CC[Si](C)(C)C#CCCCCCCCCCCCCSCCOP(=O)(O)CO[C@H](C)Cn1cnc2c(N)ncnc21] |
| VI-258 | 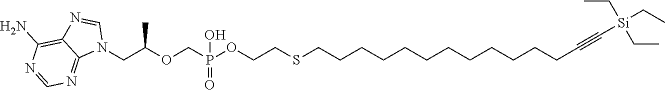 [SMILES: CC[Si](C#CCCCCCCCCCCCCSCCOP(=O)(O)CO[C@H](C)Cn1cnc2c(N)ncnc21)(CC)CC] |
| VI-259 | 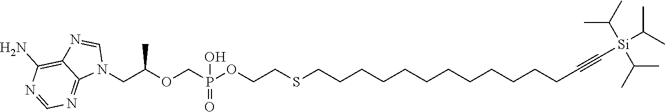 [SMILES: CC(C)[Si](C#CCCCCCCCCCCCCSCCOP(=O)(O)CO[C@H](C)Cn1cnc2c(N)ncnc21)(C(C)C)C(C)C] |
| VI-260 | 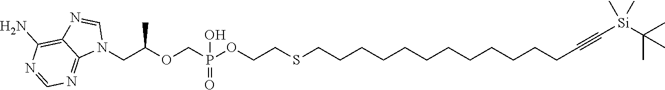 [SMILES: C[C@H](Cn1cnc2c(N)ncnc21)OCP(=O)(O)OCCSCCCCCCCCCCCCC#C[Si](C)(C)C(C)(C)C] |
| VI-261 | 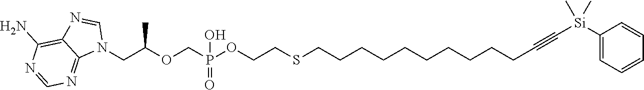 [SMILES: C[C@H](Cn1cnc2c(N)ncnc21)OCP(=O)(O)OCCSCCCCCCCCCCC#C[Si](C)(C)c1ccccc1] |
| VI-262 | 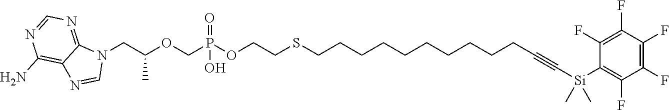 [SMILES: C[C@H](Cn1cnc2c(N)ncnc21)OCP(=O)(O)OCCSCCCCCCCCCCC#C[Si](C)(C)c1c(F)c(F)c(F)c(F)c1F] |
| VI-263 | 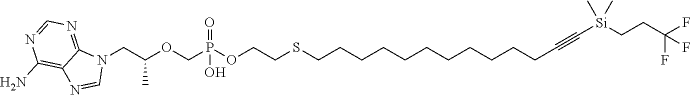 [SMILES: C[C@H](Cn1cnc2c(N)ncnc21)OCP(=O)(O)OCCSCCCCCCCCCCCC#C[Si](C)(C)CCC(F)(F)F] |
| VI-264 | 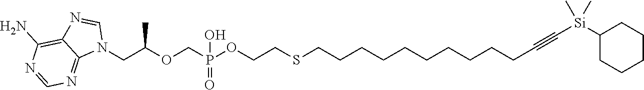 [SMILES: C[C@H](Cn1cnc2c(N)ncnc21)OCP(=O)(O)OCCSCCCCCCCCCCC#C[Si](C)(C)C1CCCCC1] |
| VI-265 | 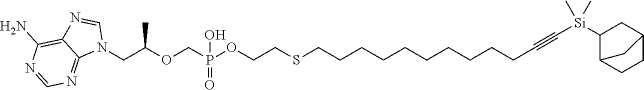 [SMILES: C[C@H](Cn1cnc2c(N)ncnc21)OCP(=O)(O)OCCSCCCCCCCCCCC#C[Si](C)(C)C1CC2CCC1C2] |
| VI-266 | 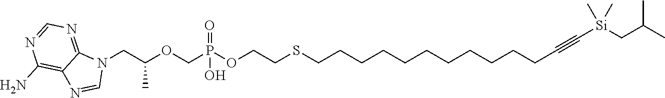 [SMILES: CC(C)C[Si](C)(C)C#CCCCCCCCCCCCSCCOP(=O)(O)CO[C@H](C)Cn1cnc2c(N)ncnc21] |
| VI-267 | 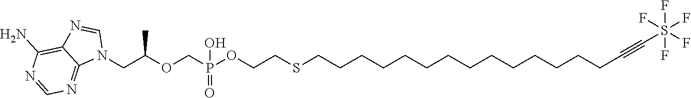 [SMILES: C[C@H](Cn1cnc2c(N)ncnc21)OCP(=O)(O)OCCSCCCCCCCCCCCCCCC#CS(F)(F)(F)(F)F] |
| VI-268 | 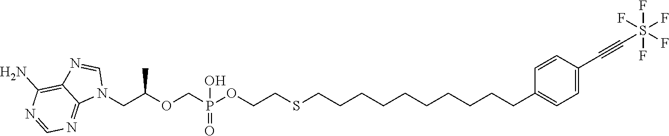 [SMILES: C[C@H](Cn1cnc2c(N)ncnc21)OCP(=O)(O)OCCSCCCCCCCCCCc1ccc(C#CS(F)(F)(F)(F)F)cc1] |
| VI-269 | 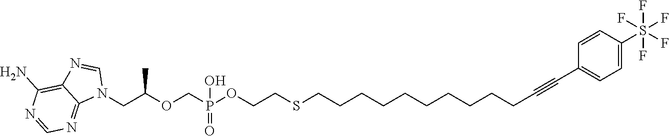 [SMILES: C[C@H](Cn1cnc2c(N)ncnc21)OCP(=O)(O)OCCSCCCCCCCCCCC#Cc1ccc(S(F)(F)(F)(F)F)cc1] |
| VI-270 | 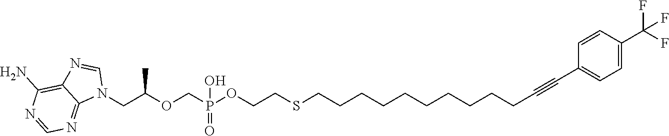 [SMILES: C[C@H](Cn1cnc2c(N)ncnc21)OCP(=O)(O)OCCSCCCCCCCCCCC#Cc1ccc(C(F)(F)F)cc1] |
| VI-271 | 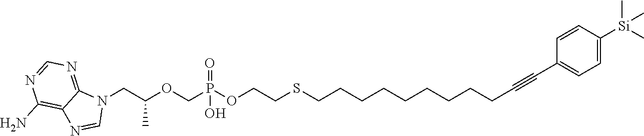 [SMILES: C[C@H](Cn1cnc2c(N)ncnc21)OCP(=O)(O)OCCSCCCCCCCCCC#Cc1ccc([Si](C)(C)C)cc1] |
| VI-272 | 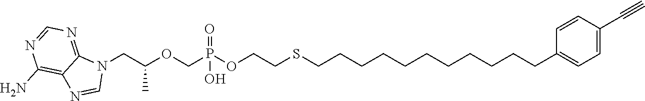 [SMILES: C#Cc1ccc(CCCCCCCCCCCSCCOP(=O)(O)CO[C@H](C)Cn2cnc3c(N)ncnc32)cc1] |
| VI-273 | 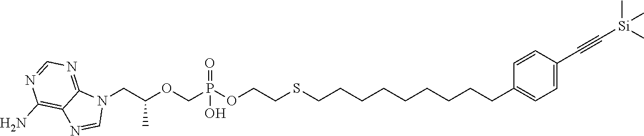 [SMILES: C[C@H](Cn1cnc2c(N)ncnc21)OCP(=O)(O)OCCSCCCCCCCCCc1ccc(C#C[Si](C)(C)C)cc1] |
| VI-274 | 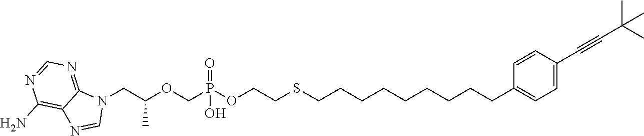 [SMILES: C[C@H](Cn1cnc2c(N)ncnc21)OCP(=O)(O)OCCSCCCCCCCCCc1ccc(C#CC(C)(C)C)cc1] |
| VI-275 | 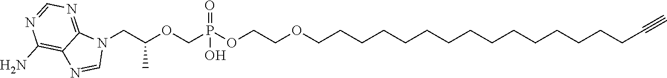 [SMILES: C#CCCCCCCCCCCCCCCCOCCOP(=O)(O)CO[C@H](C)Cn1cnc2c(N)ncnc21] |
| VI-276 | 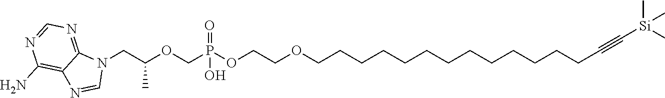 [SMILES: C[C@H](Cn1cnc2c(N)ncnc21)OCP(=O)(O)OCCOCCCCCCCCCCCCCC#C[Si](C)(C)C] |
| VI-277 | 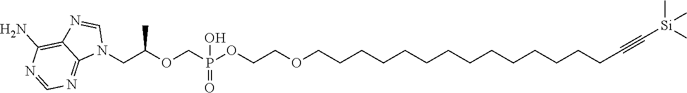 [SMILES: C[C@H](Cn1cnc2c(N)ncnc21)OCP(=O)(O)OCCOCCCCCCCCCCCCCCC#C[Si](C)(C)C] |
| VI-278 | 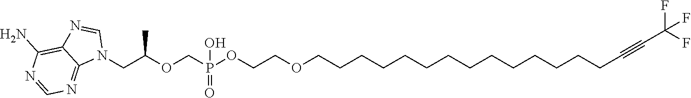 [SMILES: C[C@H](Cn1cnc2c(N)ncnc21)OCP(=O)(O)OCCOCCCCCCCCCCCCCCC#CC(F)(F)F] |
| VI-279 | 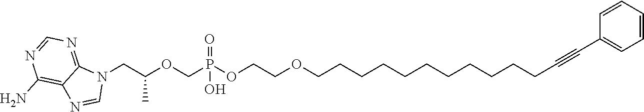 [SMILES: C[C@H](Cn1cnc2c(N)ncnc21)OCP(=O)(O)OCCOCCCCCCCCCCCC#Cc1ccccc1] |
| VI-280 | 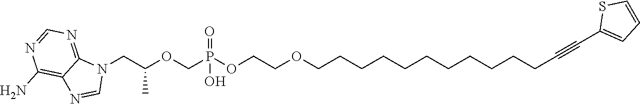 [SMILES: C[C@H](Cn1cnc2c(N)ncnc21)OCP(=O)(O)OCCOCCCCCCCCCCCC#Cc1cccs1] |
| VI-281 | 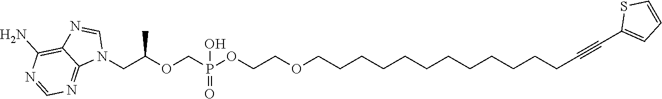 [SMILES: C[C@H](Cn1cnc2c(N)ncnc21)OCP(=O)(O)OCCOCCCCCCCCCCCCC#Cc1cccs1] |
| VI-282 | 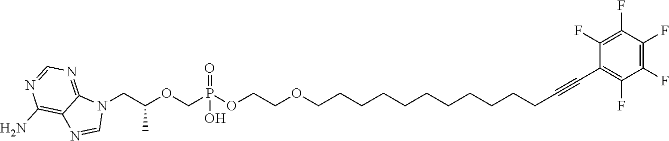 [SMILES: C[C@H](Cn1cnc2c(N)ncnc21)OCP(=O)(O)OCCOCCCCCCCCCCCC#Cc1c(F)c(F)c(F)c(F)c1F] |
| VI-283 | 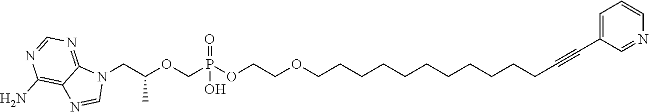 [SMILES: C[C@H](Cn1cnc2c(N)ncnc21)OCP(=O)(O)OCCOCCCCCCCCCCCC#Cc1cccnc1] |
| VI-284 | 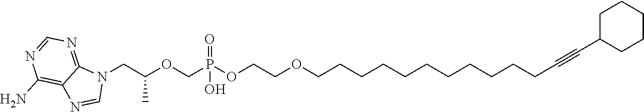 [SMILES: C[C@H](Cn1cnc2c(N)ncnc21)OCP(=O)(O)OCCOCCCCCCCCCCCC#CC1CCCCC1] |
| VI-285 | 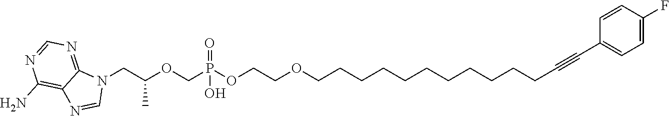 [SMILES: C[C@H](Cn1cnc2c(N)ncnc21)OCP(=O)(O)OCCOCCCCCCCCCCCC#Cc1ccc(F)cc1] |
| VI-286 | 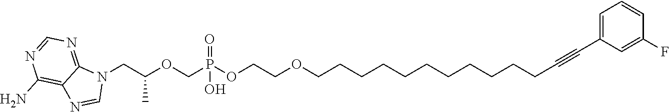 [SMILES: C[C@H](Cn1cnc2c(N)ncnc21)OCP(=O)(O)OCCOCCCCCCCCCCCC#Cc1cccc(F)c1] |
| VI-287 | 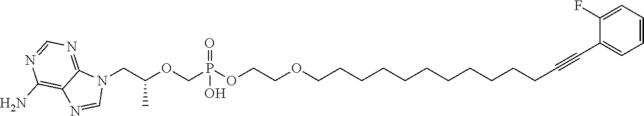 [SMILES: C[C@H](Cn1cnc2c(N)ncnc21)OCP(=O)(O)OCCOCCCCCCCCCCCC#Cc1ccccc1F] |
| VI-288 | 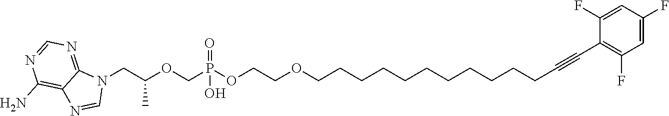 [SMILES: C[C@H](Cn1cnc2c(N)ncnc21)OCP(=O)(O)OCCOCCCCCCCCCCCC#Cc1c(F)cc(F)cc1F] |
| VI-289 | 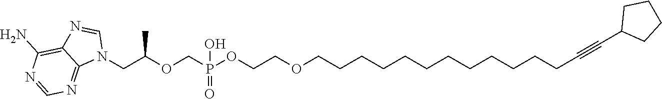 [SMILES: C[C@H](Cn1cnc2c(N)ncnc21)OCP(=O)(O)OCCOCCCCCCCCCCCCC#CC1CCCC1] |
| VI-290 | 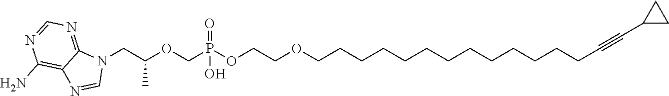 [SMILES: C[C@H](Cn1cnc2c(N)ncnc21)OCP(=O)(O)OCCOCCCCCCCCCCCCCC#CC1CC1] |
| VI-291 | 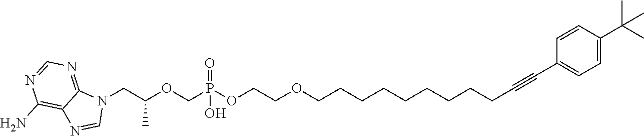 [SMILES: C[C@H](Cn1cnc2c(N)ncnc21)OCP(=O)(O)OCCOCCCCCCCCCC#Cc1ccc(C(C)(C)C)cc1] |
| VI-292 | 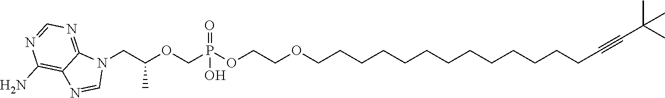 [SMILES: C[C@H](Cn1cnc2c(N)ncnc21)OCP(=O)(O)OCCOCCCCCCCCCCCCCC#CC(C)(C)C] |
| VI-293 | 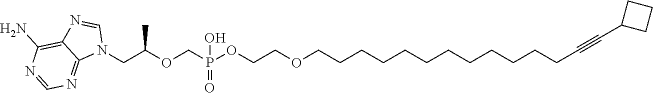 [SMILES: C[C@H](Cn1cnc2c(N)ncnc21)OCP(=O)(O)OCCOCCCCCCCCCCCCC#CC1CCC1] |
| VI-294 | 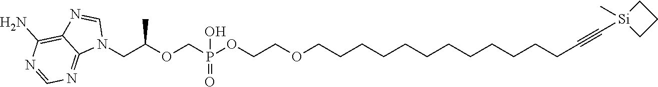 [SMILES: C[C@H](Cn1cnc2c(N)ncnc21)OCP(=O)(O)OCCOCCCCCCCCCCCCC#C[Si]1(C)CCC1] |
| VI-295 | 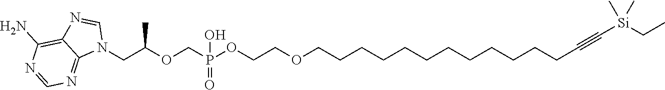 [SMILES: CC[Si](C)(C)C#CCCCCCCCCCCCCOCCOP(=O)(O)CO[C@H](C)Cn1cnc2c(N)ncnc21] |
| VI-296 | 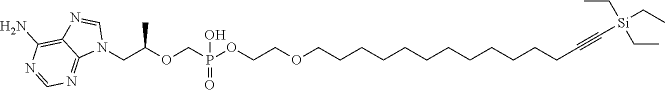 [SMILES: CC[Si](C#CCCCCCCCCCCCCOCCOP(=O)(O)CO[C@H](C)Cn1cnc2c(N)ncnc21)(CC)CC] |
| VI-297 | 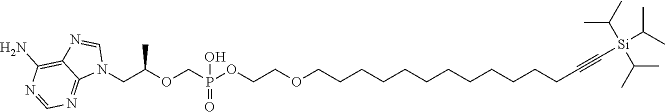 [SMILES: CC(C)[Si](C#CCCCCCCCCCCCCOCCOP(=O)(O)CO[C@H](C)Cn1cnc2c(N)ncnc21)(C(C)C)C(C)C] |
| VI-298 | 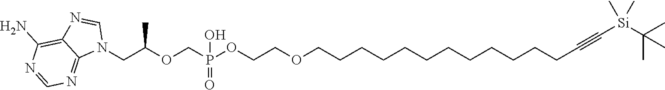 [SMILES: C[C@H](Cn1cnc2c(N)ncnc21)OCP(=O)(O)OCCOCCCCCCCCCCCCC#C[Si](C)(C)C(C)(C)C] |
| VI-299 | 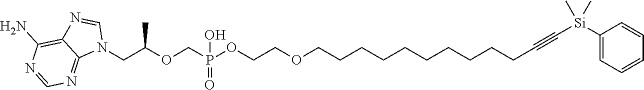 [SMILES: C[C@H](Cn1cnc2c(N)ncnc21)OCP(=O)(O)OCCOCCCCCCCCCCC#C[Si](C)(C)c1ccccc1] |
| VI-300 | 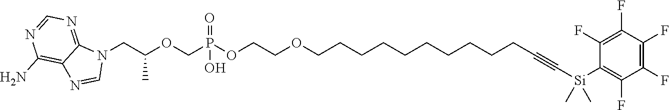 [SMILES: C[C@H](Cn1cnc2c(N)ncnc21)OCP(=O)(O)OCCOCCCCCCCCCCC#C[Si](C)(C)c1c(F)c(F)c(F)c(F)c1F] |
| VI-301 | 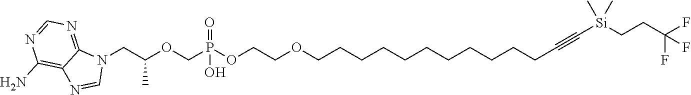 [SMILES: C[C@H](Cn1cnc2c(N)ncnc21)OCP(=O)(O)OCCOCCCCCCCCCCCC#C[Si](C)(C)CCC(F)(F)F] |
| VI-302 | 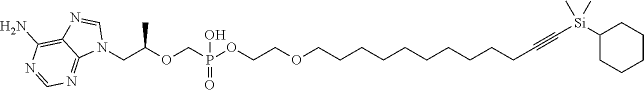 [SMILES: C[C@H](Cn1cnc2c(N)ncnc21)OCP(=O)(O)OCCOCCCCCCCCCCC#C[Si](C)(C)C1CCCCC1] |
| VI-303 | 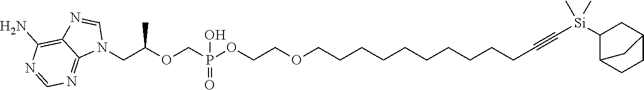 [SMILES: C[C@H](Cn1cnc2c(N)ncnc21)OCP(=O)(O)OCCOCCCCCCCCCCC#C[Si](C)(C)C1CC2CCC1C2] |
| VI-304 | 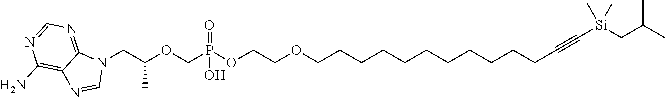 [SMILES: CC(C)C[Si](C)(C)C#CCCCCCCCCCCCOCCOP(=O)(O)CO[C@H](C)Cn1cnc2c(N)ncnc21] |
| VI-305 | 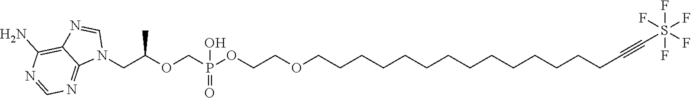 [SMILES: C[C@H](Cn1cnc2c(N)ncnc21)OCP(=O)(O)OCCOCCCCCCCCCCCCCCC#CS(F)(F)(F)(F)F] |
| VI-306 | 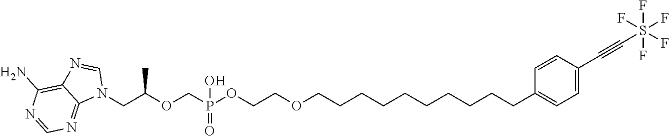 [SMILES: C[C@H](Cn1cnc2c(N)ncnc21)OCP(=O)(O)OCCOCCCCCCCCCCc1ccc(C#CS(F)(F)(F)(F)F)cc1] |
| VI-307 | 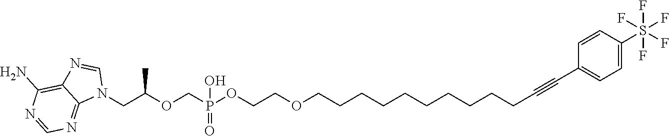 [SMILES: C[C@H](Cn1cnc2c(N)ncnc21)OCP(=O)(O)OCCOCCCCCCCCCCC#Cc1ccc(S(F)(F)(F)(F)F)cc1] |
| VI-308 | 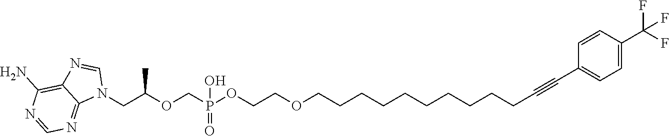 [SMILES: C[C@H](Cn1cnc2c(N)ncnc21)OCP(=O)(O)OCCOCCCCCCCCCCC#Cc1ccc(C(F)(F)F)cc1] |
| VI-309 | 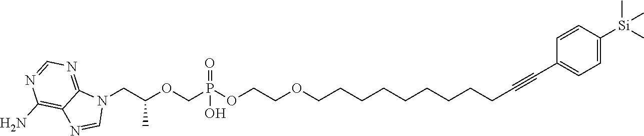 [SMILES: C[C@H](Cn1cnc2c(N)ncnc21)OCP(=O)(O)OCCOCCCCCCCCCC#Cc1ccc([Si](C)(C)C)cc1] |
| VI-310 | 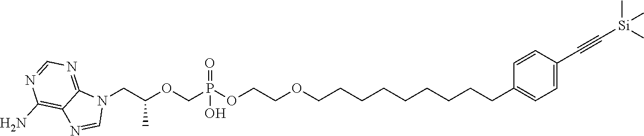 [SMILES: C[C@H](Cn1cnc2c(N)ncnc21)OCP(=O)(O)OCCOCCCCCCCCCc1ccc(C#C[Si](C)(C)C)cc1] |
| VI-311 | 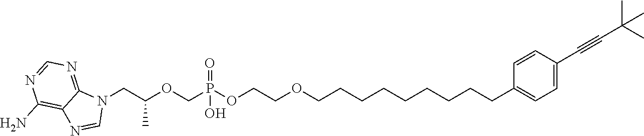 [SMILES: C[C@H](Cn1cnc2c(N)ncnc21)OCP(=O)(O)OCCOCCCCCCCCCc1ccc(C#CC(C)(C)C)cc1] |
| VI-312 | 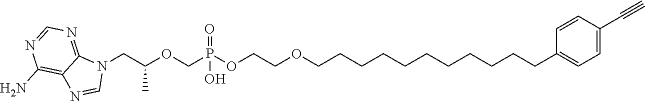 [SMILES: C#Cc1ccc(CCCCCCCCCCCOCCOP(=O)(O)CO[C@H](C)Cn2cnc3c(N)ncnc32)cc1] |

The modular synthetic routes described in the foregoing references can also be readily modified by one of skill in the art of organic synthesis to provide additional substituted adeninyl-propyloxy phosphonic acid and related compounds using strategies and reactions well known in the art, as described in, for example, “Comprehensive Organic Synthesis” (B. M. Trost & I. Fleming, eds., 1991-1992).

In certain other embodiments, the compound is

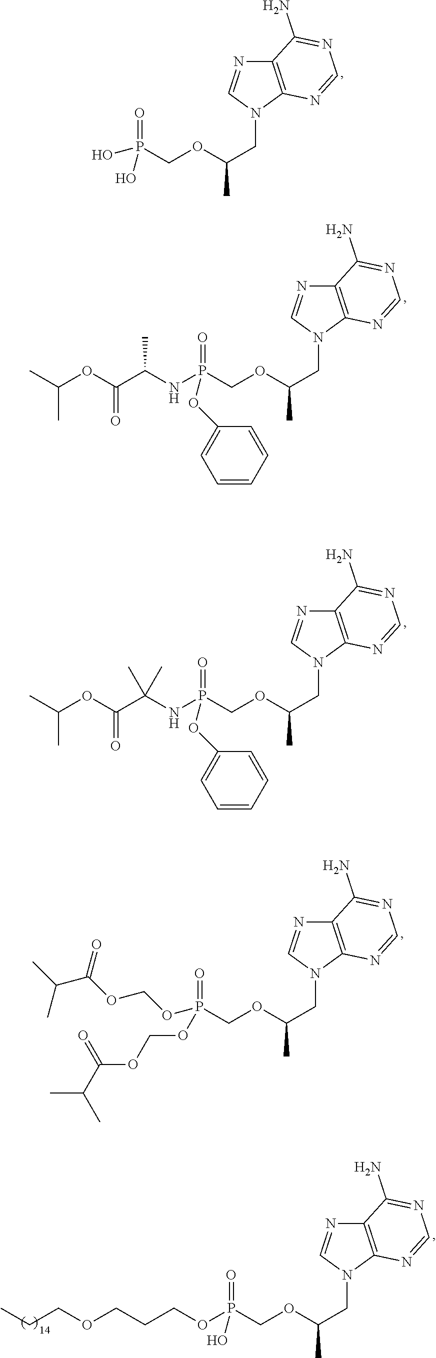 [SMILES: C[C@H](Cn1cnc2c(N)ncnc21)OCP(=O)(O)O ; CC(C)OC(=O)[C@H](C)NP(=O)(CO[C@H](C)Cn1cnc2c(N)ncnc21)Oc1ccccc1 ; CC(C)OC(=O)C(C)(C)NP(=O)(CO[C@H](C)Cn1cnc2c(N)ncnc21)Oc1ccccc1 ; CC(C)C(=O)OCOP(=O)(CO[C@H](C)Cn1cnc2c(N)ncnc21)OCOC(=O)C(C)C ; CCCOCCCOP(=O)(O)CO[C@H](C)Cn1cnc2c(N)ncnc21]

or a pharmaceutically acceptable salt thereof.

In certain other embodiments, the compound is

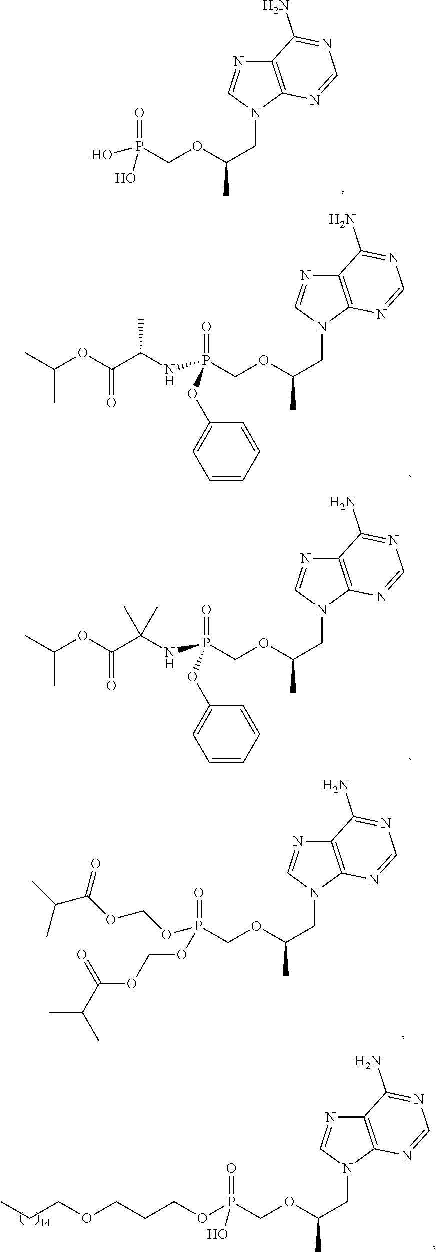

or a pharmaceutically acceptable salt thereof.

In certain other embodiments, the compound is

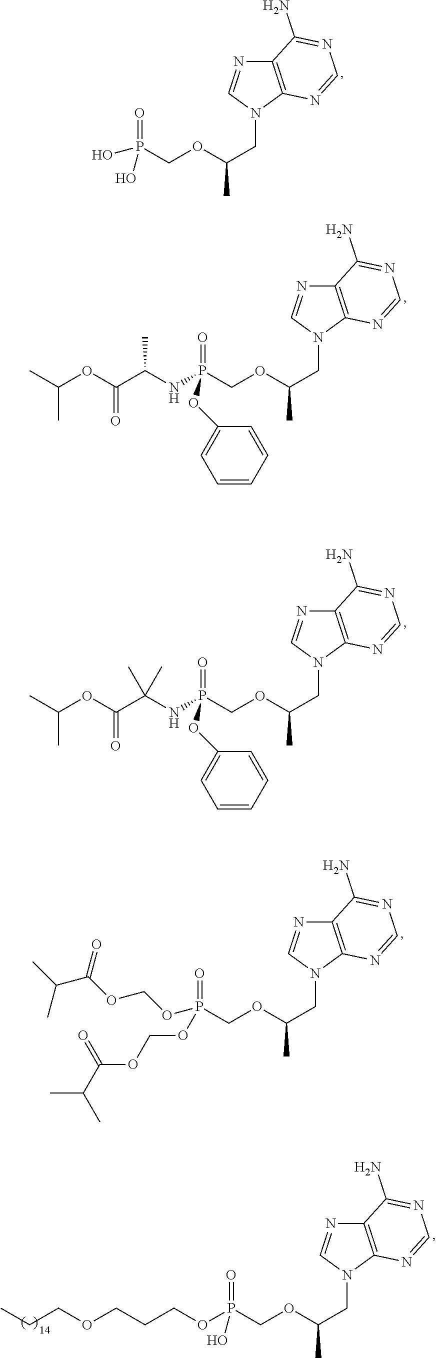 [SMILES: C[C@H](Cn1cnc2c(N)ncnc21)OCP(=O)(O)O ; CC(C)OC(=O)[C@H](C)N[P@](=O)(CO[C@H](C)Cn1cnc2c(N)ncnc21)Oc1ccccc1 ; CC(C)OC(=O)C(C)(C)N[P@](=O)(CO[C@H](C)Cn1cnc2c(N)ncnc21)Oc1ccccc1 ; CC(C)C(=O)OCOP(=O)(CO[C@H](C)Cn1cnc2c(N)ncnc21)OCOC(=O)C(C)C ; CCCOCCCOP(=O)(O)CO[C@H](C)Cn1cnc2c(N)ncnc21]

or a pharmaceutically acceptable salt thereof.

In certain other embodiments, the compound is

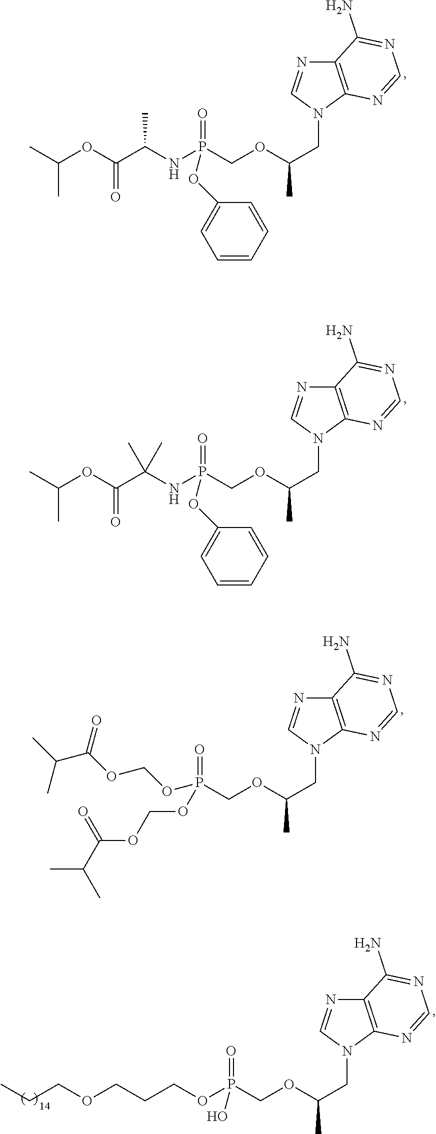 [SMILES: CC(C)OC(=O)[C@H](C)NP(=O)(CO[C@H](C)Cn1cnc2c(N)ncnc21)Oc1ccccc1 ; CC(C)OC(=O)C(C)(C)NP(=O)(CO[C@H](C)Cn1cnc2c(N)ncnc21)Oc1ccccc1 ; CC(C)C(=O)OCOP(=O)(CO[C@H](C)Cn1cnc2c(N)ncnc21)OCOC(=O)C(C)C ; CCCOCCCOP(=O)(O)CO[C@H](C)Cn1cnc2c(N)ncnc21]

or a pharmaceutically acceptable salt thereof.

In certain other embodiments, the compound is

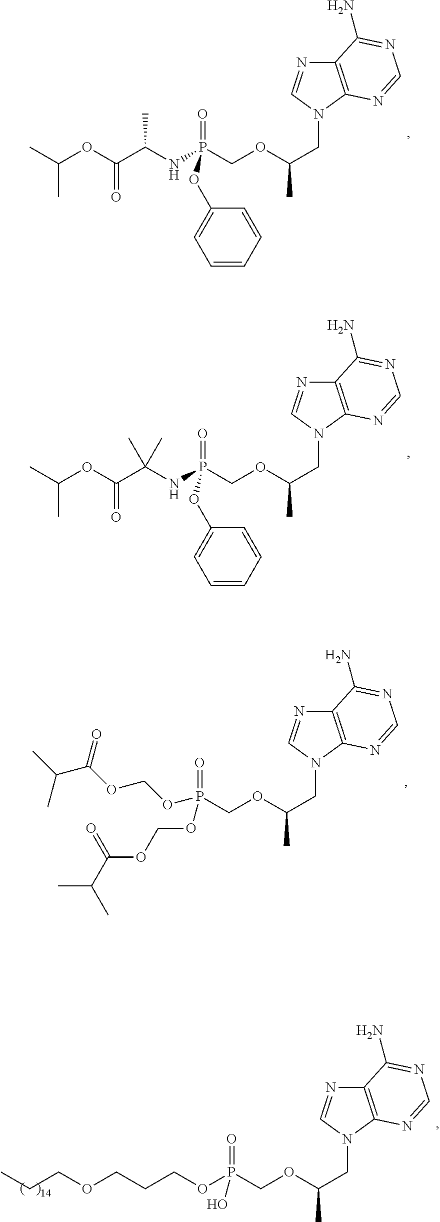

or a pharmaceutically acceptable salt thereof.

In certain other embodiments, the compound is

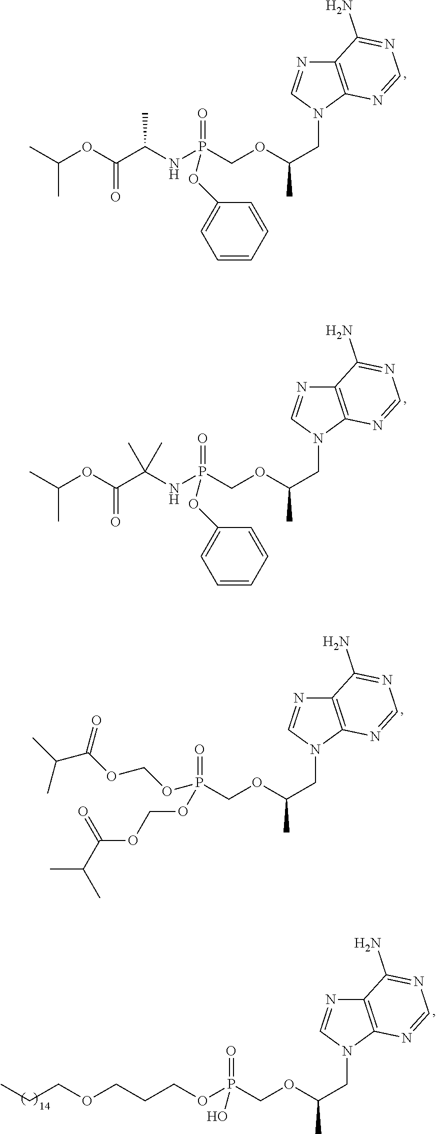 [SMILES: CC(C)OC(=O)[C@H](C)NP(=O)(CO[C@H](C)Cn1cnc2c(N)ncnc21)Oc1ccccc1 ; CC(C)OC(=O)C(C)(C)NP(=O)(CO[C@H](C)Cn1cnc2c(N)ncnc21)Oc1ccccc1 ; CC(C)C(=O)OCOP(=O)(CO[C@H](C)Cn1cnc2c(N)ncnc21)OCOC(=O)C(C)C ; CCCOCCCOP(=O)(O)CO[C@H](C)Cn1cnc2c(N)ncnc21]

or a pharmaceutically acceptable salt thereof.

In certain other embodiments, the compound is

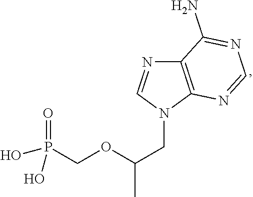 [SMILES: CC(Cn1cnc2c(N)ncnc21)OCP(=O)(O)O]

or a pharmaceutically acceptable salt thereof. In certain embodiments, the compound is

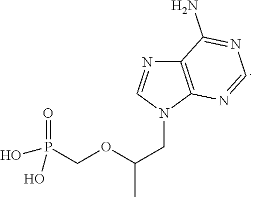 [SMILES: CC(Cn1cnc2c(N)ncnc21)OCP(=O)(O)O]

In certain embodiments, the compound is

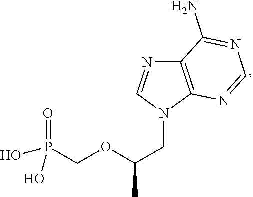 [SMILES: C[C@H](Cn1cnc2c(N)ncnc21)OCP(=O)(O)O]

or a pharmaceutically acceptable salt thereof. In certain embodiments, the compound is

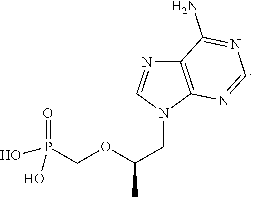 [SMILES: C[C@H](Cn1cnc2c(N)ncnc21)OCP(=O)(O)O]

In certain other embodiments, the compound is

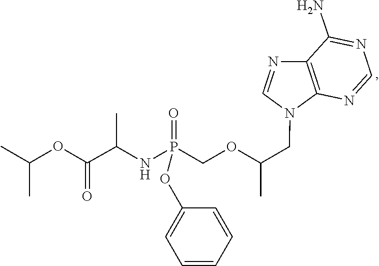 [SMILES: CC(C)OC(=O)C(C)NP(=O)(COC(C)Cn1cnc2c(N)ncnc21)Oc1ccccc1]

or a pharmaceutically acceptable salt thereof. In certain embodiments, the compound is

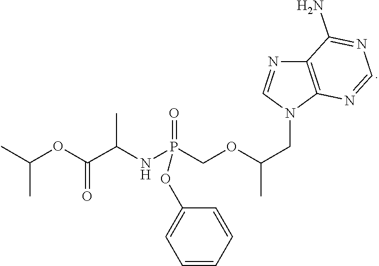 [SMILES: CC(C)OC(=O)C(C)NP(=O)(COC(C)Cn1cnc2c(N)ncnc21)Oc1ccccc1]

In certain embodiments, the compound is

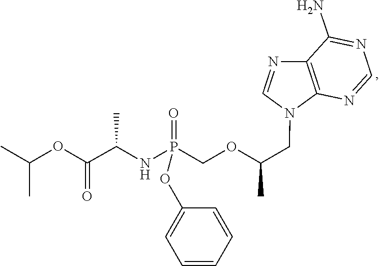 [SMILES: CC(C)OC(=O)[C@H](C)NP(=O)(CO[C@H](C)Cn1cnc2c(N)ncnc21)Oc1ccccc1]

or a pharmaceutically acceptable salt thereof. In certain embodiments, the compound is

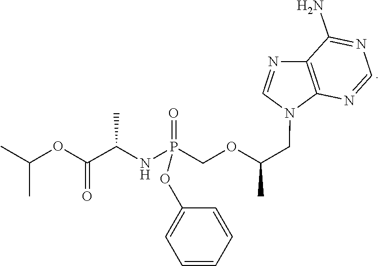 [SMILES: CC(C)OC(=O)[C@H](C)NP(=O)(CO[C@H](C)Cn1cnc2c(N)ncnc21)Oc1ccccc1]

In certain embodiments, the compound is

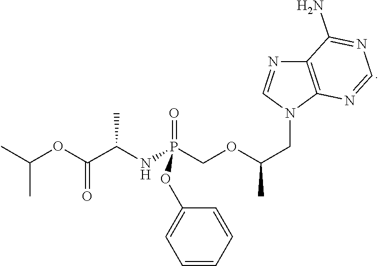 [SMILES: CC(C)OC(=O)[C@H](C)N[P@](=O)(CO[C@H](C)Cn1cnc2c(N)ncnc21)Oc1ccccc1]

or a pharmaceutically acceptable salt thereof. In certain embodiments, the compound is the hemifumarate salt of

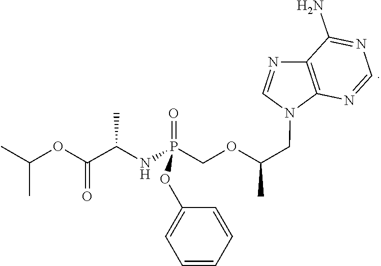 [SMILES: CC(C)OC(=O)[C@H](C)N[P@](=O)(CO[C@H](C)Cn1cnc2c(N)ncnc21)Oc1ccccc1]

In certain embodiments, the compound

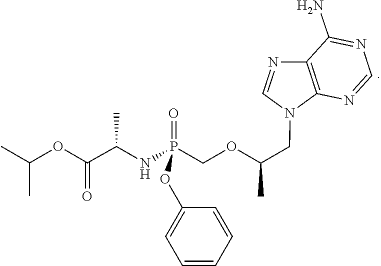 [SMILES: CC(C)OC(=O)[C@H](C)N[P@](=O)(CO[C@H](C)Cn1cnc2c(N)ncnc21)Oc1ccccc1]

In certain embodiments, the compound is

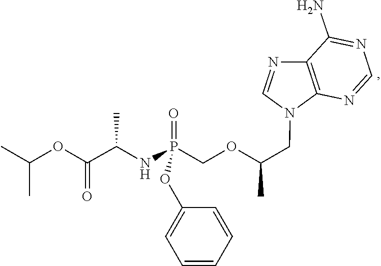 [SMILES: CC(C)OC(=O)[C@H](C)N[P@@](=O)(CO[C@H](C)Cn1cnc2c(N)ncnc21)Oc1ccccc1]

or a pharmaceutically acceptable salt thereof. In certain embodiments, the compound is

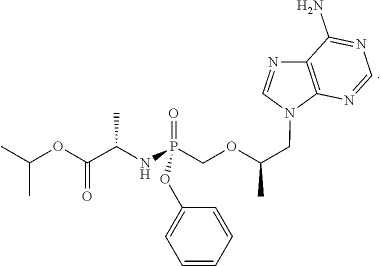 [SMILES: CC(C)OC(=O)[C@H](C)N[P@@](=O)(CO[C@H](C)Cn1cnc2c(N)ncnc21)Oc1ccccc1]

In certain other embodiments, the compound is

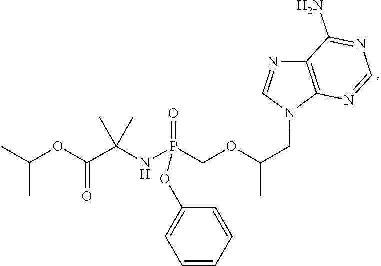 [SMILES: CC(C)OC(=O)C(C)(C)NP(=O)(COC(C)Cn1cnc2c(N)ncnc21)Oc1ccccc1]

or a pharmaceutically acceptable salt thereof. In certain embodiments, the compound is

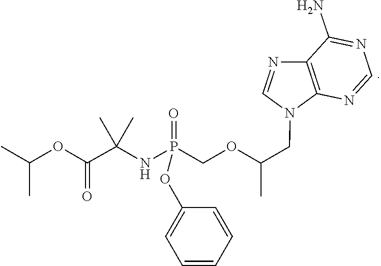 [SMILES: CC(C)OC(=O)C(C)(C)NP(=O)(COC(C)Cn1cnc2c(N)ncnc21)Oc1ccccc1]

In certain other embodiments, the compound is

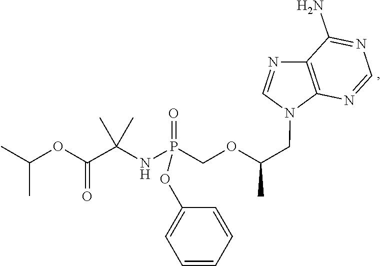 [SMILES: CC(C)OC(=O)C(C)(C)NP(=O)(CO[C@H](C)Cn1cnc2c(N)ncnc21)Oc1ccccc1]

or a pharmaceutically acceptable salt thereof. In certain embodiments, the compound is

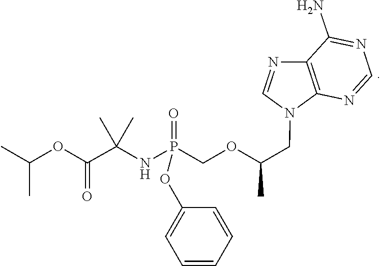 [SMILES: CC(C)OC(=O)C(C)(C)NP(=O)(CO[C@H](C)Cn1cnc2c(N)ncnc21)Oc1ccccc1]

In certain embodiments, the compound is

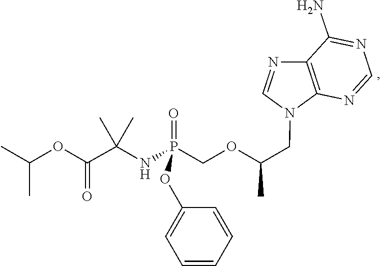 [SMILES: CC(C)OC(=O)C(C)(C)N[P@](=O)(CO[C@H](C)Cn1cnc2c(N)ncnc21)Oc1ccccc1]

or a pharmaceutically acceptable salt thereof. In certain embodiments, the compound is a fumarate salt of

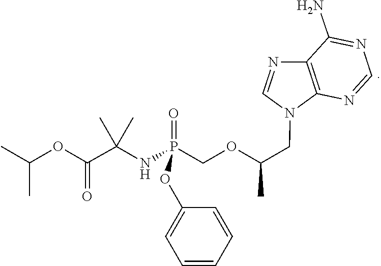 [SMILES: CC(C)OC(=O)C(C)(C)N[P@](=O)(CO[C@H](C)Cn1cnc2c(N)ncnc21)Oc1ccccc1]

In certain embodiments, the compound is

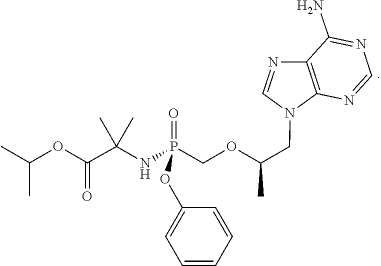 [SMILES: CC(C)OC(=O)C(C)(C)N[P@](=O)(CO[C@H](C)Cn1cnc2c(N)ncnc21)Oc1ccccc1]

In certain embodiments, the compound is

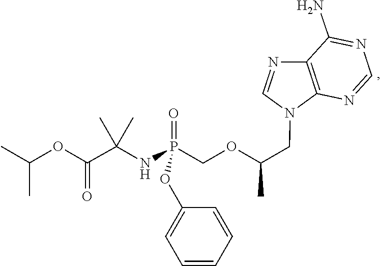 [SMILES: CC(C)OC(=O)C(C)(C)N[P@@](=O)(CO[C@H](C)Cn1cnc2c(N)ncnc21)Oc1ccccc1]

or a pharmaceutically acceptable salt thereof. In certain embodiments, the compound is a fumarate salt of

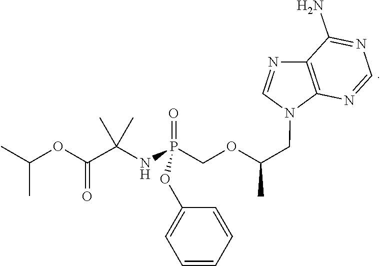 [SMILES: CC(C)OC(=O)C(C)(C)N[P@@](=O)(CO[C@H](C)Cn1cnc2c(N)ncnc21)Oc1ccccc1]

In certain embodiments, the compound is

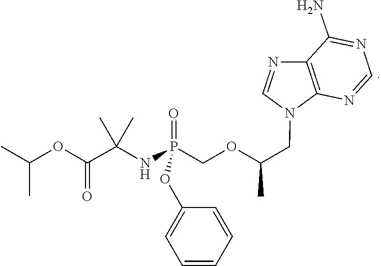 [SMILES: CC(C)OC(=O)C(C)(C)N[P@@](=O)(CO[C@H](C)Cn1cnc2c(N)ncnc21)Oc1ccccc1]

In certain other embodiments, the compound is

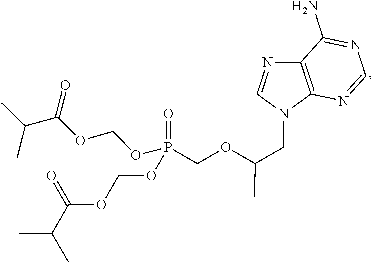 [SMILES: CC(Cn1cnc2c(N)ncnc21)OCP(=O)(OCOC(=O)C(C)C)OCOC(=O)C(C)C]

or a pharmaceutically acceptable salt thereof. In certain embodiments, the compound is

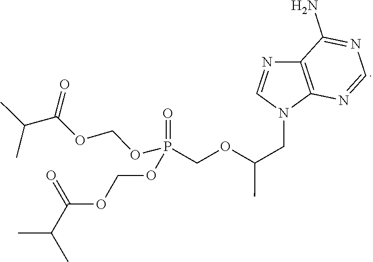 [SMILES: CC(Cn1cnc2c(N)ncnc21)OCP(=O)(OCOC(=O)C(C)C)OCOC(=O)C(C)C]

In certain embodiments, the compound is

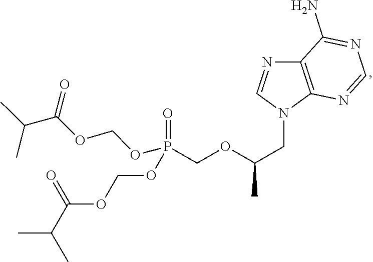 [SMILES: CC(C)C(=O)OCOP(=O)(CO[C@H](C)Cn1cnc2c(N)ncnc21)OCOC(=O)C(C)C]

or a pharmaceutically acceptable salt thereof. In certain embodiments, the compound is a fumarate, succinate, maleate, orotate, aspartate, or phosphate salt of

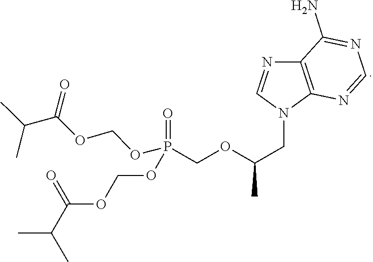 [SMILES: CC(C)C(=O)OCOP(=O)(CO[C@H](C)Cn1cnc2c(N)ncnc21)OCOC(=O)C(C)C]

In certain embodiments, the compound is a fumarate, succinate, or maleate salt of

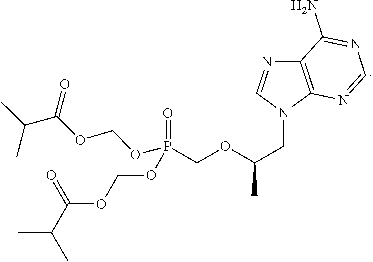 [SMILES: CC(C)C(=O)OCOP(=O)(CO[C@H](C)Cn1cnc2c(N)ncnc21)OCOC(=O)C(C)C]

In certain embodiments, the compound is

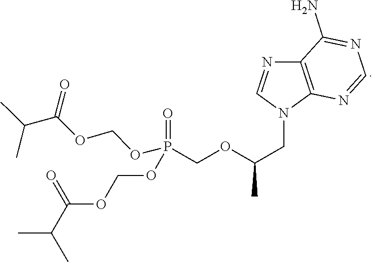 [SMILES: CC(C)C(=O)OCOP(=O)(CO[C@H](C)Cn1cnc2c(N)ncnc21)OCOC(=O)C(C)C]

In certain other embodiments, the compound is

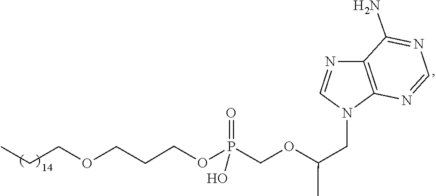

or a pharmaceutically acceptable salt thereof. In certain embodiments, the compound is

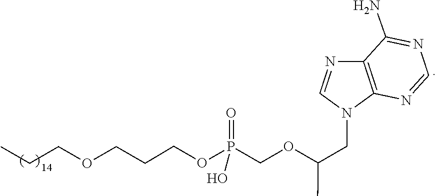

In certain embodiments, the compound is

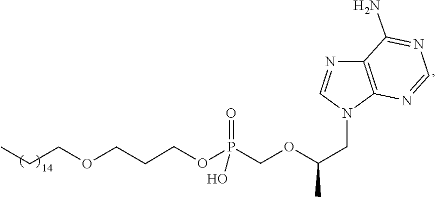

or a pharmaceutically acceptable salt thereof. In certain embodiments, the compound is

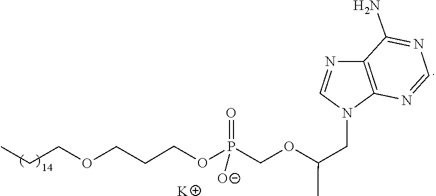

In certain embodiments, the compound is

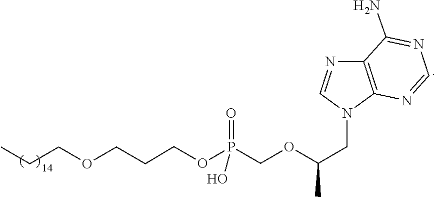

In certain embodiments, the compound is

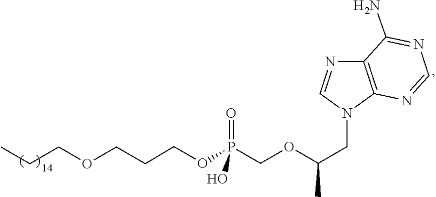

or a pharmaceutically acceptable salt thereof. In certain embodiments, the compound is

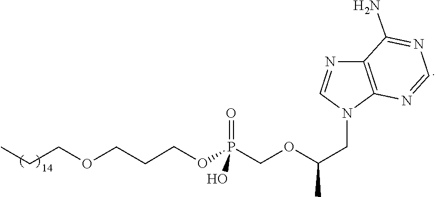

In certain embodiments, the compound is

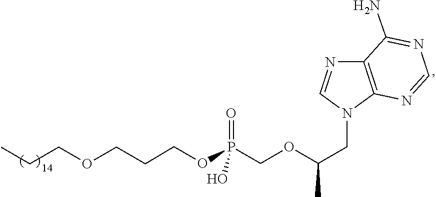

or a pharmaceutically acceptable salt thereof. In certain embodiments, the compound is

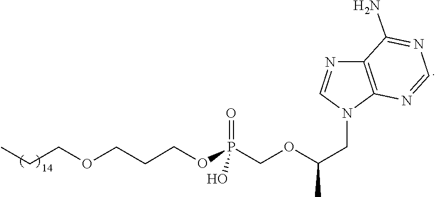

### IV. Combination Therapy

Another aspect of the invention provides for combination therapy. Substituted adeninyl-propyloxy phosphonic acids or related compounds described herein (e.g., a compound of Formula I, or other compounds in Section III) or their pharmaceutically acceptable salts may be used in combination with additional therapeutic agents to treat medical disorders (e.g., according to the methods described in Section I, with disorders such as a cancer). Accordingly, in some embodiments, a method of the invention further comprises administering an effective amount of an additional therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen more than 24 hours aparts.

The doses and dosage regimen of the active ingredients used in the combination therapy may be determined by an attending clinician. In certain embodiments, the substituted adeninyl-propyloxy phosphonic acid or related compound described herein (e.g., a compound of Formula I, or other compounds in Section III) and the additional therapeutic agent(s) (e.g. the second, third, or fourth, or fifth anti-cancer agent, described below) are administered in doses commonly employed when such agents are used as monotherapy for treating the disorder. In other embodiments, the substituted adeninyl-propyloxy phosphonic acid or related compound described herein (e.g., a compound of Formula I, or other compounds in Section III) and the additional therapeutic agent(s) (e.g. the second, third, or fourth, or fifth anti-cancer agent, described below) are administered in doses lower than the doses commonly employed when such agents are used as monotherapy for treating the disorder. In certain embodiments, the substituted adeninyl-propyloxy phosphonic acid or related compound described herein (e.g., a compound of Formula I, or other compounds in Section III) and the additional therapeutic agent(s) (e.g. the second, third, or fourth, or fifth anti-cancer agent, described below) are present in the same composition, which is suitable for oral administration.

In certain embodiments, the substituted adeninyl-propyloxy phosphonic acid or related compound described herein (e.g., a compound of Formula I, or other compounds in Section III) and the additional therapeutic agent(s) (e.g. the second, third, or fourth, or fifth anti-cancer agent, described below) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of the substituted adeninyl-propyloxy phosphonic acid or related compound described herein (e.g., a compound of Formula I, or other compounds in Section III), a pharmaceutically acceptable carrier, vehicle or diluent, and optionally at least one additional therapeutic agent listed above.

### Cancer

Accordingly, another aspect of the invention provides a method of treating cancer in a patient. The method comprises administering to a subject in need thereof (i) a therapeutically effective amount of a substituted adeninyl-propyloxy phosphonic acid or related compound described herein and (ii) a second anti-cancer agent, in order to treat the cancer.

In certain embodiments, the second anti-cancer agent is radiation therapy.

In certain embodiments, the second anti-cancer agent is a therapeutic antibody. In certain embodiments, the therapeutic antibody targets one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, interleukin-1 beta, B4GALNT1, interleukin-6, and interleukin-6 receptor.

In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, lgN311, bevacizumab, IM-2C6, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MEDI9447, daratumumab, belimumab, canakinumab, dinutuximab, siltuximab, and tocilizumab.

In certain embodiments, the second anti-cancer agent is a cytokine. In certain embodiments, the cytokine is IL-12, IL-15, GM-CSF, or G-CSF.

In certain embodiments, the second anti-cancer agent is sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), or 2-chloro-deoxyadenosine.

In certain embodiments, the second anti-cancer agent is a placental growth factor, an antibody-drug conjugate, an oncolytic virus, or an anti-cancer vaccine. In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consistent of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is an immune checkpoint inhibitor (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264. In certain embodiments, the immune checkpoint inhibitor is an agent that inhibits one or more of (i) cytotoxic T-lymphocyte-associated antigen 4 (CTLA4), (ii) programmed cell death protein 1 (PD1), (iii) PDL1, (iv) LAB3, (v) B7-H3, (vi) B7-H4, and (vii) TIM3. In certain embodiments, the immune checkpoint inhibitor is ipilumumab. In certain embodiments, the immune checkpoint inhibitor is pembrolizumab.

In certain embodiments, the second anti-cancer agent is a monoclonal antibody that targets a non-checkpoint target (e.g., herceptin). In certain embodiments, the second anti-cancer agent is a non-cytoxic agent (e.g., a tyrosine-kinase inhibitor).

In certain embodiments, the second anti-cancer agent is selected from mitomycin, ribomustin, vincristine, tretinoin, etoposide, cladribine, gemcitabine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, aminoglutethimide, amsacrine, proglumide, elliptinium acetate, ketanserin, doxifluridine, etretinate, isotretinoin, streptozocin, nimustine, vindesine, cytarabine, bicalutamide, vinorelbine, vesnarinone, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, colony stimulating factor-1, colony stimulating factor-2, denileukin diftitox, interleukin-2, leutinizing hormone releasing factor, interferon-alpha, interferon-2 alpha, interferon-beta, interferon-gamma.

In certain embodiments, the second anti-cancer agent is an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, or a WEE1 Inhibitor.

In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40, CD137, CD40, GITR, CD27, HVEM, TNFRSF25, or ICOS. In certain embodiments, the second anti-cancer agent is an agonist of OX40, CD137, CD40, or GITR. In certain embodiments, the second anti-cancer agent is an agonist of CD27, HVEM, TNFRSF25, or ICOS.

In certain embodiments, the method further comprises administering to the subject a third anti-cancer agent. In certain embodiments, the method further comprises administering to the subject a fourth anti-cancer agent. In certain embodiments, the method further comprises administering to the subject a fifth anti-cancer agent.

In certain embodiments, the third anti-cancer agent is one of the second anti-cancer agents described above. In certain embodiments, the fourth anti-cancer agent is one of the second anti-cancer agents described above. In certain embodiments, the fifth anti-cancer agent is one of the second anti-cancer agents described above.

### Inflammatory Disorders

Accordingly, another aspect of the invention provides a method of treating an inflammatory disorder in a patient. The method comprises administering to a subject in need thereof (i) a therapeutically effective amount of a substituted adeninyl-propyloxy phosphonic acid or related compound described herein and (ii) a second therapeutic agent, in order to treat the inflammatory disorder.

In certain embodiments, the second therapeutic agent is a small molecule or a recombinant biologic agents. In certain embodiments, the second therapeutic agent is selected from acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®, Neoral®), tacrolimus, sirolimus, mycophenolate, leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), anti-T cell antibodies such as Thymoglobulin, IV Immunoglobulins (IVIg), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-Bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), anti-IL36 agents such as BI655130, Dihydroorotate dehydrogenase inhibitors such as IMU-838, anti-OX40 agents such as KHK-4083, microbiome agents such as RBX2660, SER-287, Narrow spectrum kinase inhibitors such as TOP-1288, anti-CD40 agents such as BI-655064 and FFP-104, guanylate cyclase agonists such as dolcanatide, sphingosine kinase inhibitors such as opaganib, anti-IL-12/IL-23 agents such as AK-101, Ubiquitin protein ligase complex inhibitors such as BBT-401, sphingosine receptors modulators such as BMS-986166, P38MAPK/PDE4 inhibitors such as CBS-3595, CCR9 antagonists such as CCX-507, FimH antagonists such as EB-8018, HIF-PH inhibitors such as FG-6874, HIF-1α stabilizer such as GB-004, MAP3K8 protein inhibitors such as GS-4875, LAG-3 antibdies such as GSK-2831781, RIP2 kinase inhibitors such as GSK-2983559, Farnesoid X receptor agonist such as MET-409, CCK2 antagonists such as PNB-001, IL-23 Receptor antagonists such as PTG-200, Purinergic P2X7 receptor antagonists such as SGM-1019, PDE4 inhibitors such as Apremilast, ICAM-1 inhibitors such as alicaforsen sodium, Anti-IL23 agents such as guselkumab, brazikumab and mirkizumab, ant-IL-15 agents such as AMG-714, TYK-2 inhibitors such as BMS-986165, NK Cells activators such as CNDO-201, RIP-1 kinase inhibitors such as GSK-2982772, anti-NKGD2 agents such as JNJ-4500, CXCL-10 antibodies such as JT-02, IL-22 receptor agonists such as RG-7880, GATA-3 antagonists such as SB-012, and Colony-stimulating factor-1 receptor inhibitors such as edicotinib.

In certain embodiments, the method further comprises administering to the subject a third therapeutic agent. In certain embodiments, the method further comprises administering to the subject a fourth therapeutic agent. In certain embodiments, the method further comprises administering to the subject a fifth therapeutic agent.

In certain embodiments, the third therapeutic agent is one of the second therapeutic agents described above. In certain embodiments, the fourth therapeutic agent is one of the second therapeutic agents described above. In certain embodiments, the fifth therapeutic agent is one of the second therapeutic agents described above.

Immune Disorders Other than a Viral Infection

Accordingly, another aspect of the invention provides a method of treating an immune disorder other than a viral infection in a patient. The method comprises administering to a subject in need thereof (i) a therapeutically effective amount of a substituted adeninyl-propyloxy phosphonic acid or related compound described herein and (ii) a second therapeutic agent, in order to treat the immune disorder other than a viral infection.

In certain embodiments, the second therapeutic agent is pentoxifylline, propentofylline, torbafylline, cyclosporine, methotrexate, tamoxifen, forskolin and analogs thereof, tar derivatives, steroids, vitamin A and its derivatives, vitamin D and its derivatives, a cytokine, a chemokine, a stem cell growth factor, a lymphotoxin, an hematopoietic factor, a colony stimulating factor (CSF), erythropoietin, thrombopoietin, tumor necrosis factor-α (TNF), TNF-θ, granulocyte-colony stimulating factor (G-CSF), granulocyte macrophage-colony stimulating factor (GM-CSF), interferon-α, interferon-β, interferon-γ, interferon-λ, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-β, platelet-growth factor, TGF-α, TGF-β, insulin-like growth factor-I, insulin-like growth factor-II, macrophage-CSF (M-CSF), IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, or lymphotoxin.

In certain embodiments, the method further comprises administering to the subject a third therapeutic agent. In certain embodiments, the method further comprises administering to the subject a fourth therapeutic agent. In certain embodiments, the method further comprises administering to the subject a fifth therapeutic agent.

In certain embodiments, the third therapeutic agent is one of the second therapeutic agents described above. In certain embodiments, the fourth therapeutic agent is one of the second therapeutic agents described above. In certain embodiments, the fifth therapeutic agent is one of the second therapeutic agents described above.

### Neurodegenerative Disorders

Accordingly, another aspect of the invention provides a method of treating a neurodegenerative disorder in a patient. The method comprises administering to a subject in need thereof (i) a therapeutically effective amount of a substituted adeninyl-propyloxy phosphonic acid or related compound described herein and (ii) a second therapeutic agent, in order to treat the neurodegenerative disorder.

In certain embodiments, the second therapeutic agent is a dopaminergic treatment, a cholinesterase inhibitor, an antipsychotic drug, deep brain stimulation (for example, to stop tremor and refractory movement disorders), riluzole, a caffein A2A receptor antagonist, pramipexole, or rasagilin.

In certain embodiments, the method further comprises administering to the subject a third therapeutic agent. In certain embodiments, the method further comprises administering to the subject a fourth therapeutic agent. In certain embodiments, the method further comprises administering to the subject a fifth therapeutic agent.

In certain embodiments, the third therapeutic agent is one of the second therapeutic agents described above. In certain embodiments, the fourth therapeutic agent is one of the second therapeutic agents described above. In certain embodiments, the fifth therapeutic agent is one of the second therapeutic agents described above.

### V. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

In certain embodiments, the invention provides a pharmaceutical composition comprising a compound described herein (e.g., a compound of Formula I, I-A, or I-B) and a pharmaceutically acceptable carrier. In certain embodiments, the invention provides a pharmaceutical composition comprising a compound described herein (e.g., a compound of Formula I, I-A, or I-B), an additional therapeutic agent (e.g., a compound described in Section IV), and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a substituted adeninyl-propyloxy phosphonic acid or related compound described herein in a therapeutically effective amount for the treatment of a medical disorder described herein.

### EXAMPLES

The invention now being generally described, will be more readily understood by reference to the following examples, which are included merely for purposes of illustration of certain aspects and embodiments of the present invention, and are not intended to limit the invention. Starting materials described herein can be obtained from commercial sources or may be readily prepared from commercially available materials using transformations known to those of skill in the art.

### Example 1—Transient Cellular Assay for Inhibiting LINE1 Reverse Transcriptase

Exemplary compounds were tested for ability to inhibit LINE1 reverse transcriptase using a transient artificial-intron Cis LINE1 reporter assay. Assay procedures and results are described below.

### Part I—Procedure for Transient Artificial-Intron Cis LINE1 Reporter Assay

Intron-disrupted Firefly luciferase (FLuc) expression cassettes were generated as described by Xie, Y. et al. “Characterization of L1 retrotransposition with high-throughput dual-luciferase assays,” Nucleic Acid Res. (2011) Vol. 39, No. 3, e16. In addition, the plasmid contained an intact Renilla luciferase (RLuc) expression cassette on the vector backbone, in order to normalize transfection efficiency and measure potential cell toxicity.

HEK 293 cells were seeded in 96-well plates at 1,000 cells/well in 55 μL and grown for 24 hours. Cells were transfected with FuGeneHD (Promega) following the manufacturer's protocol. Each well received 0.133 ng plasmid, 0.4 μL FuGeneHD reagent, and 4.5 μL GlutaMAX-I-supplemented Opti-MEM I reduced-serum medium (Invitrogen). Cells were simultaneously treated with test compound serially diluted starting at 100 μM in a 3-fold dilution dose response.

Luminescence was measured using the Dual-Glo Luciferase Assay System (Promega) following the manufacturer's instructions. The ratio between FLuc and RLuc gene expression (each of which was normalized against its expression in a control well without test compound) was used to report LINE1 activity.

### Part II—Results

Experimental results are provided in Table 7, below. The symbol “***” indicates an IC₅₀ less than or equal to 0.05 μM. The symbol “**” indicates an IC₅₀ in the range of greater than 0.05 μM to less than or equal to 0.5 μM. The symbol “*” indicates an IC₅₀ greater than 0.5 μM.

Certain known reverse transcriptase inhibitors were found to have lower activity in this assay than islatravir, compound 8, and 4′-ethynyl-2′-deoxyadenosine. For example, each of lobucavir, AZT (zidovudine), apricitabine, and lamivudine (3TC) did not achieve a detectable IC₅₀ value in this assay when tested at concentrations up to 100 μM.

TABLE 7

| Compound No. | IC₅₀ |
|---|---|
| I-1 | *** |
| V-1 | * |
| V-7 | *** |

### Example 2—Stable Cellular Assay for Inhibiting LINE1 Reverse Transcriptase

Exemplary compounds were tested for ability to inhibit LINE1 reverse transcriptase using a stable artificial-intron Cis LINE1 reporter assay. Assay procedures and results are described below.

### Part I—Procedure for Stable Artificial-Intron Cis LINE1 Reporter Assay

A stable HeLa Tet-On 3G (Takara, cat no 631183) cell line expressing a bi-directional inducible LINE1 construct was generated as described in Xie, Y. et al. “Cell division promotes efficient retrotransposition in a stable L1 reporter cell line,” Mobile DNA (2013) 4:10. Single cell clones were screened for high Luciferase expression and the highest expression Firefly expressing clone was chosen for compound testing.

Test compounds were serially diluted in DMSO and spotted in 96-well plates. Subsequently the HeLa L1 artificial-intron reporter cells were plated into the compound-containing wells (8,000 cells/well), and the cells were induced for reporter expression with doxycycline (Sigma cat no D9891) at a final concentration of 500 ng/mL. Luminescence was measured 72 h after plating using the Dual-Glo Luciferase Assay System (Promega cat no E2940) following the manufacturer's instructions. The Firefly Luciferase activity (normalized against its activity in a control well without test compound) was used to report LINE1 activity.

### Part II—Results

Experimental results are provided in Table 8, 8-A, and 8-B, below. The symbol “***” indicates an IC₅₀ less than or equal to 0.05 μM. The symbol “**” indicates an IC₅₀ in the range of greater than 0.05 μM to less than or equal to 0.5 μM. The symbol “*” indicates an IC₅₀ greater than 0.5 μM.

TABLE 8

| Compound No. | IC₅₀ |
|---|---|
| I-1 | *** |
| I-20 | *** |
| I-21 | ** |
| Mixture of | *** |
| I-20 and I-21 | |
| V-1 | * |
| V-7 | ** |
| VI-1 | ** |

TABLE 8-A

| Compound No. | IC₅₀ |
|---|---|
| I-115 | ** |
| V-105 | * |
| V-106 | * |

TABLE 8-B

| Compound | IC₅₀ |
|---|---|
| lobucavir | * |
| AZT (zidovudine) | * |
| lamivudine (3TC) | * |

### Example 3—Biochemical Assay for Inhibiting LINE1 Reverse Transcriptase

Exemplary compounds were tested for ability to inhibit LINE1 reverse transcriptase using a homogeneous time-resolved fluorescence (HTRF) assay. Assay procedures and results are described below.

### Part I—Procedure for Homogeneous Time-Resolved Fluorescence LINE1 RT Assay

The LINE1 reverse transcriptase homogeneous time-resolved fluorescence (HTRF) assay was performed with recombinant MBP-tagged LINE1 protein (238-1061) (generated and purified according to procedures in Dai L. et al. BMC Biochemistry 2011; 12:18) in a 384-well format. Test compound was serially diluted in DMSO and further diluted in the assay buffer (50 mM Tris-HCl, 50 mM KCl, 10 mM MgCl₂, 10 mM DTT, pH 8.1) to achieve a final DMSO concentration of 1%. The serially diluted compound was mixed with 64 ng/well of LINE1 enzyme, 5 nM of pre-annealed template/biotin-primer pair (synthesized at Generay Biotechnology), 10 nM of Fluorescein-12-dATP fluorescent probe (Perkin Elmer), and 1 μM dGTP/dCTP/dTTP (Thermo Fisher Scientific) in the assay buffer. The template/biotin-primer sequences were as follows:

| | 5′ to 3′ |
|---|---|
| (SEQ ID NO: 9) | |
| | GUAACUAGAGAUCCCUCAGACCCUUUUAGUCAGAAU |
| | 5′ to 3′ |
| (SEQ ID NO: 10) | |
| | Biotin-TTCTGACTAAAAGGGTCTGAGGGAT. |

After incubating at 25° C. for 60 minutes, the detection reagent (20 mM EDTA with streptavidin-terbium cryptate, Cisbio Bioassay) in the PPI buffer (Cisbio Bioassay) was added, and the mixture was incubated at 25° C. for 30 minutes. At the end of the incubation, fluorescence was read at ex/em=337/485 nm and ex/em=337/520 nm on an Envision 2104 plate reader (Perkin Elmer). The fluorescence ratio at 520/485 nm was used for the calculation. Percent inhibition was calculated with the DMSO sample as 0% inhibition and no enzyme as 100% inhibition. The IC₅₀ was calculated by fitting the compound dose inhibition curve with a 4-parameter non-linear regression equation.

### Part II—Results

The tetra(sodium) salt of the Test Compound was found to inhibit LINE1 reverse transcriptase with an IC₅₀ of 0.071 μM. The Test Compound was:

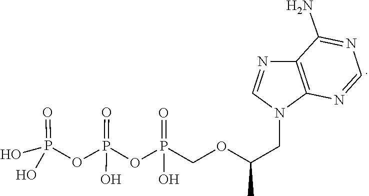 [SMILES: C[C@H](Cn1cnc2c(N)ncnc21)OCP(=O)(O)OP(=O)(O)OP(=O)(O)O]

### Example 4—Biochemical Assay for Inhibiting HERV-K Reverse Transcriptase

Exemplary compounds were tested for ability to inhibit HERV-K reverse transcriptase using a homogeneous time-resolved fluorescence (HTRF) assay. Assay procedures and results are described below.

### Part I—Procedure for Homogeneous Time-Resolved Fluorescence HERV-K RT Assay

The HERV-K reverse transcriptase homogeneous time-resolved fluorescence (HTRF) assay was performed in a 384-well format with HERV-K reverse transcriptase (2-596)-8His protein. Baculoviruses were created using Bac-to-Bac technology (Invitrogen). pFastBac donor plasmids containing HERV-K reverse transcriptase sequence (NCBI GenBank number AAC63291.1, J. Virology (1999) Vol. 73, No. 3, pp. 2365-2375) were transformed into DH10 Bac cells following the manufacturer's instructions. Recombinant bacmids were then isolated clonally and transfected into SF9 cells with lipofectin. HERV-K reverse transcriptase was expressed in the SF9 insect cells and then purified using immobilized metal affinity chromatography (IMAC) followed by size-exclusion chromatography (SEC).

Test compound was serially diluted in DMSO and further diluted in the assay buffer (50 mM Tris-HCl, 50 mM KCl, 10 mM MgCl₂, 10 mM DTT, pH 8.1) to achieve a final DMSO concentration of 1%. The serially diluted compound was mixed with 32 ng/well of HERV-K enzyme, 5 nM of pre-annealed template/biotin-primer pair (synthesized at Generay Biotechnology), 10 nM of Fluorescein-12-dATP fluorescent probe (Perkin Elmer), and 1 μM dGTP/dCTP/dTTP (Thermo Fisher Scientific) in the assay buffer. The template/biotin-primer sequences were as follows:

| | 5′ to 3′ |
|---|---|
| (SEQ ID NO: 9) | |
| | GUAACUAGAGAUCCCUCAGACCCUUUUAGUCAGAAU |
| | 5′ to 3′ |
| (SEQ ID NO: 10) | |
| | Biotin-TTCTGACTAAAAGGGTCTGAGGGAT. |

After incubating at 25° C. for 30 minutes, the detection reagent 20 mM EDTA with streptavidin-terbium cryptate (Cisbio Bioassay) in the PPI buffer (Cisbio Bioassay) was added, and the mixture was incubated at 25° C. for 60 minutes. At the end of the incubation, fluorescence was read at ex/em=337/485 nm and ex/em=337/520 nm on an Envision 2104 plate reader (Perkin Elmer). The fluorescence ratio at 520/485 nm was used for the calculation. Percent inhibition was calculated with the DMSO sample as 0% inhibition and no enzyme as 100% inhibition. The IC₅₀ was calculated by fitting the compound dose inhibition curve with a 4-parameter non-linear regression equation.

### Part II—Results

The tetra(sodium) salt of the Test Compound was found to inhibit HERV-K reverse transcriptase with an IC₅₀ of 0.020 μM. The Test Compound was:

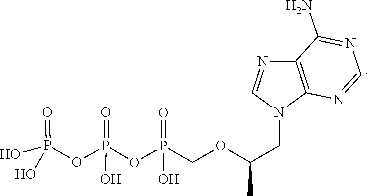 [SMILES: C[C@H](Cn1cnc2c(N)ncnc21)OCP(=O)(O)OP(=O)(O)OP(=O)(O)O]

### Example 5—Cellular Assay for Altering IFN Production in THP1 TREX1 KO Cells

THP1-Dual™ KO-TREX1 cells were purchased from Invivogen (cat #thpd-kotrex). The THP1-Dual™ KO-TREX1 cells were cultured in RPMI 1640, 10% heat-inactivated fetal bovine serum, 25 mM HEPES, 10 μg/mL Blasticidin, and 100 μg/mL Zeocin. THP1-Dual™ KO-TREX1 cells were treated with a dose titration of test compound in the presence of 1 μM 5-aza-2′-deoxycytidine (Sigma, cat #189825). Type 1 Interferon and cell viability were assessed after five days of treatment.

Stock solution of test compound was prepared in DMSO followed by a three-fold dilution in DMSO. Additional 50× dilution was prepared in cell culture media for each dilution. 10 μL of diluted test compound was then added to a 384-well plate.

THP1-Dual™ KO-TREX1 cells were treated with 1 μM 5-aza-2′-deoxycytidine. The THP1-Dual™ KO-TREX1 cells (50 μL) were added to each well of the 384-well plate containing test compound titration at 10,000 cells/well. Cells were incubated at 37° C., 5% CO₂ in a humidified incubator for five days. On day five, 20 μL of cell supernatant was transferred to a 384-well, white-walled plate, followed by addition to each well of 50 μL of QUANTI-LUC solution containing stabilizer. Luminescence was detected on a plate reader according to manufacturer's instructions. Percent inhibition of interferon was calculated using the following analysis: (Average DMSO−Sample)/(Average DMSO−Average 30 μM control reagent)*100. Percent induction of interferon was calculated using the following analysis: (Sample−Average DMSO)/(10 μM control reagent−Average DMSO)*100.

The remaining cells were assessed for cell viability by adding 30 μl of CellTiter-Glo (Promega, G9683) solution to each well, and placed on a shaker for 10 minutes at room temperature. Luminescence was detected on a plate reader, according to manufacturer's instructions. Percent inhibition of cell viability using CellTiter-Glo was calculated using the following analysis. (Average DMSO-Sample)/(Average DMSO-Average 20 μM control reagent)*100. The control reagent is Z-Leu-Leu-leucinal (see, for example, https://pubchem.ncbi.nlm.nih.gov/compound/462382),

### Part II—Results

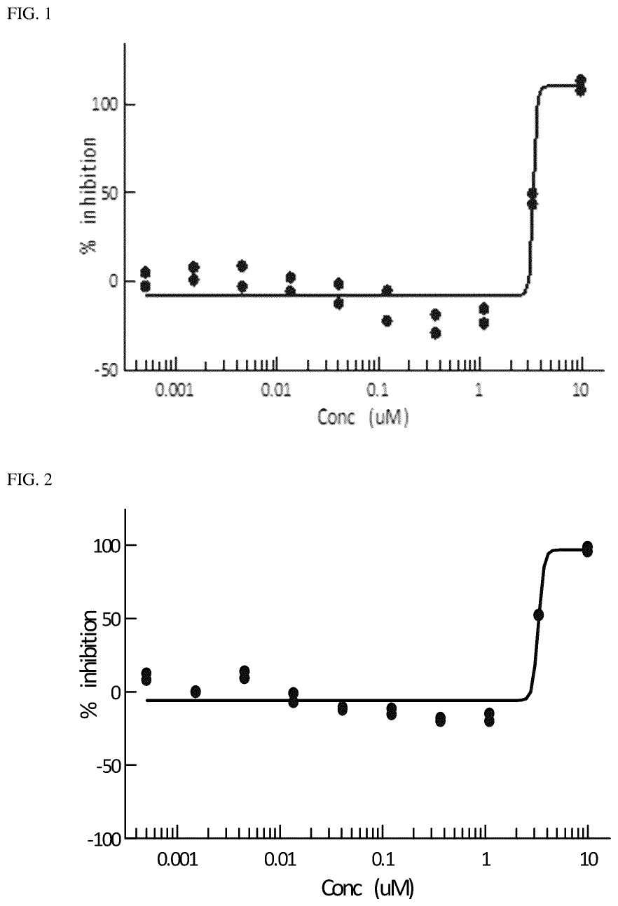

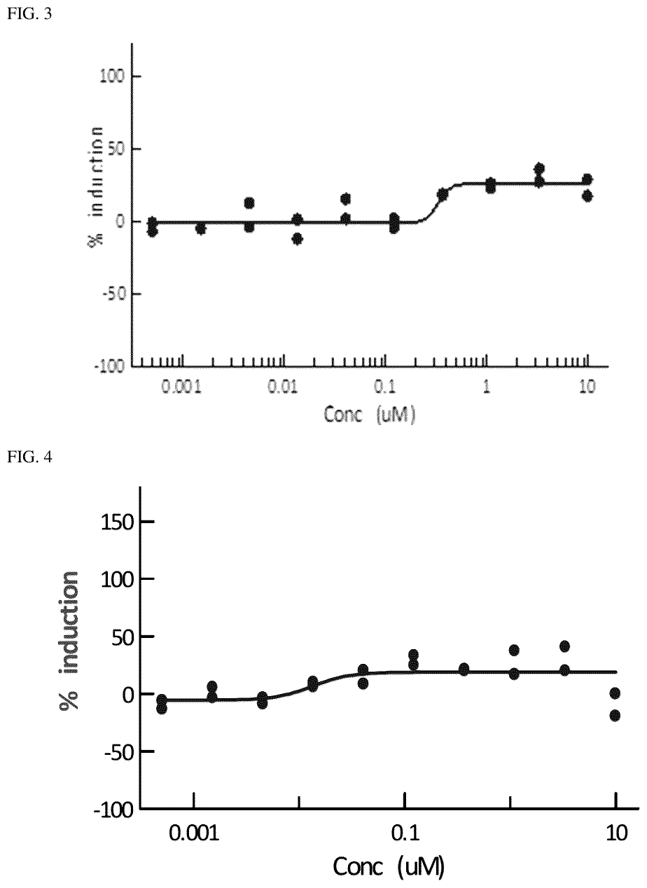

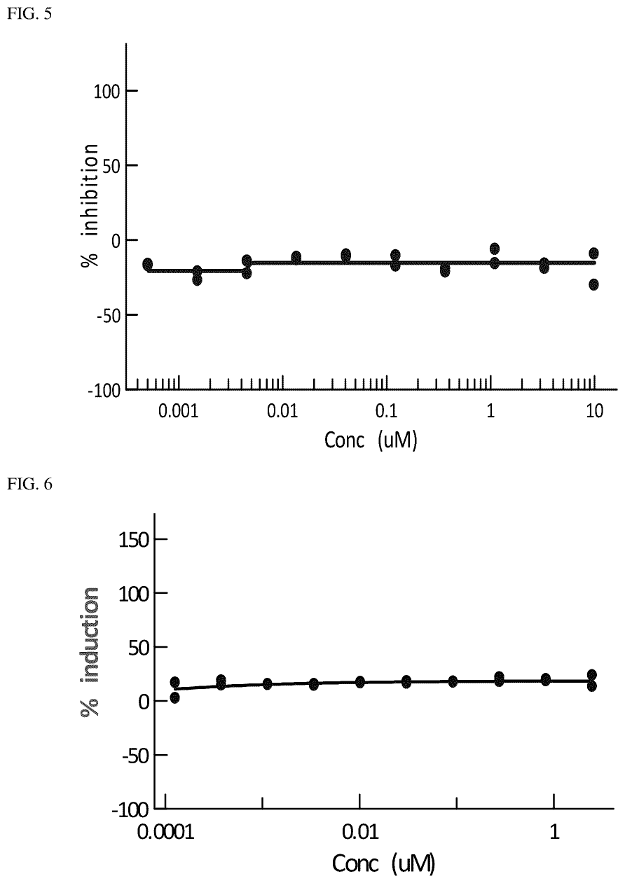

Experimental results for each of compounds I-1, I-20, I-21, I-115, V-1, and VI-1, for changes in IFN levels are provided in FIG. 1-6, respectively, as % inhibition (or % induction) versus compound concentration. CellTiter-Glo analysis demonstrated <5% change in cell viability at all concentrations of each selected compound tested in this assay, except for the highest dose of compounds I-1 and I-20. At 10 μM of compound I-1, the CellTiter-Glo analysis identified a 57% average inhibition. At 10 μM of compound I-20, the CellTiter-Glo analysis identified a 65% average inhibition.

### Example 6—Producing THP1 TREX1 KO Xenografts with Decitabine-Induced IFN

The ability to produce THP1-Dual™ KO-TREX1 xenografts in mice that displayed decitabine-dependent IFN induction was tested. Assay procedures and results are described below.

### Part I—Procedure for Producing THP1 TREX1 KO Xenografts with Decitabine-Induced IFN

CB-17 SCID female mice were inoculated subcutaneously with 10 million THP1-Dual™ KO-TREX1 cells in 200 μl PBS with Matrigel (1:1). Mice were randomized when tumor volume reached 350-400 mm³ and grouped at N═3 per treatment. Mice bearing THP1-Dual™ KO-TREX1 xenograft tumors were then administered vehicle or decitabine (DAC) at 5 mg/kg IP, once daily, starting on day 1, for 4 days. Decitabine was formulated in sterile PBS, pH 7.4. Tumors were harvested daily for 5 days starting on day 2, lysed with RIPA lysis buffer containing protease and phosphatase inhibitors, and grinded at 50 Hz for 5 min. Tumors were then centrifuged, and Pierce™ BCA Protein Assay Kit was used to measure protein concentration. Equal amounts of proteins were added to 96-well black plates, and luciferase signal was measured using the QUANTI-Luc™ detection medium according to manufacturer's instructions. Luminescence was measured using the EnVision® 2105 Multimode Plate Reader.

### Part II—Results

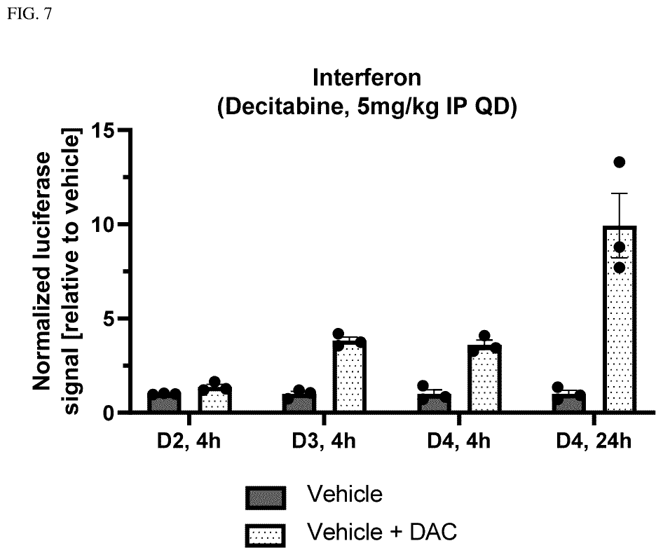

Experimental results are depicted in FIG. 7. Data was normalized relative to vehicle. In FIG. 16, “DAC” is an abbreviation for decitabine; “D2, 4 h” depicts interferon data from day 2, with tumor harvested 4 hours after decitabine dosing; “D3, 4 h” depicts interferon data from day 3, with tumor harvested 4 hours after decitabine dosing; “D4, 4 h” depicts interferon data from day 4, with tumor harvested 4 hours after decitabine dosing; and “D4, 24 h” depicts interferon data from day 5, with tumor harvested 24 hours after the final decitabine dosing on day 4.

### Example 7—Assay for Altering IFN Production in THP1 TREX1 KO Xenografts

Exemplary compounds may be tested for their ability to alter IFN levels in THP1-Dual™ KO-TREX1 xenografts in mice (produced according to the procedure described in Example 6). Assay procedures are described below.

### Part I—Procedure for Altering IFN Production in THP1 TREX1 KO Xenografts

CB-17 SCID female mice are inoculated subcutaneously with 10 million THP1-Dual™ KO-TREX1 cells in 200 μl PBS with Matrigel (1:1) and grouped when tumor volume reaches 350-400 mm³. Mice bearing THP1-Dual™ KO-TREX1 xenograft tumors are then separated into 5 groups. Three groups are administered: (1) decitabine (DAC) at 5 mg/kg IP, once daily, for 4 days, and (2) test compound at one of three doses, once daily, for 4 days. One group is administered decitabine (DAC) at 5 mg/kg IP, once daily, for 4 days, and the test compound vehicle control. The final group is administered the vehicle control from both the test compound and the vehicle control from decitabine. Decitabine is formulated in sterile PBS, pH 7.4.

Tumors are harvested daily for 5 days starting on day 2, lysed with RIPA lysis buffer containing protease and phosphatase inhibitors, and grinded at 50 Hz for 5 min. Tumors are then centrifuged, and Pierce™ BCA Protein Assay Kit is used to measure protein concentration. Equal amounts of proteins are added to 96-well black plates, and luciferase signal is measured using the QUANTI-Luc™ detection medium according to manufacturer's instructions. Luminescence is measured using the EnVision® 2105 Multimode Plate Reader.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating a disorder selected from the group consisting of an inflammatory disorder, a neurodegenerative disorder, and an immune disorder other than a viral infection, comprising administering to a subject in need thereof a therapeutically effective amount of a compound of Formula I to treat the disorder; wherein Formula I is represented by: 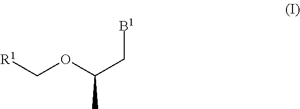
or a stereoisomer thereof; or a pharmaceutically acceptable salt of either of the foregoing; wherein:
R¹ is —P(O)(OR³)(N(R⁴)(R⁵)), —P(O)(OR³)₂, or —P(O)(N(R⁴)(R⁵))₂;
R² is hydrogen, —NH₂, or fluoro;
R³ represents independently for each occurrence:
a. phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein said phenyl, naphthyl, 5-6 membered monocyclic heteroaryl, and 8-10 membered bicyclic heteroaryl are substituted with m instances of R⁸;
b. hydrogen, —P(O)(OH)₂, —P(O)(OH)—O—P(O)(OH)₂, C₁₋₂₀ alkyl, C₁₋₂₀ haloalkyl, —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), —(C₁₋₁₀ alkylene)-Y—(C₂₋₂₀ alkynylene)-R¹¹, or —C(R⁶)₂—CO₂R¹⁰; wherein said C₁₋₂₀ alkyl, C₁₋₂₀ haloalkyl, and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene; or
c. two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸;
R⁴ and R⁹ each represents independently for each occurrence hydrogen or C₁₋₄ alkyl; or R⁴ and one occurrence of R⁶ are taken together with atoms to which they are attached to form a 4-7 membered saturated heterocyclic ring containing 1 nitrogen atom; or two instances of R⁹ are taken together with the atom to which they are attached to form a 4-7 membered saturated heterocyclic ring containing 1 nitrogen atom and optionally 1 oxygen atom;
R⁵ represents independently for each occurrence:
a. —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, —C(R¹⁰)(H)—CH₂—CO₂R¹⁰, C₁₋₂₀ alkyl, C₁₋₂₀ haloalkyl, —(C₁₋₁₀ alkylene)-X—(C₁₋₁₀ alkyl), —(C₁₋₁₀ alkylene)-phenyl, —(C₁₋₄ haloalkylene)-phenyl, —C(O)-phenyl, —C(S)-phenyl, or —C(O)-(5-6 membered monocyclic heteroaryl having 1, 2, or 3 heteroatoms independently selected from nitrogen, oxygen, and sulfur); or
b. phenyl; naphthyl; a 5-6 membered monocyclic heteroaryl having 1, 2, or 3 heteroatoms independently selected from nitrogen, oxygen, and sulfur; or an 8-10 membered bicyclic heteroaryl having 1, 2, or 3 heteroatoms independently selected from nitrogen, oxygen, and sulfur;
wherein said phenyl, naphthyl, 5-6 membered monocyclic heteroaryl, and 8-10 membered bicyclic heteroaryl are substituted with m instances of R⁸; and said C₁₋₂₀ alkyl, C₁₋₂₀ haloalkyl, and C₁₋₁₀ alkyl are optionally substituted with one hydroxyl;
R⁶ represents independently for each occurrence C₁₋₆ alkyl, C₁₋₆ haloalkyl, C₃₋₅ cycloalkyl, —CN, or hydrogen, wherein said C₁₋₆ alkyl is optionally substituted with —S—(C₁₋₄ alkyl), —SH, C₁₋₄ alkoxyl, C₁₋₄ haloalkoxyl, hydroxyl, —OCH₂CN, phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; or two instances of R⁶ are taken together with the carbon atom to which they are attached to form a 3-6 membered saturated ring having 0 or 1 oxygen atom;
R⁷ represents independently for each occurrence C₁₋₈ alkyl, C₁₋₆ haloalkyl, C₂₋₆ alkenyl, C₃₋₇ cycloalkyl, or a 4-7 membered saturated monocyclic heterocyclyl having one or two heteroatoms independently selected from nitrogen, oxygen, and sulfur; wherein said C₁₋₈ alkyl is optionally substituted with C₁₋₄ alkoxyl, phenyl, C₃₋₇ cycloalkyl, or a 4-7 membered saturated monocyclic heterocyclyl having one or two heteroatoms independently selected from nitrogen, oxygen, and sulfur;
R⁸ represents independently for each occurrence halo, C₁₋₄ alkyl, C₁₋₄ haloalkyl, C₁₋₄ alkoxyl, C₁₋₄ haloalkoxyl, —(C₀₋₃ alkylene)-CO₂R¹⁰, —CN, or —N(R⁹)₂;
R¹⁰ represents independently for each occurrence C₁₋₆ alkyl, C₃₋₇ cycloalkyl, or hydrogen;
R¹¹ represents independently for each occurrence C₁₋₂ haloalkyl, —SF₅, —Si(C₁₋₄ alkyl)₃, —Si(CH₃)₂(C₁₋₄ haloalkyl), —Si(CH₃)₂(C₃₋₇ cycloalkyl), 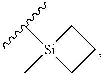 [SMILES: *[Si]1(C)CCC1]
—Si(CH₃)₂(phenyl), —S-phenyl, —O-phenyl, phenyl, thiophenyl, pyridinyl, or C₃₋₇ cycloalkyl; wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅;
X represents independently for each occurrence —O—, ψ-OC(O)—, —OC(O)O—, ψ-OC(O)—N(R⁹)—, —S—, —S—S—, or ψ-SC(O)—; wherein ψ denotes the point of attachment to C₁₋₁₀ alkylene;
Y represents independently for each occurrence —O—, —S—, or —CF₂—;
B¹ is 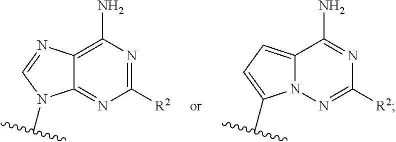
and
m and p are independently for each occurrence 0, 1, 2, or 3;
wherein one or more hydrogen atoms may be replaced with deuterium.

2-3. (canceled)

4. A method of inhibiting LINE1 reverse transcriptase activity in a subject suffering from a disorder selected from the group consisting of an inflammatory disorder, a neurodegenerative disorder, and an immune disorder other than a viral infection, comprising contacting a LINE1 reverse transcriptase with an effective amount of a compound of Formula I, in order to inhibit the activity of said LINE1 reverse transcriptase; wherein Formula I is represented by: 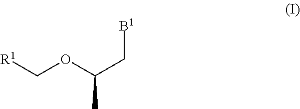
or a stereoisomer thereof; or a pharmaceutically acceptable salt of either of the foregoing; wherein:
R¹ is —P(O)(OR³)(N(R⁴)(R⁵)), —P(O)(OR³)₂, or —P(O)(N(R⁴)(R⁵))₂;
R² is hydrogen, —NH₂, or fluoro;
R³ represents independently for each occurrence:
a. phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein said phenyl, naphthyl, 5-6 membered monocyclic heteroaryl, and 8-10 membered bicyclic heteroaryl are substituted with m instances of R⁸;
b. hydrogen, —P(O)(OH)₂, —P(O)(OH)—O—P(O)(OH)₂, C₁₋₂₀ alkyl, C₁₋₂₀ haloalkyl, —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), —(C₁₋₁₀ alkylene)-Y—(C₂₋₂₀ alkynylene)-R¹¹, or —C(R⁶)₂—CO₂R¹⁰; wherein said C₁₋₂₀ alkyl, C₁₋₂₀ haloalkyl, and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene; or
c. two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸;
R⁴ and R⁹ each represents independently for each occurrence hydrogen or C₁₋₄ alkyl; or R⁴ and one occurrence of R⁶ are taken together with atoms to which they are attached to form a 4-7 membered saturated heterocyclic ring containing 1 nitrogen atom; or two instances of R⁹ are taken together with the atom to which they are attached to form a 4-7 membered saturated heterocyclic ring containing 1 nitrogen atom and optionally 1 oxygen atom;
R⁵ represents independently for each occurrence:
a. —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, —C(R¹⁰)(H)—CH₂—CO₂R¹⁰, C₁₋₂₀ alkyl, C₁₋₂₀ haloalkyl, —(C₁₋₁₀ alkylene)-X—(C₁₋₁₀ alkyl), —(C₁₋₁₀ alkylene)-phenyl, —(C₁₋₄ haloalkylene)-phenyl, —C(O)-phenyl, —C(S)-phenyl, or —C(O)-(5-6 membered monocyclic heteroaryl having 1, 2, or 3 heteroatoms independently selected from nitrogen, oxygen, and sulfur); or
b. phenyl; naphthyl; a 5-6 membered monocyclic heteroaryl having 1, 2, or 3 heteroatoms independently selected from nitrogen, oxygen, and sulfur; or an 8-10 membered bicyclic heteroaryl having 1, 2, or 3 heteroatoms independently selected from nitrogen, oxygen, and sulfur;
wherein said phenyl, naphthyl, 5-6 membered monocyclic heteroaryl, and 8-10 membered bicyclic heteroaryl are substituted with m instances of R⁸; and said C₁₋₂₀ alkyl, C₁₋₂₀ haloalkyl, and C₁₋₁₀ alkyl are optionally substituted with one hydroxyl;
R⁶ represents independently for each occurrence C₁₋₆ alkyl, C₁₋₆ haloalkyl, C₃₋₅ cycloalkyl, —CN, or hydrogen, wherein said C₁₋₆ alkyl is optionally substituted with —S—(C₁₋₄ alkyl), —SH, C₁₋₄ alkoxyl, C₁₋₄ haloalkoxyl, hydroxyl, —OCH₂CN, phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; or two instances of R⁶ are taken together with the carbon atom to which they are attached to form a 3-6 membered saturated ring having 0 or 1 oxygen atom;
R⁷ represents independently for each occurrence C₁₋₈ alkyl, C₁₋₆ haloalkyl, C₂₋₆ alkenyl, C₃₋₇ cycloalkyl, or a 4-7 membered saturated monocyclic heterocyclyl having one or two heteroatoms independently selected from nitrogen, oxygen, and sulfur; wherein said C₁₋₈ alkyl is optionally substituted with C₁₋₄ alkoxyl, phenyl, C₃₋₇ cycloalkyl, or a 4-7 membered saturated monocyclic heterocyclyl having one or two heteroatoms independently selected from nitrogen, oxygen, and sulfur;
R⁸ represents independently for each occurrence halo, C₁₋₄ alkyl, C₁₋₄ haloalkyl, C₁₋₄ alkoxyl, C₁₋₄ haloalkoxyl, —(C₀₋₃ alkylene)-CO₂R¹⁰, —CN, or —N(R⁹)₂;
R¹⁰ represents independently for each occurrence C₁₋₆ alkyl, C₃₋₇ cycloalkyl, or hydrogen;
R¹¹ represents independently for each occurrence C₁₋₂ haloalkyl, —SF₅, —Si(C₁₋₄ alkyl)₃, —Si(CH₃)₂(C₁₋₄ haloalkyl), —Si(CH₃)₂(C₃₋₇ cycloalkyl), 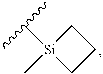 [SMILES: *[Si]1(C)CCC1]
—Si(CH₃)₂(phenyl), —S-phenyl, —O-phenyl, phenyl, thiophenyl, pyridinyl, or C₃₋₇ cycloalkyl; wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅;
X represents independently for each occurrence —O—, ψ-OC(O)—, —OC(O)O—, ψ-OC(O)—N(R⁹)—, —S—, —S—S—, or ψ-SC(O)—; wherein ψ denotes the point of attachment to C₁₋₁₀ alkylene;
Y represents independently for each occurrence —O—, —S—, or —CF₂—;
B¹ or 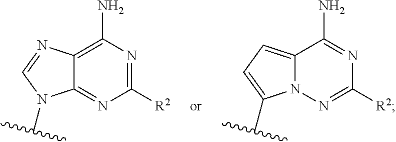
and
m and p are independently for each occurrence 0, 1, 2, or 3;
wherein one or more hydrogen atoms may be replaced with deuterium.

5. (canceled)

6. A method of inhibiting HERV-K reverse transcriptase activity in a subject suffering from a disorder selected from the group consisting of an inflammatory disorder, a neurodegenerative disorder, and an immune disorder other than a viral infection, comprising contacting a HERV-K reverse transcriptase with an effective amount of a compound of Formula I, in order to inhibit the activity of said HERV-K reverse transcriptase; wherein Formula I is represented by: 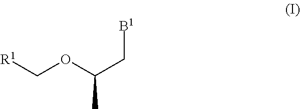
or a stereoisomer thereof; or a pharmaceutically acceptable salt of either of the foregoing; wherein:
R¹ is —P(O)(OR³)(N(R⁴)(R⁵)), —P(O)(OR³)₂, or —P(O)(N(R⁴)(R⁵))₂;
R² is hydrogen, —NH₂, or fluoro;
R³ represents independently for each occurrence:
a. phenyl, naphthyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; wherein said phenyl, naphthyl, 5-6 membered monocyclic heteroaryl, and 8-10 membered bicyclic heteroaryl are substituted with m instances of R⁸;
b. hydrogen, —P(O)(OH)₂, —P(O)(OH)—O—P(O)(OH)₂, C₁₋₂₀ alkyl, C₁₋₂₀ haloalkyl, —(C₁₋₁₀ alkylene)-X—(C₁₋₂₀ alkyl), —(C₁₋₁₀ alkylene)-Y—(C₁₋₂₀ alkylene)-R¹¹, —(C₁₋₁₀ alkylene)-Y—(C₂₋₂₀ alkynyl), —(C₁₋₁₀ alkylene)-Y—(C₂₋₂₀ alkynylene)-R¹¹, or —C(R⁶)₂—CO₂R¹⁰; wherein said C₁₋₂₀ alkyl, C₁₋₂₀ haloalkyl, and C₁₋₁₀ alkylene are optionally substituted with one hydroxyl, —SH, C₁₋₂₀ alkoxyl, or —OC(O)—N(R⁹)₂; and wherein one methylene unit in each of said C₁₋₂₀ alkyl, C₁₋₂₀ haloalkyl, and C₁₋₁₀ alkylene is optionally replaced with a C₃₋₅ cycloalkylene or phenylene; or
c. two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸;
R⁴ and R⁹ each represents independently for each occurrence hydrogen or C₁₋₄ alkyl; or R⁴ and one occurrence of R⁶ are taken together with atoms to which they are attached to form a 4-7 membered saturated heterocyclic ring containing 1 nitrogen atom; or two instances of R⁹ are taken together with the atom to which they are attached to form a 4-7 membered saturated heterocyclic ring containing 1 nitrogen atom and optionally 1 oxygen atom;
R⁵ represents independently for each occurrence:
a. —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, —C(R¹⁰)(H)—CH₂—CO₂R¹⁰, C₁₋₂₀ alkyl, C₁₋₂₀ haloalkyl, —(C₁₋₁₀ alkylene)-X—(C₁₋₁₀ alkyl), —(C₁₋₁₀ alkylene)-phenyl, —(C₁₋₄ haloalkylene)-phenyl, —C(O)-phenyl, —C(S)-phenyl, or —C(O)-(5-6 membered monocyclic heteroaryl having 1, 2, or 3 heteroatoms independently selected from nitrogen, oxygen, and sulfur); or
b. phenyl; naphthyl; a 5-6 membered monocyclic heteroaryl having 1, 2, or 3 heteroatoms independently selected from nitrogen, oxygen, and sulfur; or an 8-10 membered bicyclic heteroaryl having 1, 2, or 3 heteroatoms independently selected from nitrogen, oxygen, and sulfur;
wherein said phenyl, naphthyl, 5-6 membered monocyclic heteroaryl, and 8-10 membered bicyclic heteroaryl are substituted with m instances of R⁸; and said C₁₋₂₀ alkyl, C₁₋₂₀ haloalkyl, and C₁₋₁₀ alkyl are optionally substituted with one hydroxyl;
R⁶ represents independently for each occurrence C₁₋₆ alkyl, C₁₋₆ haloalkyl, C₃₋₅ cycloalkyl, —CN, or hydrogen, wherein said C₁₋₆ alkyl is optionally substituted with —S—(C₁₋₄ alkyl), —SH, C₁₋₄ alkoxyl, C₁₋₄ haloalkoxyl, hydroxyl, —OCH₂CN, phenyl, C₃₋₇ cycloalkyl, a 5-6 membered monocyclic heteroaryl having 1, 2, or 3 heteroatoms independently selected from nitrogen, oxygen, and sulfur, or an 8-10 membered bicyclic heteroaryl having 1, 2, or 3 heteroatoms independently selected from nitrogen, oxygen, and sulfur; or two instances of R⁶ are taken together with the carbon atom to which they are attached to form a 3-6 membered saturated ring having 0 or 1 oxygen atom;
R⁷ represents independently for each occurrence C₁₋₈ alkyl, C₁₋₆ haloalkyl, C₂₋₆ alkenyl, C₃₋₇ cycloalkyl, or a 4-7 membered saturated monocyclic heterocyclyl having one or two heteroatoms independently selected from nitrogen, oxygen, and sulfur; wherein said C₁₋₈ alkyl is optionally substituted with C₁₋₄ alkoxyl, phenyl, C₃₋₇ cycloalkyl, or a 4-7 membered saturated monocyclic heterocyclyl having one or two heteroatoms independently selected from nitrogen, oxygen, and sulfur;
R⁸ represents independently for each occurrence halo, C₁₋₄ alkyl, C₁₋₄ haloalkyl, C₁₋₄ alkoxyl, C₁₋₄ haloalkoxyl, —(C₀₋₃ alkylene)-CO₂R¹⁰, —CN, or —N(R⁹)₂;
R¹⁰ represents independently for each occurrence C₁₋₆ alkyl, C₃₋₇ cycloalkyl, or hydrogen;
R¹¹ represents independently for each occurrence C₁₋₂ haloalkyl, —SF₅, —Si(C₁₋₄ alkyl)₃, —Si(CH₃)₂(C₁₋₄ haloalkyl), —Si(CH₃)₂(C₃₋₇ cycloalkyl), 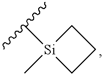 [SMILES: *[Si]1(C)CCC1]
—Si(CH₃)₂(phenyl), —S-phenyl, —O-phenyl, phenyl, thiophenyl, pyridinyl, or C₃₋₇ cycloalkyl; wherein said phenyl is optionally substituted with (i) 1 to 5 fluoro, or (ii) one occurrence of C₁₋₄ alkyl, C₂₋₆ alkynyl, —C≡C—SF₅, —C≡C—Si(CH₃)₃, —Si(CH₃)₃, —CF₃, or —SF₅;
X represents independently for each occurrence —O—, ψ-OC(O)—, —OC(O)O—, ψ-OC(O)—N(R⁹)—, —S—, —S—S—, or ψ-SC(O)—; wherein ψ denotes the point of attachment to C₁₋₁₀ alkylene;
Y represents independently for each occurrence —O—, —S—, or —CF₂—;
B¹ is 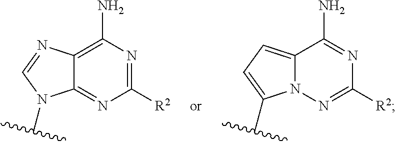
and
m and p are independently for each occurrence 0, 1, 2, or 3;
wherein one or more hydrogen atoms may be replaced with deuterium.

7-9. (canceled)

10. The method of claim 1, wherein the compound is a compound of Formula I-A: 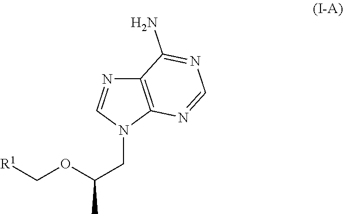
or a pharmaceutically acceptable salt thereof; wherein:
R¹ is —P(O)(OR³)(N(R⁴)(R⁵)) or —P(O)(N(R⁴)(R⁵))₂;
R³ is phenyl, naphthyl, or —C(R⁶)₂—CO₂R¹⁰; wherein said phenyl and naphthyl are substituted with m instances of R¹;
R⁴ represents independently for each occurrence hydrogen or C₁₋₄ alkyl; or R⁴ and one occurrence of R⁶ are taken together with the atoms to which they are attached to form a 4-7 membered saturated heterocyclic ring containing 1 nitrogen atom;
R⁵ represents independently for each occurrence —C(R⁶)₂—CO₂R⁷, —C(R⁶)₂—C(O)N(R⁹)₂, —C(R⁶)₂—C(O)SR¹⁰, —CH₂—C(R¹⁰)(H)—CO₂R¹⁰, or —C(R¹⁰)(H)—CH₂—CO₂R¹⁰;
R⁶ represents independently for each occurrence C₁₋₆ alkyl, C₁₋₆ haloalkyl, C₃₋₅ cycloalkyl, or hydrogen, wherein said C₁₋₆ alkyl is optionally substituted with —S—(C₁₋₄ alkyl), phenyl, or C₃₋₇ cycloalkyl; or two instances of R⁶ are taken together with the carbon atom to which they are attached to form a 3-5 membered saturated carbocyclic ring;
R⁷ represents independently for each occurrence C₁₋₆ alkyl, C₂₋₆ alkenyl, C₃₋₇ cycloalkyl, or a 4-7 membered saturated monocyclic heterocyclyl having one nitrogen or oxygen atom;
wherein said C₁₋₆ alkyl is optionally substituted with C₁₋₄ alkoxyl, phenyl, C₃₋₇ cycloalkyl, or a 4-7 membered saturated monocyclic heterocyclyl having one nitrogen or oxygen atom;
R⁸ represents independently for each occurrence halo, C₁₋₄ alkyl, C₁₋₄ haloalkyl, or C₁₋₄ alkoxyl;
R⁹ and R¹⁰ represents independently for each occurrence C₁₋₄ alkyl or hydrogen; and
m is 0, 1, 2, or 3.

11-19. (canceled)

20. The method of claim 1, wherein the compound is a compound of Formula I-B: 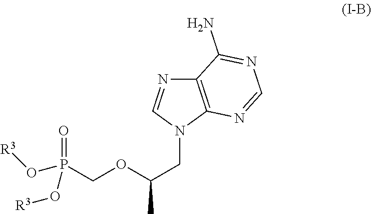
or a pharmaceutically acceptable salt thereof; wherein:
R³ represents independently for each occurrence C₁₋₂₀ alkyl, C₁₋₂₀ haloalkyl, —(C₁₋₁₀ alkylene)-X—(C₁₋₂₀ alkyl), hydrogen, —P(O)(OH)₂, or —P(O)(OH)—O—P(O)(OH)₂; wherein said C₁₋₂₀ alkyl, C₁₋₂₀ haloalkyl, and C₁₋₁₀ alkylene are optionally substituted with one hydroxyl or C₁₋₂₀ alkoxyl; and wherein one methylene unit in each of said C₁₋₂₀ alkyl, C₁₋₂₀ haloalkyl, and C₁₋₁₀ alkylene is optionally replaced with a C₃₋₅ cycloalkylene; or
two instances of R³ are taken together to form a C₂₋₄ bivalent hydrocarbon chain optionally ortho-fused to a 6-membered aromatic ring having 0, 1, or 2 nitrogen atoms; wherein said chain or ring is substituted with p instances of R⁸;
R⁸ represents independently for each occurrence halo, C₁₋₄ alkyl, C₁₋₄ haloalkyl, C₁₋₄ alkoxyl, —(C₀₋₃ alkylene)-CO₂R¹⁰, or —N(R⁹)₂;
R⁹ represents independently for each occurrence hydrogen or C₁₋₄ alkyl;
R¹⁰ represents independently for each occurrence C₁₋₆ alkyl, C₃₋₇ cycloalkyl, or hydrogen;
X represents independently for each occurrence —O—, ψ-OC(O)—, —OC(O)O—, ψ-OC(O)—N(R⁹)—, —S—, —S—S—, or ψ-SC(O)—; wherein ψ denotes the point of attachment to C₁₋₁₀ alkylene; and
p is 0, 1, 2, or 3.

21-27. (canceled)

28. The method of claim 1, wherein the compound is a compound in Table 1, 1-A, 2, 3, 4, 5, or 6 herein, or a pharmaceutically acceptable salt thereof.

29. The method of claim 1, wherein the compound is 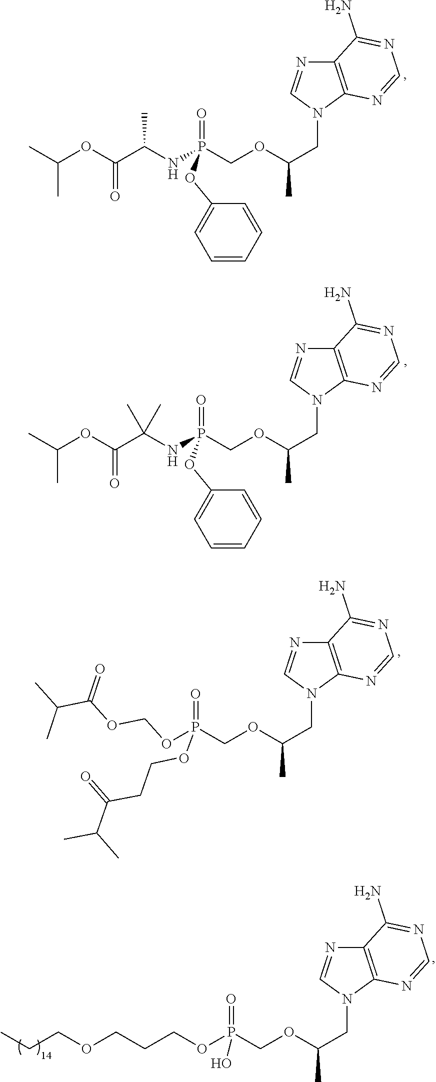
or a pharmaceutically acceptable salt thereof.

30. The method of claim 1, wherein the compound is 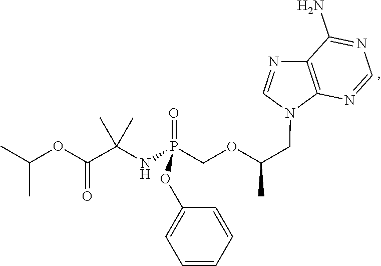 [SMILES: CC(C)OC(=O)C(C)(C)N[P@](=O)(CO[C@H](C)Cn1cnc2c(N)ncnc21)Oc1ccccc1]
or a pharmaceutically acceptable salt thereof.

31. The method of claim 1, wherein the compound is 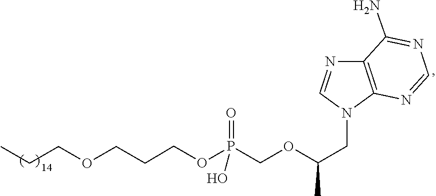
or a pharmaceutically acceptable salt thereof.

32-34. (canceled)

35. The method of claim 30, wherein the disorder is an inflammatory disorder.

36. The method of claim 35, wherein the inflammatory disorder is rheumatoid arthritis, osteoarthritis, ankylosing spondylitis, inflammatory bowel disease, Crohn's disease, ulcerative colitis, nonalcoholic steatohepatitis (NASH), non-alcoholic fatty liver disease (NAFLD), cholestatic liver disease, or sclerosing cholangitis, psoriasis, dermatitis, vasculitis, scleroderma, asthma, bronchitis, chronic obstructive pulmonary disease (COPD), pulmonary fibrosis, pulmonary hypertension, sarcoidosis, myocarditis, pericarditis, gout, myositis, Sjogren's syndrome, or systemic lupus erythematosus.

37. The method of claim 30, wherein the disorder is an immune disorder other than a viral infection.

38. The method of claim 37, wherein the immune disorder is a type 1 interferonopathy, type 1 diabetes, Aicardi-Goutieres syndrome (AGS), arthritis, psoriasis, systemic lupus erythematosus (SLE), lupus nephritis, cutaneous lupus erythematosus (CLE), familial chilblain lupus, systemic sclerosis, STING-associated vasculopathy with onset in infancy (SAVI), graft versus host disease, scleroderma, polymyositis, inflammatory bowel disease, dermatomyositis, ulcerative colitis, Crohn's disease, vasculitis, psoriatic arthritis, Reiter's syndrome, exfoliative psoriatic dermatitis, pemphigus vulgaris, Sjogren's syndrome, autoimmune uveitis, glomerulonephritis, post myocardial infarction cardiotomy syndrome, pulmonary hemosiderosis, amyloidosis, sarcoidosis, aphthous stomatitis, thyroiditis, gastritis, adrenalitis (Addison's disease), ovaritis, primary biliary cirrhosis, myasthenia gravis, gonadal failure, hypoparathyroidism, alopecia, malabsorption syndrome, pernicious anemia, hepatitis, hypopituitarism, diabetes insipidus, or sicca syndrome.

39. The method of claim 37, wherein the immune disorder is a type 1 interferonopathy, type 1 diabetes, Aicardi-Goutieres syndrome (AGS), systemic lupus erythematosus (SLE), lupus nephritis, cutaneous lupus erythematosus (CLE), dermatomyositis, or Sjogren's syndrome.

40. The method of claim 30, wherein the disorder is a neurodegenerative disorder.

41. The method of claim 40, wherein the neurodegenerative disorder is Alzheimer's disease, amyotrophic lateral sclerosis (ALS), multiple sclerosis, Parkinson's disease, Huntington's disease, peripheral neuropathy, age-related macular degeneration, Creutzfeldt-Jacob disease, stroke, prion disease, frontotemporal dementia, Pick's disease, progressive supranuclear palsy, spinocerebellar ataxias, Lewy body disease, dementia, multiple system atrophy, epilepsy, bipolar disorder, schizophrenia, an anxiety disorder, or major depression.

42. The method of claim 39, wherein the subject has (i) expression of LINE1 RNA, LINE1 ORF1 polypeptide, and/or LINE1 ORF2 polypeptide; and/or (ii) activity of LINE1 reverse transcriptase.

43. (canceled)

44. (canceled)

45. The method of claim 37, wherein the immune disorder is systemic lupus erythematosus (SLE).

46. The method of claim 37, wherein the immune disorder is cutaneous lupus erythematosus (CLE).

47. The method of claim 37, wherein the immune disorder is dermatomyositis.
